(19) United States
(12) Patent Application Publication
(10) Pub. No.: US 2009/0099075 A1
(43) Pub. Date: Apr. 16, 2009

(54) Chimeric Keratin-Binding Effector Proteins

(75) Inventors: Heiko Barg, Speyer (DE); Burghard Liebmann, Bensheim (DE); Heike Reents, Speyer (DE); Arne Ptock, Ludwigshafen (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/094,889

(22) PCT Filed: Nov. 15, 2006
(86) PCT No.: PCT/EP2006/068474

§ 371 Date: May 23, 2008

(30) Foreign Application Priority Data
Nov. 24, 2005 (EP) 05111240.7
Jun. 30, 2006 (EP) 06116399.4

(51) Int. Cl. A61K 38/17; C07K 14/00; C07H 21/00; C12N 1/20

(52) U.S. Cl. 514/12; 530/353; 536/23.1; 435/252.8; 435/252.5; 536/23.5

(57) ABSTRACT

The invention relates to chimeric keratin-binding effector proteins and their use in dermocosmetics.

The invention relates to chimeric keratin-binding effector proteins and their use in dermocosmetics.

Vertebrate cells comprise filaments, of which one group is constructed from keratins. Specific proteins, such as, for example, desmoplakin or plakophilin 1, bind to these keratins, which also occur in hair, skin and fingernails and toenails, by means of a specific sequence motif, a so-called keratin-binding domain (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus., Mol Biol Cell. 2003 May; 14(5):1978-92. Epub 2003 Jan 26; Hopkinson S B, Jones J C., The N-terminus of the transmembrane protein BP180 interacts with the N-terminal domain of BP230, thereby mediating keratin cytoskeleton anchorage to the cell surface at the site of the hemidesmosome, Mol Biol Cell. 2000 January; 11(1):277-86; Smith E. A., Fuchs E., Defining the Interactions Between Intermediate Filaments and Desmosomes, The Journal of Cell Biology, Volume 141, 1998).

The human skin is subject to certain aging processes, some of which are attributable to intrinsic processes (chronoaging) and some of which are attributable to exogenous factors (environmental, e.g. photoaging). In addition, temporary or persisting changes in the appearance of the skin can arise, such as acne, greasy or dry skin, keratoses, rosacea, photosensitive, inflammatory, erythematous, allergic or autoimmune reactions, such as dermatoses and photodermatoses.

Exogenous factors include, in particular, sunlight or artificial sources of irradiation with a comparable spectrum, and also free-radical or ionic compounds which can arise as a result of the radiation. These factors also include cigarette smoke and the reactive compounds present therein, such as ozone, free radicals, singlet oxygen and other reactive oxygen or nitrogen compounds which disturb the natural physiology or morphology of the skin.

For avoiding and treating the abovementioned damage, disorders, and for the care and decorative treatment and beautifying of the skin, hair, fingernails and toenails, a large number of cosmetic and pharmaceutical preparations have been made available by the cosmetic and pharmacological industry. The use of proteins as constituent of these preparations has been known for a long time. On account of their special properties, proteins and enzymes have not only a wide field of use in the production of such compositions, rather, on account of enzymatic activities or structure-imparting properties, they bring about positive physiological changes in skin and hair.

However, proteins are usually not able to enter into a fixed bond with the surface structures of animal organisms, i.e. binding to e.g. skin, hair is ensured only for a few proteins. Thus, as a result of applying a protein with certain physiological or decorative properties, it cannot be ensured that the proteins arrive at their site of action and remain there for an adequate time which is required for the desired physiological or decorative effect.

The German patent application with the application number DE 102005011988.3 describes the use of keratin-binding domains in cosmetic preparations. The international patent application with the application number PCT/EP/05/005599 reveals that keratin-binding domains can also be coupled with effector molecules.

It was therefore an object of the present invention to provide new types of proteins which can be used dermocosmetically for application to skin, hair, fingernails and toenails. Advantageously, proteins or polypeptides were to be identified which have a keratin-binding property, exert a dermocosmetic effect and are also suitable for producing cosmetic and/or dermocosmetic formulations or preparations.

### SUMMARY OF THE INVENTION

The invention firstly provides chimeric keratin-binding effector proteins comprising (a) at least one keratin-binding polypeptide (i) and (b) at least one further effector polypeptide (ii).

In a particularly preferred embodiment, these are keratin-binding polypeptides (i) which have a binding affinity to human skin keratin, hair keratin or nail keratin. Preferably, the keratin-binding polypeptide (i) used according to the invention comprises
a) at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215, or
b) a polypeptide which is at least 40% identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215 and is able to bind keratin.

The keratin-binding polypeptide (i) can preferably be encoded by a nucleic acid molecule comprising at least one nucleic acid molecule chosen from the group consisting of:
c) nucleic acid molecule which encodes a polypeptide comprising the sequence shown in SEQ ID Not: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215;
d) nucleic acid molecule which comprises at least one polynucleotide of the sequence shown in SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 or 214;
e) nucleic acid molecule which encodes a polypeptide according to the sequences SEQ ID No.: 2, 4, 6, 8, 10, 12, 14, 16, 16, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215;
f) nucleic acid molecule with a nucleic acid sequence corresponding to at least one of the sequences according to SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 or 214, or a nucleic acid molecule derived therefrom by substitution, deletion or insertion which encodes a polypeptide which is at least 40% identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215 and is able to bind to keratin;
g) nucleic acid molecule which encodes a polypeptide which is recognized by a monoclonal antibody directed toward a polypeptide which is encoded by the nucleic acid molecules according to (c) to (e);
h) nucleic acid molecule coding for a keratin-binding protein which, under stringent conditions, hybridizes with a nucleic acid molecule according to (c) to (e);
i) nucleic acid molecule coding for a keratin-binding protein which can be isolated from a DNA bank using a nucleic acid molecule according to (c) to (e) or part fragments thereof comprising at least 15 nt, preferably 20 nt, 30 nt, 50 nt, 100 nt, 200 nt or 500 nt as probe under stringent hybridization conditions; and
j) nucleic acid molecule which can be produced by backtranslating one of the amino acid sequences shown in the sequences SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 166, 213 or 215.

The present invention preferably provides keratin-binding effector proteins where the effector polypeptide (ii) is chosen from the group consisting of enzymes, antibodies, effector-binding proteins, fluorescent proteins, antimicrobial peptides and self-assembling proteins.

The present invention particularly preferably provides keratin-binding effector proteins which comprise, as effector polypeptides (ii), enzymes chosen from the group consisting of oxidases, peroxidases, proteases, tyrosinases, lactoperoxidase, lysozyme, amyloglycosidases, glucose oxidases, superoxide dismutases, photolyases and catalases.

Moreover, preference is given to keratin-binding effector proteins comprising, as effector polypeptide (ii), a silk protein, particularly preferably silk proteins which comprise at least one of the sequences according to SEQ ID No.: 151, 201, 202, 203, 204, 205, 206, 207, 208, 209 or 210, or correspond to a polypeptide which is at least 40% identical to at least one of the sequences according to SEQ ID No.: 151, 201, 202, 203, 204, 205, 206, 207, 208, 209 or 210.

In addition, the invention relates to those keratin-binding effector proteins comprising silk proteins which are encoded by a nucleic acid molecule comprising at least one nucleic acid molecule chosen from the group consisting of:
k) nucleic acid molecule which encodes a polypeptide comprising the sequence shown in SEQ ID No.: 151, 201, 202, 203, 204, 205, 206, 207, 208, 209 or 210;
l) nucleic acid molecule which comprises at least one polynucleotide of the sequence shown in SEQ ID No.: 150;
m) nucleic acid molecule which encodes a polypeptide according to the sequences SEQ ID No.: 151, 201, 202, 203, 204, 205, 206, 207, 208, 209 or 210;
n) nucleic acid molecule with a nucleic acid sequence according to SEQ ID No. 150 or a nucleic acid molecule derived therefrom by substitution, deletion or insertion which encodes a polypeptide which is at least 40% identical to the sequence according to SEQ ID No.: 151;
o) nucleic acid molecule which encodes a polypeptide which is recognized by a monoclonal antibody directed toward a polypeptide which is encoded by the nucleic acid molecules according to (k) to (m);
p) nucleic acid molecule coding for a keratin-binding protein which hybridizes under stringent conditions with a nucleic acid molecule according to (k) to (m);
q) nucleic acid molecule coding for a keratin-binding protein which can be isolated from a DNA bank using a nucleic acid molecule according to (k) to (m) or part fragments thereof comprising at least 15 nucleotides as probe under stringent hybridization conditions; and
r) nucleic acid molecule which can be produced by backtranslating one of the amino acid sequences shown in the sequences SEQ ID No.: 151, 201, 202, 203, 204, 205, 206, 207, 208, 209 or 210.

In a preferred embodiment of the present invention, the chimeric keratin-binding effector proteins according to the invention are proteins in which the above-described polypeptides (i) and (ii) are linked together by means of translation fusion.

The invention further preferably provides keratin-binding effector proteins in which the above-described polypeptides (i) and (ii) are linked together by means of a chemical coupling reaction. Preference is given here to those keratin-binding effector proteins in which the effector polypeptide (ii) is covalently bonded to side chains of internal amino acids the C-terminus or the N-terminus of the keratin-binding polypeptide (i).

In addition, the present invention provides the above-described keratin-binding effector proteins where the effector polypeptide (ii) and the keratin-binding polypeptide (i) are joined together by means of a spacer element. These are preferably keratin-binding effector proteins which are joined together by means of a spacer element where the spacer element is a crosslinker.

Also preferred are keratin-binding effector proteins comprising a spacer element where the spacer element is an at least bifunctional linker which covalently joins together the keratin-binding polypeptide (i) and the effector polypeptide by binding to side chains of internal amino acids, the C-terminus or the N-terminus of said polypeptides. Besides the keratin-binding effector proteins already specified, preference is also given to those in which the spacer element linking the polypeptides (i) and (ii) is a polypeptide.

The invention further provides the use of the above-described keratin-binding effector proteins in dermocosmetics, which are preferably skin protection compositions, skincare compositions, skin cleansing compositions, hair protection compositions, haircare compositions, hair cleansing compositions, hair colorants or products of decorative cosmetics.

The present invention further provides the abovementioned dermocosmetics comprising one of the above-described keratin-binding effector molecules.

In addition, the invention provides proteins according to the amino acid sequences shown in SEQ ID No.: 168, 176, 182, 188, 194 and 200.

The present invention likewise provides nucleic acid molecules according to the sequence shown in SEQ ID No.: 167, 175, 181, 187, 193 or 199.

Furthermore, the present invention provides DNA expression cassettes comprising a nucleic acid molecule with a nucleic acid sequence according to the sequence shown in SEQ ID No: 167, 175, 181, 187, 193 or 199.

The present invention likewise provides vectors comprising a DNA expression cassette comprising a nucleic acid molecule with a nucleic acid sequence according to the sequence shown in SEQ ID No.: 167, 175, 181, 187, 193 or 199.

Moreover, the present invention provides transgenic cells comprising
s) at least one of the abovementioned vectors, or
t) at least one of the abovementioned expression cassettes, or
u) at least one of the abovementioned nucleic acid molecules coding for a polypeptide comprising at least one polypeptide which is encoded by a nucleic acid molecule according to the sequence shown in SEQ ID No.: 167, 175, 181, 187, 193 or 199.

### DEFINITIONS

For the purposes of the present invention, “antibodies” are proteins which humans and jaw-bearing vertebrates produce to protect against antigens (infection pathogens or biological material alien to the body). They are a central constituent of the immune system of higher eukaryotes and are secreted by a class of white corpuscles, the B cells. They occur in the blood and in the extracellular liquid of tissue.

For the purposes of the present invention “backtranslation” means the translation of a protein sequence into a nucleic acid sequence coding for this protein. The backtranslation is thus a process of decoding an amino acid sequence into the nucleic acid sequence corresponding to it. Customary methods are based on creating codon usage tables for a certain organism, which are produced by computer-aided sequence comparisons. Using the codon usage tables it is possible to determine the codons used most frequently for a certain amino acid for a specific organism. Protein backtranslation can be carried out using computer algorithms which are known to the person skilled in the art and specifically generated for this purpose (Andrés Moreira and Alejandro Maats. TIP: protein backtranslation aided by genetic algorithms. Bioinformatics, Volume 20, Number 13 Pp. 2148-2149 (2004); G Pesole, M Attimonelli, and S Liuni. A backtranslation method based on codon usage strategy. Nucleic Acids Res. 1988 March 11; 16(5 Pt A): 1715-1728.).

For the purposes of the present invention, “chimeric keratin-binding effector proteins” means proteins comprising a keratin-binding polypeptide, protein or protein domain (i) and an effector polypeptide, effector protein, or effector protein domain (ii), where the specified polypeptides, proteins or protein domains are linked together artificially. Linked artificially means a link produced using biotechnological or chemotechnological methods, as is not realized in the living world, e.g. the organisms in which said polypeptides, proteins or protein domains occur naturally. For producing the chimeric keratin-binding effector proteins, in the case of biotechnological processes, translation fusion is preferred, and in the case of chemotechnological processes, the processes assumed under the term “chemical coupling reaction” are preferred.

“Translation fusion” is understood as meaning the production of a chimeric nucleic acid molecule in which the linking of at least two nucleic acid molecules coding for a polypeptide, protein or a protein domain is realized in such a way that, as a result of the translation event of this chimeric nucleic acid molecule, a continuous polypeptide chain can be formed.

“Decorative cosmetics” means cosmetic auxiliaries which are not primarily used for the care, but for beautifying or improving the appearance of skin, hair and/or fingernails and toenails. Auxiliaries of this type are appropriately known to the person skilled in the art and comprise, for example, kohl pencils, mascara, eye shadows, tinted day creams, powders, concealing sticks, blusher, lipsticks, lipliner sticks, make-up, nail varnish, glamour gel etc. Also included are compositions suitable for coloring skin or hair.

“Dermocosmetics”, also referred to as “cosmeceuticals” or “dermocosmetic compositions” or “dermocosmetic preparations” are compositions or preparations (i) for protecting against damage to skin, hair and/or fingernails and toenails, (ii) for treating existing damage to skin, hair and/or fingernails or toenails and (iii) for the care of skin, hair and/or fingernails or toenails, comprising skin cosmetic, nail cosmetic, hair cosmetic, dermatological, hygiene or pharmaceutical compositions, preparations and formulations and for improving the feel of the skin (sensory properties). Compositions for decorative cosmetics are explicitly included. Also included are compositions for skincare, with which the pharmaceutically dermatological intended use is achieved taking into consideration cosmetic points of view. Compositions or preparations of this type are used for helping, preventing and treating skin disorders and, besides the cosmetic effect, develop a biological effect. For the purposes of the definition given above, “dermocosmetics” comprise, in a cosmetically compatible medium, suitable auxiliaries and additives which are familiar to the person skilled in the art and can be found in cosmetics handbooks, for example Schrader, Grundlagen und Rezepturen der Kosmetika [Fundamentals and formulations of cosmetics], Hüthig Veriag, Heidelberg, 1989, ISBN 3-7785-1491-1, or Umbach, Kosmetik: Entwicklung, Herstellung und Anwendung kosmetischer Mittel [Cosmetics: development, manufacture and use of cosmetic compositions], 2nd extended edition, 1995, Georg Thieme Verlag, ISBN 3 13 712602 9.

For the purposes of the present invention, “dermocosmetic active ingredients” or “dermocosmetically active ingredients” are the active ingredients present in dermocosmetics according to the definition given above which are involved in realizing the individual mode of action of the dermocosmetics. These are thus, for example, active ingredients which bring about protection against damage to skin, hair and/or fingernails or toenails, (ii) can be used for treating existing damage to skin, hair and/or fingernails and toenails, (iii) have skin, hair and/or fingernails or toenail caring properties and (iv) are used for decorative beautification or improvement in the appearance of skin, hair and/or fingernails and toenails. Also included are active ingredients for skincare with which the pharmaceutically dermatological intended use is achieved taking cosmetic points of view into consideration. Active ingredients of this type are used for helping, preventing and treating skin disorders and, besides the cosmetic effect, develop a biological effect. Active ingredients of this type are chosen, for example, from the group of natural or synthetic polymers, pigments, humectants, oils, waxes, proteins, enzymes, minerals, vitamins, sunscreens, dyes, perfumes, antioxidants, peroxide decomposers and preservatives and pharmaceutical active ingredients which are used for helping, preventing and treating skin disorders and have a biological effect which heals, prevents damage, regenerates or improves the general condition of the skin.

For the purposes of the present invention, “expression cassette” means a nucleic acid molecule comprising a nucleic acid molecule which is linked in a functional manner to at least one genetic control element (for example a promoter) which ensures an expression in a cell or an organism, preferably prokaryotic cells, yeasts, or cell cultures of eukaryotic cells.

“Functional linking” means, for example, the sequential arrangement of a promoter with the nucleic acid molecule to be expressed (for example coding for a keratin-binding effector protein) and, if appropriate, further regulatory elements, such as, for example, a terminator, in such a way that each of the regulatory elements can fulfill its function during the transgenic expression of the nucleic acid molecule. For this, a direct linkage in the chemical sense is not necessarily required. Genetic control sequences, such as, for example, enhancer sequences, can exert their function on the target sequence also from more distant positions or even from other DNA molecules. Preference is given to arrangements in which the nucleic acid molecule to be expressed transgenically is positioned behind the sequence acting as promoter, so that both sequences are covalently bonded together. Preferably, the distance between the promoter sequence and the nucleic acid sequence to be expressed transgenically here is less than 200 base pairs, particularly preferably smaller than 100 base pairs, very particularly preferably smaller than 50 base pairs.

Producing a functional linkage and also producing an expression cassette can be realized using customary recombinant and cloning techniques, as described, for example, in Maniatis T, Fritsch E F and Sambrook J (1989) Molecular Cloning: A Laboratory Manual, Cold Spring Harbor Laboratory, Cold Spring Harbor (NY), in Silhavy T J, Berman M L and Enquist L W (1984) Experiments with Gene Fusions, Cold Spring Harbor Laboratory, Cold Spring Harbor (NY), in Ausubel F M et al. (1987) Current Protocols in Molecular Biology, Greene Publishing Assoc. and Wiley Interscience and in Gelvin et al. (1990) In: Plant Molecular Biology Manual. However, between the two sequences it is also possible for further sequences, which, for example, have the function of a linker with certain restriction enzyme cleavage sites or a signal peptide, to be positioned. The insertion of sequences can also lead to the expression of fusion proteins. Preferably, the expression cassette, consisting of a linkage of promoter and nucleic acid sequence to be expressed can be present in integrated form in a vector and be inserted by, for example, transformation, into a plant genome.

The term “cell” refers to an individual cell. The term “cells” refers to a population of cells. This population may be present in a synchronized or nonsynchronized manner. “Cell” or “cells” includes single-cell organisms and also cells as constituent of a multicellular complex or organism.

“Transgenic” in connection with a cell or an organism means, with regard to a nucleic acid molecule, the polypeptide encoded therefrom, an expression cassette or a vector comprising said nucleic acid molecule or a cell or an organism transformed with said nucleic acid molecule, expression cassette or vector, all those cells or organisms achieved by genetic engineering methods in which the nucleic acid molecule coding either
a) for the keratin-binding polypeptide (i), or
b) the effector protein, or
c) (a) and (b)
   are not located in their natural genetic environment or have been modified by genetic engineering methods, in which case the modification may, for example, be a substitution, addition, deletion or insertion of one or more nucleotide radicals. Natural genetic environment means the natural chromosomal locus in the original organism or the presence in a genomic library. In the case of a genomic library, the natural, genetic environment of the nucleic acid sequence is at least partially retained. The environment flanks the nucleic acid sequence at least on one side and has a sequence length of at least 50 bp, preferably at least 500 bp, particularly preferably at least 1000 bp, very particularly preferably at least 5000 bp. A naturally occurring expression cassette—for example the naturally occurring combination of the keratin-binding polypeptide promoter with the corresponding gene coding for the keratin-binding polypeptide—becomes a transgenic expression cassette if the latter is modified by non-natural, synthetic (“artificial”) methods such as, for example, a mutagenization. Appropriate methods are described (U.S. Pat. No. 5,565,350; WO 00/15815).

For the purposes of the present invention, “effector polypeptide” means proteinogenic dermocosmetic active ingredients which have a certain foreseeable effect, preferably a biological or physiological, protective, preventative and/or care effect on skin, hair and/or fingernails and toenails. The effector molecules are preferably proteinogenic compounds such as polypeptides, proteins or enzymes. Particular preference is given to self-assembling proteins, and very particular preference is given to silk proteins.

For the purposes of the present invention, “keratin” means intermediate filaments constructed from rope-like protein complexes. Intermediate filaments are constructed from many proteins of the same type (monomers) which position themselves in parallel to give a tube-like structure. Intermediate filaments are bound to give relatively large bundles (tonofibrils). Intermediate filaments form the cytoskeleton of the cell with the microtubules and actin filaments. A distinction is made between five types of intermediate filaments: acidic and basic keratins, desmins, neurofilaments and lamins. Of specific preference for the purposes of the present invention are the acidic and basic keratins occurring in the epithelia (single or multiple cell layers which cover all external body surfaces of multicellular animal organisms). “Keratin” or “keratins” (also: horny substance, scleroprotein) means a protein which is responsible for the stability and shape of the cells. This protein is a constituent of mammal skin, hair and nails. The strength of keratin is increased through fiber formation: the individual amino acid chains form a right-handed alpha-helix, and every three of these helixes form a left-hand superhelix (=protofibrils). Eleven protofibrils combine to give a microfibril—these combine in turn to give bundles and form macrofibrils which, for example, surround the cells of the hair.

“Keratin-binding polypeptide” means a polypeptide or a protein which has the property of binding to keratin, within the meaning of the definition given above. Keratin-binding polypeptides are thus also intermediate filament-associated proteins. These keratin-binding polypeptides have a binding affinity toward the keratin or the macrostructures consisting of keratin such as protofibrils, microfibrils or macrofibrils. In addition, keratin-binding polypeptides are understood as meaning those polypeptides which have a binding affinity to skin, hair and/or fingernails or toenails of mammals.

“Keratin-binding polypeptides” are also polypeptides which, within a mammal organism, have a biological function associated with the binding of keratin, keratin fibers, skin or hair. Keratin-binding polypeptides likewise means the binding motifs or protein domains necessary for the actual binding to the keratin, the keratin fibers, skin or hair. The binding of the keratin-binding polypeptide (ii) to keratin can be tested under the conditions described in Example 8, 9 and 10. Keratin-binding polypeptides are those polypeptides which, in the abovementioned quantitative keratin-binding tests, have about 10%, 20%, 30%, 40% or 50%, preferably 50%, 60%, 70%, 80% or 90%, particularly preferably 100%, 125%, 150%, very particularly preferably 200%, 300% or 400%, most preferably 500%, 600%, 700% or 1000% or more of the keratin-binding capacity of desmoplakin (SEQ ID No.: 2), preferably of the keratin-binding domain B of desmoplakin (SEQ ID No.: 4).

“Cosmetically compatible medium” is to be understood in the wide sense and means substances suitable for the production of cosmetic or dermocosmetic preparations, and mixtures thereof. They are preferably protein-compatible media.

Upon contact with human and/or animal skin tissue or hair, “cosmetically compatible substances” lead to no irritations or damage and have no incompatibilities with other substances. In addition, these substances have a slight allergenic potential and are approved by state registration authorities for use in cosmetic preparations. These substances are familiar to the person skilled in the art and can be found, for example, in cosmetics handbooks, for example Schrader, Grundlagen und Rezepturen der Kosmetika [Fundamentals and formulations of cosmetics], Hüthig Verlag, Heidelberg, 1989, ISBN 3-7785-1491-1.

“Nucleic acid” or “nucleic acid molecule” means deoxyribonucleotides, ribonucleotides or polymers or hybrids thereof in single-strand or double-strand form, in sense or antisense orientation. The term nucleic acid or nucleic acid molecule can be used to describe a gene, DNA, cDNA, mRNA, oligonucleotide or polynucleotide.

“Nucleic acid sequence” means a successive and linked together sequence of deoxyribonucleotides or ribonucleotides of a nucleic acid molecule according to the definition given above, as can be ascertained using available DNA/RNA sequencing techniques, and depicted or shown in a list of abbreviations, letters or words which represent nucleotides.

For the purposes of the present invention, “polypeptide” means a macromolecule constructed from amino acid molecules in which the amino acids are linked together linearly via peptide bonds. A polypeptide can be made up of a few amino acids (about 10 to 100), but also comprises proteins which are generally constructed from at least 100 amino acids, but can also comprise several thousand amino acids. Preferably, polypeptides comprise at least 20, 30, 40 or 50, particularly preferably at least 60, 70, 80 or 90, very particularly preferably at least 100, 125, 150, 175 or 200, most preferably at least more than 200 amino acids, it being possible for the upper limit to be several thousand amino acids.

“Homology” or “identity” between two nucleic acid sequences is understood as meaning the identity of the nucleic acid sequence over the entire sequence length in question, which is calculated by comparison with the help of the program algorithm GAP (Wisconsin Package Version 10.0, University of Wisconsin, Genetics Computer Group (GCG), Madison, USA; Altschul et al. (1997) Nucleic Acids Res. 25:3389ff) with the following parameter settings:

| | |
|---|---|
| Gap Weight: 50 | Length Weight: 3 |
| Average Match: 10 | Average Mismatch: 0 |

By way of example, a sequence which has a homology of at least 80% based on nucleic acid with the sequence SEQ ID NO: 1 is understood as meaning a sequence which has a homology of at least 80% when compared with the sequence SEQ ID NO: 1 according to the above program algorithm with the above set of parameters.

Homology between two polypeptides is understood as meaning the identity of the amino acid sequence over the entire sequence length in question, which is calculated by comparison with the help of the program algorithm GAP (Wisconsin Package Version 10.0, University of Wisconsin, Genetics Computer Group (GCG), Madison, USA) with the following parameter settings:

| | |
|---|---|
| Gap Weight: 8 | Length Weight: 2 |
| Average Match: 2.912 | Average Mismatch: −2.003 |

By way of example, a sequence which has a homology of at least 80% based on polypeptide with the sequence SEQ ID NO: 2 is understood as meaning a sequence which has a homology of at least 80% when compared with the sequence SEQ ID NO: 2 according to the above program algorithm with the above set of parameters.

“Hybridization conditions” is to be understood in the wide sense and means stringent or less stringent hybridization conditions depending on the application. Such hybridization conditions are described, inter alia, in Sambrook J, Fritsch E F, Maniatis T et al., in Molecular Cloning (A Laboratory Manual), 2nd edition, Cold Spring Harbor Laboratory Press, 1989, pages 9.31-9.57) or in Current Protocols in Molecular Biology, John Wiley & Sons, N.Y. (1989), 6.3.1-6.3.6. The person skilled in the art would choose hybridization conditions which would allow him to differentiate specific hybridizations from unspecific hybridizations. For example, the conditions during the washing step can be chosen from conditions with low stringency (with approximately 2×SSC at 50° C.) and those with high stringency (with approximately 0.2×SSC at 50° C., preferably at 65° C.) (20×SSC: 0.3 M sodium citrate, 3 M NaCl, pH 7.0). Moreover, the temperature during the washing step can be increased from low stringency conditions at room temperature, approximately 22° C., to higher stringency conditions at approximately 65° C. Both parameters, salt concentration and temperature, can be varied at the same time or else individually, keeping the other parameter in each case constant. During the hybridization, it is also possible to use denaturing agents such as, for example, formamide or SDS. In the presence of 50% formamide, the hybridization is preferably carried out at 42° C. Some illustrative conditions for hybridization and washing step are given below:

1. Hybridization conditions can be chosen, for example, from the following conditions:
a) 4×SSC at 65° C.,
b) 6×SSC at 45° C.,
c) 6×SSC, 100 μg/ml of denatured, fragmented fish sperm DNA at 68° C.,
d) 6×SSC, 0.5% SDS, 100 μg/ml of denatured salmon sperm DNA at 68° C.,
e) 6×SSC, 0.5% SDS, 100 μg/ml of denatured, fragmented salmon sperm DNA, 50% formamide at 42° C.,
f) 50% formamide, 4×SSC at 42° C., or
g) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or
h) 2× or 4×SSC at 50° C. (low stringency condition),
i) 30 to 40% formamide, 2× or 4×SSC at 42° C. (low stringency condition).
   500 mN of sodium phosphate buffer pH 7.2, 7% SDS (g/V), 1 mM EDTA, 10 μg/ml single-stranded DNA, 0.5% BSA (g/V) (Church and Gilbert, Genomic sequencing. Proc. Natl. Acad.Sci. U.S.A. 81:1991. 1984)
   2. Washing steps can be chosen, for example, from the following conditions:
a) 0.015 M NaCl/0.0015 M sodium citrate/0.1% SDS at 50° C.
b) 0.1×SSC at 65° C.
c) 0.1×SSC, 0.5% SDS at 68° C.
d) 0.1×SSC, 0.5% SDS, 50% formamide at 42° C.
e) 0.2×SSC, 0.1% SDS at 42° C.
f) 2×SSC at 65° C. (low stringency condition).

In one embodiment, the stringent hybridization conditions are chosen as follows:

A hybridization buffer is chosen which comprises formamide, NaCl and PEG 6000. The presence of formamide in the hybridization buffer destabilizes double-stranded nucleic acid molecules, as a result of which the hybridization temperature can be reduced to 42° C. without lowering the stringency. The use of salt in the hybridization buffer increases the renaturation rate of a duplex, or the hybridization efficiency. Although PEG increases the viscosity of the solution, which has a negative effect on renaturation rates, as a result of the presence of the polymer in the solution, the concentration of the probe in the remaining medium is increased, which increases the hybridization rate. The composition of the buffer is as follows:

TABLE 1
Hybridization buffer
Hybridization buffer

250 mM sodium phosphate buffer pH 7.2
1 mM EDTA
7% SDS (g/v)
250 mM NaCl
10 μg/ml ssDNA
5% polyethylene glycol (PEG) 6000
40% formamide

The hybridizations are carried out overnight at 42° C. The filters are washed the next morning 3× with 2×SSC+0.1% SDS for about 10 min in each case.

In connection with the description “hydroxy function-bearing effector molecule”, “hydroxy function” means free OH groups or hydroxyl groups which enable these OH group-bearing molecules to covalently link to other molecules via an esterification reaction. For the purposes of the present invention, “hydroxy functions” are also those which can be converted chemically into OH functions, such as, for example, derivatives such as methoxy, ethoxy. Here, the effector molecules according to the invention have at least one hydroxyl group. However, it is also possible to use effector molecules with two, three or more hydroxy functions.

In connection with the description, “amino function-bearing effector molecule”, “amino functions” means amino groups which allow said amino function-bearing molecules to covalently link to other molecules via an amide bond. For the purposes of the present invention, “amino functions” are also those which can be converted chemically into amino functions. Here, the effector molecules according to the invention have at least one amino function. However, it is also possible to use effector molecules with two, three or more amino functions and/or secondary amino groups.

“Coupling” in connection with the binding of a linker molecule to an effector molecule or keratin-binding protein means a covalent linking of said molecules.

“Coupling functionalities” are functional groups of a linker molecule which can enter into a covalent bond with functional groups of the effector molecule or keratin-binding protein. Nonlimiting examples which may be mentioned are: hydroxy groups, carboxyl groups, thio groups and amino groups. “Coupling functionalities” or “coupling functionality” and “anchor groups” or “anchor group” are used synonymously.

### Self-Assembling Proteins

Self-assembling proteins are proteins or peptides which can spontaneously congregate under suitable conditions to give higher molecular weight, ordered structures (spheres, films, fibrils, interalia). These may be synthetic, biomimetic or proteins and peptides of natural origin. Nonlimiting examples are structural proteins, β-sheet-rich proteins, and amphiphilic and helical peptides.

For the purposes of the present invention, “spacer element” means a molecule or macromolecule which physically separates the keratin-binding polypeptide (i) from the effector polypeptide (ii).

Spacer elements comprise both the linker molecules described below and also proteinogenic elements such as, for example, oligopeptides, polypeptides or protein domains.

Vectors are DNA molecules which can be stably established in a host cell and duplicated. Vectors are, for example, plasmids, cosmids. In addition, vectors can also be understood as meaning those DNA molecules which can transport DNA elements from one cell into another, without the cells having to necessarily belong to the same organism (e.g., phages, viruses and also agrobacteria). In an advantageous embodiment, the insertion of an expression cassette comprising a gene of interest is realized by means of plasmid vectors. Preference is given to those vectors which can be established extrachromosomally in a cell or an organism. The stable integration of the expression cassette/vector into the host genome is likewise possible.

The term “expression vector” refers to vectors which comprise a DNA molecule of interest in functional linkage with regulatory elements, and can thus ensure the expression of the DNA molecule of interest in a target organism.

### DETAILED DESCRIPTION OF THE INVENTION

The present invention provides chimeric keratin-binding effector proteins comprising (a) at least one keratin-binding polypeptide (i) and (b) at least one further effector polypeptide (ii)

In a particularly preferred embodiment, they are keratin-binding polypeptides (i) which have a binding affinity to human skin keratin, hair keratin or nail keratin.

Particular preference is given to those keratin-binding polypeptides (i) which
a) comprise at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215 or
b) correspond to a polypeptide which is at least 40%, 45% or 50%, preferably at least 55%, 60%, 65% or 70%, particularly preferably at least 75%, 80%, 85%, 90%, 91%, 92%, 93% or 94%, very particularly preferably at least 95% or 96%, identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215 and is able to bind keratin.

In a preferred embodiment of the present invention, the keratin-binding polypeptide (i) used is encoded by a nucleic acid molecule comprising at least one nucleic acid molecule chosen from the group consisting of:
c) nucleic acid molecule which encodes a polypeptide comprising the sequence shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215;
d) nucleic acid molecule which comprises at least one polynucleotide of the sequence shown in SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 or 214;
e) nucleic acid molecule which encodes a polypeptide according to the sequences SEQ ID No.: 2, 4, 6, 8, 10, 12, 14, 16, 18, 20, 22, 24, 26, 28, 30, 32, 34, 36, 38, 40, 42; 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215;
f) nucleic acid molecule with a nucleic and sequence corresponding to at least one of the sequences according to SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 or 214 or a nucleic acid molecule derived therefrom by substitution, deletion or insertion which encodes a polypeptide which is at least 40%, 45% or 50%, preferably at least 55%, 60%, 65% or 70%, particularly preferably at least 75%, 80%, 85%, 90%, 91%, 92%, 93% or 94%, very particularly preferably at least 95% or 96%, identical to at least one of the sequences according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215 and is able to bind to keratin;
g) nucleic acid molecule which encodes a polypeptide which is recognized by a monoclonal antibody directed toward a polypeptide which is encoded the nucleic acid molecules according to (c) to (e);
h) nucleic acid molecule coding for a keratin-binding protein which, under stringent conditions, hybridizes with a nucleic acid molecule according to (c) to (e);
i) nucleic acid molecule coding for a keratin-binding protein which can be isolated from a DNA bank using a nucleic acid molecule according to (c) to (e) or part fragments thereof of at least 15 nt, preferably 20 nt, 30 nt, 50 nt, 100 nt, 200 nt or 500 nt as probe under stringent hybridization conditions; and
j) nucleic acid molecule which can be produced by backtranslating one of the amino acid sequences shown in the sequences SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215.

Keratin-binding polypeptide domains suitable according to the invention are present in the polypeptide sequences of desmoplakins, plakophilins, plakoglobins, plectins, periplakins, envoplakins, trichohyalins, epiplakins or hair follicle proteins.

In a preferred embodiment of the present invention, desmoplakins or part sequences thereof according to the sequences SEQ ID No.: 2, 42, 44, 46, 48, 146, 150, 153, 156, 157, 158, 160, 162, 164 or 166, and/or plakophilins or part sequences thereof according to the sequences SEQ ID No.: 18, 20, 26, 28, 32, 34, 36, 213, 215 and/or plakoglobins or part sequences thereof according to the sequences with the SEQ ID No.: 50, 52, 54, 56, 58, 60, 62, 64, 66, 68, 70, and/or the periplakin according to the sequence with the SEQ ID No.: 86, and/or envoplakins or part sequences thereof according to the sequences with the SEQ ID No.: 90, 92, 94, 96, 98, 102, 104, 105 and/or the sequences according to SEQ ID No.: 138 and 140 are used as keratin-binding polypeptides. Preferred keratin-binding domains are the desmoplakin polypeptides shown in the sequences SEQ ID NOs: 4, 6, 8, 10, 12, 14, 146, 150, 153, 156, 157, 158, 160, 162, 164, 166, 213 or 215, and functional equivalents thereof. In a very particularly preferred embodiment of the present invention, the keratin-binding polypeptides shown in the sequences SEQ ID No.: 156, 157, 158, 160, 162, 164, 166, 213 and/or 215 are used in the method according to the invention. In an embodiment of the present invention which is preferred most of all, the keratin-binding protein shown in the sequence SEQ ID No.: 213 is used. It goes without saying that this protein can be used either with or without the histidine anchor present in the SEQ ID No.: 213. Thus, the histidine anchor (or a purification/detection system to be used analogously) can also be present C-terminally. In practical use of said keratin-binding proteins (e.g. in cosmetic preparations), a histidine anchor (or a purification/detection system to be used analogously) is not necessary. The use of said proteins without additional amino acid sequences is thus preferred.

Likewise included according to the invention are “functional equivalents” of the specifically disclosed keratin-binding polypeptides (i) and the use of these in the method according to the invention.

Within the scope of the present invention, “functional equivalents” or analogs of these specifically disclosed keratin-binding polypeptides (i) are polypeptides different therefrom which also have the desired biological activity, such as, for example, keratin binding. Thus, for example, “functional equivalents” of keratin-binding polypeptides are understood as meaning those polypeptides which, under otherwise comparable conditions, in the quantitative keratin-binding tests described in the examples, have about 10%, 20%, 30%, 40% or 50%, preferably 60%, 70%, 80% or 90%, particularly preferably 100%, 125%, 150%, very particularly preferably 200%, 300% or 400%, most preferably 500%, 600%, 700% or 1000% or more of the keratin-binding capacity of the polypeptides shown under the SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215.

According to the invention, “functional equivalents” are understood in particular as meaning also muteins which have an amino acid other than that specifically given in at least one sequence position of the abovementioned amino acid sequences but nevertheless have one of the abovementioned biological activities. “Functional equivalents” thus include the muteins obtainable by a mutation where the specified changes can arise in any sequence position provided they lead to a mutein with the profile of properties according to the invention.

For the purposes of the present invention, “mutation” means the change in the nucleic acid sequence of a gene variant in a plasmid or in the genome of an organism. Mutations can arise, for example, as a result of errors during replication, or be caused by mutagenis. The rate of spontaneous mutations in the cell genome of organisms is very low although a large number of biological, chemical or physical mutagens is known to the knowledgeable person skilled in the art.

Mutations include substitutions, insertions, deletions of one or more nucleic acid radicals. Substitutions are understood as meaning the replacement of individual nucleic acid bases, a distinction being made here between transitions (substitution of a purine base for a purine base or a pyrimidine base for a pyrimidine base) and transversions (substitution of a purine base for a pyrimidine base (or vice versa)).

Additions or insertions are understood as meaning the incorporation of additional nucleic acid radicals into the DNA, possibly resulting in shifts in the reading frame. With reading frame shifts of this type, a distinction is made between “in frame” insertions/additions and “out of frame” insertions. In the case of “in frame” insertions/additions, the reading frame is retained and a polypeptide enlarged by the number of amino acids encoded by the inserted nucleic acids arises. In the case of “out of frame” insertions/additions, the original reading frame is lost and the formation of a complete and functioning polypeptide is no longer possible.

Deletions describe the loss of one or more base pairs, which likewise lead to “in frame” or “out of frame” shifts in the reading frame and the consequences associated therewith regard to the formation of an intact protein.

The mutagenic agents (mutagens) which can be used for producing random or targeted mutations and the applicable methods and techniques are known to the person skilled in the art. Such methods and mutagens are described, for example, in A. M. van Harten [(1998), “Mutation breeding, theory and practical applications”, Cambridge University Press, Cambridge, UK], E Friedberg, G Walker, W Siede [(1995), “DNA Repair and Mutagenesis”, Blackwell Publishing], or K. Sankaranarayanan, J. M. Gentile, L. R. Ferguson [(2000) “Protocols in Mutagenesis”, Elsevier Health Sciences].

For introducing targeted mutations, customary molecular biological methods and processes such as, for example, the in vitro mutagenesis Kits, LA PCR in vitro Mutagenesis Kit (Takara Shuzo, Kyoto) or the QuikChange® Kit from Stratagene or PCR mutageneses using suitable primers can be used.

As already discussed above, there is a large number of chemical, physical and biological mutagens.

The mutagens listed below are given by way of example, but are nonlimiting.

Chemical mutagens can be subdivided according to their mechanism of action. Thus, there are base analogs (e.g. 5-bromouracil, 2-aminopurine), mono- and bifunctional alkylating agents (e.g. monofunctional ones such as ethylmethylsulfonate, dimethyl sulfate, or bifunctional ones such as dichloroethyl sulfite, mitomycin, nitrosoguanidines-dialkylnitrosamines, N-nitrosoguanidine derivatives) or intercalating substances (e.g. acridines, ethidium bromide).

Thus, for example, for the method according to the invention, it is also possible to use those polypeptides which are obtained as a result of a mutation of a polypeptide according to the invention e.g. according to SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 and/or 215.

Examples of suitable amino acid substitutions are given in the table below:

TABLE 2
Suitable amino acid substitutions

| Original radical | Examples of substitution |
|---|---|
| Ala | Ser |
| Arg | Lys |
| Asn | Gln; His |
| Asp | Glu |
| Cys | Ser or Ala |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

It is known that in SEQ ID NO: 2, the serine naturally present at position 2849 can, for example, be replaced by glycine in order to avoid a phosphorylation at this position (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus, Mol Biol Cell. 2003 May; 14(5):1978-92. Epub 2003 Jan 26).

In the above sense, “functional equivalents” are also “precursors” of the described polypeptides, and “functional derivatives” and “salts” of the polypeptides.

Here, “precursors” are natural or synthetic precursors of the polypeptides with or without desired biological activity.

The expression “salts” is understood as meaning either salts of carboxyl groups or acid addition salts of amino groups with the protein molecules according to the invention. Salts of carboxyl groups can be prepared in a manner known per se and include inorganic salts, such as, for example, sodium, calcium, ammonium, iron and zinc salts, and also salts with organic bases, such as, for example, amines such as triethylamine, arginine, lysine, piperidine and the like. Acid addition salts, such as, for example, salts with mineral acids, such as hydrochloric acid or sulfuric acid, and salts with organic acids, such as acetic acid and oxalic acid, are likewise provided by the invention.

“Functional equivalents” naturally also include polypeptides which are accessible from other organisms, and naturally occurring variants (alleles). For example, through sequence comparisons, areas of homologous sequence regions or preserved regions can be determined. Using these sequences, DNA databases (e.g. genomic or cDNA databases) can be inspected for equivalent enzymes using bioinformatic comparison programs. Suitable computer programs and databases which are accessible to the public are sufficiently known to the person skilled in the art.

These alignments of known protein sequences can be carried out, for example, using a computer program such as Vector NTI 8 (version from Sep. 25, 2002) from InforMax Inc.

Furthermore, “functional equivalents” are fusion proteins which have one of the above-mentioned polypeptide sequences or functional equivalents derived therefrom and have at least one further heterologous sequence functionally different therefrom in functional N- or C-terminal linkage (i.e. without mutual essential functional impairment of the fusion protein parts). Nonlimiting examples of such heterologous sequences are, for example, signal peptides or enzymes.

“Functional equivalents” included according to the invention are homologs to the specifically disclosed proteins. These have at least 40%, 45% or 50%, preferably at least 55%, 60%, 65% or 70%, particularly preferably at least 75%, 80%, 85%, 90%, 91%, 92%, 93% or 94%, very particularly preferably at least 95% or 96% homology to one of the specifically disclosed amino acid sequences, calculated using the computer programs and computer algorithms disclosed in the definitions.

In the case of a possible protein glycosylation, “functional equivalents” according to the invention include proteins of the type referred to above in deglycosylated or glycosylated form, and also modified forms obtainable by changing the glycosylation pattern.

In the case of a possible protein phosphorylation, “functional equivalents” according to the invention include proteins of the type referred to above in dephosphorylated or phosphorylated form, and also modified forms obtainable by changing the phosphorylation pattern.

Homologs of the polypeptides according to the invention can be identified by screening combinatorial banks of mutants, such as, for example, shortening mutants. For example, a bank of protein variants can be produced by combinatorial mutagenesis at a nucleic acid level, such as, for example, by enzymatic ligation of a mixture of synthetic oligonucleotides. There is a large number of methods which can be used for producing banks of potential homologs from a degenerated oligonucleotide sequence. The chemical synthesis of a degenerated gene sequence can be carried out in an automatic DNA synthesis machine, and the synthetic gene can then be ligated into a suitable expression vector. The use of a degenerated set of genes makes it possible to provide all of the sequences in one mixture which encode the desired set of potential protein sequences. Methods for synthesizing degenerated oligonucleotides are known to the person skilled in the art (e.g. Narang, S. A. (1983) Tetrahedron 39:3; itakura et al. (1984) Annu. Rev. Biochem. 53:323; Itakura et al., (1984) Science 198:1056; Ike et al. (1983) Nucleic Acids Res. 11:477).

In the prior art, a number of techniques for the screening of gene products of combinatorial banks which have been produced by point mutations or shortening, and for the screening of cDNA banks for gene products with a selected property are known. The most often used techniques for screening large gene banks which are subjected to analysis with a high throughput include the cloning of the gene bank in replicable expression vectors, transforming the suitable cells with the resulting vector bank and expressing the combinatorial genes under conditions under which the detection of the desired activity facilitates the isolation of the vector which encodes the gene whose product has been detected. Recursive ensemble mutagenesis (REM), a technique which increases the frequency of functional mutants in the banks can be used in combination with the screening tests in order to identify homologs (Arkin and Yourvan (1992) PNAS 89:7811-7815; Delgrave et al. (1993) Protein Engineering 6(3):327-331).

The inspection of physically available cDNA or genomic DNA libraries of other organisms using the nucleic acid sequence described under SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 and/or 214, particularly preferably 165, 212 and 214, most preferably 214, or parts thereof as probe is a method known to the person skilled in the art for identifying homologs in other ways. Here, the probes derived from the nucleic acid sequence according to SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 and/or 214, particularly preferably 165, 212 and 214, most preferably 214, have a length of at least 20 bp, preferably at least 50 bp, particularly preferably at least 100 bp, very particularly preferably at least 200 bp, most preferably at least 400 bp. The probe can also be one or more kilobases long, e.g. 1 Kb, 1.5 Kb or 3 Kb. For inspecting the libraries it may also be possible to use a sequence of complementary DNA strand described under SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 and/or 214, particularly preferably 165, 212 and 214, most preferably 214, or a fragment thereof with a length between 20 bp and several kilobases. The hybridization conditions to be used are described above.

In the method according to the invention, it is also possible to use those DNA molecules which, under standard conditions, hybridize with the nucleic acid molecules described by SEQ ID No.: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 and/or 214, particularly preferably 165, 212 and 214, most preferably 214, and encoding keratin-binding polypeptides, nucleic acid molecules complementary to these or parts of the abovementioned, and as complete sequences encode polypeptides which have the same properties as the polypeptides described under SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215.

A particularly advantageous embodiment of the invention are keratin-binding polypeptides (i) which comprise at least one of the polypeptide sequences as shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215, with the proviso that the keratin binding of said polypeptides is at least 10%, 20%, 30%, 40% or 50%, preferably 60%, 70%, 80% or 90%, particularly preferably 100%, of the value which the corresponding polypeptide sequences as shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215 have, measured in the test according to Example 9 or 10.

Preference is given to using keratin-binding polypeptides (i) which have a highly specific affinity for the desired organism. Accordingly, for uses in skin cosmetics, preference is given to using keratin-binding polypeptides (i) which have a particularly high affinity to human skin keratin. For uses in hair cosmetics, preference is given to those polypeptide sequences which have a particularly high affinity to human hair keratin.

For applications in the pet field, besides the described polypeptide sequences (SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215, preferably in SEQ ID NO: 2, 4, 6, 8, 10, 12, 14, 40, 42, 44, 46, 48, 146, 150, 153, 156, 157, 158, 160, 164, 166, 213 or 215, particularly preferably 166 and 213, most preferably 213), those keratin-binding polypeptides (i) are accordingly preferred which have a particularly high affinity to the corresponding keratin, for example canine keratin or feline keratin.

However, it is also possible to use more than one keratin-binding polypeptide (i) coupled to the effector molecule (i) according to the invention, for example a keratin-binding polypeptide (i) which has a high binding affinity to human skin keratin can be combined with an effect molecule in combination with another keratin-binding polypeptide (i) which has a high affinity to human hair keratin. It is also possible to use chimeric polypeptides which comprise two or more copies of the same (and also different) keratin-binding polypeptides (i) or keratin-binding domains thereof. For example, it was thus possible to achieve particularly effective keratin binding.

Suitable keratin-binding polypeptides (i) are known. For example, desmoplakins and plectins comprise keratin-binding domains (Fontao L, Favre B, Riou S, Geerts D, Jaunin F, Saurat J H, Green K J, Sonnenberg A, Borradori L., Interaction of the bullous pemphigoid antigen 1 (BP230) and desmoplakin with intermediate filaments is mediated by distinct sequences within their COOH terminus., Mol Biol Cell. 2003 May; 14(5):1978-92. Epub 2003 Jan 26; Hopkinson S B, Jones J C., The N-terminus of the transmembrane protein BP180 interacts with the N-terminal domain of BP230, thereby mediating keratin cytoskeleton anchorage to the cell surface at the site of the hemidesmosome, Mol Biol Cell. 2000 January; 11(1):277-86).

The keratin-binding polypeptides (i) according to the invention can also—if desired—be separated again easily from the keratin. For this, for example, a rinse containing keratin can be used, as a result of which the keratin-binding polypeptides (i) are displaced from their existing binding to the keratin and are saturated with the keratin from the rinse. Alternatively, a rinse with a high content of detergent (e.g. SDS) is also possible for the washing off.

The present invention also preferably provides the above-described keratin-binding effector proteins where the effector polypeptide (ii) is chosen from the group consisting of enzymes, antibodies, effector-binding proteins, fluorescent proteins, antimicrobial peptides and self-assembling proteins.

### Enzymes:

The enzymes to be mentioned are preferably those chosen from the group consisting of oxidases, peroxidases, proteases, tyrosinases, lactoperoxidase, lysozyme, amyloglycosidases, glucose oxidases, superoxide dismutases, photolyases and catalases.

### Antibodies

The antibodies to be mentioned are preferably those which can bring a positive cosmetic benefit with them, e.g. antibodies directed toward skin pathogens.

The effector-binding proteins to be mentioned are preferably carotenoid-binding proteins (also called CBP below), vitamin-binding, chromophore-binding, odorant-binding, sugar-binding and metal-binding proteins. Among the carotenoid-binding proteins, particular preference is given to the carotenoid-binding protein (Accession number SWISS-PROT: Q8MYA9) from the silkworm Bombyx mori. The isolation of the protein and the characterization of the carotenoid-binding properties of this protein is described in Tabunoki et al. (2002; Isolation, characterization, and cDNA sequence of a carotenoid binding protein from the silk gland of Bombyx mori larvae.; J Biol Chem 277: 32133-32140). Among the metal-binding proteins, particular preference is given to the “Lead, cadmium, zinc and mercury transporting ATPase” ZntA (SWISS-PROT: P37617) from E. coli. The isolation and characterization of the ZntA protein are described, inner alia, in Sofia et al. (1994; Analysis of the Escherichia coli genome. V. DNA sequence of the region from 76.0 to 81.5 minutes.; Nucleic Acids Res 22: 2576-2586), Rensing et al. (1997; The znta gene of Escherichia coli encodes a Zn(II)-translocating P-type ATPase.; Proc Natl Acad Sci 94; 14326-14331) and Sharma et al. (2000; The ATP hydrolytic activity of purified ZntA, a Pb(II)/Cd(II)/Zn(II)-translocating ATPase from Escherichia coli.; J Biol Chem 275: 3873-3878).

### Fluorescent Proteins:

The fluorescent proteins to be mentioned are preferably those chosen from the group consisting of Green Fluorescent Protein (GFP), enhanced Green Fluorescent Protein (eGFP), Red Fluorescent Protein (RFP), monomeric Red Fluorescent Protein (mRFP), dsRED, Blue Fluorescent Protein (BFP), Yellow Fluorescent Protein (YFP) and Cyan Fluorescent Protein (CFP). Particular preference is given to the enhanced Green Fluorescent Protein (eGFP).

The GFP proteins are proteins which are produced by some animals which can fluoresce green if they are irradiated with blue light (UV light). One example of a carrier of the GFP protein is the jellyfish Aequorea victoria. Large numbers of this jellyfish with a characteristic green emission are found in the summer months on the north Pacific coast of the USA and Canada. The preceding letter “e” describes an improved “enhanced” version of the wild type GFP. eGFP is characterized by a 35 times higher intensity of the fluorescence.

Such fluorescent proteins are described and sold, for example, by the HHMI (Howard Hughes Medical Institute) laboratory.

The use of keratin-binding effector proteins comprising fluorescent proteins serves to achieve a more healthy and luminous-looking skin shade or for the optical lightening of the skin (“skin whitening”) following application to the skin.

In addition, these fluorescent protein-comprising keratin-binding effector proteins can also be used for lightening hair or for producing special reflections or shimmers on the hair. In addition, the fluorescent protein-comprising keratin-binding effector proteins can be used in decorative cosmetics in order, for example, to produce the effect of a tattoo when irradiated with UV light.

### AMPs

The antimicrobial peptides to be mentioned are preferably those chosen from the group consisting of polypeptides which lead to the inhibition of the growth of microorganisms, such as bacteria, fungi or protozoa. Particular preference is given to the polypeptide according to SEQ ID No.: 211.

### Self-Assembling Proteins:

The self-assembling proteins to be mentioned are preferably silk proteins from various organisms, such as, for example, spiders (e.g. Araneus diadematus), silkworms (e.g. Bombyx mori), mussels (e.g. Mytilus edulis). Among the silk proteins, particular preference is given to the C16 spider silk protein, which represents a 16-fold repetition of the C modulus of the protein ADF4 from Araneus diadematus. The construction and characterization of the C16 spider silk protein is described in Huemmerich et al. (2004; Primary structure elements of spider dragline silks and their contribution to protein solubility; Biochemistry 43: 13604-13612). Particular preference is given to the following silk proteins:

Silk protein from Nephila clavipes accession number AY855102 and U37520, Araneus gemmoides accession number AY855101 and accession number AY855100, Argiope aurantia accession number AY855099 and AY855098, the synthetic spider silk protein accession number DQ001900 and accession number DQ186903.

Further highly suitable effector proteins (ii) are polypeptides which occur naturally in microorganisms, in particular in E. coli or Bacillus subtilis. Examples of such fusion partners are the sequences YaaD (Accession No. BG10075) (SEQ ID NO:197 and 198) and thioredoxin (Accession No. EG11031) (SEQ ID NO:185 and 186).

Fragments and functional equivalents (according to the definition given above) of the abovementioned proteins and polypeptides are also suitable in principle as effector proteins (ii).

A particularly preferred subject matter of the present invention is directed to keratin-binding effector proteins comprising, as effector polypeptide (ii), a silk protein, particularly preferably silk proteins which comprise at least one of the sequences according to SEQ ID No.: 151, 201, 202, 203, 204, 205, 206, 207, 208, 209 or 210, or correspond to a polypeptide which is at least 40%, 45% or 50%, preferably at least 55%, 60%, 65% or 70%, particularly preferably at least 75%, 80%, 85%, 90%, 91%, 92%, 93% or 94%, very particularly preferably at least 95% or 96% identical to at least one of the sequences according to SEQ ID No.: 151, 201, 202, 203, 204, 205, 206, 207, 208, 209 or 210.

In addition, the invention provides those keratin-binding effector proteins comprising silk proteins which are encoded by a nucleic acid molecule comprising at least one nucleic acid molecule chosen from the group consisting of:
k) nucleic acid molecule which encodes a polypeptide comprising the sequence shown in SEQ ID No.: 151, 201, 202, 203, 204, 205, 206, 207, 208, 209 or 210;
l) nucleic acid molecule which comprises at least one polynucleotide of the sequence shown in SEQ ID No.: 150;
m) nucleic acid molecule which encodes a polypeptide according to the sequences SEQ ID No.: 151, 201, 202, 203, 204, 205, 206, 207, 208, 209 or 210;
n) nucleic acid molecule with a nucleic acid sequence according to SEQ ID No.: 150 or a nucleic acid molecule derived therefrom by substitution, deletion or insertion which encodes a polypeptide which is at least 40% identical to the sequence according to SEQ ID No.: 151;
o) nucleic acid molecule which encodes a polypeptide which is recognized by a monoclonal antibody directed toward a polypeptide which is encoded by the nucleic acid molecules according to (k) to (m);
p) nucleic acid molecule coding for a keratin-binding protein which hybridizes under stringent conditions with a nucleic acid molecule according to (k) to (m);
q) nucleic acid molecule coding for a keratin-binding protein which can be isolated from a DNA bank using a nucleic acid molecule according to (k) to (m) or part fragments thereof comprising at least 15 nucleotides as probe under stringent hybridization conditions; and
r) nucleic acid molecule which can be produced by backtranslating one of the amino acid sequences shown in the sequences SEQ ID No.: 151, 201, 202, 203, 204, 205, 206, 207, 208, 209 or 210.

In a preferred embodiment of the present invention, the chimeric keratin-binding effector proteins according to the invention are proteins in which the above-described polypeptides (i) and (ii) are linked together by means of translation fusion.

Besides the abovementioned effector proteins (ii), in one form of the present invention it is also possible to use those polypeptides which are constructed from at least 3 to 10, preferably at least 11 to 50, particularly preferably at least 51 to 100 and especially preferably at least more than 100 amino acids (also called fusion partners below) and which are not naturally linked to a keratin-binding polypeptide (i) as described above.

The effector protein (ii) can be chosen from a large number of proteins or polypeptides. It is also possible for a plurality of effector proteins (ii) to be linked to a keratin-binding polypeptide (i), for example on the amino terminus and on the carboxy terminus of the keratin-binding polypeptide moiety.

The keratin-binding effector proteins specified according to the invention and the keratin-binding polypeptides (i) and the effector proteins (ii) present therein can be produced chemically by known methods of peptide synthesis, for example by solid-phase synthesis according to Merrifield (2005, Kimmerlin T, Seebach D., ‘100 years of peptide synthesis’: ligation methods for peptide and protein synthesis with applications to beta-peptide assemblies., J Pept Res. 2005 February; 65(2):229-260).

Of particular suitability, however, are genetic engineering methods in which the nucleic acid molecules coding for the keratin-binding polypeptides (i) and the nucleic acid molecules coding for the effector proteins (ii) are functionally linked with one another so that, as a result of the translation of the fused nucleic acid molecule, a single general translation product is formed (translation fusion).

Host organisms (production organisms) suitable for producing the above-described keratin-binding polypeptides (i), the effector proteins (ii) or the fusion proteins (comprising the amino acid sequences of the polypeptides (i) and (ii)) are prokaryotes (including the Archaea) and eukaryotes, preferably bacteria including halobacteria and methanococci, fungi, insect cells, plant cells and mammal cells, particularly preferably Escherichia coli, Bacillus subtilis, Bacillus. megaterium, Aspergillus oryzea, Aspergillus nidulans, Aspergillus niger, Pichia pastoris, Pseudomonas spec., Lactobacillae, Hansenula polymorpha, Trichoderma reesei, and SF9 (or related cells).

### Chemical Coupling

The invention further preferably provides keratin-binding effector proteins in which the above-described polypeptides (i) and (ii) are linked together by means of a chemical coupling reaction. During these coupling reactions, bonds can be closed chosen from the group of covalent bonds consisting of thioesters, esters, thioethers, ethers, amide bonds, sulfonic esters and sulfonamide bonds. Here, said linkages can be closed between the side chains of internal amino acids, the N-terminus or the C-terminus of the keratin-binding polypeptide (i) and the side chains of internal amino acids, the N-terminus or the C-terminus of the effector protein.

Alternatively, a direct coupling between effector molecule (ii) and the keratin-binding domain can be carried out e.g. by means of carbodiimides, glutardialdehyde or other crosslinkers known to the person skilled in the art. A selection. of such coupling reactions is given in 2005, Kimmerlin T, Seebach D., ‘100 years of peptide synthesis’. ligation methods for peptide and protein synthesis with applications to beta-peptide assemblies., J Pept Res., 65(2):229-260, and 2004, David R et al., Expressed protein ligation, Eur. J. Biochem. 271, 663-677.

Also provided by the present invention are keratin-binding effector proteins in which the effector polypeptide (ii) and the keratin-binding polypeptide (i) are joined together by means of a spacer element. The spacer element may be stable, thermally cleavable, photocleavable or enzymatically cleavable (particularly by lipases, esterases, proteases, phosphatases, hydrolases etc.). Corresponding chemical structures are known to the person skilled in the art and are integrated between the molecular moieties (i) and (ii). Examples of enzymatically cleavable linkers which can be used in the molecules according to the invention are given, for example, in WO 98/01406, to the entire contents of which reference is hereby expressly made.

The spacer elements may be crosslinkers which are known in principle to the person skilled in the art, preferably carbodiimides or glutardialdehyde. For this linkage, a virtually direct linkage between the keratin-binding polypeptide and the effector protein is ensured. Carbodiimides to be mentioned are preferably dicyclohexylcarbodiimide (DCC), diisopropylcarbodiimide (DIC), N′-(3-dimethylaminopropyl)-N-ethylcarbodiimide hydrochloride (EDC), with the use of diisopropylcarbodiimide or EDC being particularly preferred.

A further preferred subject matter of the invention is keratin-binding effector proteins in which the spacer element linking the polypeptides (i) and (ii) is a polypeptide.

For example, the nucleic acid molecules coding for the keratin-binding effector proteins can be modified by suitable biotechnological cloning methods in such a way that the translation fusion also comprises a polypeptide sequence functioning as spacer element. These polypeptide spacer elements can have cleavage sites for proteases (e.g. skin protease cathepsin D), lipases, esterases, phosphatases or hydrolases, or polypeptide sequences which permit simple purification of the fusion protein, for example so-called His tags, i.e. oligohistidine radicals.

In addition, it is also possible to insert additional amino acids at the linkage sites between the polypeptides (i) and (ii) by means of suitable genetic engineering methods. This may, for example, also arise from recognition sites for restriction endonucleases being either newly created or inactivated at the nucleic acid level. Furthermore, additional amino acids can be inserted at the linkage sites of two fusion partners in order to create a linker sequence so that the two fusion partners can be folded independently of one another to give functional polypeptide moieties. The proteins according to the invention can also be modified posttranslationally, i.e. after their translation, for example by glycosylation, phosphorylation or acylation. Such a modification can also take place by a chemical route, e.g. crosslinking with glutardialdehyde.

In a particularly preferred embodiment, the present invention provides keratin-binding effector proteins which are indirectly joined together by means of a spacer element, where the spacer element is an at least bifunctional linker which covalently joins together the keratin-binding polypeptide (i) and the effector polypeptide by binding to side chains of internal amino acids, the C-terminus or the N-terminus of said polypeptides.

A keratin-binding effector protein according to the invention can be produced by coupling an effector protein (ii) onto a keratin-binding polypeptide (i) using a linker molecule (iii) which has at least two coupling functionalities which can enter into bonds chosen from the group consisting of thioester, ester, thioether, ether, amide, sulfonic ester and sulfonamide bonds, and
(a) in a first coupling step, firstly the effector polypeptide (ii) is bonded to the linker molecule (iii) via one of said bonds, and
(b) in a further coupling step, the reaction product from (a) is coupled to the keratin-binding polypeptide (i) via a coupling functionality of the linker molecule (iii) which is still free.

In a preferred embodiment, the coupling functionalities are at least two different functional groups.

The binding of the linker molecule to the effector polypeptide (ii) takes place via a chemical coupling reaction. This can take place, for example, via the C- or N-terminal functionality or the side chains of the effector polypeptide, in particular via amino functions, hydroxy functions, carboxylate functions or thiol functions. Preference is given to a linkage via the amino functions of one or more lysine radicals, one or more thiol groups of cysteine radicals, one or more hydroxyl groups of serine, threonine or tyrosine radicals, one or more carboxyl groups of aspartic acid or glutamic acid radicals or via the N-terminal or C-terminal function of the effector polypeptide (ii).

Such chemical coupling reactions are known to the person skilled in the art and are described, for example, in: Becker, H. G. O., Organikum [Organics], 20th edition, 1996, Johann Ambrosius Barth Verlag Heidelberg or Hermanson, G. T.: Bioconjugate Techniques, 1996, Academic Press, San Diego.

Apart from the amino acid functions occurring in the primary sequence of the effector polypeptide (ii), amino acids with suitable functions (e.g. cysteines, lysines, aspartates, glutamates) can also be attached to the sequence, or amino acids of the polypeptide sequence can be substituted by such amino acid functions. Methods for the mutagenesis or manipulation of nucleic acid molecules are sufficiently known to the person skilled in the art. A few selected methods are described below.

The binding of the reaction product resulting from the above-described step (a) with the keratin-binding polypeptide (i) takes place via the second anchor group of the linker molecule which is still free. Besides the above-described coupling reactions, suitable anchor groups of this type are particularly sulfhydryl-reactive groups (e.g. maleimides, pydridyldisulfides, α-haloacetyls, vinylsulfones, sulfatoalkylsulfones (preferably sulfatoethylsulfones and also thiols), by means of which the linker can enter into a covalent bond with a cysteine radical of the keratin-binding polypeptide (i).

Preference is given to a covalent linkage of the linker molecule (iii) to the keratin-binding polypeptide (i). This can take place, for example, via the side chains of the keratin-binding polypeptide (i), in particular via amino functions, hydroxy functions, carboxylate functions or thiol functions. Preference is given to a linkage via the amino functions of one or more lysine radicals, one or more thiol groups of cysteine radicals, one or more hydroxyl groups of serine, threonine or tyrosine radicals, one or more carboxyl groups of aspartic acid or glutamic acid radicals or via the N-terminal or C-terminal function of the keratin-binding polypeptide (ii). Apart from the amino acid functions occurring in the primary sequence of the keratin-binding polypeptide (ii), amino acids with suitable functions (e.g. cysteines, lysines, aspartates, glutamates) can also be added to the sequence, or amino acids of the polypeptide sequence can be substituted by such amino acid functions. Methods for the mutagenesis or manipulation of nucleic acid molecules are sufficiently known to the person skilled in the art. A few selected methods are described below.

The success of the effector coupling can be monitored using three different tests:
(i) Ellmann test in which the number of free Cys-SH groups in the protein can be determined before and after effector coupling. Here, a considerable reduction in the free SH groups after coupling indicates good reaction progress (see Example 17).
(ii) Activity test in which the binding of the keratin-binding polypeptide with and without coupled linker effector protein to hair can be measured (see Example 16).
(iii) Determination of the molar mass of the coupled protein.

The keratin-binding polypeptides (i) according to the invention have a wide field of use in human cosmetics, in particular skin care, nail care and hair care, in animal care, leather care and leather working.

Preferably, the keratin-binding effector proteins according to the invention are used for skin cosmetics and hair cosmetics. They permit a high concentration and long action time of care or protecting effector molecules.

In a particularly preferred embodiment of the present invention, keratin-binding polypeptides are used which have a binding affinity to human skin keratin, hair keratin or nail keratin.

The present invention particularly preferably provides keratin-binding effector proteins in which
s) the keratin-binding polypeptide used comprises one of the sequences shown in the SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215, preferably in SEQ ID NO: 2, 4, 6, 8, 10, 12, 14, 40, 42, 44, 46, 48, 146, 150, 153, 156, 157, 158, 160, 164, 166, 213 or 215, particularly preferably 166 and 213, most preferably 213 and
t) the effector polypeptide (ii) is chosen from the group of silk proteins, preferably the C16 spider silk protein, which is a 16-fold repetition of the C modulus of the protein ADF4 from Araneus diadematus, optionally
u) the proteins specified under s) and t) can also be coupled together via a linker molecule.

The present invention further provides the use of the keratin-binding effector molecules produced according to the invention in dermocosmetic preparations. Preferably, the keratin-binding effector molecules according to the invention are used in skin and hair cosmetics. They permit a high concentration and long action time of skin care or skin-protecting effector substances.

In a preferred embodiment of the present invention, the keratin-binding effector proteins according to the invention are used in skin protection compositions, skincare compositions, skin cleansing compositions, hair protection compositions, haircare compositions, hair cleansing compositions, hair colorants or in products for decorative cosmetics.

In a preferred embodiment of the present invention, a keratin-binding effector protein according to the invention is added to the dermocosmetics in a concentration of from 0.001 to 1 percent by weight (% by wt.), preferably 0.01 to 0.9% by weight, particularly preferably 0.01 to 0.8% by weight or 0.01 to 0.7% by weight, very particularly preferably 0.01 to 0.6% by weight or 0.01 to 0.5% by weight, most preferably 0.01 to 0.4% by weight or 0.01 to 0.3% by weight, based on the total weight of the composition. In a further embodiment, the compositions comprise a keratin-binding effector protein according to the invention in a concentration of from 1 to 10% by weight, preferably 2 to 8% by weight, 3 to 7% by weight, 4 to 6% by weight, based on the total weight of the composition. In a likewise preferred embodiment, the compositions comprise a keratin-binding effector protein according to the invention in a concentration of from 10 to 20% by weight, preferably 11 to 19% by weight, 12 to 18% by weight, 13 to 17% by weight, 14 to 16% by weight, based on the total weight of the composition. In a moreover preferred embodiment the compositions comprise a keratin-binding effector protein according to the invention in a concentration of from 20 to 30% by weight, preferably 21 to 29% by weight, 22 to 28% by weight, 23 to 27% by weight, 24 to 26% by weight, based on the total weight of the composition.

In another preferred embodiment, the abovementioned keratin-binding effector molecules according to the invention are used in dermocosmetics in combination with (i) cosmetic auxiliaries from the field of decorative cosmetics, (ii) dermocosmetic active ingredients and (iii) suitable auxiliaries and additives. Preferably, these are active ingredients and auxiliaries and additives which are used to protect the skin, hair and/or fingernails or toenails from damage, for treating existing damage to skin, hair and/or fingernails or toenails and for caring for skin, hair and/or fingernails or toenails. These active ingredients are preferably chosen from the group of natural or synthetic polymers, pigments, humectants, oils, waxes, enzymes, minerals, vitamins, sunscreens, dyes, fragrances, antioxidants, preservatives and/or pharmaceutical active ingredients.

Suitable auxiliaries and additives for producing hair cosmetic or skin cosmetic preparations are familiar to the person skilled in the art and can be found in cosmetics handbooks, for example Schrader, Grundlagen und Rezepturen der Kosmetika [Fundamentals and formulations of cosmetics], Hüthig Verlag, Heidelberg, 1989, ISBN 3-7785-1491-1, or Umbach, Kosmetik: Entwicklung, Herstellung und Anwendung kosmetischer Mittel [Cosmetics: development, manufacture and use of cosmetic compositions], 2nd expanded edition, 1995, Georg Thieme Verlag, ISBN 3 13 712602 9.

Preferably, the keratin-binding effector molecules according to the invention are used in dermocosmetics or compositions for oral care, dental care and denture care in combination with at least one constituent different therefrom which is chosen from cosmetically active ingredients, emulsifiers, surfactants, preservatives, perfume oils, thickeners, hair polymers, hair and skin conditioners, graft polymers, water-soluble or dispersible silicone-containing polymers, photoprotective agents, bleaches, gel formers, care agents, colorants, tinting agents, tanning agents, dyes, pigments, consistency regulators, humectants, refatting agents, collagen, protein hydrolyzates, lipids, antioxidants, antifoams, antistats, emollients and softeners. The active ingredients can also be present in the cosmetic preparations in encapsulated form, as described in the patents/patent applications EP 00974775 B1, DE 2311 712, EP 0278 878>DE 1999 47147, EP 0706822B1 and WO 98/16621, to which reference is hereby expressly made.

Advantageously, the antioxidants are chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thiorodoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. sodium ascorbate, ascorbyl palmitate, Mg ascorbyl phosphate, ascorbyl acetate), tocopherol and derivatives (e.g. vitamin E acetate, tocotrienol), vitamin A and derivatives (vitamin A paimitate), and coniferyl benzoate of benzoin resin, rutinic acid and derivatives thereof α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The vitamins, provitamins or vitamin precursors of the vitamin B group or derivatives thereof and the derivatives of 2-furanone to be used with preference according to the invention include, inter alia:

Vitamin B₁, trivial name thiamine, chemical name 3-[(4′-amino-2′-methyl-5′-pyrimidinyl)methyl]-5-(2-hydroxyethyl)-4-methylthiazolium chloride.

Vitamin B2, trivial name riboflavin, chemical name 7,8-dimethyl-10-(1-D-ribityl)-benzo[g]pteridine-2,4(3H,10H)-dione. In free form, riboflavin occurs, for example, in whey, other riboflavin derivatives can be isolated from bacteria and yeasts. A stereoisomer of riboflavin which is likewise suitable according to the invention is lyxoflavin, which can be isolated from fish meal or liver and bears a D-arabityl radical instead of the D-ribityl radical.

Vitamin B3. The compounds nicotinic acid and nicotinamide (niacinamide) often bear this name. According to the invention, preference is given to nicotinamide.

Vitamin B₅ (pantothenic acid and panthenol). Preference is given to using panthenol. Derivatives of panthenol which can be used according to the invention are, in particular, the esters and ethers of panthenol, and cationically derivatized panthenols. In a further preferred embodiment of the invention, derivatives of 2-furanone can also be used in addition to pantothenic acid or panthenol. Particularly preferred derivatives are the also commercially available substances dihydro-3-hydroxy-4,4-dimethyl-2(3H)-furanone with the trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), with all stereoisomers being expressly included.

These compounds advantageously impart moisturizing and skin-calming properties to the dermocosmetics according to the invention.

Vitamin B₆, which is not understood here as meaning a uniform substance, but the derivatives of 5-hydroxymethyl-2-methylpyridin-3-ol known under the trivial names pyridoxin, pyridoxamine and pyridoxal.

Vitamin B₇ (biotin), also referred to as vitamin H or “skin vitamin”. Biotin is (3aS,4S,6aR)-2-oxohexahydrothienol[3,4-d]imidazole-4-valeric acid.

Panthenol, pantolactone, nicotinamide and biotin are very particularly preferred according to the invention.

### Dyes

Dyes which can be used are the substances approved and suitable for cosmetic purposes, as are listed, for example, in the publication “Kosmetische Färbemittel” [Cosmetic Colorants] from the Farbstoffkommission der Deutschen Forschungsgemeinschaft [Dyes Commission of the German Research Society], published by Verlag Chemie, Weinheim, 1984. These dyes are usually used in concentrations of from 0.001 to 0.1% by weight, based on the total mixture.

### Pigments

In a preferred embodiment, the compositions according to the invention comprise at least one pigment. The pigments are present in the product mass in undissolved form and may be present in an amount of from 0.01 to 25% by weight, particularly preferably from 5 to 15% by weight. The preferred particle size is 1 to 200 □m, in particular 3 to 150 □m, particularly preferably 10 to 100 □m. The pigments are colorants which are virtually insoluble in the application medium and may be inorganic or organic. Inorganic-organic mixed pigments are also possible. Preference is given to inorganic pigments. The advantage of the inorganic pigments is their excellent photostability, weather stability and thermal stability. The inorganic pigments may be of natural origin, for example prepared from chalk, ochre, umber, green earth, burnt sienna or graphite. The pigments may be white pigments, such as, for example, titanium dioxide or zinc oxide, black pigments, such as, for example, iron oxide black, colored pigments, such as, for example, ultramarine or iron oxide red, pearlescent pigments, metal effect pigments, pearlescent pigments and fluorescent or phosphorescent pigments, where preferably at least one pigment is a colored, non-white pigment. Metal oxides, hydroxides and oxide hydrates, mixed-phase pigments, sulfur-containing silicates, metal sulfides, complex metal cyanides, metal sulfates, chromates and molybdates, and the metals themselves (bronze pigments) are suitable. Of particular suitability are titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), iron blue (ferric ferrocyanide, CI 77510), carmine (cochineal). Particular preference is given to pearlescent pigments and colored pigments based on mica which are coated with a metal oxide or a metal oxychloride, such as titanium dioxide or bismuth oxychloride, and if appropriate further color-imparting substances, such as iron oxides, iron blue, ultramarine, carmine etc., and where the color can be determined by varying the layer thickness. Pigments of this type are sold, for example, under the trade names Rona®, Colorona®, Dichrona® and Timiron® (Merck). Organic pigments are, for example, the natural pigments sepia, gamboge, Cassel brown, indigo, chlorophyll and other plant pigments. Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

In one embodiment, the keratin-binding effector molecules according to the invention and/or produced according to the inventive method are used with at least one particulate substance which is present in the composition in an amount of from 0.01 to 10, preferably from 0.05 to 5% by weight. Suitable substances are, for example, substances which are solid at room temperature (25° C.) and are in the form of particles. For example, silica, silicates, aluminates, clay earths, mica, salts, in particular inorganic metal salts, metal oxides, e.g. titanium dioxide, minerals and polymer particles are suitable. The particles are present in the composition in undissolved, preferably stably dispersed form and are able, following application to the application surface and evaporation of the solvent, to be deposited in solid form. Preferred particulate substances are silica (silica gel, silicon dioxide) and metal salts, in particular inorganic metal salts, where silica is particularly preferred. Metal salts are, for example, alkali metal or alkaline earth metal halides, such as sodium chloride or potassium chloride; alkali metal or alkaline earth metal sulfates, such as sodium sulfate or magnesium sulfate.

### Pearlizing Agents

Suitable pearlizing agents are, for example: alkylene glycol esters, specifically ethylene glycol disterate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have in total at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Customary thickeners in such formulations are crosslinked polyacrylic acids and derivatives thereof polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone. Preference is given to using nonionic thickeners.

Suitable cosmetically and/or dermocosmetically active ingredients are, for example, coloring active ingredients, skin and hair pigmentation agents, tinting agents, tanning agents, bleaches, keratin-hardening substances, antimicrobial active ingredients, photofilter active ingredients, repellent active ingredients, hyperemic substances, keratolytically and keratoplastically effective substances, antidandruff active ingredients, antiphlogistics, keratinizing substances, antioxidative active ingredients and/or active ingredients which act as free-radical scavengers, skin moisturizing or humectant substances, refatting active ingredients, antierythematous or antiallergic active ingredients, branched fatty acids, such as 18-methyleicosanoic acid, and mixtures thereof.

Artificially skin-tanning active ingredients which are suitable for tanning the skin without natural or artificial radiation with UV rays are, for example, dihydroxyacetone, alloxan and walnut shell extract. Suitable keratin-hardening substances are usually active ingredients, as are also used in antiperspirants such as, for example, potassium aluminum sulfate, aluminum hydroxychloride, aluminum lactate, etc.

Antimicrobial active ingredients are used to destroy microorganisms or to inhibit their growth and thus serve both as preservative and as deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc.

Suitable preservatives to be used advantageously according to the invention are:

TABLE 3
Suitable preservatives. The E numbers listed in the table above
are the designations used in the guideline 95/2/EEC.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na salt |
| E 216 | n-Propyl p-hydroxybenzoate |
| E 217 | n-Propyl p-hydroxybenzoate Na salt |
| E 218 | Methyl p-hydroxybenzoate |
| E 219 | Methyl p-hydroxybenzoate Na salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogensulfite |
| E 228 | Potassium hydrogensulfite |
| E 230 | Biphenyl (diphenyl) |
| E 231 | Orthophenylphenol |
| E 232 | Sodium orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrite |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also suitable according to the invention are preservatives or preservative auxiliaries customary in cosmetics dibromodicyanobutane (2-bromo-2-bromomethylglutarodinitrile), 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalkonium chloride and benzyl alcohol. Also suitable as preservatives are phenyl hydroxyalkyl ethers, in particular the compound known under the name phenoxyethanol on account of its bactericidal and fungicidal effects on a number of microorganisms.

Other antimicrobial agents are likewise suitable for being incorporated into the preparations according to the invention. Advantageous substances are, for example, 2,4,4′-trichloro-2′-hydroxydiphenyl ether (irgasan), 1,6-di(4-chlorophenylbiguanido)hexane (chlorhexidine), 3,4,4′-trichlorocarbanilide, quaternary ammonium compounds, oil of cloves, mint oil, thyme oil, triethyl citrate, farnesol (3,7,11-trimethyl-2,6,10-dodecatrien-1-ol), and the active ingredients or active ingredient combinations described in the patent laid-open specifications DE-37 40 186, DE-39 38 140, DE-42 04 321, DE-42 29 707, DE-43 09 372, DE-44 11 664, DE-195 41 967, DE-195 43 695, DE-195 43 696, DE-195 47 160, DE-196 02 108, DE-196 02 110, DE-196 02 111, DE-196 31 003, DE-196 31 004 and DE-196 34 019 and the patent specifications DE-42 29 737, DE-42 37 081, DE-43 24 219, DE-44 29 467, DE-44 23 410 and DE-195 16 705. Sodium hydrogencarbonate is also to be used advantageously. Microbial polypeptides can also likewise be used.

### Perfume Oils

If appropriate, the cosmetic compositions can comprise perfume oils. Perfume oils which may be mentioned are, for example, mixtures of natural and synthetic fragrances. Natural fragrances are extracts from flowers (lily, lavender, rose, jasmine, neroli, ylang ylang), stems and leaves (geranium, patchouli, petitgrain), fruits (aniseed, coriander, caraway, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester type, ether type, aldehyde type, ketone type, alcohol type and hydrocarbon type. Fragrance compounds of the ester type are, for example, benzyl acetate, phenoxyethyl isobutyrate, 4-tert-butyl cyclohexylacetate, linalyl acetate, dimethylbenzylcarbinyl acetate, phenylethyl acetate, linalyl benzoate, benzyl formate, ethyl methylphenyl glycinate, allyl cyclohexylpropionate, styrallyl propionate and benzyl salicylate. The ethers include, for example, benzyl ethyl ether, the aldehydes include, for example, the alkanals having 8 to 18 carbon atoms, citral, citronellal, citronellyloxyacetaldehyde, cyclamenaldehyde, hydroxycitronellal lilial and bourgeonal, the ketones include, for example, the ionones, α-isomethylionene and methyl cedryl ketone, the alcohols include anethol, citronellol, eugenol, isoeugenol, geraniol, linalool, phenylethyl alcohol and terpeneol, the hydrocarbons include primarily the terpenes and balsams. However, preference is given to using mixtures of different fragrances which together produce a pleasant scent note. Essential oils of relatively low volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Preferably, bergamot oil, dihydromyrcenol, lilial, lyral, citronellol, phenylethyl alcohol, □-hexylcinnamaldehyde, geraniol, benzylacetone, cyclamenaldehyde, linalool, Boisambrene®Forte, ambroxan, indole, hedione, sandelice, lemon oil, mandarin oil, orange oil, allyl amyl glycolate, cyclovertal, lavandin oil, clary sage oil, □-damascone, geranium oil bourbon, cyclohexyl salicylate, Vertofix®Coeur, Iso-E-Super®, Fixolide®NP, evernyl, iraldein gamma, phenylacetic acid, geranyl acetate, benzyl acetate, rose oxide, romillate, irotyl and floramate, alone or in mixtures, are used.

### Oils, Fats and Waxes

Preferably, the compositions according to the invention comprise oils, fats and/or waxes. Constituents of the oil phase and/or fat phase of the compositions according to the invention are advantageously chosen from the group of lecithins and fatty acid triglycerides, namely the triglycerol esters of saturated and/or unsaturated, branched and/or unbranched alkanecarboxylic acids of chain length from 8 to 24, in particular 12 to 18, carbon atoms. The fatty acid triglycerides can, for example, advantageously be chosen from the group of synthetic, semisynthetic and natural oils, such as, for example, olive oil, sunflower oil, soya oil, peanut oil, rapeseed oil, almond oil, palm oil, coconut oil, castor oil, wheat germ oil, grapeseed oil, thistle oil, evening primrose oil, macadamia nut oil and the like. Further polar oil components can be chosen from the group of esters of saturated and/or unsaturated, branched and/or unbranched alkanecarboxylic acids of chain length from 3 to 30 carbon atoms and saturated and/or unsaturated, branched and/or unbranched alcohols of chain length from 3 to 30 carbon atoms, and from the group of esters of aromatic carboxylic acids and saturated and/or unsaturated, branched and/or unbranched alcohols of chain length from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate dicaprylylcarbonate (cetiol CC) and cocoglycerides (myritol 331), butylene glycol dicaprylate/dicaprate and dibutyl adipate, and synthetic, semisynthetic and natural mixtures of such esters, such as, for example, jojoba oil.

In addition, one or more oil components can advantageously be chosen from the group of branched and unbranched hydrocarbons and hydrocarbon waxes, silicone oils, dialkyl ethers, the group of saturated or unsaturated, branched or unbranched alcohols. Any mixtures of such oil and wax components are also to be used advantageously for the purposes of the present invention. If appropriate, it may also be advantageous to use waxes, for example cetyl palmitate, as the sole lipid component of the oil phase. According to the invention, the oil component is advantageously chosen from the group consisting of 2-ethylhexyl isostearate, octyldodecanol, isotridecyl isononanoate, isoeicosane, 2-ethylhexyl cocoate, C12-15-alkyl benzoate, caprylic/capric triglyceride, dicaprylyl ether. According to the invention, mixtures of C12-15-alkyl benzoate and 2-ethylhexyl isostearate, mixtures of C12-15-alkyl benzoate and isotridecyl isononanoate, and mixtures of C12-15-alkyl benzoate, 2-ethylhexyl isostearate and isotridecyl isononanoate are advantageous. According to the invention, the oils with a polarity of from 5 to 50 mN/m particularly preferably used are fatty acid triglycerides, in particular soya oil and/or almond oil. Of the hydrocarbons, paraffin oil, squalane and squalene are to be used advantageously for the purposes of the present invention.

In addition, the oil phase can advantageously be chosen from the group of Guerbet alcohols. Guerbet alcohols are named after Marcel Guerbet who described their preparation for the first time. They form in accordance with the reaction equation

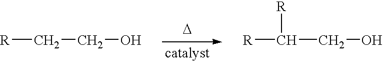

by oxidation of an alcohol to give an aldehyde, by aldol condensation of the aldehyde, elimination of water from the aldol and hydrogenation of the allyl aldehyde. Guerbet alcohols are liquid even at low temperatures and cause virtually no skin irritations. They can be used advantageously as fatting, superfatting and also refatting constituents in cosmetic compositions.

The use of Guerbet alcohols in cosmetics is known per se. Such species are then mostly characterized by the structure

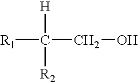

Here, R₁ and R₂ are usually unbranched alkyl radicals.

According to the invention, the Guerbet alcohol or alcohols are advantageously chosen from the group where

R₁=propyl, butyl, pentyl, hexyl, heptyl or octyl and
R₂=hexyl, heptyl, octyl, nonyl, decyl, undecyl, dodecyl, tridecyl or tetradecyl.

Guerbet alcohols preferred according to the invention are 2-butyloctanol (commercially available for example as Isofol®12 (Condea)) and 2-hexyldecanol (commercially available for example as Isofol®16 (Condea)). Mixtures of Guerbet alcohols according to the invention are also to be used advantageously according to the invention, such as, for example, mixtures of 2-butyloctanol and 2-hexyldecanol (commercially available for example as Isofol®14 (Condea)).

Any mixtures of such oil and wax components are also to be used advantageously for the purposes of the present invention. Among the polyolefins, polydecenes are the preferred substances.

The oil component can also advantageously have a content of cyclic or linear silicone oils or consist entirely of such oils, although it is preferred to use an additional content of other oil phase components apart from the silicone oil or the silicone oils. Low molecular weight silicones or silicone oils are generally defined by the following general formula:

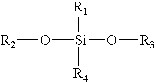

Higher molecular weight silicones or silicone oils are generally defined by the following general formula

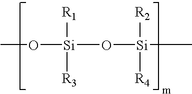

where the silicon atoms may be substituted by identical or different alkyl radicals and/or aryl radicals, which are shown here in general terms by the radicals R₁ to R₄. However, the number of different radicals is not necessarily limited to up to 4. m here can assume values from 2 to 200 000.

Cyclic silicones to be used advantageously according to the invention are generally defined by the following general formula

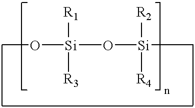

where the silicon atoms can be substituted by identical or different alkyl radicals and/or aryl radicals, which are shown here in general terms by the radicals R₁ to R₄. However, the number of different radicals is not necessarily limited to up to 4. n here can assume values from 3/2 to 20. Fractional values for n take into consideration that uneven numbers of siloxyl groups may be present in the cycle.

Advantageously, phenyltrimethicone is chosen as silicone oil. Other silicone oils, for example dimethicone, hexamethylcyclotrisiloxane, phenyldimethicone, cyclomethicone (octamethylcyclotetrasiloxane), hexamethylcyclotrisiloxane, polydimethylsiloxane, poly(methylphenylsiloxane), cetyidimethicone, behenoxydimethicone are also to be used advantageously for the purposes of the present invention. Also advantageous are mixtures of cyclomethicone and isotridecyl isononanoate, and those of cyclomethicone and 2-ethylhexyl isostearate. However, it is also advantageous to choose silicone oils of similar constitution to the compounds referred to above whose organic side chains are derivatized, for example polyethoxylated and/or polypropoxylated. These include, for example, polysiloxane-polyalkyl polyether copolymers, such as, for example, cetyldimethicone copolyol. Cyclomethicone (octamethylcyclotetrasiloxane) is advantageously used as silicone oil to be used according to the invention. Fat and/or wax components to be used advantageously according to the invention can be chosen from the group of vegetable waxes, animal waxes, mineral waxes and petrochemical waxes. For example, candelilla wax, carnauba wax, Japan wax, esparto grass wax, cork wax, guaruma wax, rice germ oil wax, sugarcane wax, berry wax, ouricury wax, montan wax, jojoba wax, shea bufter, beeswax, shellac wax, spermaceti, lanolin (wool wax), uropygial grease, ceresine, ozokerite (earth wax), paraffin waxes and microwaxes are advantageous.

Further advantageous fat and/or wax components are chemically modified waxes and synthetic waxes, such as, for example, Syncrowax®HRC (glyceryl tribehenate), and Syncrowax®AW 1 C(C₁₈₋₃₆ fatty acid) and montan ester waxes, sasol waxes, hydrogenated jojoba waxes, synthetic or modified beeswaxes (e.g. dimethicone copolyol beeswax and/or C₃₀₋₅₀-alkyl beeswax), cetyl ricinoleates such as, for example, Tegosoft®CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkylhydroxystearoyl stearate and/or glycol montanate. Furthermore, certain organosilicon compounds which have similar physical properties to the specified fat and/or wax components, such as, for example, stearoxytrimethylsilane, are also advantageous.

According to the invention, the fat and/or wax components can be used in the compositions either singly or as a mixture. Any mixtures of such oil and wax components are also to be used advantageously for the purposes of the present invention. Advantageously, the oil phase is chosen from the group consisting of 2-ethylhexyl isostearate, octyidodecanol, isotridecyl isononanoate, butylene glycol dicaprylateldicaprate, 2-ethylhexyl cocoate, C₁₂₋₁₅-alkyl benzoate, caprylic/capric triglyceride, dicaprylyl ether. Mixtures of octyldodecanol, caprylic/capric triglyceride, dicaprylyl ether, dicaprylyl carbonate, cocoglycerides or mixtures of C₁₂₋₁₅-alkyl benzoate and 2-ethylhexyl isostearate, mixtures of C₁₂₋₁₅-alkyl benzoate and butylene glycol dicaprylate/dicaprate, and mixtures of C₁₂₋₁₅-alkyl benzoate, 2-ethylhexyl isostearate and isotridecyl isononanoate are particularly advantageous. Of the hydrocarbons, paraffin oil, cycloparaffin, squalane, squalene, hydrogenated polyisobutene and polydecene are to be used advantageously for the purposes of the present invention.

The oil component is also advantageously chosen from the group of phospholipids. Phospholipids are phosphoric esters of acylated glycerols. Of greatest importance among the phosphatidylcholines are, for example, the lecithins, which are characterized by the general structure

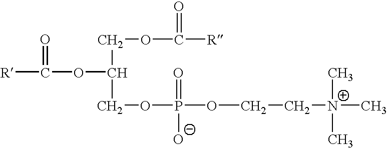

where R′ and R″ are typically unbranched aliphatic radicals having 15 or 17 carbon atoms and up to 4 cis double bonds.

According to the invention, Merkur Weissoel Pharma 40 from Merkur Vaseline, Shell Ondina® 917, Shell Ondina® 927, Shell Oil 4222, Shell Ondina®933 from Shell & DEA Oil, Pionier® 6301 S, Pionier® 2071 (Hansen & Rosenthal) can be used as paraffin oil advantageous according to the invention. Suitable cosmetically compatible oil and fat components are described in Karl-Heinz Schrader, Grundlagen und Rezepturen der Kosmetika [Fundamentals and formulations of cosmetics], 2nd edition, Verlag Hüthig, Heidelberg, pp. 319-355, to the entire scope of which reference is hereby made.

### Solvents

If the keratin-binding effector molecules according to the invention and/or produced according to the inventive method are used in cosmetic or dermatological preparations which are a solution or emulsion or dispersion, solvents which can be used are:

water or aqueous solutions; oils, such as triglycerides of capric acid or caprylic acid, but preferably castor oil; fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids; alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products. In particular, mixtures of the abovementioned solvents are used. In the case of alcoholic solvents, water may be a further constituent.

### Surfactants

According to the invention, besides the keratin-binding effector molecules according to the invention and/or produced according to the inventive method, compositions can also comprise surfactants. Such surfactants are, for example:
phosphoric esters and salts, such as, for example, DEA-oleth-10 phosphate and dilaureth-4 phosphate,
alkylsulfonates, for example sodium coconut monoglyceride sulfate, sodium C₁₂₋₁₄ olefinsulfonate, sodium lauryl sulfoacetate and magnesium PEG-3 cocamide sulfate,
carboxylic acids and derivatives, such as, for example, lauric acid, aluminum stearate, magnesium alkanolate and zinc undecylenate, ester carboxylic acids, for example calcium stearoyl lactylate, laureth-6 citrate and sodium PEG-4 lauramide carboxylate,
esters which are formed by esterification of carboxylic acids with ethylene oxide, glycerol, sorbitan or other alcohols,
ethers, for example ethoxylated alcohols, ethoxylated lanolin, ethoxylated polysiloxanes, propoxylated POE ethers and alkyl polyglycosides, such as lauryl glucoside, decyl glycoside and cocoglycoside.

### Polysorbates

According to the invention, besides the keratin-binding effector molecules according to the invention and/or produced according to the inventive method, compositions may also comprise polysorbates. Polysorbates advantageous for the purposes of the invention here are
polyoxyethylene(20) sorbitan monolaurate (Tween 20, CAS No. 9005-64-S)
polyoxyethylene(4) sorbitan monolaurate (Tween 21, CAS No. 9005-64-5)
polyoxyethylene(4) sorbitan monostearate (Tween 61, CAS No. 9005-67-8)
polyoxyethylene(20) sorbitan tristearate (Tween 65, CAS No. 9005-71-4)
polyoxyethylene(20) sorbitan monooleate (Tween 80, CAS No. 9005-65-6)
polyoxyethylene(5) sorbitan monooleate (Tween 81, CAS No. 9005-65-5)
polyoxyethylene(20) sorbitan trioleate (Tween 85, CAS No. 9005-70-3).

Particularly advantageous are, in particular,
polyoxyethylene(20) sorbitan monopalmitate (Tween 40, CAS No, 9005-66-7)
polyoxyethylene(20) sorbitan monostearate (Tween 60, CAS No. 9005-67-8).

According to the invention, these are advantageously used in a concentration of from 0.1 to 5% by weight and in particular in a concentration of from 1.5 to 2.5% by weight, based on the total weight of the composition, individually or as a mixture of two or more polysorbates.

### Conditioning Agents

In a preferred embodiment of the invention, the compositions also comprise conditioning agents. Conditioning agents preferred according to the invention are, for example, all compounds which are listed in the International Cosmetic Ingredient Dictionary and Handbook (Volume 4, editor: R. C. Pepe, J. A. Wenninger, G. N. McEwen, The Cosmetic, Toiletry, and Fragrance Association, 9th edition, 2002) under section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive and Skin Protectants, and all compounds listed in EP-A 934 956 (pp. 11-13) under “water soluble conditioning agent” and “oil soluble conditioning agent”. Further advantageous conditioning agents are, for example, the compounds referred to in accordance with INCI as Polyquaternium (in particular Polyquaternium-1 to Polyquaternium-56).

Suitable conditioning agents also include, for example, polymeric quaternary ammonium compounds, cationic cellulose derivatives and polysaccharides.

Conditioning agents advantageous according to the invention can here be chosen from the compounds shown in the table below.

TABLE 4
Conditioning agents to be used advantageously

| | | | Example |
|---|---|---|---|
| INCI name | CAS number | Type of polymer | (trade name) |
| Polyquaternium-2 | CAS 63451-27-4 | Urea, N,N′-bis[3-(dimethylamino)propyl] | Mirapol ® A-15 |
| | | polymer with 1,1′-oxybis(2-chloroethane) | |
| Polyquaternium-5 | CAS 26006-22-4 | Acrylamide, β-methacryloxyethyltriethyl- | |
| | | ammonium methosulfate | |
| Polyquaternium-6 | CAS 26062-79-3 | N,N-Dimethyl-N-2-propenyl-2- | Merquat ® 100 |
| | | propenaminium chloride | |
| Polyquaternium-7 | CAS 26590-05-6 | N,N-Dimethyl-N-2-propenyl-2- | Merquat ® S |
| | | propenaminium chloride, 2-propenamide | |
| Polyquaternium- | CAS 53568-66-4, 55353- | Quaternary ammonium salt of | Celquat ® SC- |
| 10 | 19-0, 54351-50-7, 68610- | hydroxyethylcellulose | 230M, Polymer |
| | 92-4, 81859-24-7 | | JR 400 |
| Polyquaternium- | CAS 53633-54-8 | Vinylpyrrolidone/dimethylaminoethyl | Gafquat ® |
| 11 | | methacrylate copolymer/diethyl sulfate | 755N |
| | | reaction product | |
| Polyquaternium- | CAS 29297-55-0 | Vinylpyrrolidone/vinylimidazolinum | Luviquat ® |
| 16 | | methochloride copolymer | HM552 |
| Polyquaternium- | CAS 90624-75-2 | | Mirapol ® AD-1 |
| 17 | | | |
| Polyquaternium- | CAS 110736-85-1 | Quaternized water-soluble polyvinyl | |
| 19 | | alcohol | |
| Polyquaternium- | CAS 110736-86-2 | Quaternized polyvinyl octadecyl ether | |
| 20 | | dispersible in water | |
| Polyquaternium- | | Polysiloxane | Abil ® B 9905 |
| 21 | | polydimethyldimethylammonium acetate | |
| | | copolymer | |
| Polyquaternium- | CAS 53694-17-0 | Dimethyldiallylammonium chloride/acrylic | Merquat ® 280 |
| 22 | | acid copolymer | |
| Polyquaternium- | CAS 107897-23-5 | Polymeric quaternary ammonium salt of | Quartisoft ® |
| 24 | | hydroxyethylcellulose | LM-200 |
| Polyquaternium- | CAS 131954-48-8 | Vinylpyrrolidone/methacrylamidopropyl- | Gafquat ® HS- |
| 28 | | trimethylammonium chloride copolymer | 100 |
| Polyquaternium- | CAS 92091-36-6, | Chitosan which has been reacted with | Lexquat ® CH |
| 29 | 148880-30-2 | propylene oxide and quaternized with | |
| | | epichlorohydrin | |
| Polyquaternium- | CAS 136505-02-7, | Polymeric quaternary ammonium salt | Hypan ® QT |
| 31 | 139767-67-7 | which is prepared by reacting DMAPA | 100 |
| | | acrylate/acrylic acid/acrylonitrogen acid | |
| | | copolymers and diethyl sulfate | |
| Polyquaternium- | CAS 35429-19-7 | N,N,N-Trimethyl-2-([2-methyl-1-oxo-2- | |
| 32 | | propenyl)oxy]ethanaminium chloride, | |
| | | polymer with 2-propenamide | |
| Polyquaternium- | CAS 26161-33-1 | | |
| 37 | | | |
| Polyquaternium- | | Copolymeric quaternary ammonium salt | |
| 44 | | of vinylpyrrolidone and quaternized | |
| | | imidazoline | |

Further conditioners advantageous according to the invention are cellulose derivatives and quaternized guar gum derivatives, in particular guar hydroxypropylammonium chloride (e.g. Jaguar Excel®, Jaguar C 162® (Rhodia), GAS 65497-29-2, GAS 39421-75-5).

Also, nonionic poly-N-vinylpyrrolidone/polyvinyl acetate copolymers (e.g. Luviskol®VA 64 (BASF Aktiengesellschaft)), anionic acrylate copolymers (e.g. Luviflex®Soft (BASF Aktiengesellschaft)), and/or amphoteric amide/acrylate/methacrylate copolymers (e.g. Amphomer® (National Starch)) can be used advantageously according to the invention as conditioners.

### Powder Raw Materials

An addition of powder raw materials may be generally advantageous. The use of talc is particularly preferred.

### Ethoxylated Glycerol Fatty Acid Esters

According to the invention, besides the keratin-binding effector molecules according to the invention and/or produced by the inventive method, compositions can, if appropriate, also comprise ethoxylated oils chosen from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil PEG-7 ester, PEG-6 caprylic/capric glycerides, hydrogenated palm kernel oil glyceride PEG-6 ester, PEG-20 corn oil glycerides, PEG-18 glyceryl oleate cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-35 castor oil, PEG-80 glyceryl cocoate, PEG-60 almond oil glycerides, PEG-60 evening primrose glycerides, PEG-200, hydrogenated glyceryl palmate and PEG-90 glyceryl isostearate. Preferred ethoxylated oils are PEG-7 glyceryl cocoate, PEG-9 cocoglycerides, PEG-40 hydrogenated castor oil, PEG-200 hydrogenated glyceryl palmate. Ethoxylated glycerol fatty acid esters are used in aqueous cleaning formulations for a variety of purposes. Glycerol fatty acid esters with a low degree of ethoxylation (3-12 ethylene oxide units) usually serve as refatting agents for improving the feel of the skin after drying, glycerol fatty acid esters with a degree of ethoxylation of about 30-50 serve as solubility promoters for nonpolar substances such as perfume oils. Glycerol fatty acid esters with a high degree of ethoxylation are used as thickeners. One aspect all of these substances have in common is that they produce a particular feel on the skin when used on the skin in dilution with water.

### Photoprotective Agents

The use of the keratin-binding effector molecules according to the invention and/or produced according to the inventive method in combination with photoprotective agents in dermocosmetic preparations is likewise in accordance with the invention. These cosmetic and/or dermatological photoprotective compositions are used for cosmetic and/or dermatological photoprotection, and also for the treatment and care of the skin and/or of the hair and as make-up product in decorative cosmetics. These include, for example, sun creams, sun lotions, sun milks, sun oils, sun balsams, sun gels, lip care and lipsticks, concealing creams and sticks, moisturizing creams, lotions, emulsions, face, body and hand creams, hair treatments and rinses, hair-setting compositions, styling gels, hair sprays, roll-on deodorants or eye wrinkle creams, tropicals, sunblocks, aftersun preparations. All preparations comprise at least one keratin-binding effector molecule and one of the specified UV filter substances.

Sun oils are mostly mixtures of different oils with one or more photoprotective filters and perfume oils. The oil components are chosen according to different cosmetic properties. Oils which grease well and convey a soft feel to the skin, such as mineral oils (e.g. paraffin oils) and fatty acid triglycerides (e.g. peanut oil, sesame oil, avocado oil, medium-chain triglycerides), are mixed with oils which improve the spreadability and the absorption of the sun oils into the skin, reduce the stickiness and make the oil film permeable for air and water vapor (perspiration). These include branched-chain fatty acid esters (e.g. isopropyl palmitate) and silicone oils (e.g. dimethylsilicone). When using oils based on unsaturated fatty acids, antioxidants, e.g. E-tocopherol, are added in order to prevent them from becoming rancid. Sun oils, being anhydrous formulations, usually comprise no preservatives. Sun milks and sun creams are prepared as oil-in-water (O/W) emulsions and as water-in-oil (W/O) emulsions. Depending on the type of emulsion, the properties of the preparations are very variable: O/W emulsions are readily spreadable on the skin, they mostly absorb rapidly and can almost always be readily washed off with water. W/O emulsions are more difficult to rub in, they grease the skin to a more considerable degree and thus seem to be somewhat stickier, but on the other hand better protect the skin from drying out. W/O emulsions are mostly water-resistant. In the case of O/W emulsions, the emulsion basis, the selection of suitable photoprotective substances and, if appropriate, the use of auxiliaries (e.g. polymers) determine the degree of water resistance. The bases of liquid and cream-like O/W emulsions resemble other emulsions customary in skin care in terms of their composition. Sun milks should sufficiently grease skin dried out by sun, water and wind. They must not be sticky since this is perceived as being particularly unpleasant in the heat and upon contact with sand. The photoprotective agents are generally based on a carrier which comprises at least one oil phase. However, compositions solely on an aqueous basis are also possible. Accordingly, oils, oil-in-water and water-in-oil emulsions, fatty acids such as, for example, magnesium stearate, aluminum stearate and/or zinc stearate are used. Biogenic active ingredients are understood as meaning, for example, plant extracts, protein hydrolyzates and vitamin complexes. Customary film formers are, for example, hydrocolloids, such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds.

Suitable photofilter active ingredients are substances which absorb UV rays in the UV-B and/or UV-A region. These are understood as meaning organic substances which are able to absorb ultraviolet rays and release the absorbed energy again in the form of longer-wave radiation, e.g. heat. The organic substances may be oil-soluble or water-soluble. Suitable UV filters are e.g. 2,4,6-triaryl-1,3,5-triazines in which the aryl groups can each carry at least one substituent which is preferably chosen from hydroxy, alkoxy, specifically methoxy, alkoxycarbonyl, specifically methoxycarbonyl and ethoxycarbonyl. Also suitable are p-aminobenzoic esters, cinnamic esters, benzophenones, camphor derivatives, and pigments which stop UV rays, such as titanium dioxide, talc and zinc oxide. Pigments based on titanium dioxide are particularly preferred.

Oil-soluble UV-B filters which may be used are, for example, the following substances:

3-benzylidenecamphor and derivatives thereof, e.g. 3-(4-methylbenzylidene)camphor;
4-aminobenzoic acid derivatives, preferably 2-ethylhexyl 4-(dimethylamino)benzoate, 2-octyl 4-(dimethylamino)benzoate and amyl 4-(dimethylamino)benzoate;
esters of cinnamic acid, preferably 2-ethylhexyl 4-methoxycinnamate, propyl 4-methoxycinnamate, isoamyl 4-methoxycinnamate, isopentyl 4-methoxycinnamate, 2-ethylhexyl 2-cyano-3-phenylcinnamate (octocrylene);
esters of salicylic acid, preferably 2-ethylhexyl salicylate, 4-isopropylbenzyl salicylate, homomethyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 2-ethylhexyl 4-methoxybenzmalonate;
triazine derivatives, such as, for example, 2,4,6-trianilino-(p-carbo-2′-ethyl-1′-hexyloxy)-1,3,5-triazine (octyltriazone) and dioctylbutamidotriazone (Uvasorb® HEB);
propane-1,3-diones, such as, for example, 1-(4-tert-butylphenyl)-3-(4′-methoxyphenyl)propane-1,3-dione.

Suitable water-soluble substances are:

2-phenylbenzimidazole-5-sulfonic acid and the alkali metal, alkaline earth metal, ammonium alkylammonium, alkanolammonium and glucammonium salts thereof;
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid and 2-methyl-5-(2-oxo-3-bornylidene)sulfonic acid and salts thereof.

Particular preference is given to the use of esters of cinnamic acid, preferably 2-ethylhexyl 4-methoxycinnamate, isopentyl 4-methoxycinnamate, 2-ethylhexyl 2-cyano-3-phenylcinnamate (octocrylene).

Furthermore, the use of derivatives of benzophenone, in particular 2-hydroxy-4-methoxybenzophenone, 2-hydroxy-4-methoxy-4′-methylbenzophenone, 2,2′-dihydroxy-4-methoxybenzophenone, and the use of propane-1,3-diones, such as, for example, 1-(4-tert-butylphenyl)-3-(4′-methoxyphenyl)propane-1,3-dione is preferred.

Suitable typical UV-A filters are:

derivatives of benzoylmethane, such as, for example, 1-(4′-tert-butylphenyl)-3-(4′-methoxyphenyl)propane-1,3-dione, 4-tert-butyl-4′-methoxydibenzoylmethane or 1-phenyl-3-(4′-isopropylphenyl)propane-1,3-dione;
aminohydroxy-substituted derivatives of benzophenones, such as, for example, N,N-diethylaminohydroxybenzoyl n-hexylbenzoate.

The UV-A and UV-B filters can of course also be used in mixtures.

Further suitable UV filter substances are given in the table below.

TABLE 5
Suitable photoprotective agents

| | | CAS No. |
|---|---|---|
| No. | Substance | (=acid) |
| 1 | 4-Aminobenzoic acid | 150-13-0 |
| 2 | 3-(4′-Trimethylammonium)benzylidenebornan-2-one methyl sulfate | 52793-97-2 |
| 3 | 3,3,5-Trimethylcyclohexyl salicylate | 118-56-9 |
| | (homosalate) | |
| 4 | 2-Hydroxy-4-methoxybenzophenone | 131-57-7 |
| | (oxybenzone) | |
| 5 | 2-Phenylbenzimidazole-5-sulfonic acid and its potassium, sodium | 27503-81-7 |
| | and triethanolamine salts | |
| 6 | 3,3′-(1,4-Phenylenedimethine)bis(7,7-dimethyl- | 90457-82-2 |
| | 2-oxobicyclo[2.2.1]heptane-1-methanesulfonic acid) and its salts | |
| 7 | Polyethoxyethyl 4-bis(polyethoxy)aminobenzoate | 113010-52-9 |
| 8 | 2-Ethylhexyl 4-dimethylaminobenzoate | 21245-02-3 |
| 9 | 2-Ethylhexyl salicylate | 118-60-5 |
| 10 | 2-Isoamyl 4-methoxycinnamate | 71617-10-2 |
| 11 | 2-Ethylhexyl 4-methoxycinnamate | 5466-77-3 |
| 12 | 2-Hydroxy-4-methoxybenzophenone-5-sulfonic acid | 4065-45-6 |
| | (sulisobenzone) and the sodium salt | |
| 13 | 3-(4′-Sulfobenzylidene)bornan-2-one and salts | 58030-58-6 |
| 14 | 3-Benzylidenebornan-2-one | 16087-24-8 |
| 15 | 1-(4′-Isopropylphenyl)-3-phenylpropane-1,3-dione | 63260-25-9 |
| 16 | 4-Isopropylbenzyl salicylate | 94134-93-7 |
| 17 | 3-Imidazol-4-ylacrylic acid and its ethyl ester | 104-98-3 |
| 18 | Ethyl 2-cyano-3,3-diphenylacrylate | 5232-99-5 |
| 19 | 2′-Ethylhexyl 2-cyano-3,3-diphenylacrylate | 6197-30-4 |
| 20 | Menthyl o-aminobenzoate or: | 134-09-8 |
| | 5-methyl-2-(1-methylethyl) 2-aminobenzoate | |
| 21 | Glyceryl p-aminobenzoate or: | 136-44-7 |
| | 1-glyceryl 4-aminobenzoate | |
| 22 | 2,2′-Dihydroxy-4-methoxybenzophenone (dioxybenzone) | 131-53-3 |
| 23 | 2-Hydroxy-4-methoxy-4-methylbenzophenone | 1641-17-4 |
| | (mexenone) | |
| 24 | Triethanolamine salicylate | 2174-16-5 |
| 25 | Dimethoxyphenylglyoxalic acid or: | 4732-70-1 |
| | 3,4-dimethoxyphenylglyoxal acidic sodium | |
| 26 | 3-(4′-Sulfobenzylidene)bornan-2-one and its salts | 56039-58-8 |
| 27 | 4-tert-Butyl-4′-methoxydibenzoylmethane | 70356-09-1 |
| 28 | 2,2′,4,4′-Tetrahydroxybenzophenone | 131-55-5 |
| 29 | 2,2′-Methylenebis[6-(2H-benzotriazol-2-yl)-4-(1,1,3,3,- | 103597-45-1 |
| | tetramethylbutyl)phenol] | |
| 30 | 2,2′-(1,4-Phenylene)bis-1H-benzimidazole-4,6- | 180898-37-7 |
| | disulfonic acid, Na salt | |
| 31 | 2,4-bis[4-(2-Ethylhexyloxy)-2-hydroxy]phenyl- | 187393-00-6 |
| | 6-(4-methoxyphenyl)-(1,3,5)-triazine | |
| 32 | 3-(4-Methylbenzylidene)camphor | 36861-47-9 |
| 33 | Polyethoxyethyl 4-bis(polyethoxy)paraaminobenzoate | 113010-52-9 |
| 34 | 2,4-Dihydroxybenzophenone | 131-56-6 |
| 35 | 2,2′-Dihydroxy-4,4′-dimethoxybenzophenone-5,5′- | 3121-60-6 |
| | disodium sulfonate | |
| 36 | Benzoic acid, 2-[4-(diethylamino)-2-hydroxybenzoyl], hexyl ester | 302776-68-7 |
| 37 | 2-(2H-Benzotriazol-2-yl)-4-methyl-6-[2-methyl-3-[1,3,3,3-tetramethyl-1- | 155633-54-8 |
| | [(trimethylsilyl)oxy]disiloxanyl]propyl]phenol | |
| 38 | 1,1-[(2,2′-Dimethylpropoxy)carbonyl]-4,4-diphenyl-1,3-butadiene | 363602-15-7 |

Besides the two abovementioned groups of primary photoprotective substances, it is also possible to use secondary photoprotective agents of the antioxidant type which interrupt the photochemical reaction chain which is triggered when UV radiation penetrates into the skin. Typical examples thereof are superoxide dismutase, catalase, tocopherols (vitamin E) and ascorbic acid (vitamin C).

A further group are antiirritants which have an antiinflammatory effect on skin damaged by UV light. Such substances are, for example, bisabolol, phytol and phytantriol.

Likewise in accordance with the invention is the use of the keratin-binding effector molecules according to the invention and/or produced according to the inventive method in combination with inorganic pigments which stop UV rays in dermocosmetic preparations. Preference is given to pigments based on metal oxides and/or other metal compounds which are insoluble or sparingly soluble in water and chosen from the group of oxides of zinc (ZnO), titanium (TiO₂), iron (e.g. Fe₂O₃,), zirconium (ZrO₂), silicon (SiO₂), manganese (e.g. MnO), aluminum (Al₂O₃), cerium (e.g. Ce₂O₃), mixed oxides of the corresponding metals and mixtures of such oxides.

The inorganic pigments can be present here in coated form, i.e. are treated superficially. This surface treatment can consist, for example, in providing the pigments with a thin hydrophobic layer by a method known per se, as described in DE-A-33 14 742.

Suitable repellent active ingredients are compounds which are able to repel or drive away certain animals, in particular insects, from humans. These include, for example, 2-ethyl-1,3-hexanediol, N,N-diethyl-m-toluamide etc. Suitable hyperemic substances, which stimulate the flow of blood through the skin, are e.g. essential oils, such as dwarf pine extract, lavender extract, rosemary extract, juniperberry extract, horsechest nut extract, birch leaf extract, hayflower extract, ethyl acetate, camphor, menthol, peppermint oil, eucalyptus oil, etc. Suitable keratolytic and keratoplastic substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counteract skin irritations, are, for example, allantoin, bisabolol, dragosantol, camomile extract, panthenol, etc.

The use of the keratin-binding effector molecules according to the invention and/or produced according to the inventive method in combination with at least one cosmetically or pharmaceutically acceptable polymer is likewise in accordance with the invention.

Suitable polymers are, for example, cationic polymers with the INCI name Polyquaternium, e.g. copolymers of vinylpyrrolidone/N-vinylimidazolium salts (Luviquat FC, Luviquat HM, Luviquat MS, Luviquat), copolymers of N-vinylpyrrolidone/dimethylaminoethyl methacrylate, quaternized with diethyl sulfate (Luviquat PQ 11), copolymers of N-vinylcaprolactam/N-vinylpyrrolidone/N-vinylimidazolium salts (Luviquat E Hold), cationic cellulose derivatives (Polyquaternium-4 and -10), acrylamide copolymers (Polyquaternium-7) and chitosan.

Suitable cationic (quaternized) polymers are also Merquat (polymer based on dimethyldiallylammonium chloride), Gafquat (quaternary polymers which are formed by reacting polyvinylpyrrolidone with quaternary ammonium compounds), polymer JR (hydroxyethylcellulose with cationic groups) and plant-based cationic polymers, e.g. guar polymers, such as the Jaguar grades from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example Luviflex 0 Swing (partially hydrolyzed copolymer of polyvinyl acetate and polyethylene glycol, BASF Aktiengesellschaft).

Suitable polymers are also nonionic, water-soluble or water-dispersible polymers or oligomers, such as polyvinylcaprolactam, e.g. Luviskol 0 Plus (BASF), or polyvinylpyrrolidone and copolymers thereof, in particular with vinyl esters, such as vinyl acetate, e.g. Luviskol 0 VA 37 (BASF), polyamides, e.g. based on itaconic acid and aliphatic diamines, as are described, for example, in DE-A43 33 238.

Suitable polymers are also amphoteric or zwitterionic polymers, such as the octylacrylamide/methyl methacrylate/tert-butylaminoethyl methacrylate-hydroxypropyl methacrylate copolymers obtainable under the names Amphomer (National Starch), and zwitterionic polymers, as are disclosed, for example, in the German patent applications DE39 29 973, DE 21 50 557, DE28 17 369 and DE 3708 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/methacrylate copolymers, which are commercially available under the name Amersette (AMERCHOL), and copolymers of hydroxyethyl methacrylate, methyl methacrylate, N,N-dimethylaminoethyl methacrylate and acrylic acid (Jordapon (D)).

Suitable polymers are also nonionic, siloxane-containing, water-soluble or -dispersible polymers, e.g. polyether siloxanes, such as Tegopren 0 (Goldschmidt) or Besi&commat (Wacker).

Likewise in accordance with the invention is the use of the keratin-binding effector molecules according to the invention and/or produced according to the inventive method in combination with dermocosmetic active ingredients (one or more compounds) advantageously chosen from the group consisting of acetylsalicylic acid, atropine, azulene, hydrocortisone and derivatives thereof, e.g. hydrocortisone-17-valerate, vitamins of the B and D series, in particular vitamin B₁, vitamin B₁₂, vitamin D, vitamin A or derivatives thereof, such as retinyl palmitate, vitamin E or derivatives thereof such as, for example, tocopheryl acetate, vitamin C and deriatives thereof, such as, for example, ascorbyl glucoside, but also niacinamide, panthenol, bisabolol, polydocanol, unsaturated fatty acids, such as, for example, the essential fatty acids (usually referred to as vitamin F), in particular □-linolenic acid, oleic acid, eicosapentaenoic acid, docosahexaenoic acid and derivatives thereof, chloramphenicol, caffeine, prostaglandins, thymol, camphor, squalene, extracts or other products of vegetable and animal origin, e.g. evening primrose oil, borage oil or carob seed oil, fish oils, cod-liver oil or ceramides and ceramide-like compounds, incense extract, green tea extract, water lily extract, licorice extract, hamamelis, antidandruff active ingredients (e.g. selenium disulfide, zinc pyrithione, piroctone olamine, climbazole, octopirox, polydocanol and combinations thereof), complex active ingredients, such as, for example, those of □-oryzanoland calcium salts, such as calcium pantothenate, calcium chloride, calcium acetate. It is also advantageous to choose the active ingredients from the group of refatting substances, for example purcellin oil, Eucerit® and Neocerit®. The active ingredient or active ingredients are also particularly advantageously chosen from the group of NO synthesis inhibitors, particularly if the preparations according to the invention are to be used for the treatment and prophylaxis of the symptoms of intrinsic and/or extrinsic skin aging, and for the treatment and prophylaxis of the harmful effects of ultraviolet radiation on the skin and the hair. A preferred NO synthesis inhibitor is nitroarginine. The active ingredient or active ingredients are further advantageously chosen from the group comprising catechins and bile acid esters of catechins and aqueous or organic extracts from plants or parts of plants which have a content of catechins or bile acid esters of catechins, such as, for example, the leaves of the Theaceae plant family, in particular of the species Camellia sinensis (green tea). Their typical ingredients (e.g. polyphenols or catechins, caffeine, vitamins, sugars, minerals, amino acids, lipids) are particularly advantageous. Catechins are a group of compounds which are to be understood as hydrogenated flavones or anthocyanidins and represent derivatives of “catechin” (catechol, 3,3′,4′,5,7-flavanpentaol, 2-(3,4-dihydroxyphenyl)chroman-3,5,7-triol). Epicatechin ((2R,3R)-3,3′,4′,5,7-flavanpentaol) is an advantageous active ingredient for the purposes of the present invention. Also advantageous are plant extracts with a content of catechins, in particular extracts of green tea, such as, for example, extracts from leaves of the plants of the species Camellia spec., very particularly the tea types Camellia sinenis, C. assamica, C. taliensis and C. inawadiensis and hybrids of these with, for example, Camellia japonica. Preferred active ingredients are also polyphenols and catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (often also collectively called “flavones”) are advantageous active ingredients for the purposes of the present invention. They are characterized by the following basic structure (substitution positions given):

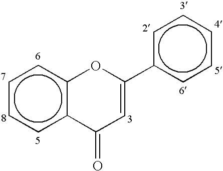

Some of the more important flavones, which can also preferably be used in preparations according to the invention are listed in Table 6 below.

TABLE 6
Flavones

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

Flavones usually occur in nature in glycosylated form.

According to the invention, the flavonoids are preferably chosen from the group of substances of the general formula

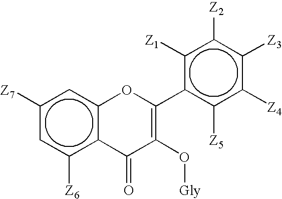

where Z₁ to Z₇, independently of one another, are chosen from the group H, OH, alkoxy and hydroxyalkoxy groups, where the alkoxy or hydroxyalkoxy groups may be branched or unbranched and have 1 to 18 carbon atoms, and where Gly is chosen from the group of mono- and oligoglycoside radicals.

Furthermore, the active ingredients (one or more compounds) can also very advantageously be chosen from the group of hydrophilic active ingredients, in particular from the following group:

□-hydroxy acids, such as lactic acid or salicylic acid or salts thereof, such as, for example, Na lactate, Ca lactate, TEA lactate, urea, allantoin, serine, sorbitol, glycerol, milk proteins, panthenol, chitosan.

The amount of such active ingredients (one or more compounds) in the preparations according to the invention is preferably 0.001 to 30% by weight, particularly preferably 0.05 to 20% by weight, in particular 1 to 10% by weight, based on the total weight of the preparation. The specified active ingredients and further active ingredients which can be used in the preparations according to the invention are given in DE 103 18 526 A1 on pages 12 to 17, to the entire scope of which reference is made at this point.

In addition, the present invention relates to the use of the abovementioned preparations for preventing undesired changes in the appearance of the skin, such as, for example acne or greasy skin, keratoses, rosaceae, photosensitive, inflammatory, erythematous, allergic or autoimmune-reactive reactions.

For use, the cosmetic preparations according to the invention are applied to the skin, hair, fingernails or toenails in the manner customary for cosmetics or dermocosmetics.

The present invention further provides dermocosmetics comprising one of the above-described keratin-binding effector proteins, particularly preferably keratin-binding effector proteins chosen from the group consisting of enzymes, antibodies, effector-binding proteins, fluorescent proteins, antimicrobial peptides and self-assembling proteins. Particular preference is given to dermocosmetics comprising a keratin-binding effector molecule as described in Example 3.

Most preferred of all are dermocosmetics comprising keratin-binding effector proteins which comprise at least one keratin-binding polypeptide (ii) according to the sequences shown in SEQ ID No.: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164 or 166, preferably in SEQ ID No: 2, 4, 6, 8, 10, 12, 14, 40, 42, 44, 46, 48, 146, 150, 153, 156, 157, 158, 160, 162, 164, 166, 213 or 215, and the effector polypeptide (ii) is a silk protein, preferably one of the in the sequences SEQ ID No.: 151, 201, 202, 203, 204, 205, 206, 207, 208, 209 or 210, particularly preferably the C16 spider silk protein, which is a 16-fold repetition of the C modulus of the protein ADF4 from Araneus diadematus.

In a preferred embodiment of the present invention, the dermocosmetics, preferably skin- and hair-treatment compositions comprise a keratin-binding effector protein according to the invention in a concentration of from 0.001 to 1 percent by weight (% by wt.), preferably 0.01 to 0.9% by weight, particularly preferably 0.01 to 0.8% by weight or 0.01 to 0.7% by weight, very particularly preferably 0.01 to 0.6% by weight or 0.01 to 0.5% by weight most preferably 0.01 to 0.4% by weight or 0.01 to 0.3% by weight, based on the total weight of the composition. In a further embodiment, the compositions comprise a keratin-binding effector protein according to the invention in a concentration of from 1 to 10% by weight, preferably 2 to 8% by weight, 3 to 7% by weight, 4 to 6% by weight, based on the total weight of the composition. In a likewise preferred embodiment, the compositions comprise a keratin-binding effector protein according to the invention in a concentration of from 10 to 20% by weight, preferably 11 to 19% by weight, 12 to 18% by weight, 13 to 17% by weight, 14 to 16% by weight, based on the total weight of the composition. In a moreover preferred embodiment, the compositions comprise a keratin-binding effector protein according to the invention in a concentration of from 20 to 30% by weight preferably 21 to 29% by weight, 22 to 28% by weight, 23 to 27% by weight, 24 to 26% by weight, based on the total weight of the composition.

The compositions according to the invention are preferably skin protection compositions, skincare compositions, skin cleansing compositions, hair protection compositions, haircare compositions, hair cleansing compositions, hair colorants, mouthwashes and mouth rinses, or preparation for decorative cosmetics, which are preferably applied in the form of ointments, creams, emulsions, suspensions, lotions, milk, pastes, gels, foams or sprays, depending on the field of application.

Besides the keratin-binding effector proteins, the dermocosmetics according to the invention can comprise all of the polymers, pigments, humectants, oils, waxes, enzymes, minerals, vitamins, sunscreens, dyes, fragrances, antioxidants, preservatives and/or pharmaceutical active ingredients which have already been listed above.

Additionally, the following applies for the dermocosmetics according to the invention:

The formulation base of compositions according to the invention preferably comprises cosmetically or dermocosmetically/pharmaceutically acceptable auxiliaries. Pharmaceutically acceptable auxiliaries are the auxiliaries which are known for use in the field of pharmacy, food technology and related fields, in particular the auxiliaries listed in the relevant pharmacopoeia (e.g. DAB Ph. Eur. BP NF), and other auxiliaries whose properties do not preclude a physiological application.

Suitable auxiliaries may be: glidants, wetting agents, emulsifying and suspending agents, preservatives, antioxidants, antiirritatives, chelating agents, emulsion stabilizers, film formers, gel formers, odor masking agents, resins, hydrocolloids, solvents, solubility promoters, neutralizing agents, permeation accelerators, pigments, quaternary ammonium compounds, refatting and superfatting agents, ointment, cream or oil base substances, silicone derivatives, stabilizers, sterilizing agents, propellants, drying agents, opacifiers, thickeners, waxes, softeners, white oil. An embodiment in this regard is based on specialist knowledge, as shown, for example, in Fiedler, H. P. Lexikon der Hilfsstoffe für Pharmazie, Kosmetik und angrenzende Gebiete [Lexicon of auxiliaries for pharmacy, cosmetics and related fields], 4th edition, Aulendorf: ECV-Editio-Kantor-Verlag, 1996.

To produce the dermocosmetic compositions according to the invention, the active ingredients can be mixed or diluted with a suitable auxiliary (excipient). Excipients may be solid, semisolid or liquid materials which can serve as vehicles, carriers or medium for the active ingredient. The admixing of further auxiliaries takes place, if desired, in the manner known to the person skilled in the art. In addition, the polymers and dispersions are suitable as auxiliaries in pharmacy, preferably as or in (a) coating composition(s) or binder(s) for solid drug forms. They can also be used in creams and as tablet coatings and tablet binders.

According to a further preferred embodiment, the compositions according to the invention are cosmetic compositions for the care and protection of the skin and hair, nailcare compositions or preparations for decorative cosmetics.

Suitable skin cosmetic compositions are, for example, face tonics, face masks, deodorants and other cosmetic lotions. Compositions for use in decorative cosmetics include, for example, concealing sticks, stage make-up, mascara and eye shadows, lipsticks, kohl pencils, eyeliners, blushers, powders and eyebrow pencils.

Furthermore, the keratin-binding effector molecules according to the invention and/or produced according to the inventive method are used in nose strips for pore cleansing, in antiacne compositions, repellents, shaving compositions, aftershave and preshave care compositions, aftersun care compositions, hair removal compositions, hair colorants, intimate care compositions, footcare compositions, and in baby care.

The skincare compositions according to the invention are, in particular, W/O or O/W skin creams, day creams and night creams, eye creams, face creams, antiwrinkle creams, sunscreen creams, moisturizing creams, bleaching creams, self-tanning creams, vitamin creams, skin lotions, care lotions and moisturizing lotions.

Skin cosmetic and dermatological compositions according to the invention can also comprise an active ingredient which decomposes free radicals as protection against oxidative processes and the associated aging processes or damage to skin and/or hair, besides the keratin-binding effector molecule according to the invention and/or produced according to the inventive method. These active ingredients are preferably the substances described in the patent applications WO/0207698 and WO/03059312, to the contents of which reference is hereby expressly made, preferably the boron-comprising compounds described therein, which can reduce peroxides or hydroperoxides to give the corresponding alcohols without the formation of free-radical subsequent states. In addition, sterically hindered amines according to the general formula 3 can be used for this purpose,

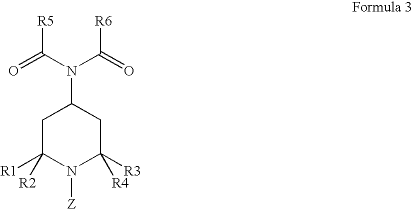

where the radical Z has the following meaning: H, C₁-C₂₂ alkyl group, preferably C₁-C₁₂ alkyl group, such as methyl, ethyl, propyl, isopropyl, butyl, isobutyl, sec-butyl, tert-butyl, pentyl, isopentyl, neopentyl, tert-pentyl, hexyl, heptyl, octyl, nonyl, decyl, undecyl, dodecyl, C₁-C₂₂-alkoxyl group, preferably C₁-C₁₂-alkoxyl group, such as alkoxy-methyl, alkoxy-ethyl, alkoxy-propyl, alkoxy-isopropyl, alkoxy-butyl, alkoxy-isobutyl, alkoxy-sec-butyl, alkoxy-tert-butyl, alkoxy-pentyl, alkoxy-isopentyl, alkoxy-neopentyl, alkoxy-tert-pentyl, alkoxy-hexyl, alkoxy-heptyl, alkoxy-octyl, alkoxy-nonyl, alkoxy-decyl, alkoxy-undecyl, alkoxy-dodecyl, C₆ to C₁₀-aryl group, such as phenyl and naphthyl, where the phenyl radical can be substituted by C₁ to C₄ alkyl radicals, C₆ to C₁₀—O-aryl group, which can be substituted by a C₁-C₂₂ alkyl or C₁-C₂₂-alkoxy group, preferably by a C₁-C₁₂ alkyl or C₁-C₁₂-alkoxy group as described above, and
the radicals R¹ to R⁶, independently of one another, have the following meaning: H, OH, O, C₁-C₂₂ alkyl group, preferably C₁-C₁₂ alkyl group, such as methyl, ethyl, propyl, isopropyl, butyl, isobutyl, sec-butyl, tert-butyl, pentyl, isopentyl, neopentyl, tert-pentyl, hexyl, heptyl, octyl, nonyl, decyl, undecyl, dodecyl, C₁-C₂₂ alkoxyl group, preferably C₁-C₁₂ alkoxyl group, such as alkoxy-methyl, alkoxyethyl, alkoxypropyl, alkoxyisopropyl, alkoxybutyl, alkoxyisobutyl, alkoxy-sec-butyl, alkoxy-tert-butyl, alkoxypentyl, alkoxyisopentyl, alkoxyneopentyl, alkoxy-tert-pentyl, alkoxyhexyl, alkoxyheptyl, alkoxyoctyl, alkoxynonyl, alkoxydecyl, alkoxyundecyl, alkoxy-dodecyl, C₆ to C₁₀ aryl group, such as phenyl and naphthyl, where the phenyl radical can be substituted by C₁ to C₄ alkyl radicals, C₆ to C₁₀ O-aryl group, which may be substituted by a C₁-C₂₂ alkyl or C₁-C₂₂ alkoxyl group, preferably by a C₁-C₁₂ alkyl or C₁-C₁₂ alkoxyl group, as described above.

Particular preference is given to the use of the sterically hindered amines 3-dodecyl-N-(2,2,6,6-tetramethyl-4-piperidinyl)succinimide, 3-dodecyl-N-(1,2,2,6,6-pentamethyl-4-piperidinyl)succinimide, 3-octyl-N-(2,2,6,6-tetramethyl-4-piperidinyl)succinimide, 3-octyl-N-(1,2,2,6,6-pentamethyl-4-piperidinyl)succinimide, 3-octenyl-N-(2,2,6,6-tetramethyl-4-piperidinyl)succinimide, 3-octenyl-N-(1,2,2,6,6-pentamethyl-4-piperidinyl)succinimide and/or Uvinul®5050H, in an amount of from 0.001 to 1 percent by weight (% by wt.) preferably 0.01 to 0.1% by weight, 0.1 to 1% by weight, based on the total weight of the composition.

Besides the abovementioned compounds according to the invention and suitable carriers, the skin cosmetic preparations can also comprise further active ingredients and auxiliaries customary in skin cosmetics, as described above. These include, preferably, emulsifiers, preservatives, perfume oils, cosmetic active ingredients, such as phytantriol, vitamin A, E and C, retinol, bisabolol, panthenol, photoprotective agents, bleaches, colorants, tinting agents, tanning agents, collagen, protein hydrolyzates, stabilizers, pH regulators, dyes, salts, thickeners, gel formers, consistency regulators, silicones, humectants, refatting agents and/or further customary additives.

Preferred oil and fat components of the skin cosmetic and dermocosmetic compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C6-C30 fatty acids, wax esters, such as, for example, jojoba oil, fatty alcohols, vaseline, hydrogenated lanolin and acetylated lanolin, and mixtures thereof.

To establish certain properties, such as, for example, improving the feel to the touch, the spreading behavior, the water resistance and/or the binding of active ingredients and auxiliaries such as pigments, the skin cosmetic and dermocosmetic preparations can additionally also comprise conditioning substances based on silicone compounds.

Suitable silicone compounds are, for example, polyalkylsiloxanes, polyarylsiloxanes, polyarylalkylsiioxanes, polyether siloxanes or silicone resins.

The cosmetic or dermocosmetic preparations are produced by customary methods known to the person skilled in the art.

Preferably, the cosmetic and dermocosmetic compositions are present in the form of emulsions, in particular as water-in-oil (W/O) or oil-in-water (O/W) emulsions.

However, it is also possible to choose other types of formulation, for example gels, oils, oleogels, multiple emulsions, for example in the form of W/O/W or O/W/O emulsions, anhydrous ointments or ointment bases, etc. Emulsifier-free formulations, such as hydrodispersions, hydrogels or a Pickering emulsion are also advantageous embodiments.

Emulsions are produced by known methods. Besides at least one keratin-binding effector molecule, the emulsions usually comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The choice of additives specific to the type of emulsion and the production of suitable emulsions is described, for example, in Schrader, Grundlagen und Rezepturen der Kosmetika [Fundamentals and formulations of cosmetics], Hüthig Buch Verlag, Heidelberg, 2nd edition, 1989, third part, or Umbach, Kosmetik: Entwickfung, Herstellung und Anwendung kosmetischer Mittel [Cosmetics: development, manufacture and use of cosmetic compositions], 2nd expanded edition, 1995, Georg Thieme Verlag, ISBN 3 13 712602 9, pages 122 ff., to which reference is hereby expressly made.

A suitable emulsion in the form of a W/O emulsion, e.g. for a skin cream etc., generally comprises an aqueous phase which is emulsified in an oil or fatty phase using a suitable emulsifier system. A polyelectrolyte complex can be used for the provision of the aqueous phase.

Preferred fatty components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil, mineral oils whose distillation start-point under atmospheric pressure is at about 250° C. and whose distillation end-point is at 410° C., such as, for example, Vaseline oil, esters of saturated or unsaturated fatty acids, such as alkyl myristates, e.g., isopropyl myristate, butyl myristate or cetyl myristate, hexadecyl stearate, ethyl or isopropyl palmitate, octanoic or decanoic acid triglycerides and cetyl ricinoleate.

The fatty phase can also comprise silicone oils which are soluble in other oils, such as dimethylpolysiloxane, methylphenylpolysiloxane and the silicone glycol copolymer, fatty acids and fatty alcohols.

Besides the above-described compounds according to the invention, the skin care compositions can also comprise waxes, such as, for example, carnauba wax, candelilia wax, beeswax, microcrystalline wax, ozokerite wax and Ca, Mg and Al creates, myristates, linoieates and stearates.

In addition, an emulsion according to the invention may be in the form of an O/w emulsion. Such an emulsion usually comprises an oil phase, emulsifiers which stabilize the oil phase in the water phase, and an aqueous phase, which is usually present in thickened form. Suitable emulsifiers are preferably O/w emulsifiers, such as polyglycerol esters, sorbitan esters or partially esterified glycerides.

According to a further preferred embodiment the compositions according to the invention are a photoprotective composition, a shower gel, a shampoo formulation or a bath preparation, with photoprotective preparations being particularly preferred.

Such formulations comprise at least one keratin-binding effector molecule according to the invention and/or produced according to the inventive method, and usually anionic surfactants as base surfactants and amphoteric and/or nonionic surfactants as cosurfactants. Further suitable active ingredients and/or auxiliaries are generally chosen from lipids, perfume oils, dyes, organic acids, preservatives and antioxidants, and thickeners/gel formers, skin conditioning agents and humectants.

These formulations advantageously comprise 2 to 50% by weight, preferably 5 to 40% by weight particularly preferably 8 to 30% by weight, of surfactants, based on the total weight of the formulation.

In the washing, shower and bath preparations, all of the anionic, neutral, amphoteric or cationic surfactants customarily used in body-cleansing compositions can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. In addition, alkylamine oxides, mono- or dialkylalkanolamides, fatty acid esters of polyethylene glycols, ethoxylated fatty acid amides, alkyl polyglycosides or sorbitan ether esters are suitable.

Furthermore, the washing, shower and bath preparations can comprise customary cationic surfactants, such as, for example, quaternary ammonium compounds, for example cetyltrimethylammonium chloride.

In addition, the shower gel/shampoo formulations can comprise thickeners, such as, for example, sodium chloride, PEG-55, propylene glycol oleate, PEG-120 methylglucose dioleate and others, and also preservatives, further active ingredients and auxiliaries and water.

### Hair Treatment Compositions

According to a further preferred embodiment, the dermocosmetics according to the invention are hair treatment compositions.

Preferably, the hair treatment compositions according to the invention are in the form of a setting foam, hair mousse, hair gel, shampoo, hair spray, hair foam, end fluid, neutralizer for permanent waves, hair colorant and bleach or hot-oil treatment. Depending on the field of use, the hair cosmetic preparations can be applied as (aerosol) spray, (aerosol) foam, gel, gel spray, cream, lotion or wax. Hair sprays include here both aerosol sprays and also pump sprays without propellant gas. Hair foams include both aerosol foams and also pump foams without propellant gas. Hair sprays and hair foams preferably include predominantly or exclusively water-soluble or water-dispersible components. If the compounds used in the hair sprays and hair foams according to the invention are dispersible in water, they can be applied in the form of aqueous microdispersions with particle diameters of usually 1 to 350 nm, preferably 1 to 250 nm. The solids contents of these preparations are here usually in a range from about 0.5 to 20% by weight. These microdispersions do not usually require emulsifiers or surfactants for their stabilization.

Further constituents are to be understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances, such as panthenol, collagen, vitamins, protein hydrolyzates, alpha- and beta-hydroxycarboxylic acids, stabilizers, pH regulators, dyes, viscosity regulators, gel formers, salts, humectants, refafting agents, complexing agents and further customary additives.

Also included here are all styling and conditioner polymers known in cosmetics which can be used in combination with the keratin-binding effector molecules according to the invention if quite specific properties are to be established.

Suitable conventional hair cosmetics polymers are, for example, the abovementioned cationic, anionic, neutral, nonionic and amphoteric polymers, to which reference is made here.

To establish certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and amino functional silicone compounds, such as amodimethicones (CTFA).

Propellants are the propellants customarily used for hair sprays or aerosol foams. Preference is given to mixtures of propane/butane, pentane, dimethyl ether, 1,1-difluoroethane (HFC-152 a), carbon dioxide, nitrogen or compressed air.

Emulsifiers which can be used are all emulsifiers customarily used in hair foams. Suitable emulsifiers may be nonionic, cationic or anionic or amphoteric. Examples of nonionic emulsifiers (INCI nomenclature) are laureths, e.g. laureth-4; ceteths, e.g. ceteth-1, polyethylene glycol cetyl ether, ceteareths, e.g. ceteareth-25, polyglycol fatty acid glycerides, hydroxylated lecithin, lactyl esters of fatty acids, alkyl polyglycosides.

Examples of cationic emulsifiers are cetyidimethyl-2-hydroxyethylammonium dihydrogenphosphate, cetyltrimonium chloride, cetyltrimonium bromide, cocotrimonium methyl sulfate, quaternium-1 to x (INCI).

Anionic emulsifiers can be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. xanthan gum, caprylicicapric triglyceride, sodium acrylate copolymers, polyquaternium-32 (and) paraffinum liquidum (INCI), sodium acrylate copolymers (and) paraffinum liquidum (and) PPG-1 trideceth-6, acrylamidopropyltrimonium chloride/acrylamide copolymers, steareth-10 alkyl ether, acrylate copolymers, polyquaternium-37 (and) paraffinum liquidum (and) PPG-1 trideceth-6, polyquaternium 37 (and) propylene glycol dicaprate dicaprylate (and) PPG-1 trideceth-6, polyquaternium-7, polyquaternium-44.

In the shampoo formulations, all of the anionic, neutral, amphoteric or cationic surfactants customarily used in shampoos can be used.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isothionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Of suitability are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecylbenzenesulfonate. Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioning agents can be used in combination with the keratin-binding effector molecules according to the invention.

These include, for example, the abovementioned cationic polymers with the INCI name Polyquaternium, in particular copolymers of vinylpyrrolidone/N-vinylimidazolium salts (Luviquat FC, HM, Luviquat MS, Luviquat Care), copolymers of N-vinylpyrrolidone/dimethylaminoethyl methacrylate, quaternized with diethyl sulfate (Luviquat D PQ 11), copolymers of N-vinylcaprolactam/N-vinylpyrrolidone/N-vinylimidazolium salts (Luviquat D Hold), cationic cellulose derivatives (Polyquaternium-4 and -10), acrylamide copolymers (Polyquaternium-7). In addition, protein hydrolyzates can be used, and also conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino-functional silicone compounds, such as amodimethicones (CTFA). In addition, cationic guar derivatives, such as guar Hydroxypropyltrimonium Chloride (INCI) can be used.

According to a further embodiment, this hair cosmetic or skin cosmetic preparation serves for the care or the protection of the skin or hair and is in the form of an emulsion, a dispersion, a suspension, an aqueous surfactant preparation, a milk, a lotion, a cream, a balsam, an ointment, a gel, a granulate, a powder, a stick preparation, such as, for example, a lipstick, a foam, an aerosol or a spray. Such formulations are highly suitable for topical preparations. Suitable emulsions are oil-in-water emulsions and water-in-oil emulsions or microemulsions.

As a rule, the hair cosmetic or skin cosmetic preparation is used for application to the skin (topical) or hair. Topical preparations are understood here as meaning those preparations which are suitable for applying the active ingredients to the skin in a fine distribution and preferably in a form which can be absorbed by the skin. Of suitability for this purpose are, for example, aqueous and aqueous-alcoholic solutions, sprays, foams, foam aerosols, ointments, aqueous gels, emulsions of the O/W or W/O type, microemulsions or cosmetic stick preparations.

According to a preferred embodiment of the cosmetic composition according to the invention, the composition comprises a carrier. A preferred carrier is water, a gas, a water-based liquid, an oil, a gel, an emulsion or microemulsion, a dispersion or a mixture thereof. The specified carriers exhibit good skin compatibility. Of particular advantage for topical preparations are aqueous gels, emulsions or microemulsions.

Emulsifiers which can be used are nonionogenic surfactants, zwitterionic surfactants, ampholytic surfactants or anionic emulsifiers. The emulsifiers may be present in the composition according to the invention in amounts of from 0.1 to 10% by weight, preferably 1 to 5% by weight, based on the composition.

The nonionogenic surfactant used may, for example, be a surfactant from at least one of the following groups:

addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
C_{12/18}-fatty acid mono- and -diesters of addition products of from 1 to 30 mol of ethylene oxide onto glycerol; glycerol mono- and diesters and sorbitan mono- and diesters of saturated and unsaturated fatty acids having 6 to 22 carbon atoms and ethylene oxide addition products thereof; alkyl mono- and oligoglycosides having 8 to 22 carbon atoms in the alkyl radical and ethoxylated analogs thereof; addition products of from 15 to 60 mol of ethylene oxide onto castor oil and/or hydrogenated castor oil; polyol and, in particular polyglycerol esters, such as, for example, polyglycerol polyricinoleate, polyglycerol poly-12-hydroxystearate or polyglycerol dimerate. Likewise suitable are mixtures of compounds from two or more of these classes of substances;
addition products of from 2 to 15 mol of ethylene oxide onto castor oil and/or hydrogenated castor oil; partial esters based on linear, branched, unsaturated or saturated C_{6/22} fatty acids, ricinoleic acid, and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose); mono-, di- and trialkyl phosphates, and mono-, di- and/or tri-PEG alkyl phosphates and salts thereof;
wool wax alcohols;
polysiloxane-polyalkyl polyether copolymers and corresponding derivatives;
mixed esters of pentaerythritol, fatty acids, citric acid and fatty alcohol as in German patent specification 1165574 and/or mixed esters of fatty acids having 6 to 22 carbon atoms, methylglucose and polyols, preferably glycerol or polyglycerol, and polyalkylene glycols.

In addition, zwitterionic surfactants can be used as emulsifiers. Zwitterionic surfactants is the term used to refer to those surface-active compounds which carry at least one quaternary ammonium group and at least one carboxylate group or a sulfonate group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxylmethyl-3-hydroxyethylimidazolines having in each case 8 to 18 carbon atoms in the alkyl or acyl group, and cocoacylaminoethylhydroxyethyl carboxymethylglycinate. Particular preference is given to the fatty acid amide derivative known under the CTFA name Cocamidopropyl Betaine.

Likewise suitable emulsifiers are ampholytic surfactants. Ampholytic surfactants are understood as meaning those surface-active compounds which, apart from a C_{8,18}-alkyl or -acyl group in the molecule, comprise at least one free amino group and at least one —COOH— or —SO₃H group, and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group.

Particularly preferred ampholytic surfactants are N-cocoalkylaminopropionate, cocoacylaminoethylaminopropionate and C_{12/18}-acylsarcosine. Besides the ampholytic emulsifiers, quaternary emulsifiers are also suitable, with those of the ester quat type, preferably methyl-quaternized difatty acid triethanolamine ester salts, being particularly preferred. Furthermore, anionic emulsifiers which may be used are alkyl ether sulfates, monoglyceride sulfates, fatty acid sulfates, sulfosuccinates and/or ether carboxylic acids.

Suitable oil substances are Guerbet alcohols based on fatty alcohols having 6 to 18, preferably 8 to 10, carbon atoms, esters of linear C₆-C₂₂-fatty acids with linear C₆-C₂₂-fatty alcohols, esters of branched C₆-C₁₃-carboxylic acids with linear C₆-C₂₂-fatty alcohols, esters of linear C₆-C₂₂-fatty acids with branched alcohols, in particular 2-ethylhexanol, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-, triglyceride mixtures based on C₆-C₁₈-fatty acids, esters of C₈-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear C₆-C₂₂-fatty alcohol carbonates, Guerbet carbonates, esters of benzoic acid with linear and/or branched C₆-C₂₂-alcohols (e.g. Finsolv® TN), dialkyl ethers, ring-opening products of epoxidized fatty acid esters with polyols, silicone oils and/or aliphatic or naphthenic hydrocarbons. Oil substances which may be used are also silicone compounds, for example dimethyl polysiloxanes, methylphenylpolysiloxanes, cyclic silicones, and amino-, fatty-acid-, alcohol-, polyether-, epoxy-, fluorine-, alkyl- and/or glycoside-modified silicone compounds, which may either be in the form of a liquid or in the form of a resin at room temperature. The oil substances may be present in the compositions according to the invention in amounts of from 1 to 90% by weight, preferably 5 to 80% by weight, and in particular 10 to 50% by weight, based on the composition.

The list of specified ingredients which can be used together with the keratin-binding effector molecules according to the invention and/or produced by the inventive method should of course not be regarded as being exhaustive or limiting. The ingredients can be used individually or in any combinations with one another.

The invention also provides the keratin-binding effector proteins shown in the sequences SEQ ID No.: 168, 176, 182, 188, 194 and 200.

The present invention likewise provides nucleic acid molecules according to the SEQ ID No.: 167, 175, 181, 187, 193 and 199 and nucleic acid molecules which code for polypeptides, comprising at least one polypeptide according to the sequences shown in SEQ ID No.: 168, 176, 182, 188, 194 and 200.

The present invention further provides DNA expression cassettes comprising at least one nucleic acid molecule with a nucleic acid sequence which encodes for a polypeptide, comprising at least one polypeptide which is encoded by a nucleic acid molecule according to the sequence shown in SEQ ID No.: 167, 175, 181, 187, 193 or 199. According to the invention, preference is given to DNA expression cassettes comprising a nucleic acid molecule with a nucleic acid sequence according to the sequence shown in SEQ ID No.: 167.

Preferably, such constructs according to the invention comprise a promoter 5′-upstream of the particular encoding sequence, and a terminator sequence 3′-downstream, and also, if appropriate, further customary regulatory elements, each operatively linked to the encoding sequence.

Regulatory elements include enhancers, targeting sequences, polyadenylation signals, selectable markers, amplification signals, replication origins and the like. Suitable regulatory sequences are described, for example, in Goeddel, Gene Expression Technology: Methods in Enzymology 185, Academic Press, San Diego, Calif. (1990).

In addition to these regulation sequences, the natural regulation of these sequences may also be present before the actual structure genes and, if appropriate, have been genetically modified such that the natural regulation has been switched off and the expression of the genes has been increased.

A preferred nucleic acid construct advantageously also comprises one or more of the “enhancer” sequences already mentioned, functionally linked to the promoter, which permit increased expression of the nucleic acid sequence. On the 3′-end of the DNA sequences it is also possible to insert additional advantageous sequences, such as further regulatory elements or terminators.

The nucleic acids according to the invention may be present in one or more copies within the construct. Within the construct, further markers, such as genes complementing antibiotic resistances or auxotrophs, if appropriate for selection on the construct, may also be present.

Advantageous regulation sequences for the method according to the invention are present, for example, in promoters such as cos, tac, trp, tet, trp-tet, Ipp, lac, Ipp, lacIq-T7, T5, T3, gal, trc, ara, rhaP(rhaPBAD) SP6, lambda-PR or imlambda-P promoter, which are advantageously used in Gram-negative bacteria. Further advantageous regulation sequences are present, for example, in the Gram-positive promoters amy and SP02, in the yeast or fungi promoters ADC1, MFalpha, AC, P-60, CYC1, GAPDH, TEF, rp28, ADH.

It is also possible to use artificial promoters for the regulation.

For expression in a host organism, the nucleic acid construct is advantageously inserted into a vector, such as, for example, a plasmid or a phage, which allows optimum expression of the genes in the host. Apart from plasmids and phages, vectors are also understood as meaning all other vectors known to the person skilled in the art, thus e.g. viruses, such as SV40, CMV, baculovirus and adenovirus, transposons, IS elements, phasmids, cosmids, and linear or circular DNA, and also the agrobacterium system.

The production of an expression cassette can be realized using customary recombinant and cloning techniques known to the person skilled in the art, as described, for example, in Maniatis T, Fritsch E F and Sambrook J (1989) Molecular Cloning: A Laboratory Manual, Cold Spring Harbor Laboratory, Cold Spring Harbor (NY), in Silhavy T J, Berman M L and Enquist L W (1984) Experiments with Gene Fusions, Cold Spring Harbor Laboratory, Cold Spring Harbor (NY), in Ausubel F M et al. (1987) Current Protocols in Molecular Biology, Greene Publishing Assoc. and Wiley Interscience. Between the two sequences it is, however, also possible to position further sequences which, for example, have the function of a linker with certain restriction enzyme restriction sites, of a signal peptide or of a protein anchor (e.g. His tag). The insertion of sequences can also lead to the expression of fusion proteins. Preferably, the expression cassette, consisting of a linkage of promoter and nucleic acid sequence to be expressed, can be present in integrated form in a vector and be inserted into the genome of a cell by, for example, transformation. The expression cassette inserted into a vector can also exist and be propagated extrachromosomally in a cell.

The nucleic acid sequences present in the expression cassettes or vectors according to the invention can be functionally linked with further genetic control sequences besides a promoter. The term genetic control sequences is to be understood in the wide sense and means all those sequences which have an effect on the coming into being or the function of the expression cassette according to the invention. Genetic control sequences modify, for example, the transcription and translation in prokaryotic or eukaryotic organisms. Preferably, the expression cassettes according to the invention comprise a promoter 5′-upstream of the particular nucleic acid sequence to be expressed transgenically, and a terminator sequence 3′-downstream as additional genetic control sequence, and, if appropriate, further customary regulatory elements, each functionally linked to the nucleic acid sequence to be expressed transgenically.

Genetic control sequences also comprise further promoters, promotor elements or minimal promoters which can modify the expression-controlling properties. In principle, it is possible to use all natural promoters with their regulation sequences which are able, in the preferred organisms, to control the gene expression of a nucleic acid molecule. Moreover, synthetic promoters can also be used advantageously.

In addition, genetic control sequences also comprise the 5′-untranslated regions, introns or noncoding 3′-region of genes. For example, it has been shown that 5′-untranslated sequences can enhance the transient expression of heterologous genes.

The expression cassette can advantageously comprise one or more so-called “enhancer sequences” functionally linked to the promoter, which permit increased transgenic expression of the nucleic acid sequence. Also on the 3′-end of the nucleic acid sequences to be expressed transgenically, additional advantageous sequences can be inserted, such as further regulatory elements or terminators. The nucleic acid sequences to be expressed transgenically can be present in the gene construct in one or more copies.

In addition, control sequences are to be understood as meaning those which allow homologous recombination and/or insertion into the genome of a host organism, or permit removal from the genome.

In the case of homologous recombination, for example, the natural promoter of a specific gene can be exchanged for a promoter with other properties.

An expression cassette and the vectors derived from it can comprise further functional elements. The term functional element is to be understood in the wide sense and means all those elements which have an effect on production, replication or function of the expression cassettes, vectors or transgenic organisms according to the invention. Mention may be made of the following nonlimiting examples:
a) Selection markers. To select successfully transformed cells it is usually necessary to additionally introduce a selectionable marker which imparts resistance to a biocide (for example a herbicide), a metabolism inhibitor, or an antiobiotic to the successfully transformed cells. The selection marker allows the selection of the transformed cells from untransformed ones and are to be chosen individually according to the desired host organisms and are known as such to the person skilled in the art. Selection markers impart, for example, resistance to a metabolism inhibitor, such as 2-deoxyglucose-6-phosphate (WO 98/45456), antibiotics or biocides, preferably herbicides, such as, for example, kanamycin, G 418, bleomycin, hygromycin or kanamycin etc. As selection marker preference is given to the aasa gene, which imparts resistance to the antibiotic apectinomycin, the streptomycin phosphotransferase (SPT) gene, which ensures resistance against streptomycin, the neomycin phosphotransferase (NPTII) gene, which imparts resistance against kanamycin or geneticidin, the hygromycin phosphotransferase (HPT) gene, which imparts resistance to hygromycin, the acetolactate synthase gene (ALS), which imparts resistance to sulfonylurea herbicides (e.g. mutated ALS variants with e.g. S4 and/or Hra mutation).
b) Reporter genes which code for readily quantifiable proteins or, via intrinsic color or enzyme activity, ensure evaluation of the transformation efficiency or of the expression site or time. Very particular preference is given here to reporter proteins (Schenborn E, Groskreutz D. Mol Biotechnol. 1999; 13(1):29-44) such as the “green fluorescence protein” (GFP) (Sheen et al.(1995) Plant Journal 8(5):777-784; Haseloff et al.(1997) Proc Natl Acad Sci USA 94(6):2122-2127; Reichel et al.(1996) Proc Natl Acad Sci USA93(12):5888-5893; Tian et al. (1997) Plant Cell Rep 16:267-271; WO 97/41228; Chui W L et al. (1996) Curr Biol 6:325-330; Leffel S M et al. (1997) Biotechniques. 23(5):912-8), the chloramphenicol transferase, a luciferase (Ow et al. (1986) Science 234:856-859; Millar et al. (1992) Plant Mol Biol Rep 10:324-414), the aequorin gene (Prasher et al. (1986) Biochem Biophys Res Commun 126(3):1259-1268), the β-galactosidase).
c) Replication origins which ensure replication of the expression cassettes or vectors according to the invention in, for example, E. coli. By way of example, mention may be made of ORI (origin of DNA replication), pBR322 ori or P15A ori (Sambrook et al.: Molecular Cloning. A Laboratory Manual, 2^{nd} ed. Cold Spring Harbor Laboratory Press, Cold Spring Harbor, N.Y., 1989).

The insertion of an expression cassette according to the invention into a cell or an organism can advantageously be realized using vectors in which the expression cassettes are present. The expression cassette can be inserted into the vector (for example a plasmid) via a suitable restriction site. The resulting plasmid is initially introduced into E. coli. Correctly transformed E. coli are selected, harvested and the recombinant plasmid is obtained using methods familiar to the person skilled in the art. Restriction analysis and sequencing can also serve to check the cloning step.

The present invention likewise provides vectors comprising an expression cassette comprising a nucleic acid molecule with a nucleic acid sequence according to the sequence shown in SEQ ID No.: 167, 175, 181, 187, 193 or 199.

For expression in a host organism, the nucleic acid construct is advantageously inserted into a vector, such as, for example, a plasmid or a phage which allows optimum expression of the genes in the host. Apart from plasmids and phages, vectors are also to be understood as meaning all other vectors known to the person skilled in the art, thus e.g. viruses, such as, SV40, CMV, baculovirus and adenovirus, transposons, IS elements, phasmids, cosmids, and linear or circular DNA, and also the agrobacterium system.

These vectors can be replicated autonomously in the host organism or be chromosomally replicated.

These vectors represent a further embodiment of the invention. Suitable plasmids are, for example, in E. coli pLG338, pQE30, pACYC184, pBR322, pUC18, pUC19, pKC30, pRep4, pHS1, pKK223-3, pDHE19.2, pHS2, pPLc236, pMBL24, pLG200, pUR290, pIN-III″3-B1, tgt11 or pBdCl, in Streptomyces pIJ101, pIJ364, pIJ702 or pIJ361, in Bacillus pUB110, pC194, pWH320, pMM1520, pMM1525 or pBD214, in Corynebacterium pSA77 or pAJ667, in fungi pALS1, pIL2 or pBB116, in yeasts 2alpha, pAG-1, YEp6, YEp13 or pEMBLYe23 or in plants pLGV23, pGHlac+, pBIN19, pAK2004 or pDH51. The specified plasmids represent a small selection of the possible plasmids. Further plasmids are known to the person skilled in the art and can be found, for example, in the book Cloning Vectors (Eds. Pouwels P. H, et al. Elsevier, Amsterdam-New York-Oxford, 1985, ISBN 0 444 904018).

Nucleic acid constructs according to the invention or the vectors comprising the nucleic acid molecules according to the invention can also advantageously be introduced into the microorganisms in the form of a linear DNA and be integrated into the genome of the host organism via heterologous or homologous recombination. This linear DNA can consist of a linearized vector such as a plasmid or only of the nucleic acid construct or the nucleic acid according to the invention.

For optimum expression of heterologous genes in organisms, it is advantageous to modify the nucleic acid sequences to correspond to the specific “codon usage” used in the organism. The “codon usage” can be determined easily using computer evaluations of other known genes of the organism in question (e.g. via: Codon usage tabulated from the international DNA sequence databases: status for the year 2000. Nakamura, Y., Gojobori, T. and Ikemura, T. (2000) Nucl. Acids Res. 28, 292., http://www.kazusa.or.jp/codon/index.html).

For expression in a suitable host organism, the recombinant nucleic acid construct or gene construct is advantageously inserted into a host-specific vector which permits optimum expression of the genes in the host. Vectors are well known to the person skilled in the art and can be found, for example, in “Cloning Vectors” (Pouwels P. H. et al., Ed., Elsevier, Amsterdam-New York-Oxford, 1985).

With the help of the vectors according to the invention, it is possible to prepare recombinant microorganisms which are, for example, transformed with at least one vector according to the invention and can be used for the production of the polypeptides according to the invention. The recombinant constructs according to the invention described above are advantageously inserted into a suitable host system and expressed. Here, preference is given to using customary cloning and transfection methods known to the person skilled in the art, such as, for example, coprecipitation, protoplast fusion, electroporation, retroviral transfection and the like, in order to cause the specified nucleic acids to be expressed in the particular expression system. Suitable systems are described, for example, in Current Protocols in Molecular Biology, F. Ausubel et al., Ed., Wiley Interscience, New York 1997, or Sambrook et al. Molecular Cloning: A Laboratory Manual. 2^{nd} edition, Cold Spring Harbor Laboratory, Cold Spring Harbor Laboratory Press, Cold Spring Harbor, N.Y., 1989.

According to the invention, it is also possible to prepare homologously recombinant microorganisms. For this purpose, a vector is prepared which comprises at least one section of a gene according to the invention or of a coding sequence in which, if appropriate, at least one amino acid deletion, addition or substitution has been introduced in order to modify, e.g. functionally disrupt, the sequence according to the invention (“knockout” vector). The inserted sequence can, for example, also be a homolog from a related microorganism or be derived from a mammal source, yeast source or insect source. The vector used for the homologous recombination can alternatively be designed so that the endogenous gene is mutated or modified in some other way during homologous recombination, but still encodes the functional protein (e.g. if the upstream regulatory region can be modified in such a way that expression of the endogenous protein is changed as a result). The modified section of the gene according to the invention is in the homologous recombination vector. The construction of suitable vectors for the homologous recombination is described, for example, in Thomas, K. R. and Capecchi, M. R. (1987) Cell 51: 503.

Suitable transgenic recombinant host organisms for the nucleic acid according to the invention or the nucleic acid construct are in principle all prokaryotic organisms (including Archaea) or eukaryotic organisms. Especially bacteria including halobacteria and methanococci, fungi, insect cells, plant cells and mammal cells. The host organisms used are advantageously microorganisms such as bacteria, fungi or yeasts, Fungi, Gram-positive or Gram-negative bacteria, preferably bacteria of the families Enterobacteriaceae, Pseudomonadaceae, Rhizobiaceae, Streptomycetaceae or Nocardiaceae, particularly preferably bacteria of the genera Escherichia, Pseudomonas, Streptomyces, Nocardia, Burkholderia, Salmonella, Agrobacterium or Rhodococcus can be used advantageously. Escherichia coli, Bacillus subtilis, Bacillus. megaterium, Pseudomonas spec., lactobacillae, Hansenula polymorpha, Aspergillus oryzea, Aspergillus nidulans, Aspergillus niger, Pichia pastoris, Trichoderma reesei and SF9 cells (or related cells) are most preferred.

The organisms used for producing the keratin-binding effector proteins according to the invention are grown or cultivated in the manner known to the person skilled in the art depending on the host organism. Microorganisms are usually grown in a liquid medium which comprises a carbon source mostly in the form of sugars, a nitrogen source mostly in the form of organic nitrogen sources such as yeast extract or salts such as ammonium sulfate, trace elements such as iron salts, manganese salts and magnesium salts and, if appropriate, vitamins, at temperatures between 0° C. and 100° C., preferably between 10° C. to 60° C. under oxygen. Here, the pH of the nutrigen liquid can be kept at a fixed value, i.e. be regulated or not regulated during the cultivation. The cultivation can be batchwise, semibatchwise or continuous. Nutrients can be initially introduced at the start of the fermentation or can be fed in afterwards semicontinuously or continuously. The enzymes can be isolated from the organisms by the process described in the examples or be used as raw extract for the reaction.

The polypeptides can thus also be produced on an industrial scale, if desired. The recombinant microorganism can be cultivated and fermented by known processes. Bacteria can be reproduced, for example, in TB or LB medium and at a temperature of from 20° C. to 40° C. and a pH of from 6 to 9. Specifically, suitable cultivation conditions are described, for example, in T. Maniatis, E. F. Fritsch and J. Sambrook, Molecular Cloning A Laboratory Manual, Cold Spring Harbor Laboratory, Cold Spring Harbor, N.Y. (1989).

### Disruption and Purification

If the polypeptides are not secreted into the culture medium, the cells are then disrupted and the product is obtained from the lyzate by known protein isolation methods. The cells can be disrupted as desired by high-frequency ultrasound, by high pressure, such as, for example, in a French pressure cell, by osmolysis, by the action of detergents, lytic enzymes or organic solvents, by homogenizers or by combining two or more of the methods fisted.

Purification of the polypeptides can be achieved using known chromatographic methods, such as molecular sieve chromatography (gel filtration), such as Q-Sepharose chromatography, ion-exchange chromatography and hydrophobic chromatography, and also with other customary methods, such as ultrafiltration, crystallization, salting out, dialysis and native gel electrophoresis. Suitable methods are described, for example, in Cooper, F.G., Biochemische Arbeitsmethoden [Biochemical working methods], Verlag Water de Gruyter, Berlin, N.Y. or in Scopes, R., Protein Purification, Springer Verlag, New York, Heidelberg, Berlin.

It may advantageous, for isolating the recombinant protein, to use vector systems or oligonucleotides which extend the cDNA by certain nucleotide sequences and thus code for modified polypeptides or fusion proteins which serve, for example, for easier purification. Such suitable modifications are, for example, so-called “tags” acting as anchors, such as, for example, the modification known as hexahistidine anchor, or epitopes which can be recognized as antigens by antibodies (described, for example, in Harlow, E. and Lane, D., 1988, Antibodies: A Laboratory Manual. Cold Spring Harbor (N.Y.) Press). Further suitable tags are, for example, HA, calmodulin-BD, GST, MBD; chitin-BD, steptavidin-BD-Avi tag, Flag tag, T7 etc. These anchors can serve to attach the proteins to a fixed support, such as, for example, a polymer matrix, which can be fed, for example, into a chromatography column, or can be used on a microtiter plate or another support. The corresponding purification protocols are available from the commercial affinity tag suppliers.

The keratin-binding effector proteins according to the invention have the desired properties of keratin-binding proteins both in their fused form, i.e. together with the fusion partner moiety, and also in isolated form. The proteins according to the invention can thus be used either directly as fusion proteins, or after cleavage and separation of the fusion partner as “pure” keratin-binding proteins.

If separation of the fusion partner is intended, it is advisable to incorporate a potential cleavage site (specific recognition site for proteases) into the fusion protein between keratin-binding protein moiety and fusion partner moiety. Suitable cleavage sites are, in particular, those peptide sequences which otherwise occur neither in the keratin-binding protein moiety nor in the fusion partner moiety, which can be ascertained easily using bioinformatic tools. Of particular suitability are, for example, BrCN cleavage on methionine, or protease-mediated cleavage with factor Xa, enterokinase, thrombin, TEV cleavage (tobacco etch virus protease).

Moreover, the present invention provides transgenic cells comprising
v) at least one of the abovementioned vectors, or
w) at least one of the abovementioned expression cassettes, or
x) at least one of the abovementioned nucleic acid molecules coding for a polypeptide comprising at least one polypeptide which is encoded by a nucleic acid molecule according to the sequence shown in SEQ ID No.: 167, 175, 181, 187, 193 or 199.

Preferably, the cells (see above) or organisms (see above) are transgenic cells or organisms which have been transformed with at least one nucleic acid molecule according to the sequence shown in SEQ ID No.: 167, 175, 181, 187, 193 or 199.

Particularly preferred transgenic organisms are Escherichia coli, Bacillus subtilis, Bacillus. megaterium, Aspergillus oryzea, Aspergillus nidulans, Aspergilius niger, Pichia pastoris, Pseudomonas spec., lactobacillae, Hansenula polymorpha, Trichoderma reesei and SF9 cells (or related cells).

Sequences

| SEQ | Sequence | |
|---|---|---|
| ID NO.: | type | Sequence description |
| 1 | Nucleic acid | Homo sapiens Desmoplakin_Accession No. NM_004415 |
| 2 | Protein | Homo sapiens Desmoplakin_Accession No. NM_004415 |
| 3 | Nucleic acid | Homo sapiens Desmoplakin_Accession No. NM_004415 domain B |
| 4 | Protein | Homo sapiens Desmoplakin_Accession No. NM_004415 domain B |
| 5 | Nucleic acid | Homo sapiens Desmoplakin_Accession No. NM_004415 domain B-1 |
| 6 | Protein | Homo sapiens Desmoplakin_Accession No. NM_004415 domain B-1 |
| 7 | Nucleic acid | Homo sapiens Desmoplakin_Accession No. NM_004415 domain B-2 |
| 8 | Protein | Homo sapiens Desmoplakin_Accession No. NM_004415 domain B-2 |
| 9 | Nucleic acid | Homo sapiens Desmoplakin_Accession No. NM_004415 domain C |
| 10 | Protein | Homo sapiens Desmoplakin_Accession No. NM_004415 domain C |
| 11 | Nucleic acid | Homo sapiens Desmoplakin_Accession No. NM_004415 domain C-1 |
| 12 | Protein | Homo sapiens Desmoplakin_Accession No. NM_004415 domain C-1 |
| 13 | Nucleic acid | Homo sapiens Desmoplakin_Accession No. NM_004415 domain C-2 |
| 14 | Protein | Homo sapiens Desmoplakin_Accession No. NM_004415 domain C-2 |
| 15 | Nucleic acid | H. sapiens_Filaggrin_Accession No. CAI19595 |
| 16 | Protein | H. sapiens_Filaggrin_Accession No. CAI19596 |
| 17 | Nucleic acid | Homo sapiens plakophilin 1 ACCESSION NM_001005337, transcript |
| | | variant 1a |
| 18 | Protein | Homo sapiens plakophilin 1 ACCESSION NM_001005337, transcript |
| | | variant 1a |
| 19 | Nucleic acid | Homo sapiens plakophilin 1 ACCESSION NM_000299, transcript variant |
| | | 1b |
| 20 | Protein | Homo sapiens plakophilin 1 ACCESSION NM_000299, transcript variant |
| | | 1b |
| 21 | Nucleic acid | Mus musculus plakophilin 2 ACCESSION NM_026163 NM_027894 |
| 22 | Protein | Mus musculus plakophilin 2 ACCESSION NM_026163 NM_027895 |
| 23 | Nucleic acid | Mus musculus plakophilin 1 ACCESSION NM_019645 |
| 24 | Protein | Mus musculus plakophilin 1 ACCESSION NM_019646 |
| 25 | Nucleic acid | Bos taurus plakophilin 1 partial mRNA, ACCESSION XM_868348 |
| 26 | Protein | Bos taurus plakophilin 1 partial mRNA, ACCESSION XM_868349 |
| 27 | Nucleic acid | Canis familiaris similar to plakophilin 1 isoform 1a, ACCESSION |
| | | XM_851528 |
| 28 | Protein | Canis familiaris similar to plakophilin 1 isoform 1a, ACCESSION |
| | | XM_851529 |
| 29 | Nucleic acid | Danio rerio similar to Plakophilin 1 ACCESSION XM_695832 |
| 30 | Protein | Danio rerio similar to Plakophilin 1 ACCESSION XM_695833 |
| 31 | Nucleic acid | Rattus norvegicus similar to plakophilin 1, ACCESSION XM_222666 |
| 32 | Protein | Rattus norvegicus similar to plakophilin 1, ACCESSION XM_222667 |
| 33 | Nucleic acid | Pan troglodytes similar to Plakophilin 1, ACCESSION XM_514091 |
| 34 | Protein | Pan troglodytes similar to Plakophilin 1, ACCESSION XM_514092 |
| 35 | Nucleic acid | Gallus gallus similar to plakophilin 1, ACCESSION XM_419240 |
| 36 | Protein | Gallus gallus similar to plakophilin 1, ACCESSION XM_419241 |
| 37 | Nucleic acid | Xenopus laevis similar to plakophilin 4, ACCESSION BI390496 |
| 38 | Protein | Xenopus laevis similar to plakophilin 4, ACCESSION BI390497 |
| 39 | Nucleic acid | Homo sapiens desmoplakin, transcript variant 2, ACCESSION |
| | | NM_001008844 |
| 40 | Protein | Homo sapiens desmoplakin, transcript variant 2, ACCESSION |
| | | NM_001008845 |
| 41 | Nucleic acid | Mus musculus desmoplakin, ACCESSION XM_621314 |
| 42 | Protein | Mus musculus desmoplakin, ACCESSION XM_621315 |
| 43 | Nucleic acid | Rattus norvegicus similar to desmoplakin isoform II, ACCESSION |
| | | XM_225259 |
| 44 | Protein | Rattus norvegicus similar to desmoplakin isoform II, ACCESSION |
| | | XM_225260 |
| 45 | Nucleic acid | Pan troglodytes desmoplakin, ACCESSION XM_518227 |
| 46 | Protein | Pan troglodytes desmoplakin, ACCESSION XM_518228 |
| 47 | Nucleic acid | Gallus gallus similar to Desmoplakin, ACCESSION XM_418957 |
| 48 | Protein | Gallus gallus similar to Desmoplakin, ACCESSION XM_418958 |
| 49 | Nucleic acid | Homo sapiens junction plakoglobin (JUP), transcript variant 2, |
| | | ACCESSION NM_021991 |
| 50 | Protein | Homo sapiens junction plakoglobin (JUP), transcript variant 2, |
| | | ACCESSION NM_021992 |
| 51 | Nucleic acid | Mus musculus, plakoglobin; gamma-catenin, ACCESSION NM_010593 |
| 52 | Protein | Mus musculus, plakoglobin; gamma-catenin, ACCESSION NM_010594 |
| 53 | Nucleic acid | Rattus norvegicus gamma-catenin (plakoglobin), ACCESSION |
| | | NM_031047 |
| 54 | Protein | Rattus norvegicus gamma-catenin (plakoglobin), ACCESSION |
| | | NM_031048 |
| 55 | Nucleic acid | Danio rerio armadillo protein family; plakoglobin, ACCESSION |
| | | NM_131177 |
| 56 | Protein | Danio rerio armadillo protein family; plakoglobin, ACCESSION |
| | | NM_131178 |
| 57 | Nucleic acid | Xenopus tropicalis junction plakoglobin, ACCESSION BC064717 |
| 58 | Protein | Xenopus tropicalis junction plakoglobin, ACCESSION BC064718 |
| 59 | Nucleic acid | Canis familiaris similar to junction plakoglobin isoform 10, ACCESSION |
| | | XM_856625 |
| 60 | Protein | Canis familiaris similar to junction plakoglobin isoform 10, ACCESSION |
| | | XM_856626 |
| 61 | Nucleic acid | Xenopus laevis Jup protein, ACCESSION BC094116 |
| 62 | Protein | Xenopus laevis Jup protein, ACCESSION BC094117 |
| 63 | Nucleic acid | Bos taurus junction plakoglobin, ACCESSION NM_001004024 |
| 64 | Protein | Bos taurus junction plakoglobin, ACCESSION NM_001004025 |
| 65 | Nucleic acid | Sus scrofa plakoglobin, ACCESSION NM_214323 |
| 66 | Protein | Sus scrofa plakoglobin, ACCESSION NM_214324 |
| 67 | Nucleic acid | Danio rerio junction plakoglobin, ACCESSION BC058305 |
| 68 | Protein | Danio rerio junction plakoglobin, ACCESSION BC058306 |
| 69 | Nucleic acid | Saccharomyces cerevisiae, plakoglobin/armadillo/beta-catenin, |
| | | ACCESSION AF005267 |
| 70 | Protein | Saccharomyces cerevisiae, plakoglobin/armadillo/beta-catenin, |
| | | ACCESSION AF005268 |
| 71 | Nucleic acid | Homo sapiens plectin 1, intermediate filament binding protein, |
| | | ACCESSION NM_201380 |
| 72 | Protein | Homo sapiens plectin 1, intermediate filament binding protein, |
| | | ACCESSION NM_201381 |
| 73 | Nucleic acid | Mus musculus plectin 1 (Plec1), transcript variant 11, mRNA, |
| | | ACCESSION NM_201394 XM |
| 74 | Protein | Mus musculus plectin 1 (Plec1), transcript variant 11, mRNA, |
| | | ACCESSION NM_201394 XM |
| 75 | Nucleic acid | Bos taurus similar to plectin 1 isoform 1 (LOC510991), ACCESSION |
| | | XM_588232 |
| 76 | Protein | Bos taurus similar to plectin 1 isoform 1 (LOC510991), ACCESSION |
| | | XM_588233 |
| 77 | Nucleic acid | Canis familiaris similar to plectin 1 isoform, ACCESSION XM_539204 |
| 78 | Protein | Canis familiaris similar to plectin 1 isoform, ACCESSION XM_539205 |
| 79 | Nucleic acid | Trypanosoma cruzi, plectin-like protein, ACCESSION XM_809849 |
| 80 | Protein | Trypanosoma cruzi, plectin-like protein, ACCESSION XM_809850 |
| 81 | Nucleic acid | Rattus norvegicus plectin, ACCESSION X59601 |
| 82 | Protein | Rattus norvegicus plectin, ACCESSION X59602 |
| 83 | Nucleic acid | Cricetulus griseus plectin, ACCESSION AF260753 |
| 84 | Protein | Cricetulus griseus plectin, ACCESSION AF260754 |
| 85 | Nucleic acid | Homo sapiens periplakin, ACCESSION NM_002705 |
| 86 | Protein | Homo sapiens periplakin, ACCESSION NM_002706 |
| 87 | Nucleic acid | Mus musculus periplakin, ACCESSION NM_008909 XM_358905 |
| 88 | Protein | Mus musculus periplakin, ACCESSION NM_008909 XM_358906 |
| 89 | Nucleic acid | Homo sapiens envoplakin, ACCESSION NM_001988 |
| 90 | Protein | Homo sapiens envoplakin, ACCESSION NM_001989 |
| 91 | Nucleic acid | Mus musculus envoplakin, ACCESSION NM_025276 XM_283024 |
| 92 | Protein | Mus musculus envoplakin, ACCESSION NM_025276 XM_283025 |
| 93 | Nucleic acid | Bos taurus similar to Envoplakin, ACCESSION XM_587641 |
| 94 | Protein | Bos taurus similar to Envoplakin, ACCESSION XM_587642 |
| 95 | Nucleic acid | Canis familiaris similar to Envoplakin, ACCESSION XM_540443 |
| 96 | Protein | Canis familiaris similar to Envoplakin, ACCESSION XM_540444 |
| 97 | Nucleic acid | Danio rerio similar to Envoplakin, ACCESSION XM_687958 |
| 98 | Protein | Danio rerio similar to Envoplakin, ACCESSION XM_687959 |
| 99 | Nucleic acid | Rattus norvegicus, similar to envoplakin, db_xref GeneID: 303687 |
| 100 | Protein | Rattus norvegicus, similar to envoplakin, db_xref GeneID: 303688 |
| 101 | Nucleic acid | Pan troglodytes similar to Envoplakin, ACCESSION XM_511692 |
| 102 | Protein | Pan troglodytes similar to Envoplakin, ACCESSION XM_511693 |
| 103 | Nucleic acid | Human bullous pemphigoid antigen, ACCESSION M63618 |
| 104 | Protein | Human bullous pemphigoid antigen, ACCESSION M63619 |
| 105 | Nucleic acid | Mus musculus bullous pemphigoid antigen 1 (Bpag1), ACCESSION |
| | | AF396877 |
| 106 | Protein | Mus musculus bullous pemphigoid antigen 1 (Bpag1), ACCESSION |
| | | AF396878 |
| 107 | Nucleic acid | Mus musculus trichohyalin-like 1, ACCESSION NM_027762 |
| 108 | Protein | Mus musculus trichohyalin-like 1, ACCESSION NM_027763 |
| 109 | Nucleic acid | Bos taurus similar to trichohyalin-like 1, ACCESSION XM_597026 |
| 110 | Protein | Bos taurus similar to trichohyalin-like 1, ACCESSION XM_597027 |
| 111 | Nucleic acid | Homo sapiens trichohyalin-like 1, ACCESSION NM_001008536 |
| | | XM_060104 |
| 112 | Protein | Homo sapiens trichohyalin-like 1, ACCESSION NM_001008536 |
| | | XM_060105 |
| 113 | Nucleic acid | Strongylocentrotus purpuratus similar to Trichohyalin, ACCESSION |
| | | XM_793822 |
| 114 | Protein | Strongylocentrotus purpuratus similar to Trichohyalin, ACCESSION |
| | | XM_793823 |
| 115 | Nucleic acid | Trypanosoma cruzi trichohyalin, putative, ACCESSION XM_809758 |
| 116 | Protein | Trypanosoma cruzi trichohyalin, putative, ACCESSION XM_809759 |
| 117 | Nucleic acid | Giardia lamblia ATCC 50803 trichohyalin, ACCESSION XM_765825 |
| 118 | Protein | Giardia lamblia ATCC 50803 trichohyalin, ACCESSION XM_765826 |
| 119 | Nucleic acid | Aspergillus fumigatus Af293, trichohyalin, ACCESSION XM_748643 |
| 120 | Protein | Aspergillus fumigatus Af293, trichohyalin, ACCESSION XM_748644 |
| 121 | Nucleic acid | O. cuniculus trichohyalin, ACCESSION Z19092 |
| 122 | Protein | O. cuniculus trichohyalin, ACCESSION Z19093 |
| 123 | Nucleic acid | Pan troglodytes similar to Trichohyalin, ACCESSION XM_526770 |
| 124 | Protein | Pan troglodytes similar to Trichohyalin, ACCESSION XM_526771 |
| 125 | Nucleic acid | Human trichohyalin (TRHY), ACCESSION L09190 |
| 126 | Protein | Human trichohyalin (TRHY), ACCESSION L09191 |
| 127 | Nucleic acid | Mus musculus small proline-rich protein 3, ACCESSION NM_011478 |
| 128 | Protein | Mus musculus small proline-rich protein 3, ACCESSION NM_011479 |
| 129 | Nucleic acid | Homo sapiens small proline-rich protein 2B (SPRR2B), ACCESSION |
| | | NM_001017418 |
| 130 | Protein | Homo sapiens small proline-rich protein 2B (SPRR2B), ACCESSION |
| | | NM_001017419 |
| 131 | Nucleic acid | Mus musculus hair follicle protein AHF, ACCESSION XM_485271 |
| 132 | Protein | Mus musculus hair follicle protein AHF, ACCESSION XM_485272 |
| 133 | Nucleic acid | Homo sapiens epiplakin 1 (EPPK1), ACCESSION NM_031308 |
| | | XM_372063 |
| 134 | Protein | Homo sapiens epiplakin 1 (EPPK1), ACCESSION NM_031308 |
| | | XM_372064 |
| 135 | Nucleic acid | Mus musculus epiplakin 1, ACCESSION NM_144848 NM_173025 |
| 136 | Protein | Mus musculus epiplakin 1, ACCESSION NM_144848 NM_173026 |
| 137 | Nucleic acid | Mus musculus structural protein FBF1, ACCESSION AF241249 |
| 138 | Protein | Mus musculus structural protein FBF1, ACCESSION AF241250 |
| 139 | Nucleic acid | Streptococcus mutans spaP gene for antigen I/II, ACCESSION X17390 |
| 140 | Protein | Streptococcus mutans spaP gene for antigen I/II, ACCESSION X17391 |
| 141 | Nucleic acid | Sequence of the PCR primer Bag 43 |
| 142 | Nucleic acid | Sequence of the PCR primer Bag 44 |
| 143 | Nucleic acid | Sequence of the PCR primer Bag 53 |
| 144 | Nucleic acid | Sequence of the PCR primer Bag 51 |
| 145 | Nucleic acid | Sequence of the PCR primer Lib197 |
| 146 | Nucleic acid | Sequence of the PCR primer Lib198 |
| 147 | Nucleic acid | Variant of the sequence according to SEQ ID No.: ID 3 mutagenisized |
| | | using the PCR-Primer Lib197 (SEQ ID No.: 145 ) and Lib198 (SEQ ID |
| | | No.: 146) |
| 148 | Nucleic acid | Sequence of the PCR primer Lib201 |
| 149 | Nucleic acid | Sequence of the PCR primer Lib2O2 |
| 150 | Nucleic acid | Nucleic acid coding for a C16 modulus of the spider silk protein ADF-4C |
| | | from Araneus diadematus |
| 151 | Protein | Translation product of the nucleic acid molecule SEQ ID No.: 150 |
| 152 | Nucleic acid | Variant of the sequence according to SEQ ID No.: ID 3; DNA fragment |
| | | which amplifies by means of the PCR primer Lib151 (SEQ ID No.: 154) |
| | | and Lib 152 (SEQ ID No.: 155) |
| 153 | Protein | Translation product of the nucleic acid molecule SEQ ID No.: 152 |
| 154 | Nucleic acid | Sequence of the PCR primer Lib151 |
| 155 | Nucleic acid | Sequence of the PCR primer Lib152 |
| 156 | Protein | KBD-B_Homo sapiens Desmoplakin_Accession No. NM_004415 domain |
| | | B-3 |
| 157 | Protein | KBD-B_Homo sapiens Desmoplakin_Accession No. NM_004415 domain |
| | | B-4 |
| 158 | Protein | KBD-B_Homo sapiens Desmoplakin_Accession No. NM_004415 domain |
| | | B-5 |
| 159 | Nucleic acid | KBD-B_Homo sapiens Desmoplakin_Accession No. NM_004415 domain |
| | | B-6 |
| 160 | Protein | KBD-B_Homo sapiens Desmoplakin_Accession No. NM_004415 domain |
| | | B-6 |
| 161 | Nucleic acid | Homo sapiens trichoplein, BC004285 |
| 162 | Protein | Homo sapiens trichoplein, BC004285 |
| 163 | Nucleic acid | Homo sapiens Desmoplakin_Accession No. NM_004415 with nucleic acid |
| | | exchange compared to SEQ ID No.: ID 1 |
| 164 | Protein | Homo sapiens Desmoplakin_Accession No. NM_004415 with amino acid |
| | | exchange at positions 905, 2687 and 2688 compared to SEQ ID No.: ID 2 |
| 165 | Nucleic acid | KBD-B_Homo sapiens Desmoplakin_Accession No. NM_004415 domain |
| | | B-7 |
| 166 | Protein | KBD-B_Homo sapiens Desmoplakin_Accession No. NM_004415 domain |
| | | B-7 |
| 167 | Nucleic acid | Chimeric nucleic acid molecule coding for the C16 modulus of the spider |
| | | silk protein ADF-4C from Araneus diadematus (SEQ ID. No.: 150) fused |
| | | with the nucleic acid molecule (SEQ ID No.: 147) coding for the KBD-B |
| | | protein. |
| 168 | Protein | Chimeric translation product of SEQ ID No.: 167 |
| 169 | Nucleic acid | Sequence of the PCR primer pLib199 |
| 170 | Nucleic acid | Sequence of the PCR primer pLib 200 |
| 171 | Nucleic acid | Nucleic acid molecule of the carotenoid binding protein (CBP) from B. mori |
| | | (Swiss prot. Q8MYA9_BOMMO) |
| 172 | Protein | Translation product of the nucleic acid molecule SEQ ID No.: 17; 1 CBP |
| | | from B. mori encoding gene |
| 173 | Nucleic acid | Sequence of the PCR primer HRe1 |
| 174 | Nucleic acid | Sequence of the PCR primer HRe2 |
| 175 | Nucleic acid | Chimeric nucleic acid molecule coding for the CBP protein (SEQ ID. |
| | | No.: 172) fused with the nucleic acid molecule (SEQ ID No.: 165) coding |
| | | for the KBD-B protein (SEQ ID. No.: 166) |
| 176 | Protein | Translation product of the nucleic acid molecule according to SEQ ID No.: |
| | | 175 |
| 177 | Nucleic acid | Sequence of the PCR primer Lib 212 |
| 178 | Nucleic acid | Sequence of the PCR primer Lib219 |
| 179 | Nucleic acid | Nucleic acid molecule coding for the metal binding protein (ZntA) from |
| | | E. coli Accession No. NP_417926 |
| 180 | Protein | Translation product of the nucleic acid molecule according to SEQ ID No.: |
| | | 179 |
| 181 | Nucleic acid | Chimeric nucleic acid molecule coding for the metal-binding protein (ZntA) |
| | | from E. coli (SEQ ID. No.: 179) fused with the nucleic acid molecule (SEQ |
| | | ID No.: 165) coding for the KBD-B protein (SEQ ID. No.: 166) |
| 182 | Protein | Translation product of the nucleic acid molecule according to SEQ ID No.: |
| | | 181 |
| 183 | Nucleic acid | Sequence of the PCR primer Bag 102 |
| 184 | Nucleic acid | Sequence of the PCR primer Bag103 |
| 185 | Nucleic acid | Nucleic acid molecule coding for the (trxA) thioredoxin gene from E. coli |
| | | Accession No. NP_418228 (Accession No. EG1103) |
| 186 | Protein | Translation product of the nucleic acid molecule according to SEQ ID No.: |
| | | 185 |
| 187 | Nucleic acid | Chimeric nucleic acid molecule coding for the thioredoxin fragment from |
| | | the vector pThioHisC (SEQ ID. No.: 185) fused with the nucleic acid |
| | | molecule (SEQ ID No.: 165) coding for the KBD-B protein (SEQ ID. No.: |
| | | 166) |
| 188 | Protein | Translation product of the nucleic acid molecule according to SEQ ID No.: |
| | | 187 |
| 189 | Nucleic acid | Sequence of the PCR primer Bag 89 |
| 190 | Nucleic acid | Sequence of the PCR primer Bag90 |
| 191 | Nucleic acid | Nucleic acid molecule coding for the eGFP fragment from the vector |
| | | pEGFP-1 (Clontech) |
| 192 | Protein | Translation product of the nucleic acid molecule according to SEQ ID No.: |
| | | 191 |
| 193 | Nucleic acid | Chimeric nucleic acid molecule coding for the eGFP fragment from the |
| | | vector pEGFP-1 (Clontech) (SEQ ID. No.: 191) fused with the nucleic acid |
| | | molecule (SEQ ID No.: 165) coding for the KBD-B protein (SEQ ID. No.: |
| | | 166) |
| 194 | Protein | Translation product of the nucleic acid molecule according to SEQ ID No.: |
| | | 193 |
| 195 | Nucleic acid | Sequence of the PCR primer Bag 93 |
| 196 | Nucleic acid | Sequence of the PCR primer Bag 94 |
| 197 | Nucleic acid | Nucleic acid molecule coding for the YaaD fragment (Accession No. |
| | | BG10075) from the vector pDX14 (OmniGene Bioproducts) |
| 198 | Protein | Translation product of the nucleic acid molecule according to SEQ ID |
| | | No.: 197 |
| 199 | Nucleic acid | Chimeric nucleic acid molecule coding for the YaaD fragment from the |
| | | vector pDX14 (OmniGene Bioproducts) (SEQ ID. No.: 197) fused with the |
| | | nucleic acid molecule (SEQ ID No.: 165) coding for the KBD-B protein |
| | | (SEQ ID. No.: 166) |
| 200 | Protein | Translation product of the nucleic acid molecule according to SEQ ID No.: |
| | | 199 |
| 201 | Protein | AAC47011 fibroin-4 from Araneus diadematus |
| 202 | Protein | Silk protein from Nephila clavipes ACCESSION AY855102 |
| 203 | Protein | Silk protein from Araneus gemmoides ACCESSION AY855101 |
| 204 | Protein | Silk protein from Araneus gemmoides ACCESSION AY855100 |
| 205 | Protein | Silk protein from Argiope aurantia ACCESSION AY855099 |
| 206 | Protein | Silk protein from Argiope aurantia ACCESSION AY855098 |
| 207 | Protein | Synthetic construct spider silk protein-like protein (SF1); accession |
| | | DQ001900 |
| 208 | Protein | Silk protein from Nephila clavipes ACCESSION U37520 |
| 209 | Protein | Synthetic construct RGD-dragline spider silk protein gene; accession |
| | | DQ186903 |
| 210 | Protein | Silk protein from Latrodectus Hesperus ACCESSION AY994149 |
| 211 | Protein | attacin = insect antibacterial protein from Bombyx mori ACCESSION |
| | | S78369 |
| 212 | Nucleic acid | KBD-D with N-terminal histidine anchor, H. sapiens plakophilin 1a |
| | | ACCESSION NM_001005337 |
| 213 | Protein | KBD-D with N-terminal histidine anchor, H. sapiens plakophilin 1a |
| | | ACCESSION NP_001005337 |
| 214 | Nucleic acid | KBD-D amino acids 1-273 with C-terminal histidine anchor, H. sapiens |
| | | plakophilin 1a ACCESSION NM_001005337 |
| 215 | Protein | BD-D amino acids 1-273 with C-termin histidine anchor, H. sapiens |
| | | plakophilin 1a ACCESSION NP_001005337 |
| 216 | Nucleic acid | Sequence of the PCR primer HRe6 |
| 217 | Nucleic acid | Sequence of the PCR primer HRe9 |
| 218 | Nucleic acid | Sequence of the PCR primer HRe7 |
| 219 | Nucleic acid | Sequence of the PCR primer HRe8 |
| 220 | Nucleic acid | Sequence of the PCR primer HRe26 |
| 221 | Nucleic acid | Sequence of the PCR primer HRe27 |
| 222 | Nucleic acid | Chimeric nucleic acid molecule consisting of nucleic acid molecule (SEQ |
| | | ID No.: 171) coding for the CBP protein (SEQ ID No.: 172) fused with the |
| | | nucleic acid molecule (SEQ ID No.: 165) coding for the KBD-B protein |
| | | (SEQ ID No.: 166), and a mutated linker sequence |
| 223 | Protein | Translation product of the nucleic acid molecule according to SEQ ID No.: |
| | | 222 |
| 224 | Protein | Mutagenized linker sequence of the nucleic acid molecule SEQ ID. No.: |
| | | 222; |
| | | Joining sequence between the nucleic acid molecules SEQ ID No.: 165 |
| | | and 171; |
| | | Mutation of the protein sequence GGTGGSELE to GGTGGSGGG |
| 225 | Nucleic acid | Sequence of the PCR primer Lib 230 |
| 226 | Nucleic acid | Sequence of the PCR primer Lib 231 |
| 227 | Nucleic acid | Chimeric nucleic acid molecule coding for the KBD-B protein (SEQ ID No.: |
| | | 166) fused with the C16 modulus of the spider silk protein ADF-4C from |
| | | Araneus diadematus (SEQ ID No.: 150) |
| 228 | Protein | Chimeric translation product of SEQ ID No.: 227 |
| 229 | Nucleic acid | Sequence of the quick change PCR primer HRe22 |
| 230 | Nucleic acid | Sequence of the quick change PCR primer HRe23 |

### EXPERIMENTAL EXAMPLES

The following examples are disclosed in order to illustrate preferred embodiments of the present invention. These examples are not to be regarded as being exhaustive or limiting the subject matter of the invention.

In the experimental description, the following abbreviations are used:

(2-amino-2-methylpropanol) AMP, (degrees Celsius) ° C., (ethylenediaminetetraacetic acid) EDTA, (hindered amine stabilizer) HAS, (1,1-difluoroethane) HFC 152, (International Nomenclature of Cosmetic Ingredients) INCI, (milliliters) ml, (minutes) min, (oil/water) O/W, (polyethylene glycol) PEG-25, (paraminobenzoic acid) PABA, (parts per million) ppm, (quantum satis) q.s., (vinylpyrrolidone) VP, (water/oil) W/O, (active ingredient) AI, (polyvinylpyrrolidone) PVP, (keratin-binding domain) KBD, (keratin-binding domain B of human desmoplakin) KBD-B, (keratin-binding domain C of human desmoplakin) KBD-C, (a construct as explained in the description, of a KBD with an effector protein/peptide, where the linkage can be brought about by a chemical reaction or have been produced directly as fusion protein, (e.g. through expression of a translation fusion DNA construct in a host organism) fusion protein-KBD, (keratin-binding domain of human plakophilin) KBD-D.

### Example 1

### Expression Vectors and Production Strains

Various expression vectors were tested for the expression of the keratin-binding domains (KBD). For this, various promoters were used (e.g. IPTG-inducible, rhamnose-inducible, arabinose-inducible, methanol-inducible, constitutive promoters, etc.). Constructs were likewise tested in which the KBD were expressed as fusion proteins (e.g. as fusion with C16 spider silk protein [Huemmerich et al.; 2004, Primary structure elements of spider dragline silks and their contribution to protein solubility; Biochemistry 43: 13604-13612] (also called C16 below), thioredoxin, or eGFP, or YaaD [B. subtilis, SWISS-PROT: P37527, PDX1], or carotenoid-binding protein [Bombyx mori, SWISS-PROT: Q8MYA9] (also called CBP below), or metal binding protein ZntA [E. coli, SWISS-PROT. P37617]) etc.). Here, both the described KBD-B (keratin-binding domain B, SEQ ID No.: 4), and KBD-C (keratin-binding domain C, SEQ ID No.: 10), and the combination of the two domains KBD-BC were expressed using the various expression systems. The vector constructs mentioned are nonlimiting for the claim.

Given by way of representative as an example is the vector map of the IPTG-inducible vectors pQE30-KBD-B (FIG. 1), pLib076 (FIG. 2) and pRee017 (FIG. 4) and pLib072 (FIG. 5). The procedure for KBD-C may also be analogous to the described vector constructions and expressions.

For the expression of the KBD, various production hosts were used, such as, for example, E. coli strains (see Ex. 2; e.g. XL10-Gold [Stratagene], BL21-CodonPlus [Stratagene], and others). However, other bacterial production hosts, such as, for example, Bacillus megaterium or Bacillus subtilis, were also used. In the case of the KBD expression in B. megaterium, the procedure was carried out analogously to: Barg, H., Malten, M. & Jahn, D. (2005). Protein and vitamin production in Bacillus megaterium. In Methods in Biotechnology-Microbial Products and Biotransformations (Barredo, J.-L., ed, 205-224).

Suitable fungal production strains are also Pichia pastoris (e.g. GS115 and KM71 [both from Invitrogen]; and others) and Aspergillus nidulans (e.g. RMS011 [Stringer, M A, Dean, R A, Sewall, T C, Timberlake, W E (1991) Rodletless, a new Aspergillus developmental mutant induced by direct gene activation. Genes Dev 5:1161-1171] and SRF200 [Karos, M, Fischer, R (1999) Molecular characterization of HymA, an evolutionarily highly conserved and highly expressed protein of Aspergillus nidulans. Mol Genet Genomics 260:510-521], and others). However, it is also possible to use other fungal production hosts, such as, for example, Aspergillus niger (KBD expression analogous to EP 0635574A1 and/or WO 98/46772) for the KBD expression.

### Example 2

### KBD Expression in E. Coli Strains with Iptg Inducible Promoters, e.g. by the Expression Plasmid pQE30-KBD-B

For the expression, various production hosts were used, such as, for example, various E. coli strains (e.g. XL10-Gold [Stratagene], BL21-CodonPlus [Stratagene], and others), Bacillus megaterium, Bacillus subtilis etc.

Described here—by way of representation as an example—is the cloning and expression of KBD-B by E. coli, transformed with pQE30-KBD-B:

Cloning of pQE30-KBD-B
Lambda-MaxiDNA (DNA-Lambda Maxi Kit, Qiagen) was prepared from a cDNA bank of human keratinocytes (BD Bioscience, Clontech, Human Keratinocyte cDNA, foreskin, primary culture in log phase, vector: □gt11).
The PCR was carried out using the following oligonucleotides:

| | |
|---|---|
| Bag 43 | |
| (5′-GGTCAGTTACGTGCAGCTGAAGG-3′) | (SEQ ID No.: 141) |
| and | |
| Bag 44 | |
| (5′ GCTGAGGCTGCCGGATCG-3′) | (SEQ ID No.: 142) |

### 50 μl PCR Mixture:

| | | |
|---|---|---|
| 10x PCR buffer Pfu Ultra High Fidelity: | 5 | μl |
| Lambda DNA (744 ng/μl) | 1 | μl (1:30 dilution) |
| dNTP's.-Mix (10 mM) | 1 | μl |
| Oligo Bag 43 (192 ng/μl) | 0.5 | μl |
| Oligo Bag 44 (181 ng/μl) | 0.5 | μl |
| Pfu Ultra High Fidelity Polymerase | 1 | μl |
| H₂O | 41 | μl |

### Temperature Program:

$$30 x \{ \begin{matrix}2 min & - 95 ° C . \\ 30 sec & - 95 ° C . \\ 30 sec & - Gradient 50 ° C . -> 60 ° C . \\ 2 min 30 sec & - 72 ° C . \\ 10 min & - 72 ° C .\end{matrix}$$

The resulting PCR product about 1102 bp in size was cut out of an agarose gel and purified.
Using the purified PCR product as template, a 2nd PCR was then carried out:

Oligonucleotides Used:

Bag 53:

(SEQ ID No.: 143)

(5′-CGCGCCTCGAGCCACATACTGGTCTGC-3′)
and
Bag 51

(SEQ ID No.: 144)

(5′-GCTTAGCTGAGGCTGCCGGATCG-3′)

### 50 μl PCR Mixture:

| | |
|---|---|
| 10x PCR buffer TAQ: | 5 μl |
| Template from above PCR | 3.5 μl |
| dNTP's.-Mix (10 mM) | 1 μl |
| Oligo Bag 53 (345 ng/μl) | 0.5 μl |
| Oligo Bag 51 (157 ng/μl) | 0.5 μl |
| TAQ Polymerase | 1 μl |
| H₂O | 39 μl |

### Temperature Program:

$$30 x \{ \begin{matrix}2 min & - 95 ° C . \\ 30 sec & - 95 ° C . \\ 30 sec & - 58 ° C . \\ 3 min & - 72 ° C . \\ 10 min & - 72 ° C .\end{matrix}$$

The resulting PCR product about 1073 bp in size was cut out of an agarose gel, purified and cloned in the following vector: pCR2.1-TOPO (Invitrogen).
The resulting vector pCR2.1-TOPO+KBD-B (5027 bp) was then transformed, amplified in E. coli, then cleaved with XhoI and EcoRI and the resulting KBD-B fragment was cloned in pBAD/HisA (Invitrogen; likewise cleaved with XhoI and EcoRI).
The newly formed vector pBAD/HisA+KBD-B (5171 bp) was again cleaved with SacI and StuI and the resulting KBD-B fragment was cloned in pQE30 (Qiagen; cleaved with SacI and SmaI). The resulting expression vector pQE30-KBD-B (4321 bp; see also FIG. 1) was used for the following KBD-B expressions.

The KBD-B (SEQ ID No.: 4) expressed by the vector pQE30-KBD-B in E. coli additionally included, on the N-terminus, the amino acids MRGSHHHHHHGSACEL, and, on the C-terminus, the amino acids GVDLQPSLIS (SEQ ID No.: 166).

Expression of KBD-B by pQE30-KBD-B in E. Coli
Precultures were inoculated from plate or glycerol culture with pQE30-KBD-B transformed E. coli strains (e.g. XL10-Gold [Stratagene]). Depending on the size of the main culture, inoculation with LB medium (about 1:100) was carried out in a tube or a small flask.
Antibiotics were used according to the strain used (for pQE30-KBD-B ampicillin 100 μg/ml).
Incubation was carried out at 250 rpm and 37° C.
The main culture was inoculated about 1:100 with preculture, main culture: LB medium or suitable minimal medium with the respective antibiotics. Incubation at 250 rpm and 37° C.
Induction was carried out with 1 mM IPTG above an OD(600 nm) of 0.5.
After induction for 4 h, the cells were centrifuged off.

In fermenters the procedure was analogous, although it was possible to carry out induction at very much higher OD units and thus to considerably increase the cell and protein yield.

### Example 3

### C16-KBD Expression in E. coli Strains with IPTG Inducible Promoters, e.g. by the Expression Plasmid pLib76

Various production hosts were used for the expression, such as, for example, various E. coli strains (e.g. XL10-Gold [Stratagene], BL21-CodonPlus [Stratagene], and others), Bacillus megaterium, Bacillus subtilis etc.

Described here is—by way of representation as an example—the cloning and expression of C16-KBD-B by E. coli, transformed with pLib76:

Cloning of pLib76
The BsgI cleavage site present in the KBD-B sequence of the vector pQE30-KBD-B (see Example 2) was removed using Quickchange XL Site-Directed Mutagenesis Kit (Stratagene) using the oligonucleotides Lib197 (5′-GAGCTCTCGACTCCTGACAATCAC-3′) (SEQ ID NO.: 145) and Lib198 (5′-GAGCTCGGTTCCTCCGGTACCGCCTCTCCTGCGCAACAATCTTAACG-3′) (SEQ ID NO.: 146) in accordance with the manufacturer's instructions (SEQ ID No.: 147). The resulting vector was called pLib50.
Plasmid DNA of the vector pLib50 served as matrix for a PCR with the oligonucleotides Lib201 (5′-CGTACTGCATGCGGCGGTAGCGGAGGAACTGCACAAGAGCTCGAGCCACATACTGGTCTGCT CTTGC-3-) (SEQ ID NO.: 148) and Lib202 (5′-CTGCAGGTCGACCCCCTCCTGAACAGACATTTC-3′) (SEQ ID NO.: 149). Via the oligonucleotide Lib201, a BsgI cleavage was introduced into the fragment.

The PCR were carried out in 50 μl reaction mixtures with the following compositions:

1 μl of plasmid DNA pLib50
1 μl of dNTP mix (each 10 mM; Eppendorf)
5 μL of 10×PCR buffer+MgCl₂ (Roche)
1 μl of Lib201 5′ primer (corresponds to 50 pmol)
1 μl of Lib202 3′ primer (corresponds to 50 pmol)

### 5 U of Two-polymerase (Roche)

top up to 50 μl with H₂O

The PCR reactions were carried out under the following cycle conditions:

Step 1: 5 minutes at 95° C. (denaturation)
Step 2: 60 seconds at 95° C.
Step 3: 45 seconds at 50° C. (annealing)
Step 4: 2 minutes at 72° C. (elongation)
30 cycles of steps 2-4
Step 5: 10 minutes at 72° C. (post-elongation)
Step 6: 4° C. (pause)
The resulting PCR product, approximately 924 bp in size, was cut out of an agarose gel, purified and cloned into the following vector: pCR2.1-TOPO (Invitrogen).
The resulting vector pLib58 was then transformed, amplified in E. coli, then cleaved with SphI/SalI and the resulting KBD-B fragment was cloned into pQE30-KBD-B (see Example 2; likewise cleaved with SphI/SalI).
A fragment of the C16 modulus of the spider silk protein ADF-4 (SEQ ID No.: 150) (Huemmerich et al. [2004; Primary structure elements of spider dragline silks and their contribution to protein solubility; Biochemistry 43: 13604-13612]) cleaved with BsgI/BseRI was ligated into the resulting vector pLib59 (cleaved with BsgI).
This cloning produced a chimeric nucleic acid molecule (SEQ ID No.:167) coding for the C16 protein (SEQ ID No.:151) fused with the KBD-B protein (SEQ ID No.:166). Ligation of the encoding nucleic acid molecules (SEQ ID No.:150 and SEQ ID No.:147) results in a translation fusion of said proteins and, after translation has taken place, leads to a protein according to SEQ ID No.:168. The resulting expression vector pLib76 (see also FIG. 2) was used for the subsequent C16-KBD-B expressions.
   Expression of C16-KBD-B by pLib76 in E. coli (See Also FIG. 2)
Precultures were inoculated from plate or glycerol culture with pLib76 transformed E. coli strains (e.g. XL10-Gold [Stratagene]). Depending on the size of the main culture, inoculation was carried out in a tube or in a small flask containing LB medium (about 1:100).
Antibiotics were used according to the strain used (for pLib76 ampicillin 100 μg/ml).
Incubation at 250 rpm and 37° C.
The main culture was inoculated about 1:100 with preculture, main culture: LB medium or suitable minimal medium with the respective antibiotics. Incubation at 250 rpm and 37° C.
Induction was carried out with 1 mM IPTG above an OD(600 nm) of 0.5. The cells were then incubated at 32° C. and 250 rpm.
After induction for 4 h, the cells were centrifuged off.
In fermenters, the procedure was analogous, except that induction was possible at very much higher OD units, and thus the cell and protein yield could be increased considerably.

FIG. 6 shows the expression of C16-KBD-B, which were investigated by antibodies directed toward the N-terminal His tag of the C16-KBD-B fusion, or directed toward the KBD-B domain, in a Western Blot. In each case, a protein of the same size was detected. This demonstrates that the protein expressed in E. coli actually consists of the C16 domain and also of the KBD-B domain. The IPTG concentrations used for inducing the expression achieved comparable results.

Overall, the data show that successful expression of C16-KBD-B in E. coli has been achieved.

### 3.a KBD-B-C16 Expression in a E. Coli Strain with IPTG Inducible Promoter by the Expression Plasmid pLib78

In contrast to the fusion protein variant described in Example 3 comprising N-terminally the C-16 silk protein and C-terminally the KBD-B protein, this example describes the cloning and expression of the reverse variant of said fusion protein.

Various production hosts were used for the expression, such as, for example, various E. coli strains (e.g. XL1 0-Gold [Stratagene], BL21-CodonPlus [Stratagene], BLR(DE3) [Novagen] and others), Bacillus megaterium, Bacillus subtilis etc.

Described here is—by way of representation as an example—the cloning and expression of KBD-B-C16 by E. coli, transformed with pLib78:

Cloning of pLib78
Plasmid DNA of the vector pLib15 comprising the KBD-B DNA sequence served as matrix for a PCR with the oligonucleotides

Lib230
(SEQ ID NO: 225)
(5′-AGATCTCATCACCATCACCATCACGAGCCACATACT-3′)
and
Lib231
(SEQ ID NO: 226)
(5′-AGATCTAGTTCCTCCGGTACCGCCGCTAATTAAGCTTGGCTGCAGGTC-3′)

In each case, a BglII cleavage site was introduced via the oligonucleotides Lib230 and Lib231, and a linker sequence was introduced into the fragment via the oligonucleotide Lib231.

The PCR was carried out in 100 μl reaction mixture which had the following composition:
5 μl of plasmid-DNA pLib15
1 μl of dNTP mix (10 mM; Eppendorf)
10 μl of herculase buffer (10×; Stratagene)
4 μl of Lib230 5′ primer (corresponds to 50 pmol)
4 μl of Lib231 3′ primer (corresponds to 50 pmol)
1 μl of herculase polymerase (5 U/μl; Stratagene)
topped up to 100 μl with H2O.

The PCR reaction were carried out under the following cycle conditions:
Step 1: 5 minutes at 95° C. (denaturation)
Step 2: 1 minute at 95° C.
Step 3: 1 minute at 60° C. (annealing)
Step 4: 1.5 minutes at 72° C. (elongation)
30 cycles of steps 2-4
Step 5: 10 minutes at 72° C. (post-elongation)
Step 6: 4° C. (pause)

The PCR product, about 945 bp in size, was cut out of an agarose gel, purified and cloned into the vector pCR2.1-TOPO (Invitrogen). The newly formed plasmid was called pLib77.

pLib77 was then transformed, amplified in E. coli, then cleaved with BglII and the resulting KBD-B fragment was cloned into the plasmid pET21a(+)C16 comprising the C16 sequence (Hümmerich et al, 2004, Biochemistry 43: 13604-13612). The receiving vector had been cleaved beforehand with BamHI.

This cloning produced a chimeric nucleic acid molecule (SEQ ID NO: 227) coding for the KBD-B protein (SEQ ID No.: 166) fused with the C16 protein (SEQ ID No.: 151). Ligation of the coding nucleic acid molecules (SEQ ID No.: 165 and SEQ ID No.: 150) results in a translation fusion of said proteins and, after translation has taken place, leads to a protein according to SEQ ID No.: 228. The resulting expression vector pLib78 (see also FIG. 11) was used for the subsequent KBD-B-C16 expressions, Expression of KBD-B-C16 by pLib78 in E. coli
Precultures were inoculated from plate or glycerol culture with the pLib78 transformed E. coli strain BLR(DE3) from Novagen. Depending on the size of the main culture, inoculation was carried out in a tube or a small flask containing LB medium (about 1:100).
The antibiotic used was ampicillin 100 μg/ml corresponding to the vector.
Incubation at 250 rpm and 37° C.
The main culture was inoculated about 1:100 with preculture
Main culture: LB medium with ampicillin 100 pg/ml. Incubation at 250 rpm and 37° C.
Induction was carried out with 100 μM IPTG above an OD (600 nm) of 0.5. The cells were then incubated for a further 3 hours.
After induction for 3 h, the cells were centrifuged off.
In fermenters, the procedure was analogous, except that induction was possible at very much higher OD units, and thus the cell and protein yield could be increased considerably.

FIG. 12 shows the expression of KBD-B-C16 which was investigated by antibodies directed toward the T7 tag, the N-terminal His tag, or toward the KBD-B domains of the KBD-B-C16 fusion in a Western Blot. In each case, a protein of the same size was detected. This demonstrates that the protein expressed in E. coli actually consists of the C16 domain and also of the KBD-B domain.

Overall, the data show that successful expression of KBD-B-C16 in E. coli has been achieved.

Purification of the protein was carried out as described in Example 11. The purified fusion protein KBD-B-C16 had the expected relative molecular mass of about 82 500. It could be demonstrated with antibodies directed toward the His tag, with antibodies directed toward the T7 tag and antibodies directed toward the KBD-B.

In order to check the functionality of the formed fusion protein KBD-B-C16 with regard to keratin-binding domain, a hair-binding test was carried out (see Example 16). In this, binding of the fusion protein to hair was demonstrated. The functionality of the C16 protein moiety in the KBD-B-C16 fusion protein was checked by means of producing microbead and film assembly forms (for method, see Example 22a). The KBD-B-C16 fusion protein assembled to films and microbeads and thus behaved like the C16-KBD-B-fusion protein.

### Example 4

### Carotenoid Binding Protein (CBP)-KBD Expression in E. coli Strains with IPTG Inducible Promoters, e.g. by the Expression Plasmid pRee017

For the expression, various production hosts were used, such as, for example, various E. coli strains (e.g. XL10-Gold [Stratagene], BL21-CodonPlus [Stratagene], and others), Bacillus megaterium, Bacillus subtilis etc.

Described here is—by way of representation as an example—the cloning and expression of CBP-KBD-B by E. coli, transformed with pRee017:

Cloning of pRee017
The DNA of the gene coding for the CBP from B. mori (SWISS-PROT: Q8MYA9) was prepared synthetically and ligated into a plasmid vector. The resulting plasmid 051794pPCR-script served as matrix for a PCR with the oligonucleotides Lib199 (5′-GAGCTCGCCGACTCTACGTCGAAAAGC-3′) (SEQ ID NO.: 169) and Lib200 (5′-GAGCTCAGAACCTCCGGTAGCACCGATTTCGGCTCTGGCCTTCGCTTGGCCAC-3′) (SEQ ID NO.: 170).

The PCR were carried out in 100 μl reaction mixtures with the following composition,
1 μl of plasmid DNA 051794 pPCR-script with CBP
1 μl of dNTP-mix (each 10 mM; Eppendorf)
10 μl of 10× Herculase buffer (Stratagene)
4 μl of Lib199 5′ primer (240 μg/ml)
4 μl of Lib200 3′ primer (730 μg/ml)
5 U of Herculase (Stratagene)
top up to 100 μl with H₂O

The PCR reactions were carried out under the following cycle conditions:
Step 1: 5 minutes at 95° C. (denaturation)
Step 2: 1 minute at 95° C.
Step 3: 1 minute at 58° C. (annealing)
Step 4: 1.5 minutes at 72° C. (elongation)
30 cycles of steps 2-4
Step 5: 10 minutes at 72° C. (post-elongation)
Step 6: 40C (pause)
The resulting PCR product, about 918 bp in size, was cut out of an agarose gel, purified and cloned into the following vector: pCR2.1-TOPO (Invitrogen).
The resulting vector pLib54 was then transformed and amplified in E. coli.

The CBP gene was amplified in the next step with the primers

HRe1

(SEQ ID No.: 173)

(5′-AAAGCATGCGCCGACTCTACGTCGAAAAGCGCG-3′)
and
HRe2

(SEQ ID No.: 174)

(5′-CCTTGAGCTCAGAACCTCCGGTACCACCGAT-3′) by a PCR.

### 50 μl of PCR Mixture:

| | |
|---|---|
| 10x of PCR buffer for Accu Prime | 5 μl |
| Polymerase (Invitrogen) | |
| pLib54 (50 ng) | 1 μl |
| dNTP's.-Mix (10 mM) | 3 μl |
| Oligo HRe1 (312 ng/μl) | 1 μl |
| Oligo HRe2 (338 ng/μl) | 1 μl |
| Accu Prime Polymerase (Invitrogen) | 1 μl |
| H₂O | 38 μl |

### Temperature Program:

$$29 x \{ \begin{matrix}1 min . & - 95 ° C . \\ 1 min . & - 95 ° C . \\ 1 min . & - 52 ° C . \\ 3 min . & - 72 ° C . \\ 8 min & - 72 ° C .\end{matrix}$$

The resulting fragment (SEQ ID No.: 171) was cleaved with SphI and SacI and cloned into pQE30-KBD-B (see Example 2; likewise cleaved with SphI and SacI). This cloning produced a chimeric nucleic acid molecule (SEQ ID No.: 175) coding for the COBP protein (SEQ ID No.:172) fused with the KBD-B protein (SEQ ID No.:166). The resulting expression vector pRee017 (FIG. 4) thus comprised the nucleic acid molecule (SEQ ID. No.:175) coding for the CBP protein (SEQ ID. No.:172) fused with the nucleic acid molecule (SEQ ID No.: 165) coding for the KBD-B protein (SEQ ID. No.: 166). Ligation of said nucleic acid molecules results in a translation fusion of said proteins and, after translation has taken place, leads to a protein according to SEQ ID No.:176).

In a further embodiment, a further variant of the chimeric nucleic acid molecule with the SEQ ID No.: 175 was produced in which the joining sequence, which joins the two nucleic acids coding for the CBP protein (SEQ ID No.: 172) and the KBD-B protein (SEQ ID No.: 166) was changed through targeted mutagenesis (Quick Change Site Directed Mutagenesis Kit, Stratagene). The procedure was carried out in accordance with the manufacturer's instructions. The oligonucleotides used were HRe22 (SEQ ID No: 229) and HRe23 (SEQ ID No: 230). The template used was pRee017 (FIG. 4).

The resulting expression vector pRee023 thus comprised the nucleic acid molecule SEQ ID No.: 222 which codes for a fusion protein consisting of the CBP protein (SEQ ID No.: 172) and the KBD-B protein (SEQ ID No.: 166), where the sequence joining the two proteins is a mutagenized linker sequence (SEQ ID No: 224). Ligation of said nucleic acid molecules results in a translation fusion of said proteins and, after translation has taken place, leads to a fusion protein according to SEQ ID No.: 223, which is characterized by particular proteolytic stability in the production strain. The resulting expression vectors pRee017 (see also FIG. 4) and pRee023 were used for the following CBP-KBD-B expressions.

Expression of CBP-KBD-B by pRee017 or pRee023 in E. coli
Precultures were inoculated from plate or glycerol culture with pRee017 or pRee023 transformed to E. coli strains. Depending on the size of the main culture, inoculation was carried out in a tube or in a small flask LB medium (about 1:100)
Antibiotics were used according to the strain used (for strains transformed with pRee017 or pRee023, 100 μg/ml of ampicillin)
Incubation at 250 rpm and 37° C.,
The main culture was inoculated about 1:100 with preculture, main culture: LB medium or suitable minimal medium with the respective antibiotics. Incubation at 250 rpm and 37° C.
Induction was carried out with 1 mM IPTG above an OD (600 nm) of 5. The cells were then incubated at 32° C. and 250 rpm.
After induction for 4 h, the cells were centrifuged off.
Purification of the protein was carried out as described in Example 11.
The purified fusion protein CBP-KBD had the expected relative molecular mass of about 70 000. It could be detected with antibodies directed toward the His tag and antibodies directed toward KBD.

### Example 5

### Metal Binding Protein (ZntA)-KBD Expression in E. coli Strains with IPTG Inducible Promoters, e.g. by the Expression Plasmid pLib72

For the expression, various production hosts were used, such as, for example, various E. coli-strains (e.g. XL10-Gold [Stratagene], BL21-CodonPlus [Stratagene], and others), Bacillus megaterium, Bacillus subtilis etc.

Described here is—by way of representation as an example—the cloning and expression of ZntA-KBD-B by E. coli, transformed with pLib72:

Cloning of pLib72
Chromosomal E. coli DNA served as matrix for PCR with the oligonucleotides Lib212 (5′-GAGCTCTCGACTCCTGAGAATCAC-3′) (SEQ ID NO: 177) and Lib219 (5′-GAGCTCGGTTCCTCCGGTACCGCCTCTCCTGCGCAACAATCTTAACG-3′) (SEQ ID No: 178).
The resulting PCR product, about 2223 bp in size, was cut out of an agarose gel, purified and cloned into the following vector: pCR2.1-TOPO (Invitrogen).
The resulting vector pLib71 was then transformed, amplified in E. coli, then cleaved with SacI and the resulting zntA fragment (SEQ ID No.: 179) was cloned into pQE30-KBD-B (see Example 2; likewise cleaved with SacI). This cloning produced a chimeric nucleic acid molecule (SEQ ID. No.:181), coding for the ZntA protein (SEQ ID. No.:180) fused with the KBD-B protein (SEQ ID. No.: 166). The resulting expression vector pLib72 (FIG. 5) thus comprised a nucleic acid molecule (SEQ ID. No.:179) coding for the ZntA protein (SEQ ID. No.:180) fused with the nucleic acid molecule (SEQ ID No.: 165) coding for the KBD-B protein (SEQ ID. No.: 166). The ligation of said nucleic acid molecules results in a translation fusion of said proteins and, after translation has taken place, leads to a protein according to SEQ ID No.:182. The resulting expression vector pLib72 (see also FIG. 5) was used for the following ZntA-KBD-B expressions.

The PCR were carried out in 50 □l reaction mixtures with the following composition:
1 □l of genom. DNA XL10-Gold (1.7 μg)
1 □l of dNTP-Mix (each 10 mM; Eppendorf)
5 □l of 10× Herculase buffer (Stratagene)
1 □l of Lib212 5′ primer (341 μg/ml)
2 □l of Lib219 3′ primer (464 μg/ml)
5 U of Herculase (Stratagene)
top up to 50 μl with H₂O

The PCR reactions were carried out under the following cycle conditions:
Step 1: 5 minutes at 95° C. (denaturation)
Step 2: 1 minute at 95° C.
Step 3: 45 seconds at 60° C. (annealing)
Step 4: 2 minutes at 72° C. (elongation)
35 cycles of steps 2-4
Step 5: 10 minutes at 72° C. (post-elongation)
Step 6: 4° C. (pause)

### Example 6

### Thioredoxin-KBD Expression in E. coli Strains with IPTG Inducible Promoters

For the expression, various production hosts were used, such as, for example, various E. coli strains (e.g. XL10-Gold [Stratagene], BL21-CodonPlus [Stratagene], and others), Bacillus megaterium, Bacillus subtilis etc.

Described here is—by way of representation as an example—the cloning and expression of thioredoxin-KBD-B by E. coli:

Firstly, the thioredoxin fragment of interest from the vector pThioHisC (Invitrogen) was amplified by PCR (PCR mixture conditions analogous to Example 2). For this purpose, the following oligonucleotides were used:

Bag 102:

(SEQ ID No.: 183)

(5′-GTAAGAATGCGGCGGCCTCCTGAACAGACATTTCTTTATTG-3′)
Bag 103:

(SEQ ID No.: 184)

(5′-GCAGATCTAGAGGATCGCATCACCATCACCATCACGGATCC-3′)

The amplified PCR product (SEQ ID No.: 185) was cut out of an agarose gel, purified, cleaved with the restriction endoribonucleases NotI and BglII and cloned into pQE30-KBD-B (see Example 2).
This cloning produced a chimeric nucleic acid molecule (SEQ ID. No.:187) coding for the thioredoxin protein (SEQ ID. No.:186) fused with the KBD-B protein (SEQ ID. No.: 166). The resulting expression vector thus comprised a nucleic acid molecule (SEQ ID. No.:185) coding for the thioredoxin protein (SEQ ID. No.:186) fused with the nucleic acid molecule (SEQ ID No.: 165) coding for the KBD-B protein (SEQ ID. No.: 166). Ligation of said nucleic acid molecules results in a translation fusion of said proteins and, after translation has taken place, leads to a protein according to SEQ ID No.:188. The resulting expression vector was used for the following thioredoxin-KBD-B expressions.

### Example 7

### eGFP-KBD Expression In E. coli Strains with IPTG Inducible Promoters

For the expression, various production hosts were used, such as, for example, various E. coli strains (e.g. XL10-Gold [Stratagene], BL21-CodonPlus [Stratagene], and others), Bacillus megaterium, Bacillus subtilis etc.

Described here—by way of representation as an example—are the cloning and expression of eGFP-KBD-B by E. coli:

Firstly, the eGFP fragment of interest was amplified from the vector pEGFP-1 (Clontech) by PCR (PCR mixture conditions analogous to Example 2). For this purpose, the following oligonucleotides were used:

| | |
|---|---|
| Bag 89: | (5′-GCGAGCTCGTGAGCAAGGGCGAGGAGC-3′) |
| Bag 90: | (5′-GGGAGCTCCTTGTACAGCTCGTCCATG-3′) |

The amplified PCR product (SEQ ID No.: 191) was cut out of an agarose gel, purified, cleaved with the restriction endoribonuclease SacI and cloned into pQE30-KBD-B (see Example 2).
This cloning produced a chimeric nucleic acid molecule (SEQ ID. No.: 193) coding for the eGFP protein (SEQ ID. No.:192) fused with the KBD-B protein (SEQ ID. No.: 166). The resulting expression vector thus comprised a nucleic acid molecule (SEQ ID. No.:191) coding for the eGFP protein (SEQ ID. No.:192) fused with the nucleic acid molecule (SEQ ID No.: 165) coding for the KBD-B protein (SEQ ID. No.: 166). Ligation of the specified nucleic acid molecules results in a translation fusion of said proteins and, after translation has taken place, leads to a protein according to SEQ ID No.:194. The resulting expression vector was used for the following eGFP-KBD-B expressions.

### Example 8

### YaaD-KBD Expression in E. coli Strains with IPTG Inducible Promoters

For the expression, various production hosts were used, such as, for example, various E. coli strains (e.g. XL10-Gold [Stratagene], BL21-CodonPlus [Stratagene], and others), Bacillus megaterium, Bacillus subtilis inter alia.

Described here is—by way of representation as an example—the cloning and expression of YaaD-KBD-B by E. coli:

Firstly, the YaaD fragment of interest from the vector pDX14 (OmniGene Bioproducts) was amplified by PCR (PCR mixture conditions analogous to Example 2). For this purpose, the following oligonucleotides were used:

Bag 93:

(SEQ ID No: 195)

(5′-GCGAGCTCGCTCAAACAGGTACTGAACG-3′)
Bag 94:

(SEQ ID No: 196)

(5′-GCGAGCTCCCAGCCGCGTTCTTGCATACG-3′)

The amplified PCR product (SEQ ID No.: 197) was cut out of an agarose gel, purified and ligated into pCR2.1 TOPO (without restriction digestion). The YaaD was cut out of the plasmid pCR2.1 TOPO-YaaD with SacI and cloned into pQE30-KBD-B (see Example 2).
This cloning produced a chimeric nucleic acid molecule (SEQ ID. No.:199) coding for the yaaD protein (SEQ ID. No.:198) fused with the KBD-B protein (SEQ ID. No.: 166). The resulting expression vector thus comprised a nucleic acid molecule (SEQ ID. No.:197) coding for the yaaD protein (SEQ ID. No.:198) fused with the nucleic acid molecule (SEQ ID No.: 165) coding for the KBD-B protein (SEQ ID. No.: 166). Ligation of the specified nucleic acid molecules results in a translation fusion of said proteins and, after translation has taken place, leads to a protein according to SEQ ID No.: 200. The resulting expression vector was used for the following yaaD-KBD-B expressions.

It goes without saying that the DNA constructs produced by way of example in Examples 3 to 8 for producing keratin-binding fusion proteins can also be produced using the vector pRee024 (FIG. 8, Ex. 18-22). The fusion proteins thus formed comprise the KBD-D protein (SEQ ID No.: 212) in the case of pRee024.

### Example 9

### Expression of KBD by Means of Aspergillus nidulans Strains Using the Inducible alcA Promoter, e.g. by the Expression Plasmid pLib 19 (Shaking Flask)

For the expression, A. nidulans wild-type strains were used, such as, for example, RMS01 1 or SRF200. Described here is—by way of representation as an example—the expression of KBD-B by A. nidulans, transformed with pLib19 (FIG. 6).
For the construction of pLib19, a KBD-B-encoding DNA fragment 922 bp (SEQ ID No.: 152) in size, was amplified by means of PCR using the oligonucleotides Lib151 (5′-CACCATGCATCACCATCACCATCACGAGCCACATACTGGTCTGCT-3′ (SEQ ID No.: 154) and Lib152 (5′-GCTAATTAAGCTTGGCTGCA-3′ (SEQ ID No.: 155) and the vector pQE30-KBD-B (Example 2, FIG. 1) as template.
The PCR were carried out in 50 □l reaction mixtures with the following composition:
1 □l of plasmid DNA pQE30-KBD-B
1 □l of dNTP-Mix (each 10 mM; Eppendorf)
5 □l of 10×PCR buffer+MgCl₂ (Roche)
1 □l of Lib151 5′ primer (corresponds to 50 pmol)
1 □l of Lib152 3′ primer (corresponds to 50 pmol)
5 U of Pwo-polymerase (Roche)
The PCR reactions were carried out under the following cycle conditions:
Step 1: 5 minutes at 95° C. (denaturation)
Step 2: 45 seconds at 95° C.
Step 3: 45 seconds at 53° C. (annealing)
Step 4: 2 minutes at 72° C. (elongation)
30 cycles of steps 2-4
Step 5: 10 minutes at 72° C. (post-elongation)
Step 6: 4° C. (pause)
The PCR product was ligated into the vector pENTR/D (pENTR™ Directional TOPO® Cloning Kit, Version E, Invitrogen). The correct KBD-B amplification was checked by sequencing.
The recombination of the KBD-B encoding DNA fragment was carried out into the vector pMT-OvE (Toews M W, Warmbold J, Konzack S, Rischitor P, Veith D, Vienken K, Vinuesa C, Wei H, Fischer R; Establishment of mRFP1 as a fluorescent marker in Aspergillus nidulans and construction of expression vectors for high-throughput protein tagging using recombination in vitro (GATEWAY). (2004) Curr Genet 45: 383-389) using the “Gateway® LR Clonase™ enzyme mix” (Invitrogen). This produced the vector pLib19 (FIG. 6).
Protoplasts of the A. nidulans wild-type strains were transformed with the circular vector pLib19 (Yelton M M, Hamer J E, Timberlake W E; Transformation of Aspergillus nidulans by using a trpC plasmid., (1984) Proc Natl Acad Sci USA 81: 1479-1474). The transformants were analyzed by means of PCR and Southern blot using chromosomal DNA.
For the preculture of KBD-B-expressing A. nidulans transformants, 100 ml of minimal medium (0.6% NaNO₃; 0.152% KH₂PO₄; 0.052% KCl [pH 6.5]; 0.8% glucose; 0.05% MgSO₄; 1 ml trace element solution [1 g/l FeSO₄×7H₂O; 8.8 g/l ZnSO₄×7H₂O; 0.4 g/l CuSO₄×5H₂O; 0.15 g/l MnSO₄×4H₂O; 0.1 g/l Na₂B₄O₇×10H₂O; 0.05 g/l (NH₄)₆Mo₇O₂₄×4H₂O], +strain-specific supplements) or 100 ml of complete medium (2% malt extract; 0.1% peptone; 2% glucose; +strain-specific supplements) were inoculated in 500 ml flasks with 10⁶-10⁷ spores and incubated for 16-24 h at 200-250 rpm and 37° C.
After the preculture, the fungal mycelium was harvested by filtration, washed with distilled water and transferred to flasks with 100-500 ml of fresh minimal medium. In this main culture medium, 0.1% fructose was used instead of glucose as the C-source. To induce the KBD expression, ethanol (1% final concentration) or glycerol (50 mM) or sodium acetate (50 mM) or ethylamine or threonine were additionally added to the medium. The additives mentioned for inducing the expression are not limiting for the claim. The main culture was incubated for a further 5-48 h at 200-250 rpm and 37° C.
After the end of the culture, the fungal mycelium was harvested with 1500-3000×g for 5 min at room temperature and disrupted by means of a Menton-Gaulin.
Besides the polypeptide sequence SEQ ID No.: 4, the KBD-B expressed in A. nidulans (SEQ ID No.: 152) (pLib19) additionally included, at the N-terminus, the amino acids MHHHHHH, and, at the C-terminus, the amino acids

GVDLQPSLISKGGRADPAFLYKVVMIRLLTKPERKLLEGGPGTQLLFPLVRVNCALGVIM-
VIAVSGVKLLSAHNSTQHTSRKHKV.

### Example 10

### Cell Disruption and Inclusion Body Purification (pQE30-KBD-B)

Solubly expressed KBD or fusion protein-KBD could be used directly following cell disruption (e.g. by means of Menton-Gaulin) or be purified by means of chromatography (see Example 11). Insolubly expressed KBD or fusion protein-KBD (e.g. in inclusion bodies) was purified as follows:
The fermenter contents were centrifuged, the pellet was suspended in 20 mM phosphate buffer pH=7.5 and disrupted by means of a Menton-Gaulin.
The disrupted cells were centrifuged again (15 000g), the pellet from this was treated with 20 mM phosphate, 500 mM NaCl and 8 M urea and so stirred. (Dissolution of the inclusion bodies)
The pH of the supernatant was adjusted to 7.5.
Centrifugation was then carried out again and the supernatant was applied to an Ni chelate Sepharose column and purified as described in Example 6.

### Example 11

### Purification of Keratin-Binding Domain B or Fusion Protein-KBD Over Ni Chelate Sepharose

The KBD or fusion protein-KBD could be purified chromatographically through the attached His-tag over an Ni column.

Column material: Ni-Sepharose High Performance
Amersham Biosciences Order No.:17-5268-02

The material was packed into a column (e.g. diameter 2.6 cm, height 10 cm) and equilibrated with buffer A+4% buffer B (corresponds to 20 mM imidazole).

The protein extract (see e.g. cell disruption and inclusion body purification) was applied to the column at pH 7.5 using a Superloop (ÄKTA system) (flow about 5 ml/min).

Following application, washing was carried out with buffer A+20 mM imidazole.

Elution was carried out with buffer B (500 mM imidazole in buffer A).

The eluate was collected in fractions using a fraction collector.

The eluate was then freed from salt (advantageous for samples which are to be concentrated). For this, the eluate was freed from salt, for example, over a Sephadex G25 medium column (Amersham). Then, for the concentration, for example an Amicon chamber (stirred ultrafiltration cell, Millipore) could be used.

| | |
|---|---|
| Buffer A: | 20 mM sodium dihydrogenphosphate |
| | 500 mM NaCl (if desired, it is also possible to use buffers |
| | with lower NaCl concentrations) |
| | 8 M urea (urea does not need to be used if “active” KBD is |
| | chromatographed which has already been solubly expressed. |
| | Without urea, no subsequent renaturation of the protein |
| | is required.) |
| | pH = 7.50 |

| | | |
|---|---|---|
| Buffer B: | 20 mM | sodium dihydrogenphosphate |
| | 500 mM | NaCl (if desired, it is also possible to use buffers |
| | | with lower NaCl concentrations) |
| | 8 M | urea |
| | 500 mM | imidazole |
| | pH = 7.50 | |

### Example 12

### Renaturation of Keratin-Binding Domain B or Fusion Protein-KBD

Insolubly expressed keratin-binding domain or fusion protein-KBD (e.g. from inclusion bodies) can be renatured and thus activated as follows:

### Method 1: Discontinuous Dialysis

6.5 ml of Cellytic IB (Sigma, order No. C5236) and 5 mM DTT were added to 6.5 ml of KBD-B inclusion bodies or fusion protein-KBD in 8 M urea (Ni chelate eluate, HiTrap). The solution to be renatured was then poured into a dialysis tube (Spectrum: Spectra Por MWCO:12-14 kD).

Carry out dialysis for about 12 hours against 1 L 6 M urea solution at 4° C. with careful stirring.

500 ml of 25 mM Tris/HCl pH=7.50 were added and dialysis was carried out like this for 9 hours at 4° C. Subsequent addition of a further 250 ml of the Tris buffer (see above) and dialysis for a further 12 hours.

500 ml of 25 mM Tris/HCl pH=7.50 were then added again and dialysis was carried out like this for 9 hours at 4° C. Subsequent addition of a further 250 ml of the Tris buffer (see above) and dialysis for a further 12 hours.

500 ml of 25 mM Tris/HCl pH=7.50 were then added again and dialysis was carried out like this for 9 hours at 4° C. The dialysis tube containing the dialyzate was then placed into 2 L: 25 mM Tris+150 mM NaCl pH=7.50. Dialysis was then carried out again at 4° C. for 12 hours.

The contents of the dialysis tube were then removed.

### Method 2: Continuous Dialysis

20 ml of KBD-B inclusion bodies (or fusion protein-KBD) in 8 M urea (Ni chelate eluate, HiTrap) were treated with 10 ml of Cellytic IB (Sigma, order No. C5236) and 5 mM DTT. The solution was then poured into a a dialysis chamber: Slide-A-Lyzer Dialyses Cassette PIERCE, MWCO: 10 kD. order No.: 66830.

Dialysis was then carried out for about 1 hour against 1 L 6 M urea solution at 4° C.

Then, over a period of 48 h, 2 l of the following buffer were metered in continuously by means of a peristaltic pump: 25 mM Tris/HCl pH=7.5.

The dialysis tube containing the dialyzate was then added to 2 L of the end buffer:

25 mM Tris+150 mM NaCl pH=7.50 and dialysis was carried out for about 12 hours at 4° C.

The contents of the dialysis tube were then removed.

### Example 13

### Binding to Skin 1 (Qualitative)

A visual qualitative test was developed in order to examine whether KBD or fusion protein-KBD binds to skin.

### Solutions Used:

Blocking solution: Western Blocking Reagent 1921673 Roche (10× solution) diluted in TBS.

### TBS: 20 mM Tris; 150 mM NaCl pH 7.5

### TTBS: TBS+0.05% Tween20

The first step is the transfer of the outer keratin layer of the skin to a stable support. For this purpose, a transparent adhesive tape is firmly applied to depilated human skin and removed again. The test can be carried out directly on the transparent adhesive strip, or the adhering keratin layer can be transferred to a glass slide through renewed adhesion. Binding was demonstrated as follows:
For incubation with the various reagents, transfer to a Falcon vessel
If appropriate addition of ethanol for degreasing, removal of ethanol and drying of the slide
Incubation with blocking buffer for 1 h at room temperature
2× washing for 5 min with TTBS
1× washing for 5 min with TBS
Incubation with the KBD or fusion protein-KBD to be tested (coupled to tag—e.g. His₆, HA etc.) or control protein in TBS/0.05% Tween 20 for 2-4 h at room temperature
Removal of the supernatant
3× washing with TBS
Incubation for 1 h at room temperature with monoclonal anti-polyhistidine (or specific KBD rabbit) antibodies, diluted 1:2000 in TBS+0.01% blocking
2× washing for 5 min with TTBS
1× washing for 5 min with TBS
Incubation for 1 h at room temperature with anti-mouse IgG alkaline-phosphatase conjugate, diluted 1:5000 in TBS+0.01% blocking
2× washing for 5 min with TTBS
1× washing for 5 min with TBS
Addition of phosphatase substrate (NBT-BCIP; Boehringer MA 1 tablet/40 ml of water 2.5 min; stop: with water)
Optical detection of the colored precipitate with the naked eye or using a microscope. A blue colored precipitate indicates that KBD or fusion protein-KBD has bound to the skin.

### Example 14

### Binding to Skin 2 (Quantitative)

A quantitative test was developed with which the hair/skin binding strength of the KBD or fusion protein-KBD can be compared with nonspecific proteins.

A 5 mm cork borer was used to bore a section out of a thawed dry piece of skin without hair (human or pig) (or in the case of a surface test a section of skin is inserted into a Falcon lid). The sample of skin was then converted to a thickness of 2-3 mm in order to remove any tissue present. The skin sample was then transferred to an Eppendorf vessel (protein low-bind) in order to carry out the binding demonstration (see also FIG. 7; alternatively, the Episkin system [reconstituted human skin] from L'Oreal can also be used):
2× washing with PBS/0.05% Tween 20
Addition of 1 ml of 1% BSA in PBS and incubation for 1 h at room temperature, gentle swinging movements (900 rpm).
Removal of the supernatant
Addition of 100 μg of KBD or fusion protein-KBD in PBS with 0.05% Tween 20; incubation for 2 h at room temperature and gentle swinging movements (900 rpm).
Removal of the supernatant
3× washing with PBS/0.05% Tween 20
Incubation with 1 ml of monoclonal mouse anti-tag (His6 or HA or specific KBD) antibodies with peroxidase conjugate (1:2000 in PBS with 0.05% Tween 20) [Monoclonal AntipolyHistidine Peroxidase Conjugate, produced in mouse, lyophilized powder, Sigma] for 2-4 h at room temperature, gentle swinging movement (900 rpm)
3× washing with PBS/0.05% Tween 20
Addition of peroxidase substrate (1 ml/Eppendorf vessel; composition see below)
Allow reaction to run until a blue coloration (about 90 seconds).
Stop the reaction with 100 μl of 2 M H₂SO₄.
The absorption was measured at 405 nm.

Peroxidase substrate (prepare shortly beforehand):

0.1 ml TMB solution (42 mM TMB in DMSO)
+10 ml substrate buffer (0.1 M sodium acetate pH 4.9)
+14.7 μl H₂O₂ 3% strength

### Example 15

### Binding to Hair (Quantitative)

In order to be able to demonstrate the binding strength of KBD or fusion protein-KBD to hair also relative to other proteins, a quantitative assay was developed (see also FIG. 7). In this test, hair was firstly incubated with KBD (or fusion protein-KBD) and excess KBD (or fusion protein-KBD) was washed off. An antibody-peroxidase conjugate was then coupled via the His tag of the KBD (or fusion protein-KBD). Nonbound antibody-peroxidase conjugate was washed off again. The bound antibody-peroxidase conjugate [Monoclonal AntipolyHistidine Peroxidase Conjugate, produced in mouse, lyophilized powder, Sigma] can convert a colorless substrate (TMB) into a colored product, which can be measured photometrically at 405 nm. The intensity of the absorption indicates the amount of bound KBD (or fusion protein-KBD) or comparison protein. The comparison protein chosen was, for example, YaaD from B. subtilis, which likewise had—as is necessary for this test—a His tag for the detection. Instead of the His tag, other specific antibodies conjugated with—peroxidase can also be used.

5 mg of hair (human) are cut into sections 5 mm in length and transferred to Eppendorf vessels (protein low-bind) in order to carry out the binding demonstration:
Addition of 1 ml of ethanol for degreasing
Centrifugation, removal of ethanol and washing of the hair with H₂O
Addition of 1 ml of 1% BSA in PBS and incubation for 1 h at room temperature, gentle swinging movements.
Centrifugation, removal of the supernatant
Addition of the keratin-binding domains (or fusion protein-KBD) to be tested (coupled to tag—e.g. His₆, HA etc.) or control protein in 1 ml of PBS/0.05% Tween 20; incubation for 16 h at 4° C. (or at least 2 h at room temperature) with gentle swinging movements.
Centrifugation, removal of the supernatant
3× washing with PBS/0.05% Tween 20
Incubation with 1 ml monoclonal mouse anti-tag (His6 or HA) antibodies with peroxidase conjugate (1:2000 in PBS/0.05% Tween 20) [Monoclonal AntipolyHistidine Peroxidase Conjugate, produced in mouse, lyophilized powder, Sigma] for 2-4 h at room temperature, gentle swinging movement
3× washing with PBS/0.05% Tween 20
Addition of peroxidase substrate (1 ml/Eppendorf vessel)
Allow reaction to proceed until blue coloration (about 2 minutes).
Stop the reaction with 100 μl of 2 M H₂SO₄.
The absorption is measured at 405 nm.

Peroxidase substrate (prepare shortly beforehand):

0.1 ml TMB solution (42 mM TMB in DMSO)
+10 ml of substrate buffer (0.1 M sodium acetate pH 4.9)
+14.7 μl H₂O₂ 3% strength
BSA=Bovine serum albumin
PBS=Phosphate buffered salt solution
Tween 20=polyoxyethylene sorbitan monolaureate, n about 20
TMB=3,5,3′,5′-tetramethylbenzidine

A binding test on hair carried out by way of example for KBD-B demonstrated considerable superiority of the binding of KBD-B (SEQ ID No.: 166) to hair compared with significantly poorer binding of the comparison protein YaaD:

TABLE 7
Quantitative KBD activity test Hair: 1) buffer; 2) comparison protein
YaaD; 3) KBD-B denatured; 4) KBD-B renatured. The table shows
the measured absorption values at 405 nm.

| | | |
|---|---|---|
| 1 | Buffer | A_{405 nm} = 0.000 |
| 2 | Comparison protein YaaD | A_{405 nm} = 0.088 |
| 3 | KBD-B denatured | A_{405 nm} = 0.254 |
| 4 | KBD-B renatured | A_{405 nm} = 1.591 |

### Example 16

### Hair Binding Activity of Fusion Protein-KBD-B

In order to check whether the fusion protein-KBD-B also binds to hair, a quantitative binding assay was carried out (see FIG. 7). in this test, hair was firstly incubated with fusion protein-KBD-B and nonbound fusion protein-KBD-B was washed off. A peroxidase was then coupled via the His tag of KBD-B. Nonbound peroxidase was washed off again. The bound peroxide can convert a colorless substrate (TMB) into a colored product, which was measured photometrically at 405 nm. The intensity of the absorption indicates the amount of bound fusion protein-KBD-B. The comparison sample chosen was KBD-B without fusion protein.

| | | |
|---|---|---|
| 1 | KBD-B reference sample | A_{405 nm} = 0.65 |
| 2 | C16-KBD-B fusion protein | A_{405 nm} = 0.59 |

Measurement of the hair-binding activity of the C16-KBD-B fusion protein in comparison to a KBD-B reference protein. As these results show, the hair-binding activity of the fusion protein (A_{405 nm}=0.59) compared to the KBD-B protein (A_{405 nm}=0.65) is virtually unchanged.

Overall, these activity tests show that the various fusion protein-KBD-B constructs have good hair binding activity, like KBD-B itself. In most cases, the hair binding activity is not reduced at all, but at least very seldomly below 20% of the binding activity of KBD-B without fusion protein.

### Example 16a

### Binding of Beta-Carotene to the CBP-KBD Fusion Protein Prepared According to Example 4

A binding test to hair carried out by way of example for the fusion protein CBP-KBD-B (SEQ ID No. 223) showed a comparable binding of CBP-KBD-B (SEQ ID No. 223) to hair compared with the protein KBD-B (SEQ ID No. 4). The same applies to a hair-binding test carried out by way of example with C16-KBD (SEQ ID No. 168) and KBD-B-C16 (SEQ ID 228).

The binding of beta-carotene to CBP-KBD (SEQ ID No. 223) compared to KBD (SEQ ID No. 4) was investigated. The UV-Vis absorption spectrum of beta-carotene has a maximum at 440 nm. This property was used for investigating the beta-carotene binding to the fusion protein. Firstly, varying concentrations (0-20 μM) of a CBP-KBD solution were admixed with identical amounts of an ethanolic beta-carotene solution. As the concentration of the fusion protein increased, the coloration of the solution increased. This observation was confirmed by photometric measurement at 440 nm.

| | 0 μM CBP- | 10 μM CBP- | 20 μM CBP- |
|---|---|---|---|
| | KBD-B | KBD-B | KBD-B |
| Average | 0.059 | 0.195 | 0.261 |
| absorption | | | |
| at 440 nm | | | |

In order to verify the binding of beta-carotene to the CBP-KBD fusion protein, in each case a KBD and CBP-KBD solution (each of identical molarity) was admixed with identical concentrations of an ethanolic beta-carotene solution and dialyzed overnight against buffer. On the next day, the binding of beta-carotene was determined through absorption measurement of the solutions at 440 nm and compared with a calibration curve of a beta-carotene solution. It was established that CBP-KBD binds twice as much beta-carotene compared to KBD despite dialysis. The results show that successful binding of beta-carotene by the fusion protein is achieved.

### Example 17

### Checking the Coupling Success (Ellmann Test)

The success of the effector coupling was monitored using two different tests:
(iv) Ellmann test in which the number of free Cys-SH groups in the protein can be determined before and after the chemical effector protein coupling. Here, a considerable reduction in the free SH groups after coupling indicates good reaction progress.
(v) Activity test in which the binding of the KBD (or fusion protein-KBD) to hair can be measured.

A good fusion protein-KBD should reduce the activity of the fusion protein-KBD compared with uncoupled KBD.

Re (iii)

### Materials Required:

Ellmann's reagent: 5,5′-dithiobis(2-nitrobenzoic acid) (DTNB); 4 mg 1 ml in 0.1 M Na phosphate buffer
0.1 M Na phosphate buffer pH 8.0
Cysteine solution (26.3 mg of cysteine hydrochloride monohydrate/100 ml Na phosphate buffer)

The solutions were and must only be prepared shortly prior to use.

1. In each case 25 μl, 50 μl, 100 μl, 150 μl, 200 μl and 250 μl of cysteine solution were pipetted into test tubes (13×100 mm) for a calibration curve. The protein samples to be determined were poured into separate test tubes (volume <=250 μl). Of the KBD to be tested, an amount of at least 1 mg per reaction mixture was dispensed. In the case of the test tubes, the total volume was then adjusted in each case to 250 μl with Na phosphate buffer. If the volume of 250 μl of sample was exceeded (on account of the required 1 mg of KBD), this was taken into consideration when topping up in point 2 with 2.5 ml of Na phosphate buffer.
2. Addition of in each case 50 μl of Ellmann's reagent and 2.5 ml of Na phosphate buffer. Briefly mix and incubate for 15 min at RT.
3. Measure the absorption at 412 nm
4. Construct the calibration curves, plot and read off the values of the protein samples to be determined.

### Example 18

### Expression of KBD-D (SEQ ID No.: 212) by Means of Escherichia Coli Strains Using the Expression Plasmid pRee024 with an IPTG Inducible Promoter (FIG. 8)

For the expression, the E. coli strain XL10 Gold [Stratagene] was used.

Described here, by way of representation as an example, is the cloning of KBD-D (SEQ ID No.: 212) and the subsequent expression of the KBD-D protein (SEQ ID No.: 213) in E. coli, transformed with pRee024 (FIG. 8);

Cloning of pRee024:
Lambda-MaxiDNA (DNA-Lambda Maxi Kit, Qiagen) was produced from a cDNA bank of human keratinocytes (BD Bioscience, Clontech, Human Keratinocyte cDNA, foreskin, primary culture in log phase, vector, λgt11).

The PCR for the amplification of the KBD-D gene was carried out in two steps. Firstly, the 5′ end and 3′ end was amplified independently. These fragments were the matrices for the amplification of the entire KBD-D gene.

The PCR for the amplification of the 5′ end was carried out as follows:

The primers had the following sequence:

HRe6:

(SEQ ID No.: 216)

5′-ATGAACCACTCGCCGCTCAAGACGCCTTG-3′
HRe9:

(SEQ ID No.: 217)

5′-CGTTCCCGGTTCTCCTCAGGAGGCTGACTG-3′

### 100 μl PCR-Mixture:

| | |
|---|---|
| 10x PCR buffer Pfu Ultra High Fidelity: | 10 μl |
| Lambda DNA (744 ng/μl) | 1 μl |
| | (1:10 dilution) |
| dNTP's.-Mix (10 mM) | 10 μl |
| HRe6 (196 ng/μl) | 1 μl |
| HRe9 (201 ng/μl) | 1 μl |
| Pfu Ultra High Fidelity Polymerase | 1 μl |
| H₂O bidist | 76 μl |

### Temperature Program:

$$30 x \{ \begin{matrix}2 min & 95 ° C . \\ 30 sec & 95 ° C . \\ 30 sec & 63 ° C . \\ 1 min 30 sec & 72 ° C .\end{matrix}$$

A fragment about 1 kb in size was detected in the agarose gel. The reaction was purified and used below as 5′ end template for the amplification of the KBD-D gene.

The PCR for the amplification of the 3′ end was carried out as follows:

The primers had the following sequence:

HRe7:

(SEQ ID No.: 218)

5′-TTAGAATCGGGAGGTGAAGTTCCTGAGGCT-3′
HRe8:

(SEQ ID No.: 219)

5′-CACCACCAACAAGCTGGAGACCCGGAG-3′

### 100 μl PCR Mixture:

| | |
|---|---|
| 10x PCR buffer Pfu Ultra High Fidelity: | 10 μl |
| Lambda DNA (744 ng/μl) | 1 μl |
| | (1:10 dilution) |
| dNTP's.-Mix (10 mM) | 10 μl |
| HRe7 (201 ng/μl) | 1 μl |
| HRe8 (209 ng/μl) | 1 μl |
| Pfu Ultra High Fidelity Polymerase | 1 μl |
| H₂O bidist. | 76 μl |

### Temperature Program:

$$30 x \{ \begin{matrix}2 min & 95 ° C . \\ 30 sec & 95 ° C . \\ 30 sec & 63 ° C . \\ 1 min 30 sec & 72 ° C .\end{matrix}$$

A fragment about 1.2 kb in size was detected in the agarose gel. The reaction was purified and used below as 3′ end template for the amplification of the KBD-D gene.
For the amplification of the KBD-D gene, the 5′ end template and the 3′ end template was used as matrix. The PCR was carried out as follows:

### 100 μl PCR Mixture:

| | |
|---|---|
| 10x PCR buffer Pfu Ultra High Fidelity: | 10 μl |
| dNTP-Mix (10 mM) | 10 μl |
| H₂O bidist. | 75 μl |
| 5′ end template | 1 μl |
| 3′ end template | 1 μl |
| Pfu Ultra High Fidelity Polymerase | 1 μl |
| H₂O | 76 μl |

### Temperature Program:

$$10 ( \{ \begin{matrix}60 sec & 94 ° C . \\ 300 sec & 72 ° C .\end{matrix}$$

After the 10 cycles, 1 μl of primer HRe6 (196 pg/ml) and HRe7 (206 pg/ml) and 1 μl of Pfu Ultra High Fidelity Polymerase was added, and the following temperature program was carried out with the reaction:

### Temperature Program:

$$30 x \{ \begin{matrix}•2 min & 95 ° C . \\ 30 sec & 95 ° C . \\ 30 sec & 63 ° C . \\ 1 min 30 sec & 72 ° C .\end{matrix}$$

1 μl of Taq polymerase was then added thereto and the mixture was incubated for 10 minutes at 72° C.
The resulting PCR product, about 2150 bp in size, was cut out of an agarose gel, purified and cloned into the following vector: pCR2.1-TOPO (Invitrogen).
The resulting vector pRee019 (6112 bp) was then transformed, amplified in E. coli and the KBD-D gene was checked by a sequencing.

The KBD-D gene was then cloned into the expression vector. For this, a further PCR was carried out with the vector pRee019 as template:

### Oligonucleotides Used:

HRe26:

(SEQ ID No.: 220)

5′-CTCGGTACCAACCACTCGCCGCTCAAGACCGCCTTGGCG-3′
HRe27:

(SEQ ID No.: 221)

5′-ATTAAGCTTTTAGAATCGGGAGGTGAAGTTCCTGAGGCT-3′

### 100 μl PCR Mixture:

| | |
|---|---|
| 10x PCR buffer Pfu Ultra High Fidelity: | 10 μl |
| pRee019 (25 ng/μl) | 1 μl |
| dNTP's.-Mix (10 mM) | 10 μl |
| HRe26 (287 ng/μl) | 1 μl |
| HRe27 (354 ng/μl) | 1 μl |
| Pfu Ultra High Fidelity Polymerase | 1 μl |
| H₂O bidist. | 76 μl |

### Temperature Program:

$$30 x \{ \begin{matrix}2 min & 95 ° C . \\ 30 sec & 95 ° C . \\ 30 sec & 79 ° C . \\ 1 min 30 sec & 72 ° C .\end{matrix}$$

A fragment about 2.2 kb in size was detected in the agarose gel. The reaction was purified and then cleaved with the restriction endonucleases KpnI and HindIII; the resulting fragment was cloned into the expression vector. This gave the vector pRee024, which was used subsequently for the KBD-D expression.

Expression of KBD-D (SEQ ID No.: 212) by pRee024 in E. Coli
Preclutures were inoculated from plate or glycerol culture with pRee024 transformed in E. coli strains (e.g. TG10). Depending on the size of the main culture, inoculation with LB medium (about 1:100) was carried out in a tube or a small flask.
Antibiotics were used according to the strains used (for E. coli TG10 transformed with pRee024, ampicillin 100 μg/ml)
Incubation at 250 rpm and 37° C.
The main culture was inoculated about 1:100 with preculture, main culture: LB medium or suitable minimal medium with the respective antibiotics. Incubation at 250 rpm and 37° C.
Induction was carried out with 1 mM IPTG above an OD_{578 nm} of 1. The incubation temperature was then lowered to room temperature (about 20° C.). The cells were centrifuged off 2 hours after induction. (See FIG. 9).

### Example 19

### Cell Disruption and Inclusion Body Purification (pRee024)

Insolubly expressed KBD-D (SEQ ID No.: 213) (e.g. in inclusion bodies) was purified as follows:

The cell sediment from Example 2 was resuspended in 20 mM phosphate buffer with 100 mM NaCl pH=7.5 and disrupted through ultrasound treatment.

The disrupted cells were centrifuged again (4° C., 12 000 g, 20 minutes). The supernatant was discarded.

The sediment was dissolved in buffer A (10 mM NaH₂PO₄, 2 mM KH₂PO₄, 100 mM NaCl, 8 M urea, 5 mM DTT). Centrifugation was then carried out again and the supernatant was applied to an Ni chelate sepharose. Following application, washing was carried out with buffer A and 20 mM imidazole. Elution from the column was carried out with buffer B (10 mM NaH₂PO₄, 2 mM KH₂PO₄, 100 mM NaCl, 8 M urea, 5 mM DTT, 500 mM imidazole). The eluate was collected in fractions and analyzed by means of SDS-PAGE. Fractions which comprised purified KBD-D were renatured, as described in Example 13.

### Example 20

### Renaturation of Keratin-Binding Domain D (SEQ ID No.: 213)

Insolubly expressed keratin-binding domain D (e.g. from inclusion bodies) could be renatured through a dialysis and thus activated. The procedure was as follows:

The fractions from Example 19 which comprised purified KBD-D were poured into a dialysis tube (MWCO 12-14 KD).

Dialysis was then carried out for about 1 hour against 1 l 8 M urea solution.

Then, over a period of 12 hours, 2 l of deionized water were metered in continuously using a peristaltic pump.

The contents of the dialysis tube were then removed. The KBD-D activated in this way was used for the following activity tests.

### Example 21

### Qualitative Binding to Skin

A visual qualitative test was used in order to examine whether the KBD-D (SEQ ID No.: 213) binds to skin.

### Solutions Used:

Blocking solution: Western blocking reagent 1921673 Roche (10× solution) diluted in TBS

### TBS: 20 mM Tris; 150 mM NaCl pH 7.5

### TTBS: TBS+0.05% Tween 20

The first step is the transfer of the outer keratin layer from the skin to a stable support. For this purpose, a transparent adhesive tape is firmly applied to depilated human skin and removed again. The test can be carried out directly on the transparent adhesive strip, or the adhering keratin layer can be transferred to a glass slide through renewed adhesion. Binding was demonstrated as follows:
for incubation with the various reagents, transfer to a Falcon vessel, if appropriate addition of ethanol for degreasing, removal of ethanol and drying of the slide
incubation with blocking buffer for 1 h at room temperature
2× washing for 5 min with TTBS
1 (washing for 5 min with TBS
incubation with the KBD to be tested (coupled to tag—e.g. His6, HA etc.) in TBS/0.05% Tween 20 for 2-4 h at room temperature
removal of the supernatant
3 (washing with TBS
incubation for 1 h at room temperature with monoclonal mouse anti-tag (His6 or HA) antibodies with peroxidase conjugate (1:2000 in TBS+0.01% blocking) [monoclonal antipolyhistidine peroxidase conjugate, produced in mouse, lyophilized powder, Sigma]
2× washing for 5 min with TTBS
1× washing for 5 min with TBS
addition of phosphatase substrate (NBT-BCIP; Boehringer MA 1 tablet/40 ml of water 2.5 min; stop: with water)
optical detection of the colored precipitate with the naked eye or using a microscope. A blue-colored precipitate, being a reaction of the antipolyhistidine-APconjugate interacting with the KBD-D, was visible on the transparent adhesive tape treated with KBD-D. As a negative control, a transparent adhesive tape was treated only with buffer. Here, no significant blue coloration could be detected. These results show that KBD-D has bound to the skin keratin on the transparent adhesive strip.

### Example 22

### Quantitative Binding to Skin and Hair

In order to investigate the binding strength of KBD-D (SEQ ID No.: 213) to skin and hair compared to KBD-B (SEQ ID No.: 166), a quantitative test was carried out. In this test, hair was firstly incubated with KBD-B or KBD-D and excess KBD-B or -D was washed off. An antibody-peroxidase conjugate was then coupled via the His tag of the KBD-B or -D. Nonbound antibody-peroxidase conjugate was washed off again. The bound antibody-peroxidase conjugate can convert a colorless substrate (TMB) into a colored product, which was measured photometrically at 405 nm. The intensity of the absorption indicates the amount of bound KBD-B or -D.

The test for binding to hair was carried out with human keratinocytes in microtiter plates as follows.
2× washing with PBS/0.05% Tween 20
Addition of 1 ml of 1% BSA in PBS and incubation for 1 h at room temperature, gentle swinging movements (900 rpm).
Removal of the supernatant
Addition of 100 μg of KBD in PBS with 0.05% Tween 20; incubation for 2 h at room temperature and gentle swinging movements (900 rpm).
Removal of the supernatant
3× washing with PBS/0.05% Tween 20
Incubation with 1 ml of monoclonal mouse anti-tag-His6 antibodies for 2-4 h at room temperature, gentle swinging movement (900 rpm)
3× washing with PBS/0.05% Tween 20
Addition of peroxidase substrate (1 ml/Eppendof vessel; composition see below) reaction until a blue coloration (about 90 seconds).
The reaction stopped with 100 μl of 2 M H₂SO₄.
The absorption was measured at 405 nm.

Peroxidase substrate (prepared shortly beforehand):

0.1 ml TMB solution (42 mM TMB in DMSO)
+10 ml of substrate buffer (0.1 M sodium acetate pH 4.9)
+14.7 μl H₂O₂ 3% strength

In order to characterize the hair binding of the KBD-D compared to the KBD-B, the following binding assay was carried out:

5 mg of hair (human) were cut into sections 5 mm in length and transferred to Eppendorf vessels (protein low-bind).
Addition of 1 ml of ethanol for degreasing
Centrifugation, removal of ethanol and washing of the hair with H₂O
Centrifugation, removal of the supernatant
Addition of the keratin-binding domain to be tested (coupled to tag—e.g. His₆, HA etc.) in 1 ml of PBS/0.05% Tween 20; incubation for 2 h at room temperature with gentle swinging movements.
Centrifugation, removal of the supernatant
3× washing with PBS/0.05% Tween 20
Incubation with 1 ml monoclonal mouse antitag (His6 or HA) antibodies with peroxidase conjugate (1:2000 in PBS/0.05% Tween 20) [monoclonal antipolyhistidine peroxidase conjugate, produced in mouse, lyophilized powder, Sigma] for 2-4 h at room temperature, gentle swinging movement
3× washing with PBS/0.05% Tween 20
Addition of peroxidase substrate (1 ml/Eppendorf vessel)
Allow reaction to proceed until blue coloration (90 seconds).
Stop the reaction with 100 μl of 2 M H₂SO₄.

The absorption was measured at 405 nm

Peroxidase substrate (prepare shortly beforehand):

0.1 ml of TMB solution (42 mM TMB in DMSO)
+10 ml of substrate buffer (0.1 M sodium acetate pH 4.9)
+14.7 μl H₂O₂ 3% strength
BSA=Bovine serum albumin
PBS=Phosphate buffered salt solution
Tween 20=Polyoxyethylene sorbitan monolaureate, n about 20

### TMB=3,5,3′,5′-Tetramethylbenzidine

TABLE 10a
Quantitative binding of KBD-D or KBD-B to skin. The absorption
values listed are values standardized to the surface (of skin)

| Keratin-binding domain | Absorption at 405 nm |
|---|---|
| KBD-D to skin | 3.69 |
| KBD-D to skin after 10% | 3.15 |
| strength SDS treatment | |
| KBD-B to skin | 0.93 |
| KBD-B to skin after 10% | 0.185 |
| strength SDS treatment | |

TABLE 10b
Quantitative binding of KBD-D to hair. The absorption
values listed are values standardized to the surface

| Keratin-binding domain | Absorption at 405 nm |
|---|---|
| KBD-D to skin | 0.88 |
| KBD-D to skin at 10% | 0.62 |
| strength SDS treatment | |

TABLE 10c
Quantitative binding of KBD-D and KBD-B to skin and hair
after 10% strength SDS treatment in % relative to the KBD-D
and KBD-B untreated hair or skin.

| | Relative absorption |
|---|---|
| | loss after 10% strength |
| Keratin-binding domain | SDS treatment in % |
| KBD-D to skin after 10% strength SDS treatment | 15 |
| KBD-B to skin after 10% strength SDS treatment | 80 |
| KBD-D to hair after 10% strength SDS treatment | 30 |
| KBD-B to hair after 10% strength SDS treatment | 86 |

These results show that the protein KBD-D can bind to hair and more strongly to skin (see Tab. 10). In contrast to the KBD-B (SEQ ID No.: 166), the binding of the KBD-D (SEQ ID No.: 168) is only influenced relatively little as a result of washing with an up to 10% strength SDS solution (see Tab. 10a).

### Example 22a

### Microbeads and Films of C16-KBD

In experimental assays, it was the intention to show whether the fusion protein C16-KBD can form assembly forms and bind to hair. Microbeads were produced from aqueous solution (5 mM KH₂PO₄ after dialysis) by precipitation with 1-3 vol. of 1 M KH₂PO₄ buffer. Pure C16 spider silk protein forms spherical microparticles with a size distribution of about 100 nm-10 μM (FIG. 9). The C16-KBD-B fusion protein also forms spherical particles (FIG. 9).

Films can likewise be produced from aqueous protein solutions (5 mM KH₂PO₄ after dialysis) or from a 10-50 mg/ml C16-KBD-B-fusion protein-comprising hexafluoroisopropanol solution. For this purpose, in the laboratory, a few ml of the solution are pipetted onto a polysterol surface (e.g. agar plate) and the solvent is evaporated. This gives a water-soluble protein film which can be removed from the surface. The special quality of the C16 spider silk protein is that this water-soluble film can be subsequently processed and thus rendered water-insoluble. For this, the film of KH₂PO₄ is subsequently treated with 100% ethanol. The C16 protein film is then no longer water-soluble. If the C16-KBD-B solution is applied to a polysterol surface, then, after drying, a film likewise forms which becomes water-insoluble following treatment with ethanol. In summary, it can be established that assembly forms can be produced from C16-KBD fusion protein.

### Example 22b

### Film Formation on Hair

The aim was to show whether film formation of the C16-KBD fusion protein takes place on hair. For this, hair was incubated in the corresponding protein solutions or without protein, dried and analyzed by means of electron microscopy (SEM) (see FIG. 10).

Following treatment of a hair surface with C16-KBD, the hair scales are significantly smoothed, which points to filming of the C16-KBD-B fusion protein on the hair surface.

Dermocosmetic preparations according to the invention comprising the keratin-binding effector molecule C16-KBD-B (according to SEQ ID No.: 168) prepared according to Example 3 are described below. The C16-KBD-B is referred to in the examples below as fusion protein-KBD by way of representation for all of the other KBD fusion proteins described above. It will be appreciated by the person skilled in the art that all of the other specified KBD fusion proteins can also be prepared using the corresponding KBD fusion protein constructs (e.g. KBD-D protein according to (SEQ ID No.: 212) in pRee024 (FIG. 8) according to Example 3, and be used in the preparations specified below.

### Example 23

### Use of the KBD in an Emulsion for Daycare—O/W Type

| | |
|---|---|
| % | Ingredient (INCI) |

Al 1%:

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 67.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 1.0 | Aqueous solution with about 5% fusion protein-KBD |
| E | q.s. | Sodium Hydroxide |

Al 5%:

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 63.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 5.0 | Aqueous solution with about 5% fusion protein-KBD |
| E | q.s. | Sodium Hydroxide |

Preparation: Heat phases A and B separately from one another to about 80° C. Stir Phase B into phase A and homogenize. Stir phase C into the combined phases A and B and homogenize again. Cool with stirring to about 40° C., add phase D, adjust the pH to about 6.5 using phase E, homogenize and cool to room temperature with stirring.
Note:
The formulation is prepared without protective gas. Bottling must take place into oxygen-impermeable packagings, e.g. aluminum tubes.

### Example 24

### Use of the KBD in a Protective Day Cream—O/W Type

| | |
|---|---|
| % | Ingredient (INCI) |

Al 1%:

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 68.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 1.0 | Aqueous solution with about 5% fusion protein-KBD |
| E | q.s. | Sodium Hydroxide |

Al 5%:

| | | |
|---|---|---|
| | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 64.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 5.0 | Aqueous solution with about 5% fusion protein-KBD |
| E | q.s. | Sodium Hydroxide |

Preparation: Heat phases A and B separately from one another to about 80° C.. Stir phase B into phase A and homogenize. Incorporate phase C into the combined phases A and B and homogenize. Cool with stirring to about 40° C.. Add phase D, adjust the pH to about 6.5 using phase E and homogenize. Cool to room temperature with stirring.

### Example 25

### Use of the KBD in a Face-Cleansing Lotion—O/W Type

| | |
|---|---|
| % | Ingredient (INCI) |

Al 1%:

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Aqueous solution with about 5% fusion protein-KBD |
| | 60.7 | Aqua dem. |

Al 5%:

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Aqueous solution with about 5% fusion protein-KBD |
| | 56.7 | Aqua dem. |

Preparation: Dissolve phase A. Stir phase B into phase A. Incorporate phase C into the combined phases A and B. Dissolve phase D, stir into the combined phases A, B and C and homogenize. After-stir for 15 min.

### Example 26

### Use of the KBD in a Daily Care Body Spray

| | |
|---|---|
| % | Ingredient (INCI) |

Al 1%:

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Aqueous solution with about 5% fusion protein-KBD |
| | 59.2 | Alcohol |

Al 5%:

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Aqueous solution with about 5% fusion protein-KBD |
| | 55.2 | Alcohol |

Preparation: Weigh in the components of phase A and dissolve until clear.

### Example 27

### Use of the KBD in a Skin Care Gel

| | |
|---|---|
| % | Ingredient (INCI) |

Al 1%:

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 1.0 | Aqueous solution with about 5% fusion protein-KBD |
| | 75.4 | Aqua dem. |
| C | 0.8 | Triethanolamine |

Al 5%:

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 5.0 | Aqueous solution with about 5% fusion protein-KBD |
| | 71.4 | Aqua dem. |
| C | 0.8 | Triethanolamine |

Preparation: Dissolve phase A until clear. Allow phase B to swell and neutralize with phase C. Stir phase A into the homogenized phase B and homogenize.

Example 28

### Use of the KBD in an Aftershave Lotion

| | |
|---|---|
| % | Ingredient (INCI) |

Al 1%:

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 1.0 | Aqueous solution with about 5% fusion protein-KBD |
| | 0.1 | Triethanolamine |
| | 63.5 | Aqua dem. |

Al 5%:

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 5.0 | Aqueous solution with about 5% fusion protein-KBD |
| | 0.1 | Triethanolamine |
| | 59.5 | Aqua dem. |

Preparation: Mix the components of phase A. Dissolve phase B, incorporate into phase A and homogenize.

### Example 29

### Use of the KBD in an Aftersun Lotion

| | |
|---|---|
| % | Ingredient (INCI) |

Al 1%:

| | | |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 1.0 | Aqueous solution with about 5% fusion protein-KBD |
| | 63.2 | Aqua dem. |
| C | 0.2 | Triethanolamine |

Al 5%:

| | | |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 5.0 | Aqueous solution with about 5% fusion protein-KBD |
| | 59.2 | Aqua dem. |
| C | 0.2 | Triethanolamine |

Preparation: Mix the components of phase A. Stir phase B into phase A with homogenization. Neutralize with phase C and homogenize again.

### Example 30

### Use of the KBD in a Sunscreen Lotion A

| | |
|---|---|
| % | Ingredient (INCI) |

Al 1%:

| | | |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 59.7 | Aqua dem. |
| D | 1.0 | Aqueous solution with about 5% fusion protein-KBD |
| | 1.0 | Phenoxyethanol, Methylparaben, Ethylparaben, Butylparaben, |
| | | Propyl-paraben, Isobutylparaben |
| | 0.3 | Bisabolol |

Al 5%:

| | | |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 55.7 | Aqua dem. |
| D | 5.0 | Aqueous solution with about 5% fusion protein-KBD |
| | 1.0 | Phenoxyethanol, Methylparaben, Ethylparaben, Butylparaben, |
| | | Propyl-paraben, Isobutylparaben |
| | 0.3 | Bisabolol |

Preparation: Heat the components of phases A and B separately from one another to about 80° C.. Stir phase B into phase A and homogenize. Heat phase C to about 80° C. and stir into the combined phases A and B with homogenization. Cool to about 40° C. with stirring, add phase D and homogenize again.

### Example 31

### Use of the KBD in a Sunscreen Lotion—O/W Type

| | |
|---|---|
| % | Ingredient (INCI) |

Al 1%:

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 60.9 | Aqua dem. |
| D | 1.0 | Aqueous solution with about 5% fusion protein-KBD |
| | 0.5 | Phenoxyethanol, Methylparaben, Butylparaben, Ethylparaben, |
| | | Propyl-paraben, Isopropylparaben |
| | 1.0 | Tocopheryl Acetate |
| | 0.2 | Bisabolol |

Al 5%:

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl Taurate |
| | | Copolymer, Squalane, Polysorbate 60 |
| | 0.2 | Disodium EDTA |
| | 5.0 | Propylene Glycol |
| | 0.5 | Panthenol |
| | 56.9 | Aqua dem. |
| D | 5.0 | Aqueous solution with about 5% fusion protein-KBD |
| | 0.5 | Phenoxyethanol, Methylparaben, Butylparaben, |
| | | Ethylparaben, Propylparaben, Isopropylparaben |
| | 1.0 | Tocopheryl Acetate |
| | 0.2 | Bisabolol |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 32

### Use of the KBD in a Sunscreen Lotion—O/W Type

| | |
|---|---|
| % | Ingredient (INCI) |

Al 1%:

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 56.3 | Aqua dem. |
| D | 1.0 | Aqueous solution with about 5% fusion protein-KBD |
| | 1.0 | Tocopheryl Acetate |
| | 0.2 | Bisabolol |
| | q.s. | Perfume oil |
| | q.s. | Preservative |

Al 5%:

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 52.3 | Aqua dem. |
| D | 5.0 | Aqueous solution with about 5% fusion protein-KBD |
| | 1.0 | Tocopheryl Acetate |
| | 0.2 | Bisabolol |
| | q.s. | Perfume oil |
| | q.s. | Preservative |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 33

### Use of the KBD in a Foot Balsam

| | |
|---|---|
| % | Ingredient (INCI) |

Al 1%:

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Aqua dem. |
| | q.s. | Preservative |
| C | 1.0 | Bisabolol |
| | 1.0 | Tocopheryl Acetate |
| D | 1.0 | Aqueous solution with about 5% fusion protein-KBD |
| | 5.0 | Witch Hazel Extract |

Al 5%:

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Aqua dem. |
| | q.s. | Preservative |
| C | 1.0 | Bisabolol |
| | 1.0 | Tocopheryl Acetate |
| D | 5.0 | Aqueous solution with about 5% fusion protein-KBD |
| | 5.0 | Witch Hazel Extract |

Preparation: Heat the components of phases A and B separately from one another to about 80° C.. Stir phase B into phase A with homogenization. Cool to about 40° C. with stirring, add phases C and D and briefly after-homogenize. Cool to room temperature with stirring.

### Example 34

### Use of the KBD in a W/O Emulsion with Bisabolol

| | |
|---|---|
| % | Ingredient (INCI) |

Al 1%:

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 55.6 | Aqua dem. |
| C | 1.0 | Aqueous solution with about 5% fusion protein-KBD |
| | 0.5 | Tocopheryl Acetate |
| | 0.6 | Bisabolol |

Al 5%:

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Aqua dem. |
| C | 5.0 | Aqueous solution with about 5% fusion protein-KBD |
| | 0.5 | Tocopheryl Acetate |

Preparation: Heat phases A and B separately from one another to about 85° C.. Stir phase B into phase A and homogenize. Cool to about 40° C. with stirring, add phase C and briefly homogenize again. Cool to room temperature with stirring.
List of formulations for patent keratin-binding domain - hair care

### Example 35

### Foam Conditioner with Setting Agent

| | |
|---|---|
| % | Ingredient (INCI) |

Al 1%

| | | |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 1.0 | Aqueous solution with about 5% fusion protein-KBD |
| | 68.1 | Aqua dem. |
| | 10.0 | Propane/Butane |

Al 5%

| | | |
|---|---|---|
| A | 10.0 | PVP/VA Copolymer |
| | 0.2 | Hydroxyethyl Cetyldimonium Phosphate |
| | 0.2 | Ceteareth-25 |
| | 0.5 | Dimethicone Copolyol |
| | q.s. | Perfume oil |
| | 10.0 | Alcohol |
| | 5.0 | Aqueous solution with about 5% fusion protein-KBD |
| | 64.1 | Aqua dem. |
| | 10.0 | Propane/Butane |

Preparation: Weigh the components of phase A together, stir until everything has dissolved and bottle.

### Example 36

### Foam Conditioner

| | |
|---|---|
| % | Ingredient (INCI) |

Al 1%

| | | |
|---|---|---|
| A | 1.0 | Polyquaternium-4 |
| | 0.5 | Hydroxyethyl Cetyldimonium Phosphate |
| | 1.0 | Aqueous solution with about 5% fusion protein-KBD |
| | q.s. | Perfume oil |
| | q.s. | Preservative |
| | 91.5 | Aqua dem. |
| | 6.0 | Propane/Butane |

Al 5%

| | | |
|---|---|---|
| A | 1.0 | Polyquaternium-4 |
| | 0.5 | Hydroxyethyl Cetyldimonium Phosphate |
| | 5.0 | Aqueous solution with about 5% fusion protein-KBD |
| | q.s. | Perfume oil |
| | q.s. | Preservative |
| | 87.5 | Aqua dem. |
| | 6.0 | Propane/Butane |

Preparation: Weigh the components of phase A together, stir until everything has dissolved to give a clear solution and bottle.

### Example 37

### Foam Conditioner

| | |
|---|---|
| % | Ingredient (INCI) |

Al 1%

| | | |
|---|---|---|
| A | 1.0 | Polyquaternium-11 |
| | 0.5 | Hydroxyethyl Cetyldimonium Phosphate |
| | 1.0 | Aqueous solution with about 5% fusion protein-KBD |
| | q.s. | Perfume oil |
| | q.s. | Preservative |
| | 91.5 | Aqua dem. |
| | 6.0 | Propane/Butane |

Al 5%

| | | |
|---|---|---|
| A | 1.0 | Polyquaternium-11 |
| | 0.5 | Hydroxyethyl Cetyldimonium Phosphate |
| | 5.0 | Aqueous solution with about 5% fusion protein-KBD |
| | q.s. | Perfume oil |
| | q.s. | Preservative |
| | 87.5 | Aqua dem. |
| | 6.0 | Propane/Butane |

Preparation: Weight the components of phase A together, stir until everything has dissolved to give a clear solution and bottle.

### Example 38

### Styling Foam

| | |
|---|---|
| % | Ingredient (INCI) |

Al 1%

| | | |
|---|---|---|
| A | 0.5 | Laureth-4 |
| | q.s. | Perfume oil |
| B | 77.3 | Aqua dem. |
| | 10.0 | Polyquaternium-28 |
| | 1.0 | Aqueous solution with about 5% fusion protein-KBD |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Al 5%

| | | |
|---|---|---|
| A | 0.5 | Laureth-4 |
| | q.s. | Perfume oil |
| B | 73.3 | Aqua dem. |
| | 10.0 | Polyquaternium-28 |
| | 5.0 | Aqueous solution with about 5% fusion protein-KBD |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### Example 39

### Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 78.5 | Aqua dem. |
| | 6.7 | Acrylates Copolymer |
| | 0.6 | AMP |
| | 1.0 | Aqueous solution with about 5% fusion protein-KBD |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Al 5%

| | | |
|---|---|---|
| A | 2.0 | Cocotrimonium Methosulfate |
| | q.s. | Perfume oil |
| B | 74.5 | Aqua dem. |
| | 6.7 | Acrylates Copolymer |
| | 0.6 | AMP |
| | 5.0 | Aqueous solution with about 5% fusion protein-KBD |
| | 0.5 | Dimethicone Copolyol |
| | 0.2 | Ceteareth-25 |
| | 0.2 | Panthenol |
| | 0.1 | PEG-25 PABA |
| | 0.2 | Hydroxyethylcellulose |
| C | 10.0 | HFC 152 A |

Preparation: Mix the components of phase A. Add the components of phase B one after the other and dissolve. Bottle with phase C.

### Example 40

### Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 7.70 | Polyquaternium-44 |
| | 1.0 | Aqueous solution with about 5% fusion protein-KBD |
| | q.s. | Preservative |
| | 79.3 | Aqua dem. |
| C | 10.0 | Propane/Butane |

Al 5%

| | | |
|---|---|---|
| A | 2.0 | Cocotrimonium Methosulfate |
| | q.s. | Perfume oil |
| B | 7.70 | Polyquaternium-44 |
| | 5.0 | Aqueous solution with about 5% fusion protein-KBD |
| | q.s. | Preservative |
| | 75.3 | Aqua dem. |
| C | 10.0 | Propane/Butane |

Preparation: Mix the components of phase A. Dissolve the components of phase B until clear, then stir phase B into phase A. Adjust the pH to 6-7, bottle with phase C.

### Example 41

### Styling Foam

| | |
|---|---|
| % | Ingredient (INCI) |

Al 1%

| | | |
|---|---|---|
| A | 2.00 | Cocotrimonium Methosulfate |
| | q.s. | Perfume oil |
| B | 72.32 | Aqua dem. |
| | 2.00 | VP/Acrylates/Lauryl Methacrylate Copolymer |
| | 0.53 | AMP |
| | 1.00 | Aqueous solution with about 5% fusion protein-KBD |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Al 5%

| | | |
|---|---|---|
| A | 2.00 | Cocotrimonium Methosulfate |
| | q.s. | Perfume oil |
| B | 68.32 | Aqua dem. |
| | 2.00 | VP/Acrylates/Lauryl Methacrylate Copolymer |
| | 0.53 | AMP |
| | 5.00 | Aqueous solution with about 5% fusion protein-KBD |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Dissolve phase C in the mixture of A and B, then adjust the pH to 6-7. Bottle with phase D.

### Example 42

### Styling Foam

| | |
|---|---|
| % | Ingredient (INCI) |

Al 1%

| | | |
|---|---|---|
| A | 2.00 | Cetrimonium Chloride |
| | q.s. | Perfume oil |
| B | 67.85 | Aqua dem. |
| | 7.00 | Polyquaternium-46 |
| | 1.00 | Aqueous solution with about 5% fusion protein-KBD |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Al 5%

| | | |
|---|---|---|
| A | 2.00 | Cetrimonium Chloride |
| | q.s. | Perfume oil |
| B | 63.85 | Aqua dem. |
| | 7.00 | Polyquaternium-46 |
| | 5.00 | Aqueous solution with about 5% fusion protein-KBD |
| | 0.20 | Ceteareth-25 |
| | 0.50 | Panthenol |
| | 0.05 | Benzophenone-4 |
| | 0.20 | Amodimethicone, Cetrimonium Chloride, Trideceth-12 |
| | 15.00 | Alcohol |
| C | 0.20 | Hydroxyethylcellulose |
| D | 6.00 | Propane/Butane |

Preparation: Mix the components of phase A. Add the components of phase B one after the other and dissolve. Dissolve phase C in the mixture of A and B, then adjust the pH to 6-7. Bottle with phase D.

### Example 43

### Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 85.5 | Aqua dem. |
| B | 7.0 | Sodium Polystyrene Sulfonate |
| | 1.0 | Aqueous solution with about 5% fusion protein-KBD |
| | 0.5 | Cetrimonium Bromide |
| | q.s. | Preservative |
| C | 6.0 | Propane/Butane |

Styling foam
Al 5%

| | | |
|---|---|---|
| A | q.s. | PEG-40 Hydrogenated Castor Oil |
| | q.s. | Perfume oil |
| | 81.5 | Aqua dem. |
| B | 7.0 | Sodium Polystyrene Sulfonate |
| | 5.0 | Aqueous solution with about 5% fusion protein-KBD |
| | 0.5 | Cetrimonium Bromide |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 40

### Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 92.0 | Aqua dem. |
| B | 0.5 | Polyquaternium-10 |
| | 1.0 | Aqueous solution with about 5% fusion protein-KBD |
| | 0.5 | Cetrimonium Bromide |
| | q.s. | Preservative |
| C | 6.0 | Propane/Butane |

Al 5%

| | | |
|---|---|---|
| A | q.s. | PEG-40 Hydrogenated Castor Oil |
| | q.s. | Perfume oil |
| | 88.0 | Aqua dem. |
| B | 0.5 | Polyquaternium-10 |
| | 5.0 | Aqueous solution with about 5% fusion protein-KBD |
| | 0.5 | Cetrimonium Bromide |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 44

### Styling Foam

| | |
|---|---|
| % | Ingredient (INCI) |

Al 1%

| | | |
|---|---|---|
| A | q.s. | PEG-40 Hydrogenated Castor Oil |
| | q.s. | Perfume oil |
| | 82.5 | Aqua dem. |
| B | 10.0 | Polyquaternium-16 |
| | 1.0 | Aqueous solution with about 5% fusion protein-KBD |
| | 0.5 | Hydroxyethyl Cetyldimonium Phosphate |
| | q.s. | Preservative |
| C | 6.0 | Propane/Butane |

Al 5%

| | | |
|---|---|---|
| A | q.s. | PEG-40 Hydrogenated Castor Oil |
| | q.s. | Perfume oil |
| | 78.5 | Aqua dem. |
| B | 10.0 | Polyquaternium-16 |
| | 5.0 | Aqueous solution with about 5% fusion protein-KBD |
| | 0.5 | Hydroxyethyl Cetyldimonium Phosphate |
| | q.s. | Preservative |
| C | 6.0 | Propane/Butane |

Preparation: Solubilize phase A. Weigh phase B into phase A and dissolve until clear. Adjust the pH to 6-7, bottle with phase C.

### Example 45

### Styling Foam

| | |
|---|---|
| % | Ingredient (INCI) |

Al 1%

| | | |
|---|---|---|
| A | 2.0 | Cocotrimonium Methosulfate |
| | q.s. | Perfume oil |
| B | 84.0 | Aqua dem. |
| | 2.0 | Chitosan |
| | 1.0 | Aqueous solution with about 5% fusion protein-KBD |
| | 0.5 | Dimethicone Copolyol |
| | 0.2 | Ceteareth-25 |
| | 0.2 | Panthenol |
| | 0.1 | PEG-25 PABA |
| C | 10.0 | HFC 152 A |

Al 5%

| | | |
|---|---|---|
| A | 2.0 | Cocotrimonium Methosulfate |
| | q.s. | Perfume oil |
| B | 80.0 | Aqua dem. |
| | 2.0 | Chitosan |
| | 5.0 | Aqueous solution with about 5% fusion protein-KBD |
| | 0.5 | Dimethicone Copolyol |
| | 0.2 | Ceteareth-25 |
| | 0.2 | Panthenol |
| | 0.1 | PEG-25 PABA |
| C | 10.0 | HFC I52 A |

Preparation: Mix the components of phase A. Add the components of phase B one after the other and dissolve. Bottle with phase C.

### Example 46

### Care Shampoo

| | |
|---|---|
| % | Ingredient (INCI) |

Al 1%

| | | |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, Cocamide MEA, |
| | | Laureth-10 |
| | 1.0 | Aqueous solution with about 5% fusion protein-KBD |
| | 7.7 | Polyquaternium-44 |
| | 2.0 | Amodimethicone |
| | q.s. | Perfume oil |
| | q.s. | Preservative |
| | 1.0 | Sodium Chloride |
| | 43.3 | Aqua dem. |
| B | q.s. | Citric Acid |

Al 5%

| | | |
|---|---|---|
| A | 30.0 | Sodium Laureth Sulfate |
| | 6.0 | Sodium Cocoamphoacetate |
| | 6.0 | Cocamidopropyl Betaine |
| | 3.0 | Sodium Laureth Sulfate, Glycol Distearate, Cocamide MEA, |
| | | Laureth-10 |
| | 5.0 | Aqueous solution with about 5% fusion protein-KBD |
| | 7.7 | Polyquaternium-44 |
| | 2.0 | Amodimethicone |
| | q.s. | Perfume oil |
| | q.s. | Preservative |
| | 1.0 | Sodium Chloride |
| | 39.3 | Aqua dem. |
| B | q.s. | Citric Acid |

Preparation: Mix the components of phase A and dissolve. Adjust the pH to 6-7 with citric acid.

### Example 47

### Shower Gel

| | |
|---|---|
| % | Ingredient (INCI) |

Al 1%

| | | |
|---|---|---|
| A | 40.0 | Sodium Laureth Sulfate |
| | 5.0 | Decyl Glucoside |
| | 5.0 | Cocamidopropyl Betaine |
| | 1.0 | Aqueous solution with about 5% fusion protein-KBD |
| | 1.0 | Panthenol |
| | q.s. | Perfume oil |
| | q.s. | Preservative |
| | 2.0 | Sodium Chloride |
| | 46.0 | Aqua dem. |
| B | q.s. | Citric Acid |

Al 5%

| | | |
|---|---|---|
| A | 40.0 | Sodium Laureth Sulfate |
| | 5.0 | Decyl Glucoside |
| | 5.0 | Cocamidopropyl Betaine |
| | 5.0 | Aqueous solution with about 5% fusion protein-KBD |
| | 1.0 | Panthenol |
| | q.s. | Perfume oil |
| | q.s. | Preservative |
| | 2.0 | Sodium Chloride |
| | 42.0 | Aqua dem. |
| B | q.s. | Citric Acid |

Preparation: Mix the components of phase A and dissolve. Adjust the pH to 6-7 with citric acid.

### Example 48

### Shampoo

| | |
|---|---|
| % | Ingredient (INCI) |

Al 1%

| | | |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 44.6 | Aqua dem. |
| | 1.0 | Aqueous solution with about 5% fusion protein-KBD |
| | 0.3 | Polyquaternium-10 |
| | 1.0 | Panthenol |
| | q.s. | Preservative |
| | 1.0 | Laureth-3 |
| | 2.0 | Sodium Chloride |

Al 5%

| | | |
|---|---|---|
| A | 40.0 | Sodium Laureth Sulfate |
| | 5.0 | Sodium C12-15 Pareth-15 Sulfonate |
| | 5.0 | Decyl Glucoside |
| | q.s. | Perfume oil |
| | 0.1 | Phytantriol |
| | 40.6 | Aqua dem. |
| | 5.0 | Aqueous solution with about 5% fusion protein-KBD |
| | 0.3 | Polyquaternium-10 |
| | 1.0 | Panthenol |
| | q.s. | Preservative |
| | 1.0 | Laureth-3 |
| | 2.0 | Sodium Chloride |

Preparation: Mix the components of phase A and dissolve. Adjust the pH to 6-7 with citric acid.

### Example 49

### Shampoo

| | |
|---|---|
| % | Ingredient (INCI) |

Al 1%

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 1.00 | Aqueous solution with about 5% fusion protein-KBD |
| | 0.15 | Guar Hydroxypropyltrimonium Chloride |
| | 2.00 | Laureth-3 |
| | 58.00 | Aqua dem. |
| | q.s. | Citric Acid |
| B | 3.00 | PEG-150 Distearate |

Al 5%

| | | |
|---|---|---|
| A | 15.00 | Cocamidopropyl Betaine |
| | 10.00 | Disodium Cocoamphodiacetate |
| | 5.00 | Polysorbate 20 |
| | 5.00 | Decyl Glucoside |
| | q.s. | Perfume oil |
| | q.s. | Preservative |
| | 5.00 | Aqueous solution with about 5% fusion protein-KBD |
| | 0.15 | Guar Hydroxypropyltrimonium Chloride |
| | 2.00 | Laureth-3 |
| | 54.00 | Aqua dem. |
| | q.s. | Citric Acid |
| B | 3.00 | PEG-150 Distearate |

Preparation: Weigh in the components of phase A and dissolve. Adjust the pH to 6-7. Add phase B and heat to about 50° C. Cool to room temperature with stirring.

### Example 50

### Moisturizing Body Care Cream

| | |
|---|---|
| % | Ingredient (INCI) |

Al 1%

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 1.0 | Aqueous solution with about 5% fusion protein-KBD |
| | 1.0 | Panthenol |
| | 0.5 | Magnesium Aluminum Silicate |
| | q.s. | Preservative |
| | 65.5 | Aqua dem. |
| C | q.s. | Perfume oil |
| D | q.s. | Citric Acid |

Al 5%

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 5.0 | Aqueous solution with about 5% fusion protein-KBD |
| | 1.0 | Panthenol |
| | 0.5 | Magnesium Aluminum Silicate |
| | q.s | Preservative |
| | 61.5 | Aqua dem. |
| C | q.s. | Perfume oil |
| D | q.s. | Citric Acid |

Preparation: Heat phases A and B separately to about 80° C. Briefly prehomogenize phase B, then stir phase B into phase A and homogenize again. Cool to about 40° C., add phase C and homogenize thoroughly again. Adjust the pH to 6-7 with citric acid.

### Example 51

### Moisturizing Body Care Cream

| | |
|---|---|
| % | Ingredient (INCI) |

Al 1%

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 62.9 | Aqua dem. |
| C | q.s. | Perfume oil |
| | 1.0 | Aqueous solution with about 5% fusion protein-KBD |

Al 5%

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 58.9 | Aqua dem. |
| C | q.s. | Perfume oil |
| | 5.0 | Aqueous solution with about 5% fusion protein-KBD |

Preparation: Heat phases A and B separately to about 80° C. Stir phase B into phase A and homogenize. Cool to about 40° C. with stirring, add phase C and homogenize again. Allow to cool to room temperature with stirring.

### Example 52

### Liquid Make-Up—O/W Type

| | |
|---|---|
| % | Ingredient (INCI) |

Al 1%

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 61.9 | Aqua dem. |
| C | 0.1 | Bisabolol |
| | 1.0 | Aqueous solution with about 5% fusion protein-KBD |
| | q.s. | Perfume oil |
| D | 5.7 | C.I. 77 891, Titanium Dioxide |
| | 1.1 | Iron Oxides |

Al 5%

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 57.9 | Aqua dem. |
| C | 0.1 | Bisabolol |
| | 5.0 | Aqueous solution with about 5% fusion protein-KBD |
| | q.s. | Perfume oil |
| D | 5.7 | C.I. 77 891, Titanium Dioxide |
| | 1.1 | Iron Oxides |

Preparation: Heat phases A and B separately to about 80° C. Stir phase B into phase A and homogenize. Cool to about 40° C with stirring, add phases C and D and thoroughly homogenize again. Allow to cool to room temperature with stirring.

### Example 53

Dermocosmetic preparations according to the invention are described below, comprising the keratin-binding effector molecule (C16-KBD-B according to SEQ ID No.: 168) prepared according to Example 3. The specified keratin-binding fusion protein is used as about 5% strength by weight aqueous solution. The following data are parts by weight.

Clear shampoo

| Ingredients (INCI) | 1 | 2 | 3 | 4 | 5 |
|---|---|---|---|---|---|
| Sodium Laureth Sulfate | 1.00 | — | 0.10 | 10.50 | — |
| Codamidopropyl Betaine | 7.50 | 7.00 | 5.00 | 5.50 | 10.00 |
| PEG-7 Glyceryl Cocoate | 2.00 | 2.50 | 3.50 | 5.00 | 2.30 |
| Perfume oil | 0.10 | 0.10 | 0.10 | 0.10 | 0.10 |
| Fusion protein-KBD | 1.0 | 5.0 | 20 | 0.5 | 10.0 |
| D-Panthenol USP | 1.00 | 1.50 | 1.80 | 1.70 | 1.40 |
| Preservative | 0.10 | 0.10 | 0.10 | 0.10 | 0.10 |
| Citric Acid | 0.10 | 0.10 | 0.10 | 0.10 | 0.10 |
| Luviquat ® Ultra Care | 1.50 | 1.00 | 1.50 | 1.20 | 1.10 |
| Sodium Chloride | 1.50 | 1.40 | 1.40 | 1.30 | 1.50 |
| Aqua dem. | ad 100 | ad 100 | ad 100 | ad 100 | ad 100 |

Shampoo

| Ingredients (INCI) | 1 | 2 | 3 | 4 | 5 |
|---|---|---|---|---|---|
| Sodium Laureth Sulfate | 1 | — | — | 0.1 | — |
| Decyl Glucoside | 5.00 | 5.50 | 4.90 | 3.50 | 7.00 |
| Cocamidopropyl Betaine | 10.00 | 5.00 | 12.50 | 7.50 | 15.00 |
| Perfume oil | 0.10 | 0.10 | 0.10 | 0.10 | 0.10 |
| Fusion protein-KBD | 1.0 | 5.0 | 0.1 | 20 | 10.0 |
| D-Panthenol USP | 0.50 | 1.00 | 0.80 | 1.50 | 0.50 |
| Preservative | 0.10 | 0.10 | 0.10 | 0.10 | 0.10 |
| Citric Acid | 0.10 | 0.10 | 0.10 | 0.10 | 0.10 |
| Laureth-3 | 0.50 | 2.00 | 0.50 | 0.50 | 2.00 |
| Sodium Chloride | 1.50 | 1.50 | 1.50 | 1.50 | 1.50 |
| Aqua dem. | ad 100 | ad 100 | ad 100 | ad 100 | ad 100 |

Clear conditioner shampoo

| Ingredients (INCI) | 1 | 2 | 3 | 4 | 5 |
|---|---|---|---|---|---|
| ® Disodium | 10.00 | 15.00 | 20.00 | 12.00 | 17.00 |
| Cocoamphodiacetate | | | | | |
| ® Decyl Glucoside | 5.00 | 6.00 | 7.00 | 8.00 | 4.00 |
| ® Cocamidopropyl | 15.00 | 12.00 | 10.00 | 18.00 | 20.00 |
| Betaine | | | | | |
| Luviquat ® FC 550 | 0.30 | 0.20 | 0.20 | 0.20 | 0.30 |
| Perfume oil | 0.10 | 0.10 | 0.10 | 0.10 | 0.10 |
| Fusion protein-KBD | 20.0 | 5.0 | 1.0 | 0.5 | 10.0 |
| Cremophor ® PS 20 | 5.00 | 1.00 | 1.00 | 7.00 | 5.00 |
| Preservative | 0.10 | 0.10 | 0.10 | 0.10 | 0.10 |
| ® Laureth-3 | 2.00 | 1.00 | 0.50 | 2.00 | 2.00 |
| Citric Acid | 0.20 | 0.20 | 0.20 | 0.20 | 0.20 |
| PEG-12 Distearate | 3.00 | 2.00 | 2.00 | 3.00 | 2.50 |
| Aqua dem. | ad 100 | ad 100 | ad 100 | ad 100 | ad 100 |

Foam O/W emulsions

| | Emulsion 1 | % by | Emulsion 2 | % by |
|---|---|---|---|---|
| | % by wt. | vol. | % by wt. | vol. |
| Stearic Acid | 5.00 | | 1.00 | |
| Cetyl Alcohol | 5.50 | | | |
| Cetearyl Alcohol | | | 2.00 | |
| PEG-40 Stearate | 8.50 | | | |
| PEG-20 Stearate | | | 1.00 | |
| Caprylic/Capric | 4.00 | | 2.00 | |
| Triglyceride | | | | |
| C12-15 Alkyl Benzoate | 10.00 | | 15.00 | |
| Cyclomethicone | 4.00 | | | |
| Dimethicone | | | 0.50 | |
| Fusion protein-KBD | 5.0 | | 10.0 | |
| Ethylhexyl Isostearate | | | 5.00 | |
| Myristyl Myristate | | | 2.00 | |
| Ceresin | 1.50 | | | |
| Glycerin | | | 3.00 | |
| Hydroxypropyl Starch | 1.00 | | 3.50 | |
| Phosphate | | | | |
| BHT | | | 0.02 | |
| Disodium EDTA | 0.50 | | 0.10 | |
| Perfume oil, Preservative | q.s. | | q.s. | |
| Colorant | q.s. | | q.s. | |
| Potassium Hydroxide | q.s. | | q.s. | |
| Aqua dem. | ad 100 | | ad 100 | |
| | adjust pH to | | adjust pH | |
| | 6.5-7.5 | | to 5.0-6.0 | |
| Emulsion 1 | | 70 | | |
| Emulsion 2 | | | | 35 |
| Nitrogen | | 30 | | |
| Propane/Butane | | | | 65 |

Conditioner Shampoo with pearlescence

| | 1 | 2 | 3 |
|---|---|---|---|
| Polyquaternium-10 | 0.50 | 0.50 | 0.40 |
| Sodium Laureth Sulfate | 0.5 | 0.1 | — |
| Codamidopropyl Betaine | 2.50 | 2.60 | 3.00 |
| Uvinul ® MS 40 | 1.50 | 0.50 | 1.00 |
| Fusion protein-KBD | 1.0 | 5.0 | 15 |
| Pearlescent solution | 2.00 | 2.50 | |
| Disodium EDTA | 0.10 | 0.15 | 0.05 |
| Preservative, Perfume oil, Thickener | q.s. | q.s. | q.s. |
| Aqua dem. | ad 100 | ad 100 | ad 100 |

adjust pH to 6.0

Clear conditioner shampoo

| | 1 | 2 | 3 |
|---|---|---|---|
| Polyquaternium-10 | 0.50 | 0.50 | 0.50 |
| Sodium Laureth Sulfate | — | 0.1 | — |
| Fusion protein-KBD | 5.0 | 10 | 3.0 |
| Uvinul M ® 40 | 1.00 | 1.50 | 0.50 |
| | 0.20 | 0.20 | 0.80 |
| Preservative, Perfume oil, Thickener | q.s. | q.s. | q.s. |
| Aqua dem. | ad 100 | ad 100 | ad 100 |

adjust pH to 6.0

Clear conditioner shampoo with volume effect

| | 1 | 2 | 3 |
|---|---|---|---|
| Sodium Laureth Sulfate | 0.2 | | |
| Uvinul ® MC 80 | 2.00 | 1.50 | 2.30 |
| Fusion protein-KBD | 10.0 | 0.5 | 0.5 |
| Cocamidopropyl Betaine | 2.50 | 2.60 | 2.20 |
| Disodium EDTA | 0.01 | 0.10 | 0.01 |
| Preservative, Perfume oil, Thickener | q.s. | q.s. | q.s. |
| Aqua dem. | ad 100 | ad 100 | ad 100 |

adjust pH to 6.0

Gel cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul ® A Plus ™ | 2.00 | 1.50 | 0.75 | 1.00 |
| UvaSorb ® k2A | | 3.00 | | |
| Ethylhexyl Bis- | | | | |
| Isopentylbenzoxazolylphenyl | | | | |
| Melamine | | | | |
| Uvinul ® MC 80 | 3.00 | | 1.00 | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Uvinul ® T 150 | 4.00 | | 3.00 | 4.00 |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutyl phenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | 3.50 | 4.00 | 7.00 | 9.00 |
| Titanium Dioxide-microfine | 1.00 | | 3.00 | |
| Zinc Oxide-microfine | | | | 0.25 |
| Fusion protein-KBD | 2.0 | 0.5 | 1.0 | 10.0 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | ad 100 | ad 100 | ad 100 | ad 100 |

adjust pH to 6.0

OW sunscreen formulation

| | 1 | 2 | 3 | 4 | 5 | 6 | 7 |
|---|---|---|---|---|---|---|---|
| Glyceryl Stearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Sodium Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Fusion protein-KBD | 10.0 | 0.5 | 3.0 | 5.0 | 0.1 | 0.02 | 7.5 |
| Uvinul ® A Plus ™ | 2.00 | 1.50 | 0.75 | 1.00 | 2.10 | 4.50 | 5.00 |
| UvaSorb ® k2A | | | | | | | |
| Ethylhexyl Bis- | | | | | | | |
| Isopentylbenzoxazolylphenyl | | | | | | | |
| Melamine | | | | | | | |
| Ethylhexyl Methoxycinnamate | | | | | 5.00 | 6.00 | 8.00 |
| Uvinul ® MC 80 | | | | | | | |
| Bis-Ethylhexyloxyphenol | | 1.50 | | 2.00 | 2.50 | | 2.50 |
| Methoxyphenyl Triazine | | | | | | | |
| Butyl Methoxydibenzoylmethane | | | 2.00 | | 2.00 | 1.50 | |
| Disodium Phenyl Dibenzimidazole | 2.50 | | 0.50 | 2.00 | | 0.30 | |
| Tetrasulfonate | | | | | | | |
| Ethyhexyl Triazone Uvinul ® T 150 | 4.00 | | 3.00 | 4.00 | | 2.00 | |
| Octocrylene | | 4.00 | | | | | 7.50 |
| Diethylhexyl Butamido Triazone | 1.00 | | | 2.00 | 1.00 | | 1.00 |
| Phenylbenzimidazole Sulfonic Acid | 0.50 | | 3.00 | | | | |
| Methylene Bis-Benzotriazolyl | 2.00 | | 0.50 | 1.50 | 2.50 | | |
| Tetramethylbutylphenol | | | | | | | |
| Ethylhexyl Salicylate | | | 3.00 | | | | 5.00 |
| Drometrizole Trisiloxane | | | 0.50 | | | 1.00 | |
| Terephthalidene Dicamphor | | 1.50 | | 1.00 | 1.00 | | 0.50 |
| Sulfonic Acid | | | | | | | |
| Diethylhexyl 2,6-Naphthalate | 3.50 | | 7.00 | | 6.00 | 9.00 | |
| Titanium Dioxide- microfine | 1.00 | | 3.00 | | 3.50 | | 1.50 |
| Zinc Oxide- microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglyceride | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soya (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium Iminodisuccinate | | | | 0.25 | 1.00 | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad100 | ad 100 |

Hydrodispersion

| | 1 | 2 | 3 | 4 | 5 |
|---|---|---|---|---|---|
| Ceteaereth-20 | 1.00 | | | 0.50 | |
| Cetyl Alcohol | | | 1.00 | | |
| Sodium Carbomer | | 0.20 | | 0.30 | |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Fusion protein-KBD | 5.0 | 0.5 | 3.0 | 0.1 | 10.0 |
| Uvinul ® A Plus ™ | 2.00 | 1.50 | 0.75 | 1.00 | 2.10 |
| UvaSorb ® k2A Ethylhexyl Bis- | | 3.50 | | | |
| Isopentylbenzoxazolylphenyl Melamine | | | | | |
| Ethylhexyl Methoxycinnamate Uvinul ® MC | | | | | 5.00 |
| 80 | | | | | |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 9.00 |
| Titanium Dioxide- microfine | 1.00 | | 3.00 | | 3.50 |
| Zinc Oxide- microfine | | | | 0.25 | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya (Soybean) Oil | | | 1.50 | | 1.00 |
| Vitamin E Acetate | 0.50 | | 0.25 | | 1.00 |
| Glucosylrutin | 0.60 | | | 0.25 | |
| Biosaccaride Gum-1 | | 2.50 | 0.50 | | 2.00 |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | ad 100 | ad 100 | ad 100 | ad 100 | ad 100 |

WO sunscreen emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Fusion protein-KBD | 5.0 | 1.0 | 10.0 | 0.5 | 0.1 |
| Uvinul ® A Plus ™ | 2.00 | 1.50 | 0.75 | 1.00 | 2.10 |
| UvaSorb ® k2A | | 2.00 | | | |
| Ethylhexyl Bis-Isopentylbenzoxazolylphenyl | | | | | |
| Melamine | | | | | |
| Ethylhexyl Methoxycinnamate Uvinul ® MC | | | | | 5.00 |
| 80 | | | | | |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 4.00 |
| Titanium Dioxide- microfine | 1.00 | | 3.00 | | 3.50 |
| Zinc Oxide- microfine | | | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soya (Soybean) Oil | | 1.00 | 1.50 | | 1.00 |
| Magnesium Sulfate MgSO4 | 1.00 | 0.50 | | 0.50 | |
| Magnesium Chloride MgCl2 | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 1.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Fusion protein-KBD | 0.5 | 3.0 | 1.0 | 5.0 |
| Uvinul ® A Plus ™ | 2.00 | 1.50 | 0.75 | 9.00 |
| UvaSorb ® k2A | | 2.00 | | 4.00 |
| Ethylhexyl Bis- | | | | |
| Isopentylbenzoxazolylphenyl | | | | |
| Melamine | | | | |
| Ethylhexyl Methoxycinnamate | | 3.00 | | |
| Uvinul ® MC 80 | | | | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Ethyhexyl Triazone Uvinul ® T | 4.00 | | 3.00 | 4.00 |
| 150 | | | | |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | | | 7.00 | |
| Titanium Dioxide-microfine | 1.00 | | 3.00 | |
| Zinc Oxide-microfine | | | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

PIT emulsion

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone | | | | 0.50 | | 1.00 | | |
| Copolyol | | | | | | | | |
| Polyglyceryl-2 Dioleate | | | | 0.75 | 0.30 | | | |
| Fusion protein-KBD | 0.1 | 5.0 | 20.0 | 0.5 | 3.0 | 0.25 | 10.0 | 3.0 |
| Uvinul ® A Plus ™ | 2.00 | 1.50 | 0.75 | 1.00 | 2.10 | 4.50 | 5.00 | 2.10 |
| UvaSorb ® k2A | | | 4.00 | | | | 1.50 | |
| Ethylhexyl Bis- | | | | | | | | |
| Isopentylbenzoxazolyl- | | | | | | | | |
| phenyl Melamine | | | | | | | | |
| Ethylhexyl | | | | | 5.00 | 6.00 | 8.00 | 5.00 |
| Methoxycinnamate | | | | | | | | |
| Uvinul ® MC 80 | | | | | | | | |
| Bis-Ethylhexyloxyphenol | | 1.50 | | 2.00 | 2.50 | | 2.50 | 2.50 |
| Methoxyphenyl Triazine | | | | | | | | |
| Butyl Methoxydibenzoyl- | | | 2.00 | | 2.00 | 1.50 | | 2.00 |
| methane | | | | | | | | |
| Disodium Phenyl | 2.50 | | 0.50 | 2.00 | | 0.30 | | |
| Dibenzimidazole | | | | | | | | |
| Tetrasulfonate | | | | | | | | |
| Ethyhexyl Triazone | 4.00 | | 3.00 | 4.00 | | 2.00 | | |
| Uvinul ® T 150 | | | | | | | | |
| Octocrylene | | 4.00 | | | | | 7.50 | |
| Diethylhexyl Butamido | 1.00 | | | 2.00 | 1.00 | | 1.00 | 1.00 |
| Triazone | | | | | | | | |
| Phenylbenzimidazole | 0.50 | | 3.00 | | | | | |
| Sulfonic Acid | | | | | | | | |
| Methylene Bis- | 2.00 | | 0.50 | 1.50 | 2.50 | | | 2.50 |
| Benzotriazolyl | | | | | | | | |
| Tetramethylbutylphenol | | | | | | | | |
| Ethylhexyl Salicylate | | | 3.00 | | | | 5.00 | |
| Drometrizole Trisiloxane | | | 0.50 | | | 1.00 | | |
| Terephthalylidene | | 1.50 | | 1.00 | 1.00 | | 0.50 | 1.00 |
| Dicamphor Sulfonic Acid | | | | | | | | |
| Diethylhexyl 2,6- | | | 7.00 | | 10.00 | 7.50 | | 8.00 |
| Naphthalate | | | | | | | | |
| Titanium | 1.00 | | 3.00 | | 3.50 | | 1.50 | 3.50 |
| Dioxide -microfine | | | | | | | | |
| Zinc Oxide - microfine | | | | 0.25 | | 2.00 | | |
| C12-15 Alkyl Benzoate | 3.50 | | | 6.35 | | | | 0.10 |
| Cocoglyceride | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl | | 0.50 | | | 2.00 | | | |
| Galactomannan | | | | | | | | |
| Hydrogenated Coco- | | | | | 3.00 | 4.00 | | |
| Glycerides | | | | | | | | |
| Behenoxy Dimethicone | | | | | | 1.50 | 2.00 | |
| VP/Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii | | 2.00 | | 3.60 | | 2.00 | | |
| (Shea Butter) | | | | | | | | |
| Iodopropyl | 0.12 | | | | 0.20 | | | |
| Butylcarbamate | | | | | | | | |
| Biosaccaride Gum-1 | | | | 0.10 | | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad |
| | | | | | | | | 100 |

Gel cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Luvigel ® EM | 1.50 | 2.50 | 2.80 | 3.50 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Titanium Dioxide-microfine | 1.00 | | 1.50 | |
| Zinc Oxide-microfine | | 2.00 | | 0.25 |
| Fusion protein-KBD | 0.5 | 10.0 | 3.0 | 5.0 |
| Dihydroxyacetone | | | 3.00 | 5.00 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | ad 100 | ad 100 | ad 100 | ad 100 |
| adjust pH to 6.0 | | | | |

OW self-tanning
formulation

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Fusion protein-KBD | 0.1 | 0.5 | 0.025 | 5.0 | 3.0 | 10.0 | 1.0 |
| Dihydroxyacetone | | | 3.00 | 5.00 | | 4 | |
| Titanium Dioxide - microfine | 1.00 | | | | 1.50 | | 1.50 |
| Zinc Oxide - microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soya (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

OW make-up

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glycerl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Fusion protein-KBD | 3.0 | 5.0 | 2.0 | 0.5 | 1.0 | 5.0 | 10.0 |
| Titanium Dioxide | 10.00 | 12.00 | 9.00 | 8.50 | 11.00 | 9.50 | 10.00 |
| Iron Oxide | 2.00 | 4.00 | 3.00 | 5.00 | 3.40 | 6.00 | 4.40 |
| Zinc Oxide | | 4.00 | | 2.00 | | 3.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Glycine Soya (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

Self-tanning hydrodispersion

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Fusion protein-KBD | 3.0 | 1.0 | 0.5 | 0.1 | 5.0 |
| Dihydroxyacetone | | | 3.00 | 5.00 | |
| Uvinul ® A Plus ™ | 2.00 | 1.50 | 0.75 | 1.00 | 2.10 |
| Titanium Dioxide - microfine | 1.00 | | 1.00 | | 1.00 |
| Zinc Oxide - microfine | | 1.90 | | 0.25 | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya (Soybean) Oil | | | 1.50 | | 1.00 |
| Vitamin E Acetate | 0.50 | | 0.25 | | 1.00 |
| Glucosylrutin | 0.60 | | | 0.25 | |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | ad 100 | ad 100 | ad 100 | ad 100 | ad 100 |

Aftersun hydrodispersion

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl | 0.50 | 0.30 | 0.40 | 0.10 | 0.50 |
| Acrylate Crosspolymer | | | | | |
| Xanthan Gum | | 0.30 | 0.15 | | |
| Fusion protein-KBD | 0.1 | 5.0 | 0.5 | 3.0 | 1.0 |
| C12-15 Alkyl Benzoate | 2.00 | 2.50 | | | |
| Dicapryl Ether | | 4.00 | | | |
| Butylene glycol | 4.00 | | 2.00 | 6.00 | |
| Dicaprylate/Dicaprate | | | | | |
| Dicapryl Carbonate | | 2.00 | 6.00 | | |
| Dimethicone | | 0.50 | 1.00 | | |
| Phenyl Trimethicone | 2.00 | | 0.50 | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya (Soybean) | | | 1.50 | | 1.00 |
| Oil | | | | | |
| Vitamin E Acetate | 0.50 | | 0.25 | | 1.00 |
| Glucosylrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume oil | 0.20 | | 0.05 | 0.40 | |
| Aqua dem. | ad 100 | ad 100 | ad 100 | ad 100 | ad 100 |

WO emulsions

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30Dipolyhydroxystearate | | | 5.00 | | |
| Fusion protein-KBD | 5.0 | 10.0 | 0.1 | 0.5 | 1.0 |
| Uvinul ® A Plus ™ | 2.00 | 1.50 | 0.75 | 1.00 | 2.10 |
| Titanium Dioxide- microfine | 1.00 | | 3.00 | | 3.50 |
| Zinc Oxide- microfine | | 0.90 | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soya (Soybean) Oil | | 1.00 | 1.50 | | 1.00 |
| Magnesium Sulfate | 1.00 | 0.50 | | 0.50 | |
| Magnesium Chloride | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 7.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | ad 100 | ad 100 | ad 100 | ad 100 | ad 100 |

Solids-stabilized emulsion
(Pickering emulsions)

| | 1 | 2 | 3 | 4 | 5 |
|---|---|---|---|---|---|
| Mineral Oil | | | 16.00 | 16.00 | |
| Octyldodecanol | 9.00 | 9.00 | 5.00 | | |
| Caprylic/Capric Triglyceride | 9.00 | 9.00 | 6.00 | | |
| C12-15 Alkyl Benzoate | | | | 5.00 | 8.00 |
| Butylene Glycol Dicaprylate/Dicaprate | | | | | 8.00 |
| Dicaprylyl Ether | 9.00 | | | 4.00 | |
| Dicaprylyl Carbonate | | 9.00 | | | |
| Hydroxyoctacosanyl Hydroxystearate | 2.00 | 2.00 | 2.20 | 2.50 | 1.50 |
| Disteardimonium Hectorite | 1.00 | 0.75 | | 0.50 | 0.25 |
| Cera Microcristallina + Paraffinum Liquidum | | 0.35 | | | 5.00 |
| Hydroxypropyl Methylcellulose | | | 0.10 | | 0.05 |
| Dimethicone | | | | | 3.00 |
| Fusion protein-KBD | 1.0 | 0.5 | 0.1 | 3.0 | 5.0 |
| Titanium Dioxide + Alumina + Simethicone + | | 3.00 | | | |
| Aqua | | | | | |
| Titanium Dioxide + Trimethoxycaprylylsilane | | 2.00 | 4.00 | 2.00 | 4.00 |
| Silica Dimethyl Silylate | 2.50 | | | 6.00 | 2.50 |
| Boron Nitride | | | 1.00 | | |
| Starch/Sodium metaphosphate Polymer | 2.00 | | | | |
| Tapioca Starch | | 0.50 | | | |
| Sodium Chloride | 5.00 | 7.00 | 8.50 | 3.00 | 4.50 |
| Glycerin | | | | 1.00 | |
| Trisodium EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methylparaben | | 0.60 | | | 0.20 |
| Propylparaben | | | | | 0.20 |
| Phenoxyathanol | | | 0.20 | | |
| Hexamidine Diisethionate | | | 0.40 | 0.50 | 0.40 |
| Diazolidinyl Urea | | | | | 0.08 |
| Ethanol | | | 0.23 | 0.20 | |
| Perfume oil | 5.00 | | 3.00 | 4.00 | |
| Aqua dem. | 0.20 | | 0.30 | 0.10 | |
| | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Fusion protein-KBD | 10.0 | 1.0 | 3.0 | 0.1 |
| Uvinul ® A Plus ™ | 2.00 | 1.50 | 0.75 | 9.00 |
| Titanium Dioxide-microfine | 1.00 | | 3.00 | |
| Zinc Oxide-microfine | | 1.00 | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

Self-tanning PIT emulsions

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone Copolyol | | | | 0.50 | | 1.00 | | |
| Polyglyceryl-2 | | | | 0.75 | 0.30 | | | |
| Fusion protein-KBD | 0.1 | 0.5 | 0.01 | 5.0 | 0.5 | 3.0 | 0.025 | 10.0 |
| Dihydroxyacetone | | | 3.00 | 5.00 | | | 4.00 | |
| Uvinul ® A Plus ™ | 2.00 | 1.50 | 0.75 | 1.00 | 2.10 | 4.50 | 5.00 | 2.10 |
| Titanium Dioxide-microfine | 1.00 | | 1.50 | | 3.50 | | 1.50 | 1.00 |
| Zinc Oxide-microfine | | 1.00 | | 0.25 | | 2.00 | | 1.50 |
| C12-15 Alkyl Benzoate | 3.50 | | | 6.35 | | | | 0.10 |
| Cocoglycerides | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl Galactomannan | | 0.50 | | | 2.00 | | | |
| Hydrogenated Coco-Glycerides | | | | | 3.00 | 4.00 | | |
| Behenoxy Dimethicone | | | | | | 1.50 | 2.00 | |
| VP/Hexadecene Copolymer | | | | 1.00 | 1.20 | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii (Shea | | 2.00 | | 3.60 | | 2.00 | | |
| Butter) | | | | | | | | |
| Iodopropyl Butylcarbamate | 0.12 | | | | 0.20 | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

Oil gel

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Myristyl Myristate | 3.50 | 3.00 | 4.00 | 3.00 |
| Quaternium-18 Bentonite | 5.00 | 5.00 | 6.00 | 6.00 |
| Propylene Carbonate | 15.00 | 20.00 | 18.00 | 19.50 |
| Fusion protein-KBD | 1.0 | 0.5 | 3.0 | 5.0 |
| Vitamin E Acetate | 0.50 | 1.00 | | |
| Ascorbyl Palmitate | 0.05 | | 0.05 | |
| Buxus Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

### Example 54

In the formulations below, cosmetic sunscreen preparations comprising a combination of at least one inorganic pigment, preferably zinc oxide and/or titanium dioxide and organic UV-A and UV-B filters are described.

The formulations specified below are prepared in customary ways known to the person skilled in the art.

The content of fusion protein C16-KBD-B (according to SEQ ID No.: 168) refers to 100% of active ingredient. The active ingredient according to the invention can either be used in pure form or else in the form of an aqueous solution. In the case of the aqueous solution, the content of Water demin in the particular formulation must be adjusted.

It will be appreciated by the person skilled in the art that all of the other KBD fusion proteins mentioned can also be produced using the corresponding KBD fusion protein constructs, e.g. KBD-D protein according to (SEQ ID No.: 212) in pRee024 (FIG. 8) according to Example 3 and can be used in the preparations given below.

The C16-KBD-B in the following examples is referred to as fusion protein-KBD by way of representation for all of the other abovedescribed KBD fusion proteins.

| | | | |
|---|---|---|---|
| A | 7.50 | Uvinul MC 80 | Ethylhexyl Cinnamate |
| | 2.00 | Uvinul M 40 | Benzophenone-3 |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 1.00 | Sepigel 305 | Polyacrylamide, C13-14 Isoparaffin, |
| | | | Laureth-7 |
| | 1% | Fusion protein-KBD | |
| | q.s. | Preservative | |

| | | | |
|---|---|---|---|
| A | 7.50 | Uvinul MC 80 | Ethylhexyl Cinnamate |
| | 2.00 | Uvinul M 40 | Benzophenone-3 |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 7.50 | Uvinul MC 80 | Ethylhexyl Cinnamate |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 4.00 | Uvinul MC 80 | Ethylhexyl Cinnamate |
| | 1.00 | Uvinul T 150 | Ethyl Hexyl Triazone |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF-S | Titanium Dioxide, Silica Hydrate, |
| | | | Alumina Hydrate, Methicone/ |
| | | | Dimethicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.70 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 0.5% | Fusion protein-KBD | |
| | q.s. | | Preservative |

| | | | |
|---|---|---|---|
| A | 4.00 | Uvinul MC 80 | Ethylhexyl Cinnamate |
| | 1.00 | Uvinul T 150 | Ethyl Hexyl Triazone |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 67.10 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 0.1% | Fusion protein-KBD | |
| | q.s. | | Preservative |

| | | | |
|---|---|---|---|
| A | 5.00 | Uvinul N 539 T | Octocrylene |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF-S | Titanium Dioxide, Silica Hydrate, |
| | | | Alumina Hydrate, Methicone/ |
| | | | Dimethicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 65.20 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 2% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide(and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.70 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, Polysorbate 60 |
| | 0.5% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 2.0 | Mexoryl SX | Terephthalidene Dicamphor Sulfonic |
| | | | Acid |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydrobenzoyl Hexyl |
| | | | Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 2.0 | Mexoryl SX | Terephthalidene Dicamphor Sulfonic |
| | | | Acid |
| | 65.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 2% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl Hexyl |
| | | | Benzoate |
| | 2.00 | Mexoryl XL | Drometrizole Trisiloxane |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor | PEG-40 Hydrogenated Castor Oil |
| | | CO 410 | |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 67.10 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 0.1% | Fusion | |
| | | protein-KBD | |
| | q.s. | | Preservative |

| | | | |
|---|---|---|---|
| A | 5.00 | Uvinul N 539 T | Octocrylene |
| | 2.00 | Tinosorb S | Bis-Ethylhexyloxyphenol |
| | | | Methoxyphenyl Triazine |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1% | Fusion protein-KBD | |
| | q.s. | | Preservative |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl Capryl |
| | | | Methicone |
| C | 4.00 | 1,2-Propylene | Propylene Glycol |
| | | Glycol Care | |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water demin. | Aqua dem. |
| | q.s. | Preservative | |
| | 0.50 | Vitamin E acetate | Tocopheryl Acetate |
| | 2% | Fusion protein-KBD | |
| | 1.00 | RetiSTAR | Caprylic/Capric Triglyceride, Sodium |
| | | | Ascorbate, Tocopherol, Retinol |

| | | | |
|---|---|---|---|
| A | 7.50 | Uvinul MC 80 | Ethylhexyl Methoxycinnamate |
| | 2.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF-S | Titanium Dioxide, Silica Hydrate, |
| | | | Alumina Hydrate, Methicone/ |
| | | | Dimethicone Copolymer |
| C | 4.00 | 1,2-Propylene | Propylene Glycol |
| | | Glycol Care | |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water demin. | Aqua dem. |
| | q.s. | Preservative | |
| | 0.50 | Vitamin E acetate | Tocopheryl Acetate |
| | 1% | Fusion protein-KBD | |
| | 2.00 | RetiSTAR | Caprylic/Capric Triglyceride, Sodium |
| | | | Ascorbate, Tocopherol, Retinol |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF | Titanium Dioxide, Alumina Hydrate, |
| | | | Dimethicone/Methicone Copolymer |
| C | 4.00 | 1,2-Propylene | Propylene Glycol |
| | | Glycol Care | |
| | 1.00 | D-Panthenol 50 P | Panthenol, Propylene Glycol |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water demin. | Aqua dem. |
| | q.s. | Preservative | |
| | 0.50 | Vitamin E acetate | Tocopheryl Acetate |
| | 1% | Fusion protein-KBD | |
| | 1.00 | RetiSTAR | Caprylic/Capric Triglyceride, Sodium |
| | | | Ascorbate, Tocopherol, Retinol |

| | | | |
|---|---|---|---|
| A | 5.50 | Uvinul MC 80 | Ethylhexyl Methoxycinnamate |
| | 2.00 | Uvinul A Plus | Diethylamino Hydroxybenzoyl Hexyl |
| | | | Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF | Titanium Dioxide, Alumina Hydrate, |
| | | | Dimethicone/Methicone Copolymer |
| C | 4.00 | 1,2-Propylene | Propylene Glycol |
| | | Glycol Care | |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water demin. | Aqua dem. |
| | q.s. | Preservative | |
| | 0.50 | Vitamin E acetate | Tocopheryl Acetate |
| | 2% | Fusion protein-KBD | |
| | 1.00 | RetiSTAR | Caprylic/Capric Triglyceride, Sodium |
| | | | Ascorbate, Tocopherol, Retinol |

| | | | |
|---|---|---|---|
| A | 1.00 | Abil Care 85 | Bis-PEG/PPG-16/16 PEG/PPG-16/16 |
| | | | Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Parsol 1789 | Butyl Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 | Cyclopentasiloxane, Cyclohexasiloxane |
| | | Fluid | |
| | 5.00 | Uvinul N 539 T | Octocrylene |

| | | | |
|---|---|---|---|
| B | 3.00 | Z-COTE MAX | Zinc Oxide (and) Diphenyl Capryl |
| | | | Methicone |
| C | 5.00 | 1,2-Propylene Glycol | Propylene Glycol |
| | 1.00 | Mexoryl SX | Terephthalidene Dicamphor |
| | | | Sulfanic Acid |
| | 0.20 | Keltrol | Xanthan Gum |
| | 0.06 | Edeta BD | Disodium EDTA |
| | 0.04 | TEA | Triethanolamine |
| | 0.30 | Carbopol Ultrez 10P | Carbomer |
| | 63.50 | Water demin. | Aqua dem. |
| | 1% | Fusion protein-KBD | |
| D | q.s. | Perfume oil | |
| | 0.50 | Glydant | DMDM Hydantoin |

| | | | |
|---|---|---|---|
| A | 5.50 | Uvinul MC 80 | Ethylhexyl Methoxycinnamate |
| | 2.00 | Uvinul A Plus | Diethylamino Hydroxybenzoyl Hexyl |
| | | | Benzoate |
| | 3.00 | Uvinul N 539 T | Octocrylene |
| | 3.00 | Emulgade | Cetearyl Glucoside, Cetearyl Alcohol |
| | | PL 68/50 | |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF-S | Titanium Dioxide, Silica Hydrate, |
| | | | Alumina Hydrate, Methicone/ |
| | | | Dimethicone Copolymer |
| C | 4.00 | 1,2-Propylene | Propylene Glycol |
| | | Glycol Care | |
| | 0.20 | Keltrol | Xanthan Gum |
| | 2.00 | Simulgel NS | Hydroxyethyl Acrylate/Sodium |
| | | | Acryloyldimethyl Taurate Copolymer, |
| | | | Squalane, Polysorbate 60 |
| | 66.30 | Water demin. | Aqua dem. |
| | q.s. | Preservative | |
| | 0.50 | Vitamin E acetate | Tocopheryl Acetate |
| | 0.5% | Fusion | |
| | | protein-KBD | |
| | 1.00 | RetiSTAR | Caprylic/Capric Triglyceride, Sodium |
| | | | Ascorbate, Tocopherol, Retinol |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl | Isopropyl Palmitate |
| | | Palmitate | |
| B | 3.00 | Z-COTE MAX | Zinc Oxide (and) Diphenyl Capryl |
| | | | Methicone |
| C | 0.20 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene | Propylene Glycol |
| | | Glycol Care | |
| | 0.5% | Fusion | |
| | | protein-KBD | |
| D | 1.00 | Vitamin E acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl | Isopropyl Palmitate |
| | | Palmitate | |
| B | 3.00 | T-Lite SF | Titanium Dioxide, Alumina Hydrate, |
| | | | Dimethicone/Methicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene | Propylene Glycol |
| | | Glycol Care | |
| | 0.5% | Fusion | |
| | | protein-KBD | |
| D | 1.00 | Vitamin E acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, Silica Hydrate, |
| | | | Alumina Hydrate, Methicone/ |
| | | | Dimethicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 64.60 | Water demin. | Aqua dem. |
| | 0.20 | Edeta BD | Disodium EDTA |
| | 5.00 | 1,2-Propylene | Propylene Glycol |
| | | Glycol Care | |
| | 1% | Fusion protein-KBD | |
| D | 1.00 | Vitamin E acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, Silica Hydrate, |
| | | | Alumina Hydrate, Methicone/ |
| | | | Dimethicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 64.10 | Water demin. | Aqua dem. |
| | 2.00 | Neoheliopan AP | Disodium Phenyl Dibenzimidazole |
| | | | Tetrasulfonate |
| | 0.20 | Edeta BD | Disodium EDTA |
| | 5.00 | 1,2-Propylene | Propylene Glycol |
| | | Glycol Care | |
| D | 1.00 | Vitamin E acetate | Tocopheryl Acetate |
| | 1% | Fusion protein-KBD | |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 7.00 | Uvinul N 539 T | Octocrylene |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, |
| | | | Silica Hydrate, Alumina |
| | | | Hydrate, |
| | | | Methicone/Dimethicone |
| | | | Copolymer |
| C | 0.50 | Keltrol | Xanthan Gum |
| | 0.50 | Simulgel NS | Hydroxyethyl Acrylate/Sodium |
| | | | Acryloyldimethyl Taurate |
| | | | Copolymer, |
| | | | Squalane, Polysorbate 60 |
| | 64.60 | Water demin. | Aqua dem. |
| | 1.00 | Mexoryl SX | Terephthalidene Dicamphor |
| | | | Sulfonic Acid |
| | 0.20 | Edeta BD | Disodium EDTA |
| | 5.00 | 1,2-Propylene Glycol Care | Propylene Glycol |
| D | 1.00 | Vitamin E acetate | Tocopheryl Acetate |
| | 1% | Fusion protein-KBD | |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 2.00 | Mexoryl XL | Drometrizole Trisiloxane |
| | 5.00 | Uvinul N 539 T | Octocrylene |
| | 1.00 | Ganex V-220 | VP/Eicosene Copolymer |
| | 7.00 | Isopropyl | Isopropyl Palmitate |
| | | Palmitate | |
| B | 3.00 | T-Lite SF | Titanium Dioxide, Alumina Hydrate, |
| | | | Dimethicone/Methicone Copolymer |
| C | 0.50 | Keltrol | Xanthan Gum |
| | 0.50 | Simulgel NS | Hydroxyethyl Acrylate/Sodium |
| | | | Acryloyldimethyl Taurate |
| | | | Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene | Propylene Glycol |
| | | Glycol Care | |
| D | 1.00 | Vitamin E acetate | Tocopheryl Acetate |
| | 0.5% | Fusion | |
| | | protein-KBD | |
| | q.s. | Preservative | |

| | | | |
|---|---|---|---|
| | 1.00 | Urea | Urea |
| | 2.00 | Neoheliopan AP | Disodium Phenyl Dibenzimidazole |
| | | | Tetrasulfonate |
| | 2.00 | Glycerin 87% | Glycerin |
| | 1.20 | Aristoflex AVC | Ammonium Acryloyldimethyltaurate/VP |
| | | | Copolymer |
| | 71.30 | Water demin. | Aqua dem. |
| B | 3.00 | Cremophor | PEG-40 Hydrogenated Castor Oil |
| | | CO 40 | |
| | q.s. | Perfume oil | |
| | 5.00 | Miglyol 812 | Caprylic/Capric Triglyceride |
| | 2.00 | Uvinul N 539 T | Octocrylene |
| | 8.00 | Z-COTE MAX | Zinc Oxide (and)Diphenyl Capryl |
| | | | Methicone |
| | 5% | Fusion | |
| | | protein-KBD | |
| | q.s. | Preservative | |

| | | |
|---|---|---|
| 5.00 | Z-COTE MAX | Zinc Oxide (and) Diphenyl |
| | | Capryl Methicone |
| 2.00 | Parsol 1789 | Butyl Methoxydibenzoylmethane |
| 1.00 | Uvinul T150 | Ethylhexyl Triazone |
| 5.00 | Uvinul MC 80 | Ethylhexyl Methoxycinnamate |
| 2.00 | Uvinul T 539 N | Octocrylene |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 LJ | Candelilla (Euphorbia Cerifera) Wax |
| 1.80 | Beeswax 3050 PH | Beeswax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E acetate | Tocopheryl Acetate |
| 0.42 | D,L-Alpha-Tocopherol | Tocopherol |
| 1% | Fusion protein-KBD | |
| 40.38 | castor oil | Castor (Ricinus Communis) Oil |

| | | |
|---|---|---|
| 5.00 | T-Lite SF | Titanium Dioxide, Alumina Hydrate, |
| | | Dimethicone/Methicone Copolymer |
| 2.00 | Parsol 1789 | Butyl Methoxydibenzoylmethane |
| 1.00 | Uvinul T150 | Ethylhexyl Triazone |
| 3.00 | Uvinul MC 80 | Ethylhexyl Methoxycinnamate |
| 2.00 | Uvinul N 539 T | Octocrylene |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 LJ | Candelilla (Euphorbia Cerifera) Wax |
| 1.80 | Beeswax 3050 PH | Beeswax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E acetate | Tocopheryl Acetate |
| 0.42 | D,L-Alpha-Tocopherol | Tocopherol |
| 1% | Fusion protein-KBD | |
| 40.38 | castor oil | Castor (Ricinus Communis) Oil |

| | | |
|---|---|---|
| 5.00 | T-Lite SF-S | Titanium Dioxide, Silica Hydrate, |
| | | Alumina Hydrate, |
| | | Methicone/Dimethicone Copolymer |
| 2.00 | Uvinul A Plus | Diethylamino Hydroxybenzoyl Hexyl |
| | | Benzoate |
| 2.00 | Mexoryl XL | Drometrizole Trisiloxane |
| 3.00 | Uvinul MC 80 | Ethylhexyl Methoxycinnamate |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 LJ | Candelilla (Euphorbia Cerifera) Wax |
| 1.80 | Beeswax 3050 PH | Beeswax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E acetate | Tocopheryl Acetate |
| 0.42 | D,L-Alpha-Tocopherol | Tocopherol |
| 2% | Fusion protein-KBD | |
| 39.38 | castor oil | Castor (Ricinus Communis) Oil |

| | | | |
|---|---|---|---|
| A | 6.00 | Cremophor WO 7 | PEG-7 Hydrogenated Castor Oil |
| | 2.00 | Elfacos ST 9 | PEG-45/Dodecyl Glycol Copolymer |
| | 3.00 | Isopropyl myristate | Isopropyl Myristate |
| | 8.00 | Jojoba oil | Simmondsia Chinensis (Jojoba) Seed |
| | | | Oil |
| | 4.00 | Uvinul MC 80 | Ethylhexyl Methoxycinnamate |
| | 2.00 | Uvinul A Plus | Diethylamino Hydroxybenzoyl Hexyl |
| | | | Benzoate |
| | 1.00 | Abil 350 | Dimethicone |
| B | 5.00 | Z-COTE MAX | Zinc Oxide (and) Diphenyl Capryl |
| | | | Methiconee |
| | 3.00 | T-Lite SF | Titanium Dioxide, Alumina Hydrate, |
| | | | Dimethicone/Methicone Copolymer |
| C | 0.20 | Edeta BD | Disodium EDTA |
| | 5.00 | Glycerin 87% | Glycerin |
| | 0.30 | Chemag 2000 | Imidazolidinyl Urea |
| | 59.00 | Water demin. | Aqua dem. |
| D | q.s. | Perfume oil | |
| | 1% | Fusion protein-KBD | |
| | q.s. | Preservative | |

| | | | |
|---|---|---|---|
| | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Uvinul T 150 | Ethythexyl Triazone |
| | 1.00 | Ganex V-220 | VP/Eicosene Copolymer |
| | 7.00 | Isopropyl myristate | Isopropyl Myristate |
| B | 5.00 | Z-COTE MAX | Zinc Oxide (and) Diphenyl |
| | | | Capryl Methicone |
| C | 0.20 | Keltrol | Xanthan Gum |
| | 0.50 | Simulgel NS | Hydroxyethyl |
| | | | Acrylate/Sodium |
| | | | Acryloyldimethyl Taurate |
| | | | Copolymer, |
| | | | Squalane, Polysorbate 60 |
| | 61.10 | Water demin. | Aqua dem. |
| | 0.20 | Edeta BD | Disodium EDTA |
| | 5.00 | 1,2-Propylene Glycol | Propylene Glycol |
| | | Care | |
| D | 1.00 | Vitamin E acetate | Tocopheryl Acetate |
| | 1.5% | Fusion protein-KBD | |
| | q.s. | Preservative | |

| | | | |
|---|---|---|---|
| A | 2.00 | Luvitol EHO | Cetearyl Ethylhexanoate |
| | 5.00 | Paraffin oil | Mineral Oil |
| | 5.00 | Z-COTE MAX | Zinc Oxide (and) Diphenyl |
| | | | Capryl |
| | | | Methicone |
| B | 1.00 | Cremophor A 6 | Ceteareth-6, Stearyl Alcohol |
| | 2.00 | Cremophor A 25 | Ceteareth-25 |
| | 3.00 | Glycerin monostearate | Glyceryl Stearate |
| | 4.00 | Cetylstearyl alcohol | Cetearyl Alcohol |
| C | 5.00 | 1,2-Propylene Glycol Care | Propylene Glycol |
| | 7.50 | Luviquat Care | Polyquaternium-44 |
| | q.s. | Preservative | |
| | q.s. | Perfume oil | |
| | 1% | Fusion protein-KBD | |
| | 64.50 | Water demin. | Aqua dem. |

| | | |
|---|---|---|
| 5.00 | T-Lite SF | Titanium Dioxide, Alumina Hydrate, |
| | | Dimethicone/Methicone Copolymer |
| 10.00 | Z-COTE MAX | Zinc Oxide (and) Diphenyl Capryl |
| | | Methicone |
| 12.00 | Beeswax 3044 PH | Beeswax |
| 8.00 | Candelilla wax LT 281 LJ | Candelilla (Euphorbia Cerifera) Wax |
| 5.00 | Tegin | Glyceryl Stearate SE |
| 5.00 | Dow Corning 345 Fluid | Cyclopentasiloxane, |
| | | Cyclohexasiloxane |
| 5.00 | Witconol APM | PPG-3 Myristyl Ether |
| 5.00 | Softisan 154 | Hydrogenated Palm Oil |
| 8.00 | Paraffin oil, high-viscosity | Mineral Oil |
| 3.00 | Vaseline | Petrolatum |
| 2% | Fusion protein-KBD | |
| 32.00 | castor oil | Castor (Ricinus Communis) Oil |

| | | |
|---|---|---|
| 3.00 | Uvinul MC 80 | Ethylhexyl Methoxycinnamate |
| 2.00 | Uvinul T 150 | Ethylhexyl Triazone |
| 2.00 | Uvinul A Plus | Diethylamino Hydroxybenzoyl |
| | | Hexyl |
| | | Benzoate |
| 10.00 | Z-COTE MAX | Zinc Oxide (and) Diphenyl Capryl |
| | | Methicone |
| 12.00 | Beeswax 3044 PH | Beeswax |
| 3.00 | Vaseline | Petrolatum |
| 8.00 | Candelilla wax LT 281 LJ | Candelilla (Euphorbia Cerifera) |
| | | Wax |
| 8.00 | Paraffin oil, high-viscosity | Mineral Oil |
| 5.00 | Tegin | Glyceryl Stearate SE |
| 5.00 | Softisan 154 | Hydrogenated Palm Oil |
| 5.00 | Witconol APM | PPG-3 Myristyl Ether |
| 5.00 | Dow Corning 345 Fluid | Cyclopentasiloxane, |
| | | Cyclohexasiloxane |
| 0.1% | Fusion protein-KBD | |
| 28.90 | castor oil | Castor (Ricinus Communis) Oil |

| | | | |
|---|---|---|---|
| | 5.00 | T-Lite SF | Titanium Dioxide, Alumina |
| | | | Hydrate, Dimethicone/Methicone |
| | | | Copolymer |
| | 6.00 | Finsolv TN | C12-15 Alkyl Benzoate |
| | 10.00 | Uvinul MC 80 | Ethylhexyl Methoxycinnamate |
| | 6.00 | Miglyol 812 | Caprylic/Capric Triglyceride |
| | 5.00 | Arlacel P 135 | PEG-30 Dipolyhydroxystearate |
| | 2.00 | Ganex V 216 | PVP/Hexadecene Copolymer |
| | 2.00 | Elfacos ST 9 | PEG-45/Dodecyl Glycol |
| | | | Copolymer |
| B | 3.00 | 1,2-Propylene | Propylene Glycol |
| | | Glycol Care | |
| | 0.10 | Edeta BD | Disodium EDTA |
| | 1.00 | Magnesium | Magnesium Sulfate |
| | | sulfate 7-hydrate | |
| | 59.40 | Water demin. | Aqua dem. |
| | 0.5% | Fusion protein-KBD | |
| | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan |
| | | | Sesquioleate, Cera Alba, Aluminum |
| | | | Stearates, Dicocoyl Pentaerythrityl |
| | | | Distearyl Citrate |
| | 1.00 | Dehymuls | Polyglyceryl-2 Dipolyhydroxystearate |
| | | PGPH | |
| | 8.00 | Finsolv TN | C12-15 Alkyl Benzoate |
| | 4.00 | Miglyol 812 | Caprylic/Capric Triglyceride |
| | 4.00 | Uvinul MC 80 | Ethylhexyl Methoxycinnamate |
| | 2.00 | Uvinul N 539 T | Octocrylene |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate 7-hydrate | |
| | ad 100 | Water demin. | Aqua dem. |
| | 1.5% | Fusion | |
| | | protein-KBD | |
| D | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan Sesquioleate, |
| | | | Cera Alba, Aluminum |
| | | | Stearates, Dicocoyl Pentaerythrityl |
| | | | Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 4.00 | Uvinul MC 80 | Ethylhexyl Methoxycinnamate |
| | 2.00 | Uvinul N 539 T | Octocrylene |
| B | 5.00 | T-Lite SF-S | Titanium Dioxide, |
| | | | Silica Hydrate, Alumina |
| | | | Hydrate, Methicone/Dimethicone |
| | | | Copolymer |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate 7-hydrate | |
| | 1% | Fusion | |
| | | protein-KBD | |
| | ad 100 | Water demin. | Aqua dem. |
| D | q.s. | preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan Sesquioleate, |
| | | | Cera Alba, Aluminum |
| | | | Stearates, Dicocoyl Pentaerythrityl |
| | | | Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl hexyl |
| | | | Benzoate |
| | 2.00 | Uvinul N 539 T | Octocrylene |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate 7-hydrate | |
| | 0.1% | Fusion | |
| | | protein-KBD | |
| | ad 100 | Water demin. | Aqua dem. |
| | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, Sorbitan |
| | | | Sesquioleate, Cera Alba, Aluminum |
| | | | Stearates, Dicocoyl Pentaerythrityl |
| | | | Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl Hexyl |
| | | | Benzoate |
| | 2.00 | Uvinul N 539 T | Octocrylene |
| B | 5.00 | T-Lite SF | Titanium Dioxide, Alumina |
| | | | Hydrate, Dimethicone/Methicone |
| | | | Copolymer |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | ad 100 | Water demin. | Aqua dem. |
| | 0.2% | Fusion protein-KBD | |
| | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, Sorbitan |
| | | | Sesquioleate, Cera Alba, Aluminum |
| | | | Stearates, Dicocoyl Pentaerythrityl |
| | | | Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl hexyl |
| | | | Benzoate |
| | 2.00 | Uvinul N 539 T | Octocrylene |
| B | 5.00 | T-Lite SF-S | Titanium Dioxide, Silica Hydrate, Alumina |
| | | | Hydrate, Methicone/Dimethicone |
| | | | Copolymer |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | 0.5% | Fusion protein-KBD | |
| | ad 100 | Water demin. | Aqua dem. |
| | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, Sorbitan |
| | | | Sesquioleate, Cera Alba, Aluminum |
| | | | Stearates, Dicocoyl Pentaerythrityl |
| | | | Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 7.00 | Uvinul MC 80 | Ethylhexyl Methoxycinnamate |
| | 2.00 | Mexoryl XL | Drometrizole Trisiloxane |
| B | 5.00 | Z-COTE MAX | Titanium Dioxide, Alumina Hydrate, |
| | | | Dimethicone/Methicone Copolymer |
| C | 3.00 | 1,2-Propylene Glycol Care | Propylene Glycol |
| | 0.30 | Abiol | Imidazolidinyl Urea |
| | 1.00 | Magnesium sulfate 7-hydrate | Magnesium Sulfate |
| | ad 100 | Water demin. | Aqua dem. |
| | 0.01% | Fusion protein-KBD | |
| | q.s. | Preservative | |

| | | | |
|---|---|---|---|
| A | 7.50 | Uvinul MC 80 | Ethylhexyl Methoxycinnamate |
| | 3.00 | Uvinul N 539 T | Octocrylene |
| | 2.00 | Uvinul A Plus | Diethylamino Hydroxybenzoyl Hexyl |
| | | | Benzoate |
| | 1.00 | Cremophor CO 40 | PEG-40 Hydrogenated Castor Oil |
| | 10.00 | Miglyol 812 | Caprylic/Capric Triglyceride |
| | 1.50 | Dow Corning 345 | Cyclopentasiloxane, |
| | | Fluid | Cyclohexasiloxane |
| B | 3.50 | Luvigel EM | Caprylic/Capric Triglyceride, Sodium |
| | | | Acrylates Copolymer |
| C | 46.00 | Water demin. | Aqua dem. |
| D | 5.00 | 1,2-Propylene | Propylene Glycol |
| | | Glycol Care | |
| | 0.50 | Cremophor A 25 | Ceteareth-25 |
| | 0.05% | Fusion protein-KBD | |
| | 20.00 | Ethanol 96% | Alcohol |

| | | | |
|---|---|---|---|
| A | 1.00 | Uvinul A Plus | Diethylamino Hydroxybenzoyl Hexyl |
| | | | Benzoate |
| | 1.00 | Tinosorb S | Bis-Ethylhexyloxyphenol |
| | | | Methoxyphenyl Triazine |
| | 3.00 | Uvinul MC 80 | Ethylhexyl Methoxycinnamate |
| | 8.00 | Miglyol 812 | Caprylic/Capric Triglyceride |
| | 1.50 | Dow Corning 350 | Dimethicone |
| | | Fluid | |
| | 3.00 | Z-COTE MAX | Titanium Dioxide, Alumina Hydrate, |
| | | | Dimethicone/Methicone Copolymer |
| | 3.00 | Finsolv TN | C12-15 Alkyl Benzoate |
| | 1.00 | Cremophor CO 40 | PEG-40 Hydrogenated Castor Oil |
| B | 2.00 | Luvigel EM | Caprylic/Capric Triglyceride, Sodium |
| | | | Acrylates Copolymer |
| C | 54.80 | Water demin. | Aqua dem. |
| D | 15.00 | Ethanol 96% | Alcohol |
| | 5.00 | 1,2-Propylene Glycol | Propylene Glycol |
| | | Care | |
| | 0.50 | Cremophor A 25 | Ceteareth-25 |
| | 1% | Fusion protein-KBD | |
| | 1.00 | Vitamin E acetate | Tocopheryl Acetate |
| | 0.20 | Bisabolol rac. | Bisabolol |

| | | | |
|---|---|---|---|
| A | 4.00 | Dehymuls SBL | Polyglyceryl-2 Dipolyhydroxy- |
| | | | stearate, Dicaprylyl Ether, |
| | | | Cocoglycerides, Sorbitan |
| | | | Sesquioleate, Cera Alba, Aluminum |
| | | | Stearates, Dicocoyl Pentaerythrityl |
| | | | Distearyl Citrate |
| | 1.00 | Dehymuls PGPH | Polyglyceryl-2 |
| | | | Dipolyhydroxystearate |
| | 6.00 | Finsolv TN | C12-15 Alkyl Benzoate |
| | 6.00 | Miglyol 812 | Caprylic/Capric Triglyceride |
| | 5.00 | Uvinul MC 80 | Ethylhexyl Methoxycinnamate |
| | 3.00 | Neoheliopan HMS | Homosalate |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Chemag 2000 | Imidazolidinyl Urea |
| | 1.00 | Magnesium sulfate | Magnesium Sulfate |
| | | 7-hydrate | |
| | 65.10 | Water demin. | Aqua dem. |
| | 0.1% | Fusion protein-KBD | |
| | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Uvinul N 539 T | Octocrylene |
| | 2.00 | Uvinul T 150 | Ethylhexyl Triazone |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | T-Lite SF | Titanium Dioxide, Alumina |
| | | | Hydrate, Dimethicone/Methicone |
| | | | Copolymer |
| C | 60.00 | Water demin. | Water |
| | 5.00 | Glycerin 87% | Glycerin |
| | 1.00 | Lanette E | Sodium Cetearyl Sulfate |
| | 0.30 | Keltrol | Xanthan Gum |
| | 0.2% | Fusion protein-KBD | |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Uvinul N 539 T | Octocrylene |
| | 2.00 | Neoheliopan HMS | Homosalate |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | T-Lite SF | Titanium Dioxide, Alumina Hydrate, |
| | | | Dimethicone/Methicone Copolymer |
| C | 59.20 | Water demin. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| | 1% | Fusion protein-KBD | |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Neoheliopan OS | Octisalate |
| | 2.00 | Neoheliopan HMS | Homosalate |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | Z-COTE MAX | Zinc Oxide (and) Diphenyl Capryl |
| | | | Methicone |
| C | 60.15 | Water demin. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| | 0.05% | Fusion protein-KBD | |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul N 539 T | Octocrylene |
| | 3.00 | Neoheliopan OS | Octisalate |
| | 2.00 | Neoheliopan HMS | Homosalate |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | Z-COTE MAX | Zinc Oxide (and) Diphenyl Capryl |
| | | | Methicone |
| C | 60.19 | Water demin. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| | 0.01% | Fusion protein-KBD | |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul N 539 T | Octocrylene |
| | 3.00 | Neoheliopan OS | Octisalate |
| | 2.00 | Neoheliopan HMS | Homosalate |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | T-Lite SF-S | Titanium Dioxide, Silica Hydrate, |
| | | | Alumina Hydrate, |
| | | | Methicone/Dimethicone Copolymer |
| C | 59.20 | Water demin. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| | 1% | Fusion protein-KBD | |
| D | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb S | Bis-Ethylhexyloxyphenol |
| | | | Methoxyphenyl Triazine |
| | 1.00 | Vitamin E acetate | Tocopheryl Acetate |
| B | 4.00 | Z-COTE MAX | Zinc Oxide (and) Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1.50 | Veegum Ultra | Magnesium Aluminum Silicate |
| | 49.45 | Water demin. | Aqua dem. |
| | 1% | Fusion protein-KBD | |
| D | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb S | Bis-Ethylhexyloxyphenol |
| | | | Methoxyphenyl Triazine |
| | 1.00 | Vitamin E acetate | Tocopheryl Acetate |
| B | 4.00 | T-Lite SF | Titanium Dioxide, Alumina Hydrate, |
| | | | Dimethicone/Methicone Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1.50 | Veegum Ultra | Magnesium Aluminum Silicate |
| | 50.25 | Water demin. | Aqua dem. |
| D | 0.50 | Citric acid | Citric Acid |
| | q.s. | Perfume oil | |
| | 0.2% | Fusion protein-KBD | |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul T 150 | Ethylhexyl Triazone |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 2.50 | T-Lite SF | Titanium Dioxide, Alumina Hydrate, |
| | | | Dimethicone/Methicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 42.00 | Water demin. | Aqua dem. |
| D | 5.00 | Eusolex 232 | Phenylbenzimidazole Sulfonic Acid |
| | 10.00 | Water demin. | Aqua dem. |
| E | 0.70 | Sodium hydroxide | Sodium Hydroxide |
| | 1% | Fusion protein-KBD | |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul A Plus | Diethylamino Hydrobenzoyl Hexyl |
| | | | Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 2.50 | T-Lite SF | Titanium Dioxide, Alumina Hydrate, |
| | | | Dimethicone/Methicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 41.00 | Water demin. | Aqua dem. |
| D | 5.00 | Eusolex 232 | Phenylbenzimidazole Sulfonic Acid |
| | 2% | Fusion protein-KBD | |
| | 10.00 | Water demin. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul A Plus | Diethylamino Hydrobenzoyl Hexyl |
| | | | Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 2.50 | Z-COTE MAX | Zinc Oxide (And) Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 42.50 | Water demin. | Aqua dem. |
| D | 5.00 | Eusolex 232 | Phenylbenzimidazole Sulfonic Acid |
| | 0.5% | Fusion protein-KBD | |
| | 10.00 | Water demin. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 7.50 | Uvinul N 539 T | Octocrylene |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 5.00 | Z-COTE MAX | Zinc Oxide (And) Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 40.50 | Water demin. | Aqua dem. |
| D | 5.00 | Eusolex 232 | Phenylbenzimidazole Sulfonic Acid |
| | 1% | Fusion protein-KBD | |
| | 10.00 | Water demin. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 5.50 | Uvinul N 539 T | Octocrylene |
| | 2.00 | Uvinul A Plus | Diethylamino Hydrobenzoyl Hexyl |
| | | | Benzoate |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 5.00 | Z-COTE MAX | Zinc Oxide (And) Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 41.50 | Water demin. | Aqua dem. |
| D | 5.00 | Eusolex 232 | Phenylbenzimidazole Sulfonic Acid |
| | 0.5% | Fusion protein-KBD | |
| | 9.50 | Water demin. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 5.50 | Uvinul N 539 T | Octocrylene |
| | 2.00 | Uvinul A Plus | Diethylamino Hydrobenzoyl Hexyl |
| | | | Benzoate |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 5.00 | T-Lite SF-S | Titanium Dioxide, Silica Hydrate, |
| | | | Alumina Hydrate, |
| | | | Methicone/Dimethicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 40.50 | Water demin. | Aqua dem. |
| D | 5.00 | Eusolex 232 | Phenylbenzimidazole Sulfonic Acid |
| | 1% | Fusion protein-KBD | |
| | 10.00 | Water demin. | Aqua dem. |
| E | 0.70 | Sodium hydroxide | Sodium Hydroxide |
| | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 10.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb S | Bis-Ethylhexyloxyphenol |
| | | | Methoxyphenyl Triazine |
| | 1.00 | Uvinul T 150 | Ethylhexyl Triazone |
| | 1.00 | Vitamin E acetate | Tocopheryl Acetate |
| B | 5.00 | T-Lite SF | Titanium Dioxide, Alumina Hydrate, |
| | | | Dimethicone/Methicone Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 2% | Fusion protein-KBD | |
| | 1.50 | Veegum Ultra | Magnesium Aluminum Silicate |
| | ad 100 | Water demin. | Aqua dem. |
| D | q.s. | Perfume oil | |
| | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 10.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb S | Bis-Ethylhexyloxyphenol |
| | | | Methoxyphenyl triazine |
| | 1.00 | Uvinul T 150 | Ethylhexyl Triazone |
| | 1.00 | Vitamin E acetate | Tocopheryl Acetate |
| B | 5.00 | Z-COTE MAX | Zinc Oxide (and) Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1% | Fusion protein-KBD | |
| | 1.50 | Veegum Ultra | Magnesium Aluminum Silicate |
| | ad 100 | Water demin. | Aqua dem. |
| D | q.s. | Perfume oil | |
| | q.s. | Preservative | |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 57.00 | Water demin. | Aqua dem. |
| B | 5.00 | T-Lite SF-S | Titanium Dioxide, Silica Hydrate, |
| | | | Alumina Hydrate, |
| | | | Methicone/Dimethicone Copolymer |
| C | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 2% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 58.00 | Water demin. | Aqua dem. |
| B | 5.00 | Z-COTE MAX | Zinc Oxide (and) Diphenyl |
| | | | Capryl Methicone |
| C | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 Fluid | Cyclopentasiloxane, |
| | | | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 1% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 58.50 | Water demin. | Aqua dem. |
| B | 5.00 | T-Lite SF-S | Titanium Dioxide, Silica Hydrate, |
| | | | Alumina Hydrate, |
| | | | Methicone/Dimethicone Copolymer |
| C | 7.50 | Uvinul MC 80 | Ethylhexyl Methoxycinnamate |
| | 5.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 0.5% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Mexoryl SL | Terephthalidene Dicamphor |
| | | | Sulfonic Acid |
| | 58.40 | Water demin. | Aqua dem. |
| B | 6.00 | T-Lite SF-S | Titanium Dioxide, Silica Hydrate, |
| | | | Alumina Hydrate, |
| | | | Methicone/Dimethicone Copolymer |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 0.1% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Mexoryl SL | Terephthalidene Dicamphor |
| | | | Sulfonic Acid |
| | 58.45 | Water demin. | Aqua dem. |
| B | 6.00 | Z-COTE MAX | Zinc Oxide (and) Diphenyl Capryl |
| | | | Methicone |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 0.05% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Neoheliopan AP | Disodium Phenyl Dibenzimidazole |
| | | | Tetrasulfonate |
| | 57.50 | Water demin. | Aqua dem. |
| B | 6.00 | Z-COTE MAX | Zinc Oxide (and) Dipheny Capryl |
| | | | Methicone |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 1.0% | Fusion protein-KBD | |
| | 0.20 | Bisabolol rac. | Bisabolol |

| | | | |
|---|---|---|---|
| A | 25.00 | Dow Corning 345 | Cyclopentasiloxane, |
| | | Fluid | Cyclohexasiloxane |
| | 20.00 | Dow Corning 245 | Cyclopentasiloxane |
| | | Fluid | |
| | 8.00 | Uvinul MC 80 | Ethylhexyl Methoxycinnamate |
| | 4.00 | Abil EM 90 | Cetyl PEG/PPG-10/1 Dimethicone |
| | 7.00 | T-Lite SF | Titanium Dioxide, Alumina Hydrate, |
| | | | Dimethicone/Methicone Copolymer |
| B | 17.00 | Ethanol 95% | Alcohol |
| | 10.00 | Z-COTE MAX | Zinc Oxide (and) Diphenyl Capryl |
| | | | Methicone |
| | 4.50 | Water demin. | Aqua dem. |
| | 3.00 | Glycerin 87% | Glycerin |
| | 0.5% | Fusion protein-KBD | |
| | 1.00 | talc | Talc |

| | | | |
|---|---|---|---|
| A | 25.00 | Dow Corning 345 | Cyclopentasiloxane, |
| | | Fluid | Cyclohexasiloxane |
| | 20.00 | Dow Corning 245 | Cyclopentasiloxane |
| | | Fluid | |
| | 8.00 | Uvinul MC 80 | Ethylhexyl Methoxycinnamate |
| | 4.00 | Abil EM 90 | Cetyl PEG/PPG-10/1 Dimethicone |
| | 7.00 | T-Lite SF-S | Titanium Dioxide, Silica Hydrate, |
| | | | Alumina Hydrate, |
| | | | Methicone/Dimethicone Copolymer |
| B | 17.00 | Ethanol 95% | Alcohol |
| | 9.99 | Z-COTE MAX | Zinc Oxide (and) Diphenyl Capryl |
| | | | Methicone |
| | 5.00 | Water demin. | Aqua dem. |
| | 3.00 | Glycerin 87% | Glycerin |
| | 0.01% | Fusion protein-KBD | |
| | 1.00 | talc | Talc |

| | | | |
|---|---|---|---|
| A | 20.00 | Dow Corning 345 | Cyclopentasiloxane, |
| | | Fluid | Cyclohexasiloxane |
| | 10.00 | Dow Corning 245 | Cyclopentasiloxane |
| | | Fluid | |
| | 7.50 | Uvinul MC 80 | Ethylhexyl Methoxycinnamate |
| | 2.00 | Uvinul T 150 | Ethylhexyl Triazone |
| | 12.00 | Cosmacol EMI | Di-C12-13 Alkyl Malate |
| | 4.00 | Abil EM 90 | Cetyl PEG/PPG-10/1 Dimethicone |
| B | 7.00 | T-Lite SF | Titanium Dioxide, Alumina Hydrate, |
| | | | Dimethicone/Methicone Copolymer |
| C | 15.00 | Ethanol 95% | Alcohol |
| | 10.00 | Z-COTE MAX | Zinc Oxide (and) Diphenyl Capryl |
| | | | Methicone |
| | 3.00 | Glycerin 87% | Glycerin |
| | 1.5% | Fusion protein-KBD | |
| | 8.00 | Water demin. | Aqua dem. |

| | | | |
|---|---|---|---|
| A | 20.00 | Dow Corning 345 | Cyclopentasiloxane, |
| | | Fluid | Cyclohexasiloxane |
| | 10.00 | Dow Corning 245 | Cyclopentasiloxane |
| | | Fluid | |
| | 7.50 | Uvinul MC 80 | Ethylhexyl Methoxycinnamate |
| | 2.00 | Tinosorb S | Bis-Ethylhexyloxyphenol |
| | | | Methoxyphenyl Triazine |
| | 12.00 | Cosmacol EMI | Di-C12-13 Alkyl Malate |
| | 4.00 | Abil EM 90 | Cetyl PEG/PPG-10/1 Dimethicone |
| B | 7.00 | T-Lite SF | Titanium Dioxide, Alumina Hydrate, |
| | | | Dimethicone/Methicone Copolymer |
| C | 15.00 | Ethanol 95% | Alcohol |
| | 10.00 | Z-COTE MAX | Zinc Oxide (and) Diphenyl Capryl |
| | | | Methicone |
| | 3.00 | Glycerin 87% | Glycerin |
| | 0.5% | Fusion protein-KBD | |
| | 9.00 | Water demin. | Aqua dem. |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | T-Lite SF | Titanium Dioxide, Alumina Hydrate, |
| | | | Dimethicone/Methicone Copolymer |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 61.30 | Water demin. | Aqua dem. |
| D | 1.00 | Vitamin E acetate | Tocopheryl Acetate |
| | 1% | Fusion protein-KBD | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | Z-COTE MAX | Zinc Oxide (and) Diphenyl Capryl |
| | | | Methicone |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 62.20 | Water demin. | Aqua dem. |
| D | 1.00 | Vitamin E acetate | Tocopheryl Acetate |
| | 0.1% | Fusion protein-KBD | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | Z-COTE MAX | Zinc Oxide (and) Diphenyl Capryl |
| | | | Methicone |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 60.30 | Water demin. | Aqua dem. |
| D | 1.00 | Vitamin E acetate | Tocopheryl Acetate |
| | 2% | Fusion protein-KBD | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | Z-COTE MAX | Zinc Oxide (and) Diphenyl Capryl |
| | | | Methicone |
| | 4.00 | Isopropyl Palmitate | Isopropyl Palmitate |
| | 1.00 | Abil 350 | Dimethicone |
| B | 4.00 | Uvinul N 539 T | Octocrylene |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 62.10 | Water demin. | Aqua dem. |
| D | 1.00 | Vitamin E acetate | Tocopheryl Acetate |
| | 0.2% | Fusion protein-KBD | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | Z-COTE MAX | Zinc Oxide (and) Diphenyl Capryl |
| | | | Methicone |
| | 4.00 | Isopropyl Palmitate | Isopropyl Palmitate |
| | 1.00 | Abil 350 | Dimethicone |
| B | 3.00 | Uvinul N 639 T | Octocrylene |
| | 1.00 | Mexoryl XL | Drometrizole Trisiloxane |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 1% | Fusion protein-KBD | |
| | 61.30 | Water demin. | Aqua dem. |
| D | 1.00 | Vitamin E acetate | Tocopheryl Acetate |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | T-Lite SF-S | Titanium Dioxide, Silica Hydrate, |
| | | | Alumina Hydrate, |
| | | | Methicone/Dimethicone Copolymer |
| | 4.00 | Isopropyl Palmitate | Isopropyl Palmitate |
| | 1.00 | Abil 350 | Dimethicone |
| B | 3.00 | Uvinul N 539 T | Octocrylene |
| | 1.00 | Mexoryl XL | Drometrizole trisiloxane |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 61.80 | Water demin. | Aqua dem. |
| D | 1.00 | Vitamin E acetate | Tocopheryl Acetate |
| | 0.5% | Fusion protein-KBD | |
| | q.s. | Preservative | |

| | | | |
|---|---|---|---|
| A | 4.50 | Uvinul MC 80 | Ethylhexyl Methoxycinnamate |
| | 3.00 | Uvinul N 539 T | Octocrylene |
| | 1.00 | Uvinul T 150 | Ethylhexyl Triazone |
| | 2.50 | Cosmacol EMI | Di-C12-13 Alkyl Malate |
| B | 0.50 | Vitamin E acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | Z-COTE MAX | Zinc Oxide (and) Diphenyl Capryl |
| | | | Methicone |
| D | 44.00 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1% | Fusion protein-KBD | |
| | 0.50 | Keltrol | Xanthan Gum |
| E | 1.00 | Phenonip | |

| | | | |
|---|---|---|---|
| A | 4.50 | Uvinul MC 80 | Ethylhexyl Methoxycinnamate |
| | 3.00 | Uvinul N 539 T | Octocrylene |
| | 1.00 | Tinosorb S | Bis-Ethoxylhexyloxyphenol |
| | | | Methoxyphenyl Triazine |
| | 2.50 | Cosmacol EMI | Di-C12-13 Alkyl Malate |
| B | 0.50 | Vitamin E acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexedecane | Isohexadecane |
| | 2.50 | Cosmacol EMI | Di-C12-13 Alkyl Malate |
| | 5.00 | Z-COTE MAX | Zinc Oxide (and) Diphenyl Capryl |
| | | | Methicone |
| D | 44.90 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 0.1% | Fusion protein-KBD | |
| | 0.50 | Keltrol | Xanthan Gum |
| E | 1.00 | Phenonip | |

| | | | |
|---|---|---|---|
| A | 1.50 | Mexoryl XL | Drometrizole Trisiloxane |
| | 5.00 | Uvinul N 539 T | Octocrylene |
| | 1.00 | Tinosorb S | Bis-Ethylhexyloxyphenol |
| | | | Methoxyphenyl Triazine |
| | 2.50 | Cosmacol EMI | Di-C12-13 Alkyl Malate |
| B | 0.50 | Vitamin E acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | Z-COTE MAX | Zinc Oxide (and) Diphenyl Capryl |
| | | | Methicone |
| D | 44.50 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1.5% | Fusion protein-KBD | |
| | 0.50 | Keltrol | Xanthan Gum |
| E | 1.00 | Phenonip | |

| | | | |
|---|---|---|---|
| A | 1.50 | Mexoryl XL | Drometrizole Trisiloxane |
| | 5.00 | Uvinul N 539 T | Octocrylene |
| | 1.00 | Tinosorb S | Bis-Ethylhexyloxyphenol |
| | | | Methoxyphenyl Triazine |
| | 2.50 | Cosmacol EMI | Di-C12-13 Alkyl Malate |
| B | 0.50 | Vitamin E acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | T-Lite SF | Titanium Dioxide, Alumina Hydrate, |
| | | | Dimethicone/Methicone Copolymer |
| D | 45.00 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1% | Fusion protein-KBD | |
| | 0.50 | Keltrol | Xanthan Gum |
| E | 1.00 | Phenonip | |

| | | | |
|---|---|---|---|
| A | 1.00 | Abil Care 85 | Bis-PEG/PPG-16/16 PEG/PPG-16/16 |
| | | | Dimethicone, Caprylic/Capric Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 5.00 | Uvinul MC 80 | Ethylhexyl Methoxycinnamate |
| | 10.00 | Witconol APM | PPG-3 Myristyl Ether |
| | 2.00 | Uvinul T 150 | Ethylhexyl Triazone |
| | 1.00 | Dow Corning 345 Fluid | Cyclopentasiloxane, Cyclohexasiloxane |
| | 5.00 | Uvinul N 539 T | Octocrylene |
| B | 5.00 | Z-COTE MAX | Zinc Oxide (and) Diphenyl Capryl Methicone |
| C | 5.00 | 1,2-Propylene glycol | Propylene Glycol |
| | 0.20 | Keltrol | Xanthan Gum |
| | 0.10 | Edeta BD | Disodium EDTA |
| | 2.0% | Fusion protein-KBD | |
| | 1.50 | Simulgel 600 | Acrylamide/Sodium Acryloyldimethyltaurate |
| | | | Copolymer, Isohexadecane, Polysorbate 80 |
| | 58.40 | Water demin. | Aqua dem. |
| D | q.s. | Perfume oil | |
| | 0.50 | Glydant | DMDM Hydantoin |

| | | | |
|---|---|---|---|
| A | 2.00 | Abil Care 85 | Bis-PEG/PPG-16/16 PEG/PPG-16/16 |
| | | | Dimethicone, Caprylic/Capric |
| | | | Triglyceride |
| | 4.00 | Finsolv TN | C12-15 Alkyl Benzoate |
| | 1.50 | Miglyol 812 | Caprylic/Capric Triglyceride |
| | 0.50 | Vitamin E acetate | Tocopheryl Acetate |
| | 7.50 | Uvinul MC 80 | Ethylhexyl Methoxycinnamate |
| | 4.00 | Cetiol B | Dibutyl Adipate |
| | 3.00 | Luvitol EHO | Cetearyl Ethylhexanoate |
| | 1.00 | Cremophor CO 40 | PEG-40 Hydrogenated Castor Oil |
| | 1.00 | Paraffin oil, low | Mineral Oil |
| | | viscosity | |
| | 3.00 | Plantacare 2000 | Decyl Glucoside |
| | 0.50 | Phenonip | |
| | q.s. | Perfume oil | |
| B | 4.00 | Z-COTE MAX | Zinc Oxide (and) Diphenyl Capryl |
| | | | Methicone |
| C | 2.00 | Simulgel NS | Hydroxyethyl Acrylate/Sodium |
| | | | Acryloyldimethyl Taurate |
| | | | Copolymer, Squalane, |
| | | | Polysorbate 60 |
| | 0.20 | Keltrol | Xanthan Gum |
| | 0.10 | Edeta BD | Disodium EDTA |
| | 1% | Fusion protein-KBD | |
| | 64.20 | Water demin. | Aqua dem. |

| | | | |
|---|---|---|---|
| A | 6.00 | Gilugel SIL 5 | Cyclomethicone (and) Aluminum/ |
| | | | Magnesium Hydroxide |
| | | | Stearate |
| | 5.00 | Uvinul MC 80 | Ethylhexyl Methoxycinnamate |
| | 2.00 | Uvinul A Plus | Diethylamino Hydroxybenzoyl Hexyl |
| | | | Benzoate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, Cetyl PEG/ |
| | | | PPG-10/1 Dimethicone, Hexyl Laurate |
| | 2.00 | Cosmacol EMI | Di-C12-13 Alkyl Malate |
| | 3.00 | Isopro palmitate | Isopropyl Palmitate |
| | 5.00 | Abil B 8839 | Cyclopentasiloxane, Cyclohexasiloxane |
| | 0.50 | Abil 350 | Dimethicone |
| B | 0.50 | Sodium chloride | Sodium Chloride |
| | 0.20 | Edeta BD | Disodium EDTA |
| | 0.2% | Fusion | |
| | | protein-KBD | |
| | 62.10 | Water demin. | Aqua dem. |
| C | 1.00 | Vitamin E acetate | Tocopheryl Acetate |
| | 0.50 | Phenonip | |

| | | | |
|---|---|---|---|
| A | 4.50 | Uvinul MC 80 | Ethylhexyl Methoxycinnamate |
| | 2.00 | Tinosorb S | Bis-Ethylhexyloxyphenol Methoxyphenyl |
| | | | Triazine |
| | 3.00 | Uvinul N 539 T | Octocrylene |
| | 2.50 | Cosmacol EMI | Di-C12-13 Alkyl Malate |
| | 0.50 | Vitamin E acetate | Tocopheryl Acetate |
| | 4.00 | Tego Care 450 | Polyglyceryl-3 Methyl Glucose Distearate |
| B | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 3.00 | T-Lite SF | Titanium Dioxide, Alumina Hydrate, |
| | | | Dimethicone/Methicone Copolymer |
| C | 5.00 | Glycerin 87% | Glycerin |
| | 1.00 | Lanette E | Sodium Cetearyl Sulfate |
| | 0.50 | Keltrol | Xanthan Gum |
| | 1% | Fusion protein-KBD | |
| | 59.70 | Water demin. | Aqua dem. |
| D | 1.00 | Phenonip | |
| | 0.30 | Bisabolol rac. | Bisabolol |

| | | | |
|---|---|---|---|
| A | 2.00 | Abil Care 85 | Bis-PEG/PPG-16/16 PEG/PPG-16/16 |
| | | | Dimethicone, Caprylic/Capric |
| | | | Triglyceride |
| | 4.00 | Finsolv TN | C12-15 Alkyl Benzoate |
| | 1.50 | Miglyol 812 | Caprylic/Capric Triglyceride |
| | 0.50 | Vitamin E acetate | Tocopheryl Acetate |
| | 7.50 | Uvinul MC 80 | Ethylhexyl Methoxycinnamate |
| | 4.00 | Cetiol B | Dibutyl Adipate |
| | 1.00 | Luvitol EHO | Cetearyl Ethylhexanoate |
| | 1.00 | Cremophor CO 40 | PEG-40 Hydrogenated Castor Oil |
| | 1.00 | Paraffin oil, | Mineral Oil |
| | | low viscosity | |
| | 3.00 | Plantacare 2000 | Decyl Glucoside |
| | 1.00 | Uvinul A Plus | Diethylamino Hydroxybenzoyl Hexyl |
| | | | Benzoate |
| | 0.50 | Phenonip | |
| | 2.50 | Uvinul T 150 | Ethylhexyl Triazone |
| | q.s. | Perfume oil | |
| B | 4.00 | Z-COTE MAX | Zinc Oxide (and) Diphenyl Capryl |
| | | | Methicone |
| C | 2.00 | Simulgel NS | Hydroxyethyl Acrylate/Sodium |
| | | | Acryloyldimethyl Taurate |
| | | | Copolymer, Squalane, Polysorbate 60 |
| | 0.10 | Keltrol | Xanthan Gum |
| | 0.10 | Edeta BD | Disodium EDTA |
| | 0.5% | Fusion protein-KBD | |
| | 59.90 | Water demin. | Aqua dem. |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol 331 | Cocoglycerides |
| | 8.00 | Finsolv TN | C12-15 Alkyl Benzoate |
| | 8.00 | Cetiol B | Dibutyl Adipate |
| B | 6.00 | T-Lite SF | Titanium Dioxide, Alumina Hydrate, |
| | | | Dimethicone/Methicone Copolymer |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 2% | Fusion protein-KBD | |
| | 0.30 | Keltrol | Xanthan Gum |
| | 45.10 | Water demin. | Water |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol 331 | Cocoglycerides |
| | 8.00 | Finsolv TN | C12-15 Alkyl Benzoate |
| | 8.00 | Cetiol B | Dibutyl Adipate |
| B | 6.00 | Uvinul N 539 T | Octocrylene |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 0.30 | Carbopol Ultrez 10 P | Carbomer |
| | 2% | Fusion protein-KBD | |
| | 46.10 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 10.00 | Myritol 331 | Cocoglycerides |
| | 8.00 | Finsolv TN | C12-15 Alkyl Benzoate |
| | 8.00 | Cetiol B | Dibutyl Adipate |
| B | 2.00 | Uvinul A Plus | Diethylamino Hydroxybenzoyl |
| | | | Hexyl Benzoate |
| | 5.00 | Z-COTE MAX | Zinc Oxide (and) Diphenyl |
| | | | Capryl Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 5.0% | Fusion protein-KBD | |
| | 0.30 | Carbopol Ultrez 10 P | Carbomer |
| | | add Water demin. | Water |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 3.50 | Cremophor A 6 | Ceteareth-6, Stearyl Alcohol |
| | 1.50 | Cremophor A 25 | Ceteareth-25 |
| | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 | Cyclopentasiloxane, |
| | | | Cyclohexasiloxane Fluid |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| B | 5.00 | T-Lite SF-S Titanium | Titanium Dioxide, Silica |
| | | | Hydrate, Alumina Hydrate, |
| | | | Methicone/Dimethicone Copolymer |
| C | 3.00 | Glycerin 87% | Glycerin |
| | 0.20 | Edeta BD | Disodium EDTA |
| | 0.30 | Keltrol T | Xanthan Gum |
| | 1.00 | Plantacare 2000 | Decyl Glucoside |
| | 1% | Fusion protein-KBD | |
| | 57.30 | Water demin. | Aqua dem. |
| D | 1.00 | Vitamin E acetate | Tocopheryl Acetate |
| | 0.20 | Bisabolol rac. | Bisabolol |

| | | | |
|---|---|---|---|
| A | 10.00 | Uvinul A Plus B | Ethylhexyl Methoxycinnamate, |
| | | | Diethylamino Hydroxybenzoyl |
| | | | Hexyl Benzoate |
| | 10.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 3.00 | Z-COTE MAX | Zinc Oxide (and) Diphenyl |
| | | | Capryl Methicone |
| C | 35.08 | Water demin. | Aqua dem. |
| | 0.38 | Citric acid | Citric Acid |
| | 2.9 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 0.1% | Fusion protein-KBD | |
| | 1.50 | Veegum Ultra | Magnesium Aluminum Silicate |
| D | 0.50 | Phenonip | |
| | 1.00 | Vitamin E acetate | Tocopheryl Acetate |

| | | | |
|---|---|---|---|
| A | 0.70 | Cremophor A 25 | Ceteareth-25 |
| | 1.70 | Cremophor A 6 | Ceteareth-6, Stearyl Alcohol |
| | 2.00 | Uvinul A Plus | Diethylamino Hydroxybenzoyl Hexyl |
| | | | Benzoate |
| | 3.00 | Uvinul N 539 T | Octocrylene |
| | 3.00 | Z-COTE MAX | Zinc Oxide (and) Diphenyl Capryl |
| | | | methicone |
| | 2.00 | Abil B 8843 | PEG-14 Dimethicone |
| | 3.60 | Lanette O | Cetearyl Alcohol |
| | 4.00 | Uvinul MC 80 | Ethylhexyl Methoxycinnamate |
| | 2.00 | Cetiol B | Dibutyl Adipate |
| B | 4.00 | Glycerin 87% | Glycerin |
| | 0.20 | Edeta BD | Disodium EDTA |
| | 71.00 | Water demin. | Panthenol |
| C | 4.00 | Luvigel EM | Caprylic/Capric Triglyceride, Sodium |
| | | | Acrylates Copolymer |
| D | 1.00 | Vitamin E acetate | Tocopheryl Acetate |
| | 1% | Fusion protein-KBD | |
| | 0.20 | Bisabolol rac. | Bisabolol |
| | q.s. | Preservative | |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl Capryl |
| | | | Methicone |
| | 0.03 | Sicomet Blue P 77 | C.I. 77 007, Ultramarines |
| | | 007 | |
| C | 4.00 | 1,2-Propylene | Propylene Glycol |
| | | Glycol Care | |
| | 2.00 | D-Panthenol 50 P | Panthenol, Propylene Glycol |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | 0.5% | Fusion protein-KBD | |
| | 65.27 | Water demin. | Aqua dem. |
| | q.s. | Preservative | |
| | 0.50 | Vitamin E acetate | Tocopheryl Acetate |

| | | | |
|---|---|---|---|
| A | 6.00 | Gilugel SIL 5 | Cyclomethicone (and) Aluminum/ |
| | | | Magnesium Hydroxide Stearate |
| | 5.00 | Uvinul N 539 T | Octocrylene |
| | 2.00 | Mexoryl XL | Drometrizole Trisiloxane |
| | 1.00 | Uvinul T 150 | Ethylhexyl Triazone |
| | 3.00 | T-Lite SF-S | Titanium Dioxide, Silica Hydrate, |
| | | | Alumina Hydrate, Methicone/ |
| | | | Dimethicone Copolymer |
| | 5.00 | Finsolv TN | C12-15 Alkyl Benzoate |
| | 4.00 | Abil WE 09 | Polyglyceryl-4 Isostearate, |
| | | | Cetyl PEG/PPG-10/1 Dimethicone, |
| | | | Hexyl Laurate |
| | 2.00 | Cosmacol EMI | Di-C12-13 Alkyl Malate |
| | 3.00 | Isopro palmitate | Isopropyl Palmitate |
| | 5.00 | Abil B 8839 | Cyclopentasiloxane, Cyclohexasiloxane |
| | 0.50 | Abil 350 | Dimethicone |
| B | 0.50 | Sodium chloride | Sodium Chloride |
| | 0.20 | Edeta BD | Disodium EDTA |
| | 1% | Fusion protein-KBD | |
| | 61.30 | Water demin. | Aqua dem. |
| C | 1.00 | Vitamin E acetate | Tocopheryl Acetate |
| | 0.50 | Phenonip | |

| | | | |
|---|---|---|---|
| A | 1.00 | Abil Care 85 | Bis-PEG/PPG-16/16 PEG/PPG- |
| | | | 16/16 Dimethicone, Caprylic/ |
| | | | Capric Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 5.00 | Uvinul N 539 T | Octocrylene |
| | 10.00 | Witconol APM | PPG-3 Myristyl Ether |
| | 2.00 | Uvinul T 150 | Ethylhexyl Triazone |
| | 1.00 | Dow Corning 345 Fluid | Cyclopentasiloxane, |
| | | | Cyclohexasiloxane |
| | 2.00 | Uvinul A Plus | Diethylamino Hydroxybenzoyl |
| | | | Hexyl Benzoate |
| B | 5.00 | Z-COTE MAX | Zinc Oxide (and) Diphenyl Capryl |
| | | | Silane |
| C | 5.00 | 1,2-Propylene glycol | Propylene Glycol |
| | 0.20 | Keltrol | Xanthan Gum |
| | 0.10 | Edeta BD | Disodium EDTA |
| | 1% | Fusion protein-KBD | |
| | 1.50 | Simulgel 600 | Acrylamide/Sodium |
| | | | Acryloyldimethyltaurate |
| | | | Copolymer, Isohexadecane, |
| | | | Polysorbate 80 |
| | 59.40 | Water demin. | Aqua dem. |
| D | q.s. | Perfume oil | |
| | 0.50 | Glydant | DMDM Hydantoin |

| | | | |
|---|---|---|---|
| A | 7.00 | Uvinul A Plus B | Ethylhexyl Methoxycinnamate, |
| | | | Diethylamino Hydroxybenzoyl |
| | | | Hexyl Benzoate |
| | 1.00 | Tinosorb S | Bis-Ethylhexyloxyphenol |
| | | | Methoxyphenyl Triazine |
| | 1.00 | Uvinul T 150 | Ethylhexyl Triazone |
| | 7.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 5.00 | Z-COTE MAX | Zinc Oxide (and) Diphenyl |
| | | | Capryl Methicone |
| C | 35.35 | Water demin. | Aqua dem. |
| | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 0.1% | Fusion protein-KBD | |
| | 1.50 | Veegum Ultra | Magnesium Aluminum Silicate |
| D | 0.50 | Phenonip | |
| | 1.00 | Vitamin E acetate | Tocopheryl Acetate |

| | | | |
|---|---|---|---|
| A | 7.00 | Uvinul A Plus B | Ethyhexyl Methoxycinnamate, |
| | | | Diethylamino Hydroxybenzoyl |
| | | | Hexyl Benzoate |
| | 1.00 | Tinosorb S | Bis-Ethylhexyloxyphenol |
| | | | Methoxyphenyl Triazine |
| | 1.00 | Uvinul T 150 | Ethylhexyl Triazone |
| | 7.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 5.00 | T-Lite SF | Titanium Dioxide, Alumina Hydrate, |
| | | | Dimethicone/Methicone Copolymer |
| C | 30.45 | Water demin. | Aqua dem. |
| | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 5% | Fusion protein-KBD | |
| | 1.50 | Veegum Ultra | Magnesium Aluminum Silicate |
| D | 0.50 | Phenonip | |
| | 1.00 | Vitamin E acetate | Tocopheryl Acetate |

| | | |
|---|---|---|
| 5.00 | Z-COTE MAX | Zinc Oxide (and) Diphenyl Capryl |
| | | Methicone |
| 2.00 | Uvinul A Plus | Diethylamino Hydroxybenzoyl Hexyl |
| | | Benzoate |
| 2.00 | Mexoryl XL | Drometrizole Trisiloxane |
| 3.00 | Uvinul MC 80 | Ethylhexyl Methoxycinnamate |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 | Candelilla (Euphorbia Cerifera) Wax |
| | LJ | |
| 1.80 | Beeswax 3050 PH | Beeswax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E acetate | Tocopheryl Acetate |
| 1% | Fusion protein-KBD | |
| 0.42 | D,L-Alpha-Tocopherol | Tocopherol |
| 40.38 | castor oil | Castor (Ricinus Communis) Oil |

| | | | |
|---|---|---|---|
| A | 1.00 | Abil Care 85 | Bis-PEG/PPG-16/16 PEG/PPG- |
| | | | 16/16 Dimethicone, Caprylic/ |
| | | | Capric Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning | Cyclopentasiloxane, |
| | | 345 Fluid | Cyclohexasiloxane |
| | 5.00 | Uvinul N 539 T | Octocrylene |
| B | 3.00 | T-Lite SF-S | Titanium Dioxide, Silica Hydrate, |
| | | | Alumina Hydrate, Methicone/ |
| | | | Dimethicone Copolymer |
| C | 5.00 | 1,2-Propylene glycol | Propylene Glycol |
| | 1.00 | Mexoryl SX | Terephthalidene Dicamphor |
| | | | Sulfanic Acid |
| | 0.20 | Keltrol | Xanthan Gum |
| | 0.10 | Edeta BD | Disodium EDTA |
| | 1% | Fusion protein-KBD | |
| | 1.50 | Simulgel 600 | Acrylamide/Sodium |
| | | | Acryloyldimethyltaurate Copolymer, |
| | | | Isohexadecane, Polysorbate 80 |
| | 69.50 | Water demin. | Aqua dem. |
| D | q.s. | Perfume oil | |
| | 0.50 | Glydant | DMDM Hydantoin |

| | | | |
|---|---|---|---|
| A | 1.00 | Abil Care 85 | Bis-PEG/PPG-16/16 PEG/PPG- |
| | | | 16/16 Dimethicone, Caprylic/ |
| | | | Capric Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Parsol 1789 | Butyl Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning | Cyclopentasiloxane, |
| | | 345 Fluid | Cyclohexasiloxane |
| | 5.00 | Uvinul N 539 T | Octocrylene |
| B | 3.00 | T-Lite SF-S | Titanium Dioxide, Silica Hydrate, |
| | | | Alumina Hydrate, Methicone/ |
| | | | Dimethicone Copolymer |
| C | 5.00 | 1,2-Propylene | Propylene Glycol |
| | | glycol | |
| | 1.00 | Mexoryl SX | Terephthalidene Dicamphor |
| | | | Sulfanic Acid |
| | 0.20 | Keltrol | Xanthan Gum |
| | 0.10 | Edeta BD | Disodium EDTA |
| | 0.2% | Fusion protein-KBD | |
| | 1.50 | Simulgel 600 | Acrylamide/Sodium |
| | | | Acryloyldimethyltaurate Copolymer, |
| | | | Isohexadecane, Polysorbate 80 |
| | 68.30 | Water demin. | Aqua dem. |
| D | q.s. | Perfume oil | |
| | 0.50 | Glydant | DMDM Hydantoin |

| A | 0.70 Cremophor A 25 | Ceteareth-25 |
|---|---|---|
| | 1.70 Cremophor A 6 | Ceteareth-6, Stearyl Alcohol |
| | 2.00 Parsol 1789 | Butyl Methoxydibenzoylmethane |
| | 5.00 Uvinul N 539 T | Octocrylene |
| | 4.00 Z-COTE MAX | Zinc Oxide (and) Diphenyl Capryl |
| | | methicone |
| | 2.00 Abil B 8843 | PEG-14 Dimethicone |
| | 3.60 Lanette O | Cetearyl Alcohol |
| | 1.00 Uvinul T150 | Ethylhexyl Triazone |
| | 2.00 Cetiol B | Dibutyl Adipate |
| B | 4.00 Glycerin 87% | Glycerin |
| | 0.20 Edeta BD | Disodium EDTA |
| | 71.00 Water demin. | Panthenol |
| C | 4.00 Luvigel EM | Caprylic/Capric Triglyceride, |
| | | Sodium Acrylates Copolymer |
| 1% Fusion protein-KBD | | |
| D | 1.00 Vitamin E acetate | Tocopheryl Acetate |
| | 0.20 Bisabolol rac. | Bisabolol |
| | q.s. Preservative | |

| A | 1.00 Abil Care 85 | Bis-PEG/PPG-16/16 PEG/PPG-16/16 |
|---|---|---|
| | | Dimethicone, Caprylic/Capric |
| | | Triglyceride |
| | 3.00 Cremophor CO 40 | PEG-40 Hydrogenated Castor Oil |
| | 0.30 Cremophor WO 7 | PEG-7 Hydrogenated Castor Oil |
| | 2.00 Parsol 1789 | Butyl Methoxydibenzoylmethane |
| | 2.00 Mexoryl XL | Drometrizole Trisiloxane |
| | 10.00 Witconol APM | PPG-3 Myristyl Ether |
| | 1.00 Uvinul T 150 | Ethylhexyl Triazone |
| | 1.00 Dow Corning 345 | Cyclopentasiloxane, |
| | Fluid | Cyclohexasiloxane |
| | 5.00 Uvinul N 539 T | Octocrylene |
| B | 3.00 Z-COTE MAX | Zinc Oxide (and) Diphenyl Capryl |
| | | Methicone |
| C | 5.00 1,2-Propylene | Propylene Glycol |
| | glycol | |
| | 1.00 Mexoryl SX | Terephthalidene Dicamphor |
| | | Sulfanic Acid |
| | 0.20 Keltrol | Xanthan Gum |
| | 0.10 Edeta BD | Disodium EDTA |
| | 1.50 Simulgel 600 | Acrylamide/Sodium |
| | | Acryloyldimethyltaurate Copolymer, |
| | | Isohexadecane, Polysorbate 80 |
| 0.5% Fusion protein-KBD | | |
| | 68.00 Water demin. | Aqua dem. |
| D | q.s. Perfume oil | |
| | 0.50 Glydant | DMDM Hydantoin |

| A | 2.00 Uvinul A Plus | Diethylamino Hydroxybenzoyl Hexyl |
|---|---|---|
| | | Benzoate |
| | 3.00 Uvinul N 539 T | Octocrylene |
| | 3.00 Emulgade PL 68/50 | Cetearyl Glucoside, Cetearyl Alcohol |
| | 2.00 Dracorin 100 SE | Glyceryl Stearate, PEG-100 Stearate |
| | 1.00 Fitoderm | Squalane |
| | 0.5 Cremophor WO 7 | PEG-7 Hydrogenated Castor Oil |
| | 0.5 Cremophor PS 20 | Polysorbate 20 |
| | 2.00 Dry Flo Pure | Aluminum Starch Octenylsuccinate |
| B | 5.00 Z-COTE MAX | Zinc Oxide (and) Diphenyl Capryl |
| | | methicone |
| C | 4.00 1,2-Propylene | Propylene Glycol |
| | glycol Care | |
| | 0.20 Keltrol | Xanthan Gum |
| | 0.50 Simulgel 600 | Acrylamide/Sodium |
| | | Acryloyldimethyltaurate Copolymer, |
| | | Isohexadecane, Polysorbate 80 |
| | 65.80 Water demin. | Aqua dem. |
| D | 0.50 Euxyl K 300 | |
| | 0.50 Vitamin E acetate | Tocopheryl Acetate |
| 1% Fusion protein-KBD | | |
| | 1.00 RetiSTAR | Caprylic/Capric Triglyceride, Sodium |
| | | Ascorbate, Tocopherol, Retinol |

Dermocosmetic preparations according to the invention comprising the keratin-binding effector molecule C16-KBD-D (according to SEQ ID No.: 212 in pRee024 (FIG. 8)) prepared analogously to Example 3 are described below. The C16-KBD-D is referred to in the examples below as fusion protein-KDB by way of representation for all of the other KBD fusion proteins described above. It will be appreciated by the person skilled in the art that all of the other specified KBD fusion proteins can also be prepared using the corresponding KBD fusion protein constructs according to Example 3, and be used in the preparations specified below.

### Example 55

### Use of the KBD in an Emulsion for Daycare—O/W Type

| | |
|---|---|
| % | Ingredient (INCI) |

Al 1%:

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 67.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 1.0 | Aqueous solution with about 5% fusion protein-KBD |
| E | q.s. | Sodium Hydroxide |

Al 5%:

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 63.8 | Aqua dem. |
| C | 4.0 | Caprylic/Capric Triglyceride, Sodium Acrylates Copolymer |
| D | 0.2 | Sodium Ascorbyl Phosphate |
| | 1.0 | Tocopheryl Acetate |
| | 0.2 | Bisabolol |
| | 1.0 | Caprylic/Capric Triglyceride, Sodium Ascorbate, Tocopherol, |
| | | Retinol |
| | 5.0 | Aqueous solution with about 5% fusion protein-KBD |
| E | q.s. | Sodium Hydroxide |

Preparation: Heat phases A and B separately from one another to about 80° C.. Stir Phase B into phase A and homogenize. Stir phase C into the combined phases A and B and homogenize again. Cool with stirring to about 40° C., add phase D, adjust the pH to about 6.5 using phase E, homogenize and cool to room temperature with stirring.
Note:
The formulation is prepared without protective gas. Bottling must take place into oxygen-impermeable packagings, e.g. aluminum tubes.

### Example 56

### Use of the KBD in a Protective Day Cream—O/W Type

| | |
|---|---|
| % | Ingredient (INCI) |

Al 1%:

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 68.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 1.0 | Aqueous solution with about 5% fusion protein-KBD |
| E | q.s. | Sodium Hydroxide |

Al 5%:

| | | |
|---|---|---|
| A | 1.7 | Ceteareth-6, Stearyl Alcohol |
| | 0.7 | Ceteareth-25 |
| | 2.0 | Diethylamino Hydroxybenzoyl Hexyl Benzoate |
| | 2.0 | PEG-14 Dimethicone |
| | 3.6 | Cetearyl Alcohol |
| | 6.0 | Ethylhexyl Methoxycinnamate |
| | 2.0 | Dibutyl Adipate |
| B | 5.0 | Glycerin |
| | 0.2 | Disodium EDTA |
| | 1.0 | Panthenol |
| | q.s. | Preservative |
| | 64.6 | Aqua dem. |
| C | 4.0 | Caprylic/Capric Triglyceride, Sodium Acrylates Copolymer |
| D | 1.0 | Sodium Ascorbyl Phosphate |
| | 1.0 | Tocopheryl Acetate |
| | 0.2 | Bisabolol |
| | 5.0 | Aqueous solution with about 5% fusion protein-KBD |
| E | q.s. | Sodium Hydroxide |

Preparation: Heat phases A and B separately from one another to about 80° C. Stir phase B into phase A and homogenize. Incorporate phase C into the combined phases A and B and homogenize. Cool with stirring to about 40° C. Add phase D, adjust the pH to about 6.5 using phase E and homogenize. Cool to room temperature with stirring.

### Example 57

### Use of the KBD in a Face-Cleansing Lotion—O/W Type

| | |
|---|---|
| % | Ingredient (INCI) |

Al 1%:

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasilosane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 1.0 | Aqueous solution with about 5% fusion protein-KBD |
| | 60.7 | Aqua dem. |

Al 5%:

| | | |
|---|---|---|
| A | 10.0 | Cetearyl Ethylhexanoate |
| | 10.0 | Caprylic/Capric Triglyceride |
| | 1.5 | Cyclopentasiloxane, Cyclohexasiloxane |
| | 2.0 | PEG-40 Hydrogenated Castor Oil |
| B | 3.5 | Caprylic/Capric Triglyceride, Sodium Acrylates Copolymer |
| C | 1.0 | Tocopheryl Acetate |
| | 0.2 | Bisabolol |
| | q.s. | Preservative |
| | q.s. | Perfume oil |
| D | 3.0 | Polyquaternium-44 |
| | 0.5 | Cocotrimonium Methosulfate |
| | 0.5 | Ceteareth-25 |
| | 2.0 | Panthenol, Propylene Glycol |
| | 4.0 | Propylene Glycol |
| | 0.1 | Disodium EDTA |
| | 5.0 | Aqueous solution with about 5% fusion protein-KBD |
| | 56.7 | Aqua dem. |

Preparation: Dissolve phase A, Stir phase B into phase A. Incorporate phase C into the combined phases A and B. Dissolve phase D, stir into the combined phases A, B and C and homogenize. After-stir for 15 min.

### Example 58

### Use of the KBD in a Daily Care Body Spray

| | |
|---|---|
| % | Ingredient (INCI) |

Al 1%:

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 1.0 | Aqueous solution with about 5% fusion protein-KBD |
| | 59.2 | Alcohol |

Al 5%:

| | | |
|---|---|---|
| A | 3.0 | Ethylhexyl Methoxycinnamate |
| | 2.0 | Diethylamino Hydroxybenzoyl Hexyl Benzoate |
| | 1.0 | Polyquaternium-44 |
| | 3.0 | Propylene Glycol |
| | 2.0 | Panthenol, Propylene Glycol |
| | 1.0 | Cyclopentasiloxane, Cyclohexasiloxane |
| | 10.0 | Octyldodecanol |
| | 0.5 | PVP |
| | 10.0 | Caprylic/Capric Triglyceride |
| | 3.0 | C12-15 Alkyl Benzoate |
| | 3.0 | Glycerin |
| | 1.0 | Tocopheryl Acetate |
| | 0.3 | Bisabolol |
| | 5.0 | Aqueous solution with about 5% fusion protein-KBD |
| | 55.2 | Alcohol |

Preparation: Weigh in the components of phase A and dissolve until clear.

### Example 59

### Use of the KBD in a Skin Care Gel

| | |
|---|---|
| % | Ingredient (INCI) |

Al 1%:

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 1.0 | Aqueous solution with about 5% fusion protein-KBD |
| | 75.4 | Aqua dem. |
| C | 0.8 | Triethanolamine |

Al 5%:

| | | |
|---|---|---|
| A | 3.6 | PEG-40 Hydrogenated Castor Oil |
| | 15.0 | Alcohol |
| | 0.1 | Bisabolol |
| | 0.5 | Tocopheryl Acetate |
| | q.s. | Perfume oil |
| B | 3.0 | Panthenol |
| | 0.6 | Carbomer |
| | 5.0 | Aqueous solution with about 5% fusion protein-KBD |
| | 71.4 | Aqua dem. |
| C | 0.8 | Triethanolamine |

Preparation: Dissolve phase A until clear. Allow phase B to swell and neutralize with phase C. Stir phase A into the homogenized phase B and homogenize.

### Example 60

### Use of the KBD in an Aftershave Lotion

| | |
|---|---|
| % | Ingredient (INCI) |

Al 1%:

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 1.0 | Aqueous solution with about 5% fusion protein-KBD |
| | 0.1 | Triethanolamine |
| | 63.5 | Aqua dem. |

Al 5%:

| | | |
|---|---|---|
| A | 10.0 | Cetearyl Ethylhexanoate |
| | 5.0 | Tocopheryl Acetate |
| | 1.0 | Bisabolol |
| | 0.1 | Perfume oil |
| | 0.3 | Acrylates/C10-30 Alkyl Acrylate Crosspolymer |
| B | 15.0 | Alcohol |
| | 1.0 | Panthenol |
| | 3.0 | Glycerin |
| | 5.0 | Aqueous solution with about 5% fusion protein-KBD |
| | 0.1 | Triethanolamine |
| | 59.5 | Aqua dem. |

Preparation: Mix the components of phase A. Dissolve phase B, incorporate into phase A and homogenize.

### Example 61

### Use of the KBD in an Aftersun Lotion

| | |
|---|---|
| % | Ingredient (INCI) |

Al 1%:

| | | |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 1.0 | Aqueous solution with about 5% fusion protein-KBD |
| | 63.2 | Aqua dem. |
| C | 0.2 | Triethanolamine |

Al 5%:

| | | |
|---|---|---|
| A | 0.4 | Acrylates/C10-30 Alkyl Acrylate Crosspolymer |
| | 15.0 | Cetearyl Ethylhexanoate |
| | 0.2 | Bisabolol |
| | 1.0 | Tocopheryl Acetate |
| | q.s. | Perfume oil |
| B | 1.0 | Panthenol |
| | 15.0 | Alcohol |
| | 3.0 | Glycerin |
| | 5.0 | Aqueous solution with about 5% fusion protein-KBD |
| | 59.2 | Aqua dem. |
| C | 0.2 | Triethanolamine |

Preparation: Mix the components of phase A. Stir phase B into phase A with homogenization. Neutralize with phase C and homogenize again.

### Example 62

### Use of the KBD in a Sunscreen Lotion

| | |
|---|---|
| % | Ingredient (INCI) |

Al 1%:

| | | |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 59.7 | Aqua dem. |
| D | 1.0 | Aqueous solution with about 5% fusion protein-KBD |
| | 1.0 | Phenoxyethanol, Methylparaben, Ethylparaben, Butylparaben, |
| | | Propyl-paraben, Isobutylparaben |
| | 0.3 | Bisabolol |

Al 5%:

| | | |
|---|---|---|
| A | 4.5 | Ethylhexyl Methoxycinnamate |
| | 2.0 | Diethylamino Hydroxybenzoyl Hexyl Benzoate |
| | 3.0 | Octocrylene |
| | 2.5 | Di-C12-13 Alkyl Malate |
| | 0.5 | Tocopheryl Acetate |
| | 4.0 | Polyglyceryl-3 Methyl Glucose Distearate |
| B | 3.5 | Cetearyl Isononanoate |
| | 1.0 | VP/Eicosene Copolymer |
| | 5.0 | Isohexadecane |
| | 2.5 | Di-C12-13 Alkyl Malate |
| | 3.0 | Titanium Dioxide, Trimethoxycaprylylsilane |
| C | 5.0 | Glycerin |
| | 1.0 | Sodium Cetearyl Sulfate |
| | 0.5 | Xanthan Gum |
| | 55.7 | Aqua dem. |
| D | 5.0 | Aqueous solution with about 5% fusion protein-KBD |
| | 1.0 | Phenoxyethanol, Methylparaben, Ethylparaben, Butylparaben, |
| | | Propyl-paraben, Isobutylparaben |
| | 0.3 | Bisabolol |

Preparation: Heat the components of phases A and B separately from one another to about 80° C.. Stir phase B into phase A and homogenize. Heat phase C to about 80° C. and stir into the combined phases A and B with homogenization. Cool to about 40° C. with stirring, add phase D and homogenize again.

### Example 63

### Use of the KBD in a Sunscreen Lotion—O/W Type

| | |
|---|---|
| % | Ingredient (INCI) |

Al 1%:

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl |
| | | Taurate Copolymer, Squalane, Polysorbate 60 |
| | 0.2 | Disodium EDTA |
| | 5.0 | Propylene Glycol |
| | 0.5 | Panthenol |
| | 60.9 | Aqua dem. |
| D | 1.0 | Aqueous solution with about 5% fusion protein-KBD |
| | 0.5 | Phenoxyethanol, Methylparaben, Butylparaben, |
| | | Ethylparaben, Propyl-paraben, Isopropylparaben |
| | 1.0 | Tocopheryl Acetate |
| | 0.2 | Bisabolol |

Al 5%:

| | | |
|---|---|---|
| A | 2.0 | Ceteareth-6, Stearyl Alcohol |
| | 2.0 | Ceteareth-25 |
| | 3.0 | Tribehenin |
| | 2.0 | Cetearyl Alcohol |
| | 2.0 | Cetearyl Ethylhexanoate |
| | 5.0 | Ethylhexyl Methoxycinnamate |
| | 1.0 | Ethylhexyl Triazone |
| | 1.0 | VP/Eicosene Copolymer |
| | 7.0 | Isopropyl Myristate |
| B | 5.0 | Zinc Oxide, Triethoxycaprylylsilane |
| C | 0.2 | Xanthan Gum |
| | 0.5 | Hydroxyethyl Acrylate/Sodium Acryloyldimethyl |
| | | Taurate Copolymer, Squalane, Polysorbate 60 |
| | 0.2 | Disodium EDTA |
| | 5.0 | Propylene Glycol |
| | 0.5 | Panthenol |
| | 56.9 | Aqua dem. |
| D | 5.0 | Aqueous solution with about 5% fusion protein-KBD |
| | 0.5 | Phenoxyethanol, Methylparaben, Butylparaben, |
| | | Ethylparaben, Propylparaben, Isopropylparaben |
| | 1.0 | Tocopheryl Acetate |
| | 0.2 | Bisabolol |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 64

### Use of the KBD in a Sunscreen Lotion—O/W Type

| | |
|---|---|
| % | Ingredient (INCI) |

Al 1%:

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 56.3 | Aqua dem. |
| D | 1.0 | Aqueous solution with about 5% fusion protein-KBD |
| | 1.0 | Tocopheryl Acetate |
| | 0.2 | Bisabolol |
| | q.s. | Perfume oil |
| | q.s. | Preservative |

Al 5%:

| | | |
|---|---|---|
| A | 3.5 | Ceteareth-6, Stearyl Alcohol |
| | 1.5 | Ceteareth-25 |
| | 7.5 | Ethylhexyl Methoxycinnamate |
| | 2.0 | Diethylamino Hydroxybenzoyl Hexyl Benzoate |
| | 2.0 | Cyclopentasiloxane, Cyclohexasiloxane |
| | 0.5 | Beeswax |
| | 3.0 | Cetearyl Alcohol |
| | 10.0 | Caprylic/Capric Triglyceride |
| B | 5.0 | Titanium Dioxide, Silica, Methicone, Alumina |
| C | 3.0 | Glycerin |
| | 0.2 | Disodium EDTA |
| | 0.3 | Xanthan Gum |
| | 1.0 | Decyl Glucoside |
| | 2.0 | Panthenol, Propylene Glycol |
| | 52.3 | Aqua dem. |
| D | 5.0 | Aqueous solution with about 5% fusion protein-KBD |
| | 1.0 | Tocopheryl Acetate |
| | 0.2 | Bisabolol |
| | q.s. | Perfume oil |
| | q.s. | Preservative |

Preparation: Heat phase A to about 80° C., stir in phase B and homogenize for 3 min. Likewise heat phase C to 80° C. and stir into the combined phases A and B with homogenization. Cool to about 40° C., stir in phase D and homogenize again.

### Example 65

### Use of the KBD in a Foot Balsam

| | |
|---|---|
| % | Ingredient (INCI) |

Al 1%:

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 69.3 | Aqua dem. |
| | q.s. | Preservative |
| C | 1.0 | Bisabolol |
| | 1.0 | Tocopheryl Acetate |
| D | 1.0 | Aqueous solution with about 5% fusion protein-KBD |
| | 5.0 | Witch Hazel Extract |

Al 5%:

| | | |
|---|---|---|
| A | 2.0 | Ceteareth-6, Stearyl Alcohol |
| | 2.0 | Ceteareth-25 |
| | 5.0 | Cetearyl Ethylhexanoate |
| | 4.0 | Cetyl Alcohol |
| | 4.0 | Glyceryl Stearate |
| | 5.0 | Mineral Oil |
| | 0.2 | Menthol |
| | 0.5 | Camphor |
| B | 65.3 | Aqua dem. |
| | q.s. | Preservative |
| C | 1.0 | Bisabolol |
| | 1.0 | Tocopheryl Acetate |
| D | 5.0 | Aqueous solution with about 5% fusion protein-KBD |
| | 5.0 | Witch Hazel Extract |

Preparation: Heat the components of phases A and B separately from one another to about 80° C.. Stir phase B into phase A with homogenization. Cool to about 40° C. with stirring, add phases C and D and briefly after-homogenize. Cool to room temperature with stirring.

### Example 66

### Use of the KBD in a W/O Emulsion with Bisabolol

| | |
|---|---|
| % | Ingredient (INCI) |

Al 1%:

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 55.6 | Aqua dem. |
| C | 1.0 | Aqueous solution with about 5% fusion protein-KBD |
| | 0.5 | Tocopheryl Acetate |
| | 0.6 | Bisabolol |

Al 5%:

| | | |
|---|---|---|
| A | 6.0 | PEG-7 Hydrogenated Castor Oil |
| | 8.0 | Cetearyl Ethylhexanoate |
| | 5.0 | Isopropyl Myristate |
| | 15.0 | Mineral Oil |
| | 0.3 | Magnesium Stearate |
| | 0.3 | Aluminum Stearate |
| | 2.0 | PEG-45/Dodecyl Glycol Copolymer |
| B | 5.0 | Glycerin |
| | 0.7 | Magnesium Sulfate |
| | 51.6 | Aqua dem. |
| C | 5.0 | Aqueous solution with about 5% fusion protein-KBD |
| | 0.5 | Tocopheryl Acetate |

Preparation: Heat phases A and B separately from one another to about 85° C.. Stir phase B into phase A and homogenize. Cool to about 40° C. with stirring, add phase C and briefly homogenize again. Cool to room temperature with stirring.
List of formulations for patent keratin-binding domain - hair care

### Example 67

### Shower Gel

| | |
|---|---|
| % | Ingredient (INCI) |

Al 1%

| | | |
|---|---|---|
| A | 40.0 | Sodium Laureth Sulfate |
| | 5.0 | Decyl Glucoside |
| | 5.0 | Cocamidopropyl Betaine |
| | 1.0 | Aqueous solution with about 5% fusion protein-KBD |
| | 1.0 | Panthenol |
| | q.s. | Perfume oil |
| | q.s. | Preservative |
| | 2.0 | Sodium Chloride |
| | 46.0 | Aqua dem. |
| B | q.s. | Citric Acid |

Al 5%

| | | |
|---|---|---|
| A | 40.0 | Sodium Laureth Sulfate |
| | 5.0 | Decyl Glucoside |
| | 5.0 | Cocamidopropyl Betaine |
| | 5.0 | Aqueous solution with about 5% fusion protein-KBD |
| | 1.0 | Panthenol |
| | q.s. | Perfume oil |
| | q.s. | Preservative |
| | 2.0 | Sodium Chloride |
| | 42.0 | Aqua dem. |
| B | q.s. | Citric Acid |

Preparation: Mix the components of phase A and dissolve. Adjust the pH to 6-7 with citric acid.

### Example 68

### Moisturizing Body Care Cream

| | |
|---|---|
| % | Ingredient (INCI) |

Al 1%

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 1.0 | Aqueous solution with about 5% fusion protein-KBD |
| | 1.0 | Panthenol |
| | 0.5 | Magnesium Aluminum Silicate |
| | q.s | Preservative |
| | 65.5 | Aqua dem. |
| C | q.s. | Perfume oil |
| D | q.s. | Citric Acid |

Al 5%

| | | |
|---|---|---|
| A | 2.0 | Ceteareth-25 |
| | 2.0 | Ceteareth-6, Stearyl Alcohol |
| | 3.0 | Cetearyl Ethylhexanoate |
| | 1.0 | Dimethicone |
| | 4.0 | Cetearyl Alcohol |
| | 3.0 | Glyceryl Stearate SE |
| | 5.0 | Mineral Oil |
| | 4.0 | Simmondsia Chinensis (Jojoba) Seed Oil |
| | 3.0 | Mineral Oil, Lanolin Alcohol |
| B | 5.0 | Propylene Glycol |
| | 5.0 | Aqueous solution with about 5% fusion protein-KBD |
| | 1.0 | Panthenol |
| | 0.5 | Magnesium Aluminum Silicate |
| | q.s | Preservative |
| | 61.5 | Aqua dem. |
| C | q.s. | Perfume oil |
| D | q.s. | Citric Acid |

Preparation: Heat phases A and B separately to about 80° C.. Briefly prehomogenize phase B, then stir phase B into phase A and homogenize again. Cool to about 40° C., add phase C and homogenize thoroughly again. Adjust the pH to 6-7 with citric acid.

### Example 69

### Moisturizing Body Care Cream

| | |
|---|---|
| % | Ingredient (INCI) |

Al 1%

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 62.9 | Aqua dem. |
| C | q.s. | Perfume oil |
| | 1.0 | Aqueous solution with about 5% fusion protein-KBD |

Al 5%

| | | |
|---|---|---|
| A | 6.0 | PEG-7 Hydrogenated Castor Oil |
| | 10.0 | Cetearyl Ethylhexanoate |
| | 5.0 | Isopropyl Myristate |
| | 7.0 | Mineral Oil |
| | 0.5 | Shea Butter (Butyrospermum Parkii) |
| | 0.5 | Aluminum Stearate |
| | 0.5 | Magnesium Stearate |
| | 0.2 | Bisabolol |
| | 0.7 | Quaternium-18-Hectorite |
| B | 5.0 | Dipropylene Glycol |
| | 0.7 | Magnesium Sulfate |
| | q.s. | Preservative |
| | 58.9 | Aqua dem. |
| C | q.s. | Perfume oil |
| | 5.0 | Aqueous solution with about 5% fusion protein-KBD |

Preparation: Heat phases A and B separately to about 80° C. Stir phase B into phase A and homogenize. Cool to about 40° C. with stirring, add phase C and homogenize again. Allow to cool to room temperature with stirring.

### Example 70

### Liquid Make-Up—O/W Type

| | |
|---|---|
| % | Ingredient (INCI) |

Al 1%

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 61.9 | Aqua dem. |
| C | 0.1 | Bisabolol |
| | 1.0 | Aqueous solution with about 5% fusion protein-KBD |
| | q.s. | Perfume oil |
| D | 5.7 | C.I. 77 891, Titanium Dioxide |
| | 1.1 | Iron Oxides |

Al 5%

| | | |
|---|---|---|
| A | 2.0 | Ceteareth-6, Stearyl Alcohol |
| | 2.0 | Ceteareth-25 |
| | 6.0 | Glyceryl Stearate |
| | 1.0 | Cetyl Alcohol |
| | 8.0 | Mineral Oil |
| | 7.0 | Cetearyl Ethylhexanoate |
| | 0.2 | Dimethicone |
| B | 3.0 | Propylene Glycol |
| | 1.0 | Panthenol |
| | q.s. | Preservative |
| | 57.9 | Aqua dem. |
| C | 0.1 | Bisabolol |
| | 5.0 | Aqueous solution with about 5% fusion protein-KBD |
| | q.s. | Perfume oil |
| D | 57 | C.I. 77 891, Titanium Dioxide |
| | 1.1 | Iron Oxides |

Preparation: Heat phases A and B separately to about 80° C.. Stir phase B into phase A and homogenize. Cool to about 40° C. with stirring, add phases C and D and thoroughly homogenize again. Allow to cool to room temperature with stirring.

### Example 71

Dermocosmetic preparations according to the invention are described below, comprising the keratin-binding effector molecule C16-KBD-D (according to SEQ ID No.: 212 in pRee024 (FIG. 8)) prepared according to Example 3. The specified keratin-binding fusion protein is used as about 5% strength by weight aqueous solution. The following data are parts by weight.

Gel cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Uvinul ® A Plus ™ | 2.00 | 1.50 | 0.75 | 1.00 |
| UvaSorb ® k2A | | 3.00 | | |
| Ethylhexyl Bis- | | | | |
| Isopentylbenzoxazolylphenyl | | | | |
| Melamine | | | | |
| Uvinul ® MC 80 | 3.00 | | 1.00 | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Uvinul ® T 150 | 4.00 | | 3.00 | 4.00 |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutyl phenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | 3.50 | 4.00 | 7.00 | 9.00 |
| Titanium Dioxide-microfine | 1.00 | | 3.00 | |
| Zinc Oxide-microfine | | | | 0.25 |
| Fusion protein-KBD | 2.0 | 0.5 | 1.0 | 10.0 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | ad 100 | ad 100 | ad 100 | ad 100 |
| adjust pH to 6.0 | | | | |

OW sunscreen formulation

| | 1 | 2 | 3 | 4 | 5 | 6 | 7 |
|---|---|---|---|---|---|---|---|
| Glyceryl Stearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Sodium Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Fusion protein-KBD | 10.0 | 0.5 | 3.0 | 5.0 | 0.1 | 0.02 | 7.5 |
| Uvinul ® A Plus ™ | 2.00 | 1.50 | 0.75 | 1.00 | 2.10 | 4.50 | 5.00 |
| UvaSorb ® k2A | | | | | | | |
| Ethylhexyl Bis- | | | | | | | |
| Isopentylbenzoxazolylphenyl | | | | | | | |
| Melamine | | | | | | | |
| Ethylhexyl Methoxycinnamate | | | | | 5.00 | 6.00 | 8.00 |
| Uvinul ® MC 80 | | | | | | | |
| Bis-Ethylhexyloxyphenol | | 1.50 | | 2.00 | 2.50 | | 2.50 |
| Methoxyphenyl Triazine | | | | | | | |
| Butyl Methoxydibenzoylmethane | | | 2.00 | | 2.00 | 1.50 | |
| Disodium Phenyl Dibenzimidazole | 2.50 | | 0.50 | 2.00 | | 0.30 | |
| Tetrasulfonate | | | | | | | |
| Ethyhexyl Triazone Uvinul ® T 150 | 4.00 | | 3.00 | 4.00 | | 2.00 | |
| Octocrylene | | 4.00 | | | | | 7.50 |
| Diethylhexyl Butamido Triazone | 1.00 | | | 2.00 | 1.00 | | 1.00 |
| Phenylbenzimidazole Sulfonic Acid | 0.50 | | 3.00 | | | | |
| Methylene Bis-Benzotriazolyl | 2.00 | | 0.50 | 1.50 | 2.50 | | |
| Tetramethylbutylphenol | | | | | | | |
| Ethylhexyl Salicylate | | | 3.00 | | | | 5.00 |
| Drometrizole Trisiloxane | | | 0.50 | | | 1.00 | |
| Terephthalidene Dicamphor | | 1.50 | | 1.00 | 1.00 | | 0.50 |
| Sulfonic Acid | | | | | | | |
| Diethylhexyl 2,6-Naphthalate | 3.50 | | 7.00 | | 6.00 | 9.00 | |
| Titanium Dioxide-microfine | 1.00 | | 3.00 | | 3.50 | | 1.50 |
| Zinc Oxide-microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglyceride | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soya (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium Iminodisuccinate | | | | 0.25 | 1.00 | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

Hydrodispersion

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Sodium Carbomer | | 0.20 | | 0.30 | |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Fusion protein-KBD | 5.0 | 0.5 | 3.0 | 0.1 | 10.0 |
| Uvinul ® A Plus ™ | 2.00 | 1.50 | 0.75 | 1.00 | 2.10 |
| UvaSorb ® k2A Ethylhexyl Bis- | | 3.50 | | | |
| Isopentylbenzoxazolylphenyl Melamine | | | | | |
| Ethylhexyl Methoxycinnamate Uvinul ® MC | | | | | 5.00 |
| 80 | | | | | |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethyhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 9.00 |
| Titanium Dioxide-microfine | 1.00 | | 3.00 | | 3.50 |
| Zinc Oxide-microfine | | | | 0.25 | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya (Soybean) Oil | | | 1.50 | | 1.00 |
| Vitamin E Acetate | 0.50 | | 0.25 | | 1.00 |
| Glucosylrutin | 0.60 | | | 0.25 | |
| Biosaccaride Gum-1 | | 2.50 | 0.50 | | 2.00 |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | ad 100 | ad 100 | ad 100 | ad 100 | ad 100 |

WO sunscreen emulsion

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30 Dipolyhydroxystearate | | | 5.00 | | |
| Fusion protein-KBD | 5.0 | 1.0 | 10.0 | 0.5 | 0.1 |
| Uvinul ® A Plus ™ | 2.00 | 1.50 | 0.75 | 1.00 | 2.10 |
| UvaSorb ® k2A | | 2.00 | | | |
| Ethylhexyl Bis-Isopentylbenzoxazolylphenyl | | | | | |
| Melamine | | | | | |
| Ethylhexyl Methoxycinnamate Uvinul ® MC | | | | | 5.00 |
| 80 | | | | | |
| Bis-Ethylhexyloxyphenol Methoxyphenyl | | 1.50 | | 2.00 | 2.50 |
| Triazine | | | | | |
| Butyl Methoxydibenzoylmethane | | | 2.00 | | 2.00 |
| Disodium Phenyl Dibenzimidazole | 2.50 | | 0.50 | 2.00 | |
| Tetrasulfonate | | | | | |
| Ethylhexyl Triazone Uvinul ® T 150 | 4.00 | | 3.00 | 4.00 | |
| Octocrylene | | 4.00 | | | |
| Diethylhexyl Butamido Triazone | 1.00 | | | 2.00 | 1.00 |
| Phenylbenzimidazole Sulfonic Acid | 0.50 | | 3.00 | | |
| Methylene Bis-Benzotriazolyl | 2.00 | | 0.50 | 1.50 | 2.50 |
| Tetramethylbutylphenol | | | | | |
| Ethylhexyl Salicylate | | | 3.00 | | |
| Drometrizole Trisiloxane | | | 0.50 | | |
| Terephthalidene Dicamphor Sulfonic Acid | | 1.50 | | 1.00 | 1.00 |
| Diethylhexyl 2,6-Naphthalate | | | 7.00 | | 4.00 |
| Titanium Dioxide-microfine | 1.00 | | 3.00 | | 3.50 |
| Zinc Oxide-microfine | | | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soya (Soybean) Oil | | 1.00 | 1.50 | | 1.00 |
| Magnesium Sulfate MgSO4 | 1.00 | 0.50 | | 0.50 | |
| Magnesium Chloride MgCl2 | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 1.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Fusion protein-KBD | 0.5 | 3.0 | 1.0 | 5.0 |
| Uvinul ® A Plus ™ | 2.00 | 1.50 | 0.75 | 9.00 |
| UvaSorb ® k2A | | 2.00 | | 4.00 |
| Ethylhexyl Bis- | | | | |
| Isopentylbenzoxazolylphenyl | | | | |
| Melamine | | | | |
| Ethylhexyl Methoxycinnamate | | 3.00 | | |
| Uvinul ® MC 80 | | | | |
| Bis-Ethylhexyloxyphenol | | 1.50 | | 2.00 |
| Methoxyphenyl Triazine | | | | |
| Butyl Methoxydibenzoylmethane | | | 2.00 | |
| Disodium Phenyl Dibenzimidazole | 2.50 | | 0.50 | 2.00 |
| Tetrasulfonate | | | | |
| Ethylhexyl Triazone | 4.00 | | 3.00 | 4.00 |
| Uvinul ® T 150 | | | | |
| Octocrylene | | 4.00 | | |
| Diethylhexyl Butamido Triazone | 1.00 | | | 2.00 |
| Phenylbenzimidazole Sulfonic | 0.50 | | 3.00 | |
| Acid | | | | |
| Methylene Bis-Benzotriazolyl | 2.00 | | 0.50 | 1.50 |
| Tetramethylbutylphenol | | | | |
| Ethylhexyl Salicylate | | | 3.00 | |
| Drometrizole Trisiloxane | | | 0.50 | |
| Terephthalidene Dicamphor | | 1.50 | | 1.00 |
| Sulfonic Acid | | | | |
| Diethylhexyl 2,6-Naphthalate | | | 7.00 | |
| Titanium Dioxide-microfine | 1.00 | | 3.00 | |
| Zinc Oxide-microfine | | | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

PIT emulsion

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone | | | | 0.50 | | 1.00 | | |
| Copolyol | | | | | | | | |
| Polyglyceryl-2 Dioleate | | | | 0.75 | 0.30 | | | |
| Fusion protein-KBD | 0.1 | 5.0 | 20.0 | 0.5 | 3.0 | 0.25 | 10.0 | 3.0 |
| Uvinul ® A Plus ™ | 2.00 | 1.50 | 0.75 | 1.00 | 2.10 | 4.50 | 5.00 | 2.10 |
| UvaSorb ® k2A | | | 4.00 | | | | 1.50 | |
| Ethylhexyl Bis- | | | | | | | | |
| Isopentylbenzoxazolyl- | | | | | | | | |
| phenyl Melamine | | | | | | | | |
| Ethylhexyl | | | | | 5.00 | 6.00 | 8.00 | 5.00 |
| Methoxycinnamate | | | | | | | | |
| Uvinul ® MC 80 | | | | | | | | |
| Bis-Ethylhexyloxyphenol | | 1.50 | | 2.00 | 2.50 | | 2.50 | 2.50 |
| Methoxyphenyl Triazine | | | | | | | | |
| Butyl Methoxydibenzoyl- | | | 2.00 | | 2.00 | 1.50 | | 2.00 |
| methane | | | | | | | | |
| Disodium Phenyl | 2.50 | | 0.50 | 2.00 | | 0.30 | | |
| Dibenzimidazole | | | | | | | | |
| Tetrasulfonate | | | | | | | | |
| Ethylhexyl Triazone | 4.00 | | 3.00 | 4.00 | | 2.00 | | |
| Uvinul ® T 150 | | | | | | | | |
| Octocrylene | | 4.00 | | | | | 7.50 | |
| Diethylhexyl Butamido | 1.00 | | | 2.00 | 1.00 | | 1.00 | 1.00 |
| Triazone | | | | | | | | |
| Phenylbenzimidazole | 0.50 | | 3.00 | | | | | |
| Sulfonic Acid | | | | | | | | |
| Methylene Bis- | 2.00 | | 0.50 | 1.50 | 2.50 | | | 2.50 |
| Benzotriazolyl | | | | | | | | |
| Tetramethylbutylphenol | | | | | | | | |
| Ethylhexyl Salicylate | | | 3.00 | | | | 5.00 | |
| Drometrizole Trisiloxane | | | 0.50 | | | 1.00 | | |
| Terephthalylidene | | 1.50 | | 1.00 | 1.00 | | 0.50 | 1.00 |
| Dicamphor Sulfonic Acid | | | | | | | | |
| Diethylhexyl 2,6- | | | 7.00 | | 10.00 | 7.50 | | 8.00 |
| Naphthalate | | | | | | | | |
| Titanium | 1.00 | | 3.00 | | 3.50 | | 1.50 | 3.50 |
| Dioxide-microfine | | | | | | | | |
| Zinc Oxide-microfine | | | | 0.25 | | 2.00 | | |
| C12-15 Alkyl Benzoate | 3.50 | | | 6.35 | | | | 0.10 |
| Cocoglyceride | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl | | 0.50 | | | 2.00 | | | |
| Galactomannan | | | | | | | | |
| Hydrogenated Coco- | | | | | 3.00 | 4.00 | | |
| Glycerides | | | | | | | | |
| Behenoxy Dimethicone | | | | | | 1.50 | 2.00 | |
| VP/Hexadecene | | | | 1.00 | 1.20 | | | |
| Copolymer | | | | | | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii | | 2.00 | | 3.60 | | 2.00 | | |
| (Shea Butter) | | | | | | | | |
| Iodopropyl | 0.12 | | | | 0.20 | | | |
| Butylcarbamate | | | | | | | | |
| Biosaccaride Gum-1 | | | | 0.10 | | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

Gel cream

| | 1 | 2 | 3 | 4 |
|---|---|---|---|---|
| Acrylates/C10-30 Alkylacrylate | 0.40 | 0.35 | 0.40 | 0.35 |
| Crosspolymer | | | | |
| Carbomer | 0.20 | 0.22 | 0.20 | 0.22 |
| Luvigel ® EM | 1.50 | 2.50 | 2.80 | 3.50 |
| Xanthan Gum | 0.10 | 0.13 | 0.10 | 0.13 |
| Cetearyl Alcohol | 3.00 | 2.50 | 3.00 | 2.50 |
| C12-15 Alkyl Benzoate | 4.00 | 4.50 | 4.00 | 4.50 |
| Caprylic/Capric Triglyceride | 3.00 | 3.50 | 3.00 | 3.50 |
| Titanium Dioxide-microfine | 1.00 | | 1.50 | |
| Zinc Oxide-microfine | | 2.00 | | 0.25 |
| Fusion protein-KBD | 0.5 | 10.0 | 3.0 | 5.0 |
| Dihydroxyacetone | | | 3.00 | 5.00 |
| Cyclomethicone | 5.00 | 5.50 | 5.00 | 5.50 |
| Dimethicone | 1.00 | 0.60 | 1.00 | 0.60 |
| Glycerin | 1.00 | 1.20 | 1.00 | 1.20 |
| Sodium Hydroxide | q.s. | q.s. | q.s. | q.s. |
| Preservative | 0.30 | 0.23 | 0.30 | 0.23 |
| Perfume oil | 0.20 | | 0.20 | |
| Aqua dem. | ad 100 | ad 100 | ad 100 | ad 100 |
| adjust pH to 6.0 | | | | |

OW self-tanning formulation

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Fusion protein-KBD | 0.1 | 0.5 | 0.025 | 5.0 | 3.0 | 10.0 | 1.0 |
| Dihydroxyacetone | | | 3.00 | 5.00 | | 4 | |
| Titanium Dioxide-microfine | 1.00 | | | | 1.50 | | 1.50 |
| Zinc Oxide-microfine | | | | 0.25 | | 2.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Biosaccaride Gum-1 | | 3.00 | 10.00 | | | | |
| Glycine Soya (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

OW make-up

| | 1 | 2 | 3 | 4 | 5 | 6 | 7 |
|---|---|---|---|---|---|---|---|
| Glyceryl Monostearate SE | 0.50 | 1.00 | 3.00 | | | 1.50 | |
| Glyceryl Stearate Citrate | 2.00 | | 1.00 | 2.00 | 4.00 | | |
| Stearic Acid | | 3.00 | | 2.00 | | | |
| PEG-40 Stearate | 0.50 | | | | | 2.00 | |
| Cetyl Phosphate | | | | | | 1.00 | |
| Cetearyl Sulfate | | | | | | | 0.75 |
| Stearyl Alcohol | | | 3.00 | | | 2.00 | 0.60 |
| Cetyl Alcohol | 2.50 | 1.10 | | 1.50 | 0.60 | | 2.00 |
| Fusion protein-KBD | 3.0 | 5.0 | 2.0 | 0.5 | 1.0 | 5.0 | 10.0 |
| Titanium Dioxide | 10.00 | 12.00 | 9.00 | 8.50 | 11.00 | 9.50 | 10.00 |
| Iron Oxide | 2.00 | 4.00 | 3.00 | 5.00 | 3.40 | 6.00 | 4.40 |
| Zinc Oxide | | 4.00 | | 2.00 | | 3.00 | |
| C12-15 Alkyl Benzoate | | 0.25 | | | 4.00 | 7.00 | |
| Dicapryl Ether | | | 3.50 | | 2.00 | | |
| Butylene Glycol | 5.00 | | 6.00 | | | | |
| Dicaprylate/Dicaprate | | | | | | | |
| Cocoglycerides | | | 6.00 | | 2.00 | | |
| Dimethicone | 0.50 | | 1.00 | | 2.00 | | |
| Cyclomethicone | 2.00 | | 0.50 | | 0.50 | | |
| Butyrospermum Parkii (Shea | | 2.00 | | | | | |
| Butter) | | | | | | | |
| VP/Hexadecene Copolymer | 0.20 | | | 0.50 | | 1.00 | |
| Glycerin | 3.00 | 7.50 | | 7.50 | 5.00 | | 2.50 |
| Xanthan Gum | 0.15 | | 0.05 | | | 0.30 | |
| Sodium Carbomer | | 0.20 | | 0.15 | 0.25 | | |
| Vitamin E Acetate | 0.60 | | 0.23 | | 0.70 | 1.00 | |
| Glycine Soya (Soybean) Oil | | | | 0.50 | | 1.50 | 1.00 |
| Ethylhexylglycerin | 0.30 | | | | | | |
| DMDM Hydantoin | | 0.60 | 0.40 | 0.20 | | | |
| Iodopropynyl Butylcarbamate | | | | 0.18 | 0.20 | | |
| Methylparaben | 0.15 | | 0.25 | | 0.50 | | |
| Phenoxyethanol | 1.00 | 0.40 | | | 0.40 | 0.50 | 0.40 |
| Trisodium EDTA | 0.02 | | 0.05 | | | | |
| Tetrasodium | | | | 0.25 | 1.00 | | |
| Iminodisuccinate | | | | | | | |
| Ethanol | 2.00 | 1.50 | | 3.00 | | 1.20 | 5.00 |
| Perfume oil | 0.10 | 0.25 | 0.30 | | 0.40 | 0.20 | |
| Aqua dem. | ad 100 | ad 100 | ad 100 | ad 100 | ad 100 | ad 100 | ad 100 |

Self-tanning hydrodispersion

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl Acrylate | 0.50 | | 0.40 | 0.10 | 0.50 |
| Crosspolymer | | | | | |
| Xanthan Gum | | 0.30 | 0.15 | | |
| Fusion protein-KBD | 3.0 | 1.0 | 0.5 | 0.1 | 5.0 |
| Dihydroxyacetone | | | 3.00 | 5.00 | |
| Uvinul ® A Plus ™ | 2.00 | 1.50 | 0.75 | 1.00 | 2.10 |
| Titanium Dioxide-microfine | 1.00 | | 1.00 | | 1.00 |
| Zinc Oxide-microfine | | 1.90 | | 0.25 | |
| C12-15 Alkyl Benzoate | 2.00 | 2.50 | | | |
| Dicapryl Ether | | 4.00 | | | |
| Butylene Glycol Dicaprylate/Dicaprate | 4.00 | | 2.00 | 6.00 | |
| Dicapryl Carbonate | | 2.00 | 6.00 | | |
| Dimethicone | | 0.50 | 1.00 | | |
| Phenyl Trimethicone | 2.00 | | 0.50 | | |
| Butyrospermum Parkii (Shea Butter) | | 2.00 | | 5.00 | |
| VP/Hexadecene Copolymer | 0.50 | | | 0.50 | 1.00 |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya (Soybean) Oil | | | 1.50 | | 1.00 |
| Vitamin E Acetate | 0.50 | | 0.25 | | 1.00 |
| Glucosylrutin | 0.60 | | | 0.25 | |
| DMDM Hydantoin | | 0.60 | 0.45 | 0.25 | |
| Iodopropynyl Butylcarbamate | 0.20 | | | | |
| Methylparaben | 0.50 | | 0.25 | 0.15 | |
| Phenoxyethanol | 0.50 | 0.40 | | 1.00 | |
| Trisodium EDTA | | 0.01 | 0.05 | | 0.10 |
| Ethanol | 3.00 | 2.00 | 1.50 | | 7.00 |
| Perfume oil | 0.20 | | 0.05 | 0.40 | |
| Aqua dem. | ad 100 | ad 100 | ad 100 | ad 100 | ad 100 |

Aftersun hydrodispersion

| | 1 | 2 | 3 | 4 | 5 |
|---|---|---|---|---|---|
| Ceteareth-20 | 1.00 | | | 0.50 | |
| Cetyl Alcohol | | | 1.00 | | |
| Luvigel ® EM | | 2.00 | | 2.50 | 2.00 |
| Acrylates/C10-30 Alkyl | 0.50 | 0.30 | 0.40 | 0.10 | 0.50 |
| Acrylate Crosspolymer | | | | | |
| Xanthan Gum | | 0.30 | 0.15 | | |
| Fusion protein-KBD | 0.1 | 5.0 | 0.5 | 3.0 | 1.0 |
| C12-15 Alkyl Benzoate | 2.00 | 2.50 | | | |
| Dicapryl Ether | | 4.00 | | | |
| Butylene glycol | 4.00 | | 2.00 | 6.00 | |
| Dicaprylate/Dicaprate | | | | | |
| Dicapryl Carbonate | | 2.00 | 6.00 | | |
| Dimethicone | | 0.50 | 1.00 | | |
| Phenyl Trimethicone | 2.00 | | 0.50 | | |
| Tricontanyl PVP | 0.50 | | 1.00 | | |
| Ethylhexylglycerin | | | 1.00 | | 0.80 |
| Glycerin | 3.00 | 7.50 | | 7.50 | 8.50 |
| Glycine Soya (Soybean) | | | 1.50 | | 1.00 |
| Oil | | | | | |
| Vitamin E Acetate | 0.50 | | 0.25 | | 1.00 |
| Glucosylrutin | 0.60 | | | 0.25 | |
| Trisodium EDTA | | 0.01 | 0.05 | | 0.10 |
| Ethanol | 15.00 | 10.00 | 8.00 | 12.00 | 9.00 |
| Perfume oil | 0.20 | | 0.05 | 0.40 | |
| Aqua dem. | ad 100 | ad 100 | ad 100 | ad 100 | ad 100 |

WO emulsions

| | 1 | 2 | 3 | 4 | 5 |
|---|---|---|---|---|---|
| Cetyl Dimethicone | | 2.50 | | 4.00 | |
| Polyglyceryl-2 Dipolyhydroxystearate | 5.00 | | | | 4.50 |
| PEG-30Dipolyhydroxystearate | | | 5.00 | | |
| Fusion protein-KBD | 5.0 | 10.0 | 0.1 | 0.5 | 1.0 |
| Uvinul ® A Plus ™ | 2.00 | 1.50 | 0.75 | 1.00 | 2.10 |
| Titanium Dioxide-microfine | 1.00 | | 3.00 | | 3.50 |
| Zinc Oxide-microfine | | 0.90 | | 0.25 | |
| Mineral Oil | | 12.00 | 10.00 | | 8.00 |
| C12-15 Alkyl Benzoate | | | | 9.00 | |
| Dicaprylyl Ether | 10.00 | | | | 7.00 |
| Butylene Glycol Dicaprylate/Dicaprate | | | 2.00 | 8.00 | 4.00 |
| Dicaprylyl Carbonate | 5.00 | | 6.00 | | |
| Dimethicone | | 4.00 | 1.00 | 5.00 | |
| Cyclomethicone | 2.00 | 25.00 | | | 2.00 |
| Butyrospermum Parkii (Shea Butter) | | | 3.00 | | |
| Petrolatum | | 4.50 | | | |
| VP/Hexadecene Copolymer | 0.50 | | | 0.50 | 1.00 |
| Ethylhexylglycerin | | 0.30 | 1.00 | | 0.50 |
| Glycerin | 3.00 | 7.50 | | 7.50 | 8.50 |
| Glycine Soya (Soybean) Oil | | 1.00 | 1.50 | | 1.00 |
| Magnesium Sulfate | 1.00 | 0.50 | | 0.50 | |
| Magnesium Chloride | | | 1.00 | | 0.70 |
| Vitamin E Acetate | 0.50 | | 0.25 | | 1.00 |
| Ascorbyl Palmitate | 0.50 | | | 2.00 | |
| Biosaccaride Gum-1 | | | | 3.50 | 7.00 |
| DMDM Hydantoin | | 0.60 | 0.40 | 0.20 | |
| Methylparaben | 0.50 | | 0.25 | 0.15 | |
| Phenoxyethanol | 0.50 | 0.40 | | 1.00 | |
| Trisodium EDTA | 0.12 | 0.05 | | 0.30 | |
| Ethanol | 3.00 | | 1.50 | | 5.00 |
| Perfume oil | 0.20 | | 0.40 | 0.35 | |
| Aqua dem. | ad 100 | ad 100 | ad 100 | ad 100 | ad 100 |

Solids-stabilized emulsion
(Pickering emulsions)

| | 1 | 2 | 3 | 4 | 5 |
|---|---|---|---|---|---|
| Mineral Oil | | | 16.00 | 16.00 | |
| Octyldodecanol | 9.00 | 9.00 | 5.00 | | |
| Caprylic/Capric Triglyceride | 9.00 | 9.00 | 6.00 | | |
| C12-15 Alkyl Benzoate | | | | 5.00 | 8.00 |
| Butylene Glycol Dicaprylate/Dicaprate | | | | | 8.00 |
| Dicaprylyl Ether | 9.00 | | | 4.00 | |
| Dicaprylyl Carbonate | | 9.00 | | | |
| Hydroxyoctacosanyl Hydroxystearate | 2.00 | 2.00 | 2.20 | 2.50 | 1.50 |
| Disteardimonium Hectorite | 1.00 | 0.75 | | 0.50 | 0.25 |
| Cera Microcristallina + Paraffinum Liquidum | | 0.35 | | | 5.00 |
| Hydroxypropyl Methylcellulose | | | 0.10 | | 0.05 |
| Dimethicone | | | | | 3.00 |
| Fusion protein-KBD | 1.0 | 0.5 | 0.1 | 3.0 | 5.0 |
| Titanium Dioxide + Alumina + Simethicone + | | 3.00 | | | |
| Aqua | | | | | |
| Titanium Dioxide + Trimethoxycaprylylsilane | | 2.00 | 4.00 | 2.00 | 4.00 |
| Silica Dimethyl Silylate | 2.50 | | | 6.00 | 2.50 |
| Boron Nitride | | | 1.00 | | |
| Starch/Sodium metaphosphate Polymer | 2.00 | | | | |
| Tapioca Starch | | 0.50 | | | |
| Sodium Chloride | 5.00 | 7.00 | 8.50 | 3.00 | 4.50 |
| Glycerin | | | | 1.00 | |
| Trisodium EDTA | 1.00 | 1.00 | 1.00 | 1.00 | 1.00 |
| Vitamin E Acetate | 5.00 | 10.00 | 3.00 | 6.00 | 10.00 |
| Ascorbyl Palmitate | 1.00 | 1.00 | | 1.00 | |
| Methylparaben | | 0.60 | | | 0.20 |
| Propylparaben | | | | | 0.20 |
| Phenoxyethanol | | | 0.20 | | |
| Hexamidine Diisethionate | | | 0.40 | 0.50 | 0.40 |
| Diazolidinyl Urea | | | | | 0.08 |
| Ethanol | | | 0.23 | 0.20 | |
| Perfume oil | 5.00 | | 3.00 | 4.00 | |
| Aqua dem. | 0.20 | | 0.30 | 0.10 | |
| | ad 100 | ad 100 | ad 100 | ad 100 | ad 100 |

Sticks

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol Dicaprylate/ | | | | 12.00 |
| Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Cetearyl Alcohol | 8.00 | 11.00 | 9.00 | 7.00 |
| Myristyl Myristate | 3.50 | 3.00 | 4.00 | 3.00 |
| Beeswax | 5.00 | 5.00 | 6.00 | 6.00 |
| Copernicia Cerifera (Carnauba) | 1.50 | 2.00 | 2.00 | 1.50 |
| Wax | | | | |
| Cera Alba | 0.50 | 0.50 | 0.50 | 0.40 |
| C16-40 Alkyl Stearate | | 1.50 | 1.50 | 1.50 |
| Fusion protein-KBD | 10.0 | 1.0 | 3.0 | 0.1 |
| Uvinul ® A Plus ™ | 2.00 | 1.50 | 0.75 | 9.00 |
| Titanium Dioxide-microfine | 1.00 | | 3.00 | |
| Zinc Oxide-microfine | | 1.00 | | 0.25 |
| Vitamin E Acetate | 0.50 | 1.00 | | |
| Ascorbyl Palmitate | 0.05 | | 0.05 | |
| Buxux Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

Self-tanning PIT emulsions

| | 1 | 2 | 3 | 4 | 5 | 6 | 7 | 8 |
|---|---|---|---|---|---|---|---|---|
| Glyceryl Monostearate SE | 0.50 | 2.00 | 3.00 | 5.00 | | 0.50 | 4.00 | |
| Glyceryl Isostearate | | | | | 3.50 | 4.00 | 2.00 | |
| Isoceteth-20 | | 0.50 | | | 2.00 | | | |
| Ceteareth-12 | | 5.00 | | 1.00 | | | 3.50 | 5.00 |
| Ceteareth-20 | | 5.00 | | 1.00 | | | | 3.50 |
| PEG-100 Stearate | | | | 2.80 | | 2.30 | 3.30 | |
| Cetyl Alcohol | 5.20 | | 1.20 | 1.00 | 1.30 | | 0.50 | 0.30 |
| Cetyl Palmitate | 2.50 | 1.20 | | 1.50 | | 0.50 | | 1.50 |
| Cetyl Dimethicone Copolyol | | | | 0.50 | | 1.00 | | |
| Polyglyceryl-2 | | | | 0.75 | 0.30 | | | |
| Fusion protein-KBD | 0.1 | 0.5 | 0.01 | 5.0 | 0.5 | 3.0 | 0.025 | 10.0 |
| Dihydroxyacetone | | | 3.00 | 5.00 | | | 4.00 | |
| Uvinul ® A Plus ™ | 2.00 | 1.50 | 0.75 | 1.00 | 2.10 | 4.50 | 5.00 | 2.10 |
| Titanium Dioxide-microfine | 1.00 | | 1.50 | | 3.50 | | 1.50 | 1.00 |
| Zinc Oxide-microfine | | 1.00 | | 0.25 | | 2.00 | | 1.50 |
| C12-15 Alkyl Benzoate | 3.50 | | | 6.35 | | | | 0.10 |
| Cocoglycerides | | 3.00 | | 3.00 | | | | 1.00 |
| Dicapryl Ether | 4.50 | | | | | | | |
| Dicaprylyl Carbonate | | 4.30 | | 3.00 | | | | 7.00 |
| Dibutyl Adipate | | | | 0.50 | | | | 0.30 |
| Phenyl Trimethicone | 2.00 | | | 3.50 | | 2.00 | | |
| Cyclomethicone | | 3.00 | | | | | | |
| C1-5 Alkyl Galactomannan | | 0.50 | | | 2.00 | | | |
| Hydrogenated Coco-Glycerides | | | | | 3.00 | 4.00 | | |
| Behenoxy Dimethicone | | | | | | 1.50 | 2.00 | |
| VP/Hexadecene Copolymer | | | | 1.00 | 1.20 | | | |
| Glycerin | 4.00 | 6.00 | 5.00 | | 8.00 | 10.00 | | |
| Vitamin E Acetate | 0.20 | 0.30 | 0.40 | | 0.30 | | | |
| Butyrospermum Parkii (Shea | | 2.00 | | 3.60 | | 2.00 | | |
| Butter) | | | | | | | | |
| Iodopropyl Butylcarbamate | 0.12 | | | | 0.20 | | | |
| DMDM Hydantoin | 0.10 | | | | 0.12 | | 0.13 | |
| Methylparaben | | 0.50 | 0.30 | | 0.35 | | | |
| Phenoxyethanol | 0.50 | 0.40 | | 1.00 | | | | |
| Ethylhexylglycerin | | 0.30 | | | 1.00 | | 0.35 | |
| Ethanol | 2.00 | | 2.00 | | | 5.00 | | |
| Trisodium EDTA | 0.40 | | 0.15 | | | 0.20 | | |
| Perfume oil | 0.20 | | 0.20 | | 0.24 | 0.16 | 0.10 | 0.10 |
| Aqua dem. | ad 100 | ad 100 | ad 100 | ad 100 | ad 100 | ad 100 | ad 100 | ad 100 |

Oil gel

| | 1 | 2 | 3 | 4 |
|---|---|---|---|---|
| Caprylic/Capric Triglyceride | 12.00 | 10.00 | 6.00 | |
| Octyldodecanol | 7.00 | 14.00 | 8.00 | 3.00 |
| Butylene Glycol | | | | 12.00 |
| Dicaprylate/Dicaprate | | | | |
| Pentaerythrityl Tetraisostearate | 10.00 | 6.00 | 8.00 | 7.00 |
| Polyglyceryl-3 Diisostearate | 2.50 | | | |
| Bis-Diglyceryl Polyacyladipate-2 | 9.00 | 8.00 | 10.00 | 8.00 |
| Myristyl Myristate | 3.50 | 3.00 | 4.00 | 3.00 |
| Quaternium-18 Bentonite | 5.00 | 5.00 | 6.00 | 6.00 |
| Propylene Carbonate | 15.00 | 20.00 | 18.00 | 19.50 |
| Fusion protein-KBD | 1.0 | 0.5 | 3.0 | 5.0 |
| Vitamin E Acetate | 0.50 | 1.00 | | |
| Ascorbyl Palmitate | 0.05 | | 0.05 | |
| Buxus Chinensis (Jojoba) Oil | 2.00 | 1.00 | | 1.00 |
| Perfume oil, BHT | 0.10 | 0.25 | | 0.35 |
| Ricinus Communis (Castor) Oil | ad 100 | ad 100 | ad 100 | ad 100 |

### Example 72

In the formulations below, cosmetic sunscreen preparations comprising a combination of at least one inorganic pigment, preferably zinc oxide and/or titanium dioxide and organic UV-A and UV-B filters are described.

The formulations specified below are prepared in customary ways known to the person skilled in the art.

The content of keratin-binding effector molecule C16-KBD-D (according to SEQ ID No.: 212 in pRee024 (FIG. 8)) prepared according to Example 3 refers to 100% of active ingredient. The active ingredient according to the invention can either be used in pure form or else in the form of an aqueous solution. In the case of the aqueous solution, the content of Water demin. in the particular formulation must be adjusted.

| | | | |
|---|---|---|---|
| A | 7.50 | Uvinul MC 80 | Ethylhexyl Cinnamate |
| | 2.00 | Uvinul M 40 | Benzophenone-3 |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 1.00 | Sepigel 305 | Polyacrylamide, C13-14 Isoparaffin, |
| | | | Laureth-7 |
| | 1% | Fusion protein-KBD | |
| | q.s. | | Preservative |

| | | | |
|---|---|---|---|
| A | 7.50 | Uvinul MC 80 | Ethylhexyl Cinnamate |
| | 2.00 | Uvinul M 40 | Benzophenone-3 |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1% | Fusion protein-KBD | |
| | q.s. | | Preservative |

| | | | |
|---|---|---|---|
| A | 7.50 | Uvinul MC 80 | Ethylhexyl Cinnamate |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) |
| | | | Diphenyl Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/ |
| | | | Sodium Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) |
| | | | Diphenyl Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/ |
| | | | Sodium Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/ |
| | | | Sodium Acryloyldimethyl Taurate |
| | | | Copolymer, Squalane, Polysorbate 60 |
| | 1% | Fusion protein-KBD | |
| | q.s. | | Preservative |

| | | | |
|---|---|---|---|
| A | 4.00 | Uvinul MC 80 | Ethylhexyl Cinnamate |
| | 2.00 | Uvinul T 150 | Ethyl Hexyl Triazone |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1% | Fusion protein-KBD | |
| | q.s. | | Preservative |

| | | | |
|---|---|---|---|
| A | 4.00 | Uvinul MC 80 | Ethylhexyl Cinnamate |
| | 1.00 | Uvinul T 150 | Ethyl Hexyl Triazone |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF-S | Titanium Dioxide, Silica Hydrate, |
| | | | Alumina Hydrate, Methicone/ |
| | | | Dimethicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.70 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, |
| | | | Polysorbate 60 |
| | 0.5% | Fusion protein-KBD | |
| | q.s. | | Preservative |

| | | | |
|---|---|---|---|
| A | 4.00 | Uvinul MC 80 | Ethylhexyl Cinnamate |
| | 1.00 | Uvinul T 150 | Ethyl Hexyl Triazone |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 67.10 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, |
| | | | Polysorbate 60 |
| | 0.1% | Fusion protein-KBD | |
| | q.s. | | Preservative |

| | | | |
|---|---|---|---|
| A | 5.00 | Uvinul N 539 T | Octocrylene |
| | 2.00 | Parsol 1789 | Butyl Methoxydibenzoylmethane |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF-S | Titanium Dioxide, Silica Hydrate, |
| | | | Alumina Hydrate, Methicone/ |
| | | | Dimethicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | T-Lite SF | Titanium Dioxide, Alumina Hydrate, |
| | | | Dimethicone/Methicone Copolymer |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 65.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 2% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.70 | Water demin. | Aqua dem. |
| D | 2.00 | Simulgel NS | Hydroxyethyl Acrylate/ |
| | | | Sodium Acryloyldimethyl Taurate |
| | | | Copolymer, Squalane, Polysorbate 60 |
| | 0.5% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 2.0 | Mexoryl SX | Terephthalidene Dicamphor |
| | | | Sulfonic Acid |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydrobenzoyl |
| | | | Hexyl Benzoate |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 2.0 | Mexoryl SX | Terephthalidene Dicamphor Sulfonic |
| | | | Acid |
| | 65.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 2% | Fusion protein-KBD | |
| | q.s. | | Preservative |

| | | | |
|---|---|---|---|
| A | 5.00 | Uvinul N 539 T | Octocrylene |
| | 2.00 | Uvinul A Plus | Diethylamino Hydroxybenzoyl |
| | | | Hexyl Benzoate |
| | 2.00 | Mexoryl XL | Drometrizole Trisiloxane |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl Capryl |
| | | | Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 67.10 | Water demin. | Aqua dem. |
| D | 2.00 | Simulgel NS | Hydroxyethyl Acrylate/Sodium |
| | | | Acryloyldimethyl Taurate |
| | | | Copolymer, Squalane, |
| | | | Polysorbate 60 |
| | 0.1% | Fusion protein-KBD | |
| | q.s. | | Preservative |

| | | | |
|---|---|---|---|
| A | 5.00 | Uvinul N 539 T | Octocrylene |
| | 2.00 | Tinosorb S | Bis-Ethylhexyloxyphenol |
| | | | Methoxyphenyl Triazine |
| | 0.80 | Rylo PG 11 | Polyglyceryl Dimer Soyate |
| | 1.00 | Span 60 | Sorbitan Stearate |
| | 0.50 | Vitamin E acetate | Tocopheryl Acetate |
| | 3.00 | Dracorin 100 SE | Glyceryl Stearate, PEG-100 Stearate |
| | 1.00 | Cremophor CO 410 | PEG-40 Hydrogenated Castor Oil |
| B | 3.00 | Z-COTE MAX | Zinc Oxide (and) Diphenyl |
| | | | Capryl Methicone |
| | 1.00 | Cetiol SB 45 | Butyrospermum Parkii (Shea Butter) |
| | 6.50 | Finsolv TN | C12-15 Alkyl Benzoate |
| C | 5.00 | Butylene Glycol | Butylene Glycol |
| | 0.30 | Keltrol | Xanthan Gum |
| | 0.10 | Edeta BD | Disodium EDTA |
| | 0.10 | Allantoin | Allantoin |
| | 66.20 | Water demin. | Aqua dem. |
| D | 2.00 | Simulgel NS | Hydroxyethyl Acrylate/Sodium |
| | | | Acryloyldimethyl Taurate Copolymer, |
| | | | Squalane, Polysorbate 60 |
| | 1% | Fusion protein-KBD | |
| | q.s. | | Preservative |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl |
| | | | Capryl Methicone |
| C | 4.00 | 1,2-Propylene | Propylene Glycol |
| | | Glycol Care | |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water demin. | Aqua dem. |
| | q.s. | Preservative | |
| | 0.50 | Vitamin E acetate | Tocopheryl Acetate |
| | 2% | Fusion protein-KBD | |
| | 1.00 | RetiSTAR | Caprylic/Capric Triglyceride, Sodium |
| | | | Ascorbate, Tocopherol, Retinol |

| | | | |
|---|---|---|---|
| A | 7.50 | Uvinul MC 80 | Ethylhexyl Methoxycinnamate |
| | 2.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF-S | Titanium Dioxide, Silica |
| | | | Hydrate, Alumina Hydrate, |
| | | | Methicone/Dimethicone Copolymer |
| C | 4.00 | 1,2-Propylene | Propylene Glycol |
| | | Glycol Care | |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water demin. | Aqua dem. |
| | q.s. | Preservative | |
| | 0.50 | Vitamin E acetate | Tocopheryl Acetate |
| | 1% | Fusion protein-KBD | |
| | 2.00 | RetiSTAR | Caprylic/Capric Triglyceride, Sodium |
| | | | Ascorbate, Tocopherol, Retinol |

| | | | |
|---|---|---|---|
| A | 7.50 | Uvinul MC 80 | Ethylhexyl Methoxycinnamate |
| | 5.09 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF | Titanium Dioxide, Alumina Hydrate, |
| | | | Dimethicone/Methicone Copolymer |
| C | 4.00 | 1,2-Propylene | Propylene Glycol |
| | | Glycol Care | |
| | 1.00 | D-Panthenol 50 P | Panthenol, Propylene Glycol |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water demin. | Aqua dem. |
| | q.s. | Preservative | |
| | 0.50 | Vitamin E acetate | Tocopheryl Acetate |
| | 1% | Fusion protein-KBD | |
| | 1.00 | RetiSTAR | Caprylic/Capric Triglyceride, |
| | | | Sodium Ascorbate, Tocopherol, |
| | | | Retinol |

| | | | |
|---|---|---|---|
| A | 5.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF | Titanium Dioxide, Alumina Hydrate, |
| | | | Dimethicone/Methicone Copolymer |
| C | 4.00 | 1,2-Propylene | Propylene Glycol |
| | | Glycol Care | |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | ad 100 | Water demin. | Aqua dem. |
| | q.s. | Preservative | |
| | 0.50 | Vitamin E acetate | Tocopheryl Acetate |
| | 2% | Fusion protein-KBD | |
| | 1.00 | RetiSTAR | Caprylic/Capric Triglyceride, |
| | | | Sodium Ascorbate, Tocopherol, |
| | | | Retinol |

| | | | |
|---|---|---|---|
| A | 1.00 | Abil Care 85 | Bis-PEG/PPG-16/16 PEG/PPG-16/16 |
| | | | Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Parsol 1789 | Butyl Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 | Cyclopentasiloxane, |
| | | Fluid | Cyclohexasiloxane |
| | 5.00 | Uvinul N 539 T | Octocrylene |
| B | 3.00 | Z-COTE MAX | Zinc Oxide (and) Diphenyl |
| | | | Capryl Methicone |
| C | 5.00 | 1,2-Propylene Glycol | Propylene Glycol |
| | 1.00 | Mexoryl SX | Terephthalidene Dicamphor |
| | | | Sulfanic Acid |
| | 0.20 | Keltrol | Xanthan Gum |
| | 0.06 | Edeta BD | Disodium EDTA |
| | 0.04 | TEA | Triethanolamine |
| | 0.30 | Carbopol Ultrez 10P | Carbomer |
| | 63.50 | Water demin. | Aqua dem. |
| | 1% | Fusion protein-KBD | |
| D | q.s. | Perfume oil | |
| | 0.50 | Glydant | DMDM Hydantoin |

| | | | |
|---|---|---|---|
| A | 5.50 | Uvinul MC 80 | Ethylhexyl Methoxycinnamate |
| | 2.00 | Uvinul A Plus | Diethylamino Hydroxybenzoyl Hexyl Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | T-Lite SF-S | Titanium Dioxide, Silica Hydrate, Alumina |
| | | | Hydrate, Methicone/Dimethicone Copolymer |
| C | 4.00 | 1,2-Propylene Glycol Care | Propylene Glycol |
| | 0.20 | Keltrol | Xanthan Gum |
| | 2.00 | Simulgel NS | Hydroxyethyl Acrylate/Sodium Acryloyldimethyl |
| | | | Taurate Copolymer, Squalane, Polysorbate 60 |
| | 66.30 | Water demin. | Aqua dem. |
| | q.s. | Preservative | |
| | 0.50 | Vitamin E acetate | Tocopheryl Acetate |
| | 0.5% | Fusion protein-KBD | |
| | 1.00 | RetiSTAR | Caprylic/Capric Triglyceride, Sodium Ascorbate, |
| | | | Tocopherol, Retinol |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | Z-COTE MAX | Zinc Oxide (and) Diphenyl Capryl Methicone |
| C | 0.20 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 65.10 | Water demin. | Aqua dem. |
| | 0.20 | Edeta BD | Disodium EDTA |
| | 5.00 | 1,2-Propylene Glycol Care | Propylene Glycol |
| | 0.5% | Fusion protein-KBD | |
| D | 1.00 | Vitamin E acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | T-Lite SF | Titanium Dioxide, Alumina Hydrate, |
| | | | Dimethicone/Methicone Copolymer |
| C | 0.50 | Keltrol | Xanthan Gum |
| | 0.50 | Simulgel NS | Hydroxyethyl Acrylate/Sodium |
| | | | Acryloyldimethyl Taurate |
| | | | Copolymer, Squalane, |
| | | | Polysorbate 60 |
| | 65.10 | Water demin. | Aqua dem. |
| | 0.20 | Edeta BD | Disodium EDTA |
| | 5.00 | 1,2-Propylene | Propylene Glycol |
| | | Glycol Care | |
| | 0.5% | Fusion protein-KBD | |
| D | 1.00 | Vitamin E acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, Silica Hydrate, Alumina |
| | | | Hydrate, Methicone/Dimethicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 64.60 | Water demin. | Aqua dem. |
| | 0.20 | Edeta BD | Disodium EDTA |
| | 5.00 | 1,2-Propylene Glycol Care | Propylene Glycol |
| | 1% | Fusion protein-KBD | |
| D | 1.00 | Vitamin E acetate | Tocopheryl Acetate |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, Silica Hydrate, Alumina |
| | | | Hydrate, Methicone/Dimethicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 64.10 | Water demin. | Aqua dem. |
| | 2.00 | Neoheliopan AP | Disodium Phenyl Dibenzimidazole |
| | | | Tetrasulfonate |
| | 0.20 | Edeta BD | Disodium EDTA |
| | 5.00 | 1,2-Propylene Glycol Care | Propylene Glycol |
| D | 1.00 | Vitamin E acetate | Tocopheryl Acetate |
| | 1% | Fusion protein-KBD | |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 7.00 | Uvinul N 539 T | Octocrylene |
| | 1.00 | Ganex V-220 | VP/Eicosene Copolymer |
| | 7.00 | Isopropyl Palmitate | Isopropyl Palmitate |
| B | 3.00 | T-Lite SF-S | Titanium Dioxide, Silica Hydrate, Alumina |
| | | | Hydrate, Methicone/Dimethicone Copolymer |
| C | 0.50 | Keltrol | Xanthan Gum |
| | 0.50 | Simulgel NS | Hydroxyethyl Acrylate/Sodium |
| | | | Acryloyldimethyl Taurate Copolymer, |
| | | | Squalane, Polysorbate 60 |
| | 64.60 | Water demin. | Aqua dem. |
| | 1.00 | Mexoryl SX | Terephthalidene Dicamphor Sulfonic Acid |
| | 0.20 | Edeta BD | Disodium EDTA |
| | 5.00 | 1,2-Propylene Glycol Care | Propylene Glycol |
| D | 1.00 | Vitamin E acetate | Tocopheryl Acetate |
| | 1% | Fusion protein-KBD | |
| | q.s. | Preservative | |

| | | | |
|---|---|---|---|
| A | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 2.00 | Mexoryl XL | Drometrizole Trisiloxane |
| | 5.00 | Uvinul N 539 T | Octocrylene |
| | 1.00 | Ganex V-220 | VP/Eicosene Copolymer |
| | 7.00 | Isopropyl Palmitate | Isopropyl Palmitate |
| B | 3.00 | T-Lite SF | Titanium Dioxide, Alumina Hydrate, |
| | | | Dimethicone/Methicone Copolymer |
| C | 0.50 | Keltrol | Xanthan Gum |
| | 0.50 | Simulgel NS | Hydroxyethyl Acrylate/Sodium |
| | | | Acryloyldimethyl Taurate |
| | | | Copolymer, Squalane, |
| | | | Polysorbate 60 |
| | 65.10 | Water demin. | Aqua dem. |
| | 0.20 | Edeta BD | Disodium EDTA |
| | 5.00 | 1,2-Propylene | Propylene Glycol |
| D | 1.00 | Vitamin E acetate | Tocopheryl Acetate |
| | 0.5% | Fusion protein-KBD | |
| | q.s. | Preservative | |

| | | | |
|---|---|---|---|
| | 1.00 | Urea | Urea |
| | 2.00 | Neoheliopan AP | Disodium Phenyl Dibenzimidazole |
| | | | Tetrasulfonate |
| | 2.00 | Glycerin 87% | Glycerin |
| | 1.20 | Aristoflex AVC | Ammonium Acryloyldimethyltaurate/ |
| | | | VP Copolymer |
| | 71.30 | Water demin. | Aqua dem. |
| B | 3.00 | Cremophor CO 40 | PEG-40 Hydrogenated Castor Oil |
| | q.s. | Perfume oil | |
| | 5.00 | Miglyol 812 | Caprylic/Capric Triglyceride |
| | 2.00 | Uvinul N 539 T | Octocrylene |
| | 8.00 | Z-COTE MAX | Zinc Oxide (and) Diphenyl Capryl |
| | | | Methicone |
| | 5% | Fusion protein-KBD | |
| | q.s. | Preservative | |

| | | |
|---|---|---|
| 5.00 | Z-COTE MAX | Zinc Oxide (and) Diphenyl Capryl |
| | | Methicone |
| 2.00 | Parsol 1789 | Butyl Methoxydibenzoylmethane |
| 1.00 | Uvinul T150 | Ethylhexyl Triazone |
| 5.00 | Uvinul MC 80 | Ethylhexyl Methoxycinnamate |
| 2.00 | Uvinul T 539 N | Octocrylene |
| 0.50 | Abil 350 | Dimethicone |
| 2.75 | Carnico wax LT 20 | Carnauba (Copernica Cerifera) |
| | | Wax,Paraffin |
| 3.70 | Candelilla wax LT 281 LJ | Candelilla (Euphorbia Cerifera) |
| 1.80 | Beeswax 3050 PH | Wax Beeswax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E acetate | Tocopheryl Acetate |
| 0.42 | D,L-Alpha-Tocopherol | Tocopherol |
| 1% | Fusion protein-KBD | |
| 40.38 | castor oil | Castor (Ricinus Communis) Oil |

| | | |
|---|---|---|
| 5.00 | T-Lite SF | Titanium Dioxide, Alumina Hydrate, |
| | | Dimethicone/Methicone Copolymer |
| 2.00 | Parsol 1789 | Butyl Methoxydibenzoylmethane |
| 1.00 | Uvinul T150 | Ethylhexyl Triazone |
| 3.00 | Uvinul MC 80 | Ethylhexyl Methoxycinnamate |
| 2.00 | Uvinul N 539 T | Octocrylene |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 | Candelilla (Euphorbia Cerifera) Wax |
| | LJ | |
| 1.80 | Beeswax 3050 PH | Beeswax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E acetate | Tocopheryl Acetate |
| 0.42 | D,L-Alpha-Tocopherol | Tocopherol |
| 1% | Fusion protein-KBD | |
| 40.38 | castor oil | Castor (Ricinus Communis) Oil |

| | | |
|---|---|---|
| 5.00 | T-Lite SF-S | Titanium Dioxide, Silica Hydrate, |
| | | Alumina Hydrate, |
| | | Methicone/Dimethicone Copolymer |
| 2.00 | Uvinul A Plus | Diethylamino Hydroxybenzoyl Hexyl |
| | | Benzoate |
| 2.00 | Mexoryl XL | Drometrizole Trisiloxane |
| 3.00 | Uvinul MC 80 | Ethylhexyl Methoxycinnamate |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 | Candelilla (Euphorbia Cerifera) Wax |
| | LJ | |
| 1.80 | Beeswax 3050 PH | Beeswax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E acetate | Tocopheryl Acetate |
| 0.42 | D,L-Alpha-Tocopherol | Tocopherol |
| 2% | Fusion protein-KBD | |
| 39.38 | castor oil | Castor (Ricinus Communis) Oil |

| | | | |
|---|---|---|---|
| A | 6.00 | Cremophor WO 7 | PEG-7 Hydrogenated Castor Oil |
| | 2.00 | Elfacos ST 9 | PEG-45/Dodecyl Glycol Copolymer |
| | 3.00 | Isopropyl myristate | Isopropyl Myristate |
| | 8.00 | Jojoba oil | Simmondsia Chinensis (Jojoba) Seed Oil |
| | 4.00 | Uvinul MC 80 | Ethylhexyl Methoxycinnamate |
| | 2.00 | Uvinul A Plus | Diethylamino Hydroxybenzoyl Hexyl Benzoate |
| | 1.00 | Abil 350 | Dimethicone |
| B | 5.00 | Z-COTE MAX | Zinc Oxide (and) Diphenyl Capryl Methicone |
| | 3.00 | T-Lite SF | Titanium Dioxide, Alumina Hydrate, |
| | | | Dimethicone/Methicone Copolymer |
| C | 0.20 | Edeta BD | Disodium EDTA |
| | 5.00 | Glycerin 87% | Glycerin |
| | 0.30 | Chemag 2000 | Imidazolidinyl Urea |
| | 59.00 | Water demin. | Aqua dem. |
| D | q.s. | Perfume oil | |
| | 1% | Fusion protein-KBD | |
| | q.s. | Preservative | |

| | | | |
|---|---|---|---|
| | 2.00 | Cremophor A 6 | Ceteareth-6, Stearyl Alcohol |
| | 2.00 | Cremophor A 25 | Ceteareth-25 |
| | 3.00 | Syncrowax HRC | Tribehenin |
| | 2.00 | Lanette O | Cetearyl Alcohol |
| | 2.00 | Luvitol EHO | Cetearyl Ethylhexanoate |
| | 5.00 | Uvinul MC 80 | Ethylhexyl Methoxycinnamate |
| | 1.00 | Uvinul T 150 | Ethylhexyl Triazone |
| | 1.00 | Ganex V-220 | VP/Eicosene Copolymer |
| | 7.00 | Isopropyl myristate | Isopropyl Myristate |
| B | 5.00 | Z-COTE MAX | Zinc Oxide (and) Diphenyl Capryl |
| | | | Methicone |
| C | 0.20 | Keltrol | Xanthan Gum |
| | 0.50 | Simulgel NS | Hydroxyethyl Acrylate/Sodium |
| | | | Acryloyldimethyl Taurate |
| | | | Copolymer, Squalane, |
| | | | Polysorbate 60 |
| | 61.10 | Water demin. | Aqua dem. |
| | 0.20 | Edeta BD | Disodium EDTA |
| | 5.00 | 1,2-Propylene | Propylene Glycol |
| | | Glycol Care | |
| D | 1.00 | Vitamin E acetate | Tocopheryl Acetate |
| | 1.5% | Fusion protein-KBD | |
| | q.s. | Preservative | |

| | | | |
|---|---|---|---|
| A | 2.00 | Luvitol EHO | Cetearyl Ethylhexanoate |
| | 5.00 | Paraffin oil | Mineral Oil |
| | 5.00 | Z-COTE MAX | Zinc Oxide (and) Diphenyl Capryl |
| | | | Methicone |
| B | 1.00 | Cremophor A 6 | Ceteareth-6, Stearyl Alcohol |
| | 2.00 | Cremophor A 25 | Ceteareth-25 |
| | 3.00 | Glycerin monostearate | Glyceryl Stearate |
| | 4.00 | Cetylstearyl alcohol | Cetearyl Alcohol |
| C | 5.00 | 1,2-Propylene | Propylene Glycol |
| | | Glycol Care | |
| | 7.50 | Luviquat Care | Polyquaternium-44 |
| | q.s. | Preservative | |
| | q.s. | Perfume oil | |
| | 1% | Fusion protein-KBD | |
| | 64.50 | Water demin. | Aqua dem. |

| | | |
|---|---|---|
| 5.00 | T-Lite SF | Titanium Dioxide, Alumina Hydrate, |
| | | Dimethicone/Methicone Copolymer |
| 10.00 | Z-COTE MAX | Zinc Oxide (and) Diphenyl Capryl |
| | | Methicone |
| 12.00 | Beeswax 3044 PH | Beeswax |
| 8.00 | Candelilla wax LT 281 LJ | Candelilla (Euphorbia Cerifera) Wax |
| 5.00 | Tegin | Glyceryl Stearate SE |
| 5.00 | Dow Corning 345 Fluid | Cyclopentasiloxane, |
| | | Cyclohexasiloxane |
| 5.00 | Witconol APM | PPG-3 Myristyl Ether |
| 5.00 | Softisan 154 | Hydrogenated Palm Oil |
| 8.00 | Paraffin oil, high-viscosity | Mineral Oil |
| 3.00 | Vaseline | Petrolatum |
| 2% | Fusion protein-KBD | |
| 32.00 | castor oil | Castor (Ricinus Communis) Oil |

| | | |
|---|---|---|
| 3.00 | Uvinul MC 80 | Ethylhexyl Methoxycinnamate |
| 2.00 | Uvinul T 150 | Ethylhexyl Triazone |
| 2.00 | Uvinul A Plus | Diethylamino Hydroxybenzoyl Hexyl |
| | | Benzoate |
| 10.00 | Z-COTE MAX | Zinc Oxide (and) Diphenyl Capryl |
| | | Methicone |
| 12.00 | Beeswax 3044 PH | Beeswax |
| 3.00 | Vaseline | Petrolatum |
| 8.00 | Candelilla wax LT 281 LJ | Candelilla (Euphorbia Cerifera) Wax |
| 8.00 | Paraffin oil, high-viscosity | Mineral Oil |
| 5.00 | Tegin | Glyceryl Stearate SE |
| 5.00 | Softisan 154 | Hydrogenated Palm Oil |
| 5.00 | Witconol APM | PPG-3 Myristyl Ether |
| 5.00 | Dow Corning 345 Fluid | Cyclopentasiloxane, |
| | | Cyclohexasiloxane |
| 0.1% | Fusion protein-KBD | |
| 28.90 | castor oil | Castor (Ricinus Communis) Oil |

| | | | |
|---|---|---|---|
| | 5.00 | T-Lite SF | Titanium Dioxide, Alumina |
| | | | Hydrate, Dimethicone/Methicone |
| | | | Copolymer |
| | 6.00 | Finsolv TN | C12-15 Alkyl Benzoate |
| | 10.00 | Uvinul MC 80 | Ethylhexyl Methoxycinnamate |
| | 6.00 | Miglyol 812 | Caprylic/Capric Triglyceride |
| | 5.00 | Arlacel P 135 | PEG-30 Dipolyhydroxystearate |
| | 2.00 | Ganex V 216 | PVP/Hexadecene Copolymer |
| | 2.00 | Elfacos ST 9 | PEG-45/Dodecyl Glycol Copolymer |
| B | 3.00 | 1,2-Propylene | Propylene Glycol |
| | | Glycol Care | |
| | 0.10 | Edeta BD | Disodium EDTA |
| | 1.00 | Magnesium sulfate | Magnesium Sulfate |
| | | 7-hydrate | |
| | 59.40 | Water demin. | Aqua dem. |
| | 0.5% | Fusion protein-KBD | |
| | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 4.00 | Uvinul MC 80 | Ethylhexyl Methoxycinnamate |
| | 2.00 | Uvinul N 539 T | Octocrylene |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate 7-hydrate | |
| | ad 100 | Water demin. | Aqua dem. |
| | 1.5% | Fusion protein- | |
| | | KBD | |
| D | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 4.00 | Uvinul MC 80 | Ethylhexyl Methoxycinnamate |
| | 2.00 | Uvinul N 539 T | Octocrylene |
| B | 5.00 | T-Lite SF-S | Titanium Dioxide, Silica Hydrate, |
| | | | Alumina Hydrate, Methicone/ |
| | | | Dimethicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate 7-hydrate | |
| | 1% | Fusion protein- | |
| | | KBD | |
| | ad 100 | Water demin. | Aqua dem. |
| D | q.s. | preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 8.00 | Uvinul A | Ethylhexyl Methoxycinnamate and |
| | | Plus B | Diethylamino Hydroxybenzoyl hexyl |
| | | | Benzoate |
| | 2.00 | Uvinul N 539 T | Octocrylene |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate 7-hydrate | |
| | 0.1% | Fusion protein- | |
| | | KBD | |
| | ad 100 | Water demin. | Aqua dem. |
| | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls | Polyglyceryl-2 Dipolyhydroxystearate |
| | | PGPH | |
| | 8.00 | Finsolv TN | C12-15 Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl Hexyl |
| | | | Benzoate |
| | 2.00 | Uvinul N 539 T | Octocrylene |
| B | 5.00 | T-Lite SF | Titanium Dioxide, Alumina |
| | | | Hydrate, Dimethicone/Methicone |
| | | | Copolymer |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate | |
| | | 7-hydrate | |
| | ad 100 | Water demin. | Aqua dem. |
| | 0.2% | Fusion protein- | |
| | | KBD | |
| | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 8.00 | Uvinul A Plus B | Ethylhexyl Methoxycinnamate and |
| | | | Diethylamino Hydroxybenzoyl hexyl |
| | | | Benzoate |
| | 2.00 | Uvinul N 539 T | Octocrylene |
| B | 5.00 | T-Lite SF-S | Titanium Dioxide, Silica Hydrate, |
| | | | Alumina Hydrate, Methicone/ |
| | | | Dimethicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate 7-hydrate | |
| | 0.5% | Fusion protein- | |
| | | KBD | |
| | ad 100 | Water demin. | Aqua dem. |
| | q.s. | Preservative | |

| | | | |
|---|---|---|---|
| A | 4.00 | Dehymuls SBL | Polyglyceryl-2 Dipolyhydroxystearate, |
| | | | Dicaprylyl Ether, Cocoglycerides, |
| | | | Sorbitan Sesquioleate, Cera Alba, |
| | | | Aluminum Stearates, Dicocoyl |
| | | | Pentaerythrityl Distearyl Citrate |
| | 1.00 | Dehymuls PGPH | Polyglyceryl-2 Dipolyhydroxystearate |
| | 8.00 | Finsolv TN | C12-15 Alkyl Benzoate |
| | 4.00 | Miglyol 812 | Caprylic/Capric Triglyceride |
| | 7.00 | Uvinul MC 80 | Ethylhexyl Methoxycinnamate |
| | 2.00 | Mexoryl XL | Drometrizole Trisiloxane |
| B | 5.00 | Z-COTE MAX | Titanium Dioxide, Alumina Hydrate, |
| | | | Dimethicone/Methicone Copolymer |
| C | 3.00 | 1,2-Propylene | Propylene Glycol |
| | | Glycol Care | |
| | 0.30 | Abiol | Imidazolidinyl Urea |
| | 1.00 | Magnesium | Magnesium Sulfate |
| | | sulfate 7-hydrate | |
| | ad 100 | Water demin. | Aqua dem. |
| | 0.01% | Fusion protein- | |
| | | KBD | |
| | q.s. | Preservative | |

| | | | |
|---|---|---|---|
| A | 7.50 | Uvinul MC 80 | Ethylhexyl Methoxycinnamate |
| | 3.00 | Uvinul N 539 T | Octocrylene |
| | 2.00 | Uvinul A Plus | Diethylamino Hydroxybenzoyl Hexyl |
| | | | Benzoate |
| | 1.00 | Cremophor CO 40 | PEG-40 Hydrogenated Castor Oil |
| | 10.00 | Miglyol 812 | Caprylic/Capric Triglyceride |
| | 1.50 | Dow Corning 345 | Cyclopentasiloxane, |
| | | Fluid | Cyclohexasiloxane |
| B | 3.50 | Luvigel EM | Caprylic/Capric Triglyceride, Sodium |
| | | | Acrylates Copolymer |
| C | 46.00 | Water demin. | Aqua dem. |
| D | 5.00 | 1,2-Propylene | Propylene Glycol |
| | | Glycol Care | |
| | 0.50 | Cremophor A 25 | Ceteareth-25 |
| | 0.05% | Fusion protein-KBD | |
| | 20.00 | Ethanol 96% | Alcohol |

| | | | |
|---|---|---|---|
| A | 1.00 | Uvinul A Plus | Diethylamino Hydroxybenzoyl Hexyl |
| | | | Benzoate |
| | 1.00 | Tinosorb S | Bis-Ethylhexyloxyphenol |
| | | | Methoxyphenyl Triazine |
| | 3.00 | Uvinul MC 80 | Ethylhexyl Methoxycinnamate |
| | 8.00 | Miglyol 812 | Caprylic/Capric Triglyceride |
| | 1.50 | Dow Corning 350 | Dimethicone |
| | | Fluid | |
| | 3.00 | Z-COTE MAX | Titanium Dioxide, Alumina Hydrate, |
| | | | Dimethicone/Methicone Copolymer |
| | 3.00 | Finsolv TN | C12-15 Alkyl Benzoate |
| | 1.00 | Cremophor CO 40 | PEG-40 Hydrogenated Castor Oil |
| B | 2.00 | Luvigel EM | Caprylic/Capric Triglyceride, Sodium |
| | | | Acrylates Copolymer |
| C | 54.80 | Water demin. | Aqua dem. |
| D | 15.00 | Ethanol 96% | Alcohol |
| | 5.00 | 1,2-Propylene Glycol | Propylene Glycol |
| | | Care | |
| | 0.50 | Cremophor A 25 | Ceteareth-25 |
| | 1% | Fusion protein-KBD | |
| | 1.00 | Vitamin E acetate | Tocopheryl Acetate |
| | 0.20 | Bisabolol rac. | Bisabolol |

| | | | |
|---|---|---|---|
| A | 4.00 | Dehymuls SBL | Polyglyceryl-2 Dipolyhydroxy- |
| | | | stearate, Dicaprylyl Ether, |
| | | | Cocoglycerides, Sorbitan |
| | | | Sesquioleate, Cera Alba, Aluminum |
| | | | Stearates, Dicocoyl Pentaerythrityl |
| | | | Distearyl Citrate |
| | 1.00 | Dehymuls PGPH | Polyglyceryl-2 |
| | | | Dipolyhydroxystearate |
| | 6.00 | Finsolv TN | C12-15 Alkyl Benzoate |
| | 6.00 | Miglyol 812 | Caprylic/Capric Triglyceride |
| | 5.00 | Uvinul MC 80 | Ethylhexyl Methoxycinnamate |
| | 3.00 | Neoheliopan HMS | Homosalate |
| B | 5.00 | Z-COTE MAX | Zinc Oxide (and) Diphenyl Capryl |
| | | | Methicone |
| C | 3.00 | 1,2-Propylene | Propylene Glycol |
| | | Glycol Care | |
| | 0.30 | Chemag 2000 | Imidazolidinyl Urea |
| | 1.00 | Magnesium sulfate | Magnesium Sulfate |
| | | 7-hydrate | |
| | 65.10 | Water demin. | Aqua dem. |
| | 0.1% | Fusion protein-KBD | |
| | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Uvinul N 539 T | Octocrylene |
| | 2.00 | Uvinul T 150 | Ethylhexyl Triazone |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | T-Lite SF | Titanium Dioxide, Alumina |
| | | | Hydrate, Dimethicone/Methicone |
| | | | Copolymer |
| C | 60.00 | Water demin. | Water |
| | 5.00 | Glycerin 87% | Glycerin |
| | 1.00 | Lanette E | Sodium Cetearyl Sulfate |
| | 0.30 | Keltrol | Xanthan Gum |
| | 0.2% | Fusion protein- | |
| | | KBD | |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Uvinul N 539 T | Octocrylene |
| | 2.00 | Neoheliopan HMS | Homosalate |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | T-Lite SF | Titanium Dioxide, Alumina Hydrate, |
| | | | Dimethicone/Methicone Copolymer |
| C | 59.20 | Water demin. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| | 1% | Fusion protein-KBD | |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul MC 80 | Ethylhexyl Methoxycinnamate |
| | 3.00 | Neoheliopan OS | Octisalate |
| | 2.00 | Neoheliopan HMS | Homosalate |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | Z-COTE MAX | Zinc Oxide (and) Diphenyl Capryl |
| | | | Methicone |
| C | 60.15 | Water demin. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| | 0.05% | Fusion protein-KBD | |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul N 539 T | Octocrylene |
| | 3.00 | Neoheliopan OS | Octisalate |
| | 2.00 | Neoheliopan HMS | Homosalate |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | Z-COTE MAX | Zinc Oxide (and) Diphenyl Capryl |
| | | | Methicone |
| C | 60.19 | Water demin. | Aqua dem. |
| | 5.00 | Glycerin 87% | Glycerin |
| | 1.00 | Lanette E | Sodium Cetearyl Sulfate |
| | 0.30 | Keltrol | Xanthan Gum |
| | 0.01% | Fusion protein-KBD | |
| D | q.s. | Preservative | |

| | | | |
|---|---|---|---|
| A | 4.00 | Tego Care 450 | Polyglyceryl-3 Methyl Glucose |
| | | | Distearate |
| | 4.50 | Uvinul N 539 T | Octocrylene |
| | 3.00 | Neoheliopan OS | Octisalate |
| | 2.00 | Neoheliopan HMS | Homosalate |
| | 0.50 | Vitamin E acetate | Tocopheryl Acetate |
| B | 5.00 | Isohexadecane | Isohexadecane |
| | 5.00 | Cosmacol EMI | Di-C12-13 Alkyl Malate |
| | 3.50 | Cetiol SN | Cetearyl Isononanoate |
| | 1.00 | Ganex V-220 | VP/Eicosene Copolymer |
| | 5.00 | T-Lite SF-S | Titanium Dioxide, Silica Hydrate, |
| | | | Alumina Hydrate, |
| | | | Methicone/Dimethicone Copolymer |
| C | 59.20 | Water demin. | Aqua dem. |
| | 5.00 | Glycerin 87% | Glycerin |
| | 1.00 | Lanette E | Sodium Cetearyl Sulfate |
| | 0.3 | Keltrol | Xanthan Gum |
| | 1% | Fusion protein-KBD | |
| D | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb S | Bis-Ethylhexyloxyphenol |
| | | | Methoxyphenyl Triazine |
| | 1.00 | Vitamin E acetate | Tocopheryl Acetate |
| B | 4.00 | Z-COTE MAX | Zinc Oxide (and) Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1.50 | Veegum Ultra | Magnesium Aluminum Silicate |
| | 49.45 | Water demin. | Aqua dem. |
| | 1% | Fusion protein-KBD | |
| D | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 3.00 | Tinosorb S | Bis-Ethylhexyloxyphenol |
| | | | Methoxyphenyl Triazine |
| | 1.00 | Vitamin E acetate | Tocopheryl Acetate |
| B | 4.00 | T-Lite SF | Titanium Dioxide, Alumina Hydrate, |
| | | | Dimethicone/Methicone Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1.50 | Veegum Ultra | Magnesium Aluminum Silicate |
| | 50.25 | Water demin. | Aqua dem. |
| D | 0.50 | Citric acid | Citric Acid |
| | q.s. | Perfume oil | |
| | 0.2% | Fusion protein-KBD | |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul T 150 | Ethylhexyl Triazone |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 2.50 | T-Lite SF | Titanium Dioxide, Alumina Hydrate, |
| | | | Dimethicone/Methicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 42.00 | Water demin. | Aqua dem. |
| D | 5.00 | Eusolex 232 | Phenylbenzimidazole Sulfonic Acid |
| | 10.00 | Water demin. | Aqua dem. |
| E | 0.70 | Sodium hydroxide | Sodium Hydroxide |
| | 1% | Fusion protein-KBD | |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul A Plus | Diethylamino Hydrobenzoyl Hexyl |
| | | | Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 2.50 | T-Lite SF | Titanium Dioxide, Alumina Hydrate, |
| | | | Dimethicone/Methicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 41.00 | Water demin. | Aqua dem. |
| D | 5.00 | Eusolex 232 | Phenylbenzimidazole Sulfonic Acid |
| | 2% | Fusion protein-KED | |
| | 10.00 | Water demin. | Aqua dem. |
| E | 0.7 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 4.50 | Uvinul MC 80 | Ethylhexyl Methoxycinnamate |
| | 3.00 | Uvinul N 539 T | Octocrylene |
| | 2.00 | Uvinul A Plus | Diethylamino Hydrobenzoyl Hexyl |
| | | | Benzoate |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 2.50 | Z-COTE MAX | Zinc Oxide (And) Diphenyl Capryl |
| | | | Methicone |
| C | 5.00 | Glycerin 87% | Glycerin |
| | 2.00 | Lanette E | Sodium Cetearyl Sulfate |
| | 0.3 | Keltrol | Xanthan Gum |
| | 1.00 | Pationic 138 C | Sodium Lauroyl Lactylate |
| | 1.00 | Pationic SSL | Sodium Stearoyl Lactylate |
| | 42.50 | Water demin. | Aqua dem. |
| D | 5.00 | Eusolex 232 | Phenylbenzimidazole Sulfonic Acid |
| | 0.5% | Fusion protein-KBD | |
| | 10.00 | Water demin. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 7.50 | Uvinul N 539 T | Octocrylene |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 5.00 | Z-COTE MAX | Zinc Oxide (And) Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 40.50 | Water demin. | Aqua dem. |
| D | 5.00 | Eusolex 232 | Phenylbenzimidazole Sulfonic Acid |
| | 1% | Fusion protein-KBD | |
| | 10.00 | Water demin. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 5.50 | Uvinul N 539 T | Octocrylene |
| | 2.00 | Uvinul A Plus | Diethylamino Hydrobenzoyl Hexyl |
| | | | Benzoate |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 5.00 | Z-COTE MAX | Zinc Oxide (And) Diphenyl Capryl |
| | | | Methicone |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 41.50 | Water demin. | Aqua dem. |
| D | 5.00 | Eusolex 232 | Phenylbenzimidazole Sulfonic Acid |
| | 0.5% | Fusion protein-KBD | |
| | 9.50 | Water demin. | Aqua dem. |
| E | 0.70 | Sodium hydroxide | Sodium Hydroxide |
| | q.s. | Preservative | |

| | | | |
|---|---|---|---|
| A | 5.00 | Cosmacol EMI | Di-C12-13 Alkyl Malate |
| | 5.50 | Uvinul N 539 T | Octocrylene |
| | 2.00 | Uvinul A Plus | Diethylamino Hydrobenzoyl Hexyl |
| | | | Benzoate |
| | 2.00 | Mexoryl XL | Drometrizole Trisiloxane |
| B | 4.00 | Tego Care 450 | Polyglyceryl-3 Methyl Glucose |
| | | | Distearate |
| | 5.00 | Isohexadecane | Isohexadecane |
| | 3.50 | Cetiol SN | Cetearyl Isononanoate |
| | 0.50 | Vitamin E acetate | Tocopheryl Acetate |
| | 1.00 | Ganex V-220 | VP/Eicosene Copolymer |
| | 5.00 | T-Lite SF-S | Titanium Dioxide, Silica Hydrate, |
| | | | Alumina Hydrate, |
| | | | Methicone/Dimethicone Copolymer |
| C | 5.00 | Glycerin 87% | Glycerin |
| | 2.00 | Lanette E | Sodium Cetearyl Sulfate |
| | 0.30 | Keltrol | Xanthan Gum |
| | 1.00 | Pationic 138 C | Sodium Lauroyl Lactylate |
| | 1.00 | Pationic SSL | Sodium Stearoyl Lactylate |
| | 40.50 | Water demin. | Aqua dem. |
| D | 5.00 | Eusolex 232 | Phenylbenzimidazole Sulfonic Acid |
| | 1% | Fusion protein-KBD | |
| | 10.00 | Water demin. | Aqua dem. |
| E | 0.70 | Sodium hydroxide | Sodium Hydroxide |
| | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 10.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb S | Bis-Ethylhexyloxyphenol |
| | | | Methoxyphenyl Triazine |
| | 1.00 | Uvinul T 150 | Ethylhexyl Triazone |
| | 1.00 | Vitamin E acetate | Tocopheryl Acetate |
| B | 5.00 | T-Lite SF | Titanium Dioxide, Alumina Hydrate, |
| | | | Dimethicone/Methicone Copolymer |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 2% | Fusion protein-KBD | |
| | 1.50 | Veegum Ultra | Magnesium Aluminum Silicate |
| | | ad 100 Water | Aqua dem. |
| | | demin. | |
| D | q.s. | Perfume oil | |
| | q.s. | Preservative | |

| | | | |
|---|---|---|---|
| A | 8.00 | Cetiol B | Dibutyl Adipate |
| | 8.00 | Finsolv TN | C12-15 Alkyl Benzoate |
| | 10.00 | Myritol 331 | Cocoglycerides |
| | 1.00 | Lanette E | Sodium Cetearyl Sulfate |
| | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 1.00 | Tinosorb S | Bis-Ethylhexyloxyphenol |
| | | | Methoxyphenyl triazine |
| | 1.00 | Uvinul T 150 | Ethylhexyl Triazone |
| | 1.00 | Vitamin E acetate | Tocopheryl Acetate |
| B | 5.00 | Z-COTE MAX | Zinc Oxide (and) Diphenyl Capryl |
| | | | Methicone |
| C | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 1% | Fusion protein-KBD | |
| | 1.50 | Veegum Ultra | Magnesium Aluminum Silicate |
| | | ad 100 Water demin. | Aqua dem. |
| D | q.s. | Perfume oil | |
| | q.s. | Preservative | |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 57.00 | Water demin. | Aqua dem. |
| B | 5.00 | T-Lite SF-S | Titanium Dioxide, Silica Hydrate, |
| | | | Alumina Hydrate, |
| | | | Methicone/Dimethicone Copolymer |
| C | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 2% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 58.00 | Water demin. | Aqua dem. |
| B | 5.00 | Z-COTE MAX | Zinc Oxide (and) Diphenyl Capryl |
| | | | Methicone |
| C | 7.50 | Uvinul MC 80 | Ethylhexyl Methoxycinnamate |
| | 2.00 | Uvinul A Plus | Diethylamino Hydroxybenzoyl |
| | | | Hexyl Benzoate |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 1% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 58.50 | Water demin. | Aqua dem. |
| B | 5.00 | T-Lite SF-S | Titanium Dioxide, Silica Hydrate, |
| | | | Alumina Hydrate, |
| | | | Methicone/Dimethicone Copolymer |
| C | 7.50 | Uvinul MC 80 | Ethylhexyl Methoxycinnamate |
| | 5.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 0.5% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Mexoryl SL | Terephthalidene Dicamphor |
| | | | Sulfonic Acid |
| | 58.40 | Water demin. | Aqua dem. |
| B | 6.00 | T-Lite SF-S | Titanium Dioxide, Silica Hydrate, |
| | | | Alumina Hydrate, |
| | | | Methicone/Dimethicone Copolymer |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 0.1% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Mexoryl SL | Terephthalidene Dicamphor |
| | | | Sulfonic Acid |
| | 58.45 | Water demin. | Aqua dem. |
| B | 6.00 | Z-COTE MAX | Zinc Oxide (and) Diphenyl Capryl |
| | | | Methicone |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 0.05% | Fusion protein-KBD | |
| | 0.20 | Bisabolol rac. | Bisabolol |

| | | | |
|---|---|---|---|
| A | 3.00 | Glycerin 87% | Glycerin |
| | 0.20 | Edeta BD | Disodium EDTA |
| | 0.30 | Abiol | Imidazolidinyl Urea |
| | 1.00 | Plantacare 2000 | Decyl Glucoside |
| | 0.30 | Keltrol T | Xanthan Gum |
| | 2.00 | Neoheliopan AP | Disodium Phenyl Dibenzimidazole |
| | | | Tetrasulfonate |
| | 57.50 | Water demin. | Aqua dem. |
| B | 6.00 | Z-COTE MAX | Zinc Oxide (and) Diphenyl Capryl |
| | | | Methicone |
| | 6.00 | Uvinul N 539 T | Octocrylene |
| | 2.00 | Parsol 1789 | Butyl Methoxydibenzoylmethane |
| | 2.00 | Dow Corning 345 | Cyclopentasiloxane, |
| | | Fluid | Cyclohexasiloxane |
| | 3.50 | Cremophor A 6 | Ceteareth-6, Stearyl Alcohol |
| | 1.50 | Cremophor A 25 | Ceteareth-25 |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| D | 1.00 | Vitamin E acetate | Tocopheryl Acetate |
| | 1.0% | Fusion protein-KBD | |
| | 0.20 | Bisabolol rac. | Bisabolol |

| | | | |
|---|---|---|---|
| A | 25.00 | Dow Corning 345 | Cyclopentasiloxane, |
| | | Fluid | Cyclohexasiloxane |
| | 20.00 | Dow Corning 245 | Cyclopentasiloxane |
| | | Fluid | |
| | 8.00 | Uvinul MC 80 | Ethylhexyl Methoxycinnamate |
| | 4.00 | Abil EM 90 | Cetyl PEG/PPG-10/1 Dimethicone |
| | 7.00 | T-Lite SF | Titanium Dioxide, Alumina Hydrate, |
| | | | Dimethicone/Methicone Copolymer |
| B | 17.00 | Ethanol 95% | Alcohol |
| | 10.00 | Z-COTE MAX | Zinc Oxide (and) Diphenyl Capryl |
| | | | Methicone |
| | 4.50 | Water demin. | Aqua dem. |
| | 3.00 | Glycerin 87% | Glycerin |
| | 0.5% | Fusion protein-KBD | |
| | 1.00 | talc | Talc |

| | | | |
|---|---|---|---|
| A | 25.00 | Dow Corning 345 | Cyclopentasiloxane, |
| | | Fluid | Cyclohexasiloxane |
| | 20.00 | Dow Corning 245 | Cyclopentasiloxane |
| | | Fluid | |
| | 8.00 | Uvinul MC 80 | Ethylhexyl Methoxycinnamate |
| | 4.00 | Abil EM 90 | Cetyl PEG/PPG-10/1 Dimethicone |
| | 7.00 | T-Lite SF-S | Titanium Dioxide, Silica Hydrate, |
| | | | Alumina Hydrate, |
| | | | Methicone/Dimethicone Copolymer |
| B | 17.00 | Ethanol 95% | Alcohol |
| | 9.99 | Z-COTE MAX | Zinc Oxide (and) Diphenyl Capryl |
| | | | Methicone |
| | 5.00 | Water demin. | Aqua dem. |
| | 3.00 | Glycerin 87% | Glycerin |
| | 0.01% | Fusion protein-KBD | |
| | 1.00 | talc | Talc |

| | | | |
|---|---|---|---|
| A | 20.00 | Dow Corning 345 | Cyclopentasiloxane, |
| | | Fluid | Cyclohexasiloxane |
| | 10.00 | Dow Corning 245 | Cyclopentasiloxane |
| | | Fluid | |
| | 7.50 | Uvinul MC 80 | Ethylhexyl Methoxycinnamate |
| | 2.00 | Uvinul T 150 | Ethylhexyl Triazone |
| | 12.00 | Cosmacol EMI | Di-C12-13 Alkyl Malate |
| | 4.00 | Abil EM 90 | Cetyl PEG/PPG-10/1 Dimethicone |
| B | 7.00 | T-Lite SF | Titanium Dioxide, Alumina Hydrate, |
| | | | Dimethicone/Methicone Copolymer |
| C | 15.00 | Ethanol 95% | Alcohol |
| | 10.00 | Z-COTE MAX | Zinc Oxide (and) Diphenyl Capryl |
| | | | Methicone |
| | 3.00 | Glycerin 87% | Glycerin |
| | 1.5% | Fusion protein-KBD | |
| | 8.00 | Water demin. | Aqua dem. |

| | | | |
|---|---|---|---|
| A | 20.00 | Dow Corning 345 | Cyclopentasiloxane, |
| | | Fluid | Cyclohexasiloxane |
| | 10.00 | Dow Corning 245 | Cyclopentasiloxane |
| | | Fluid | |
| | 7.50 | Uvinul MC 80 | Ethylhexyl Methoxycinnamate |
| | 2.00 | Tinosorb S | Bis-Ethylhexyloxyphenol |
| | | | Methoxyphenyl Triazine |
| | 12.00 | Cosmacol EMI | Di-C12-13 Alkyl Malate |
| | 4.00 | Abil EM 90 | Cetyl PEG/PPG-10/1 Dimethicone |
| B | 7.00 | T-Lite SF | Titanium Dioxide, Alumina Hydrate, |
| | | | Dimethicone/Methicone Copolymer |
| C | 15.00 | Ethanol 95% | Alcohol |
| | 10.00 | Z-COTE MAX | Zinc Oxide (and) Diphenyl Capryl |
| | | | Methicone |
| | 3.00 | Glycerin 87% | Glycerin |
| | 0.5% | Fusion protein-KBD | |
| | 9.00 | Water demin. | Aqua dem. |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | T-Lite SF | Titanium Dioxide, Alumina Hydrate, |
| | | | Dimethicone/Methicone Copolymer |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 61.30 | Water demin. | Aqua dem. |
| D | 1.00 | Vitamin E acetate | Tocopheryl Acetate |
| | 1% | Fusion protein-KBD | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | Z-COTE MAX | Zinc Oxide (and) Diphenyl Capryl |
| | | | Methicone |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 62.20 | Water demin. | Aqua dem. |
| D | 1.00 | Vitamin E acetate | Tocopheryl Acetate |
| | 0.1% | Fusion protein-KBD | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 3.00 | Z-COTE MAX | Zinc Oxide (and) Diphenyl Capryl |
| | | | Methicone |
| | 4.00 | Isopropyl Palmitate | Isopropyl Palmitate |
| | 1.00 | Abil 350 | Dimethicone |
| B | 7.00 | Uvinul MC 80 | Ethylhexyl Methoxycinnamate |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 60.30 | Water demin. | Aqua dem. |
| D | 1.00 | Vitamin E acetate | Tocopheryl Acetate |
| | 2% | Fusion protein-KBD | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | Z-COTE MAX | Zinc Oxide (and) Diphenyl Capryl |
| | | | Methicone |
| | 4.00 | Isopropyl Palmitate | Isopropyl Palmitate |
| | 1.00 | Abil 350 | Dimethicone |
| B | 4.00 | Uvinul N 539 T | Octocrylene |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 62.10 | Water demin. | Aqua dem. |
| D | 1.00 | Vitamin E acetate | Tocopheryl Acetate |
| | 0.2% | Fusion protein-KBD | |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | Z-COTE MAX | Zinc Oxide (and) Diphenyl Capryl |
| | | | Methicone |
| | 4.00 | Isopropyl Palmitate | Isopropyl Palmitate |
| | 1.00 | Abil 350 | Dimethicone |
| B | 3.00 | Uvinul N 539 T | Octocrylene |
| | 1.00 | Mexoryl XL | Drometrizole Trisiloxane |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 1% | Fusion protein-KBD | |
| | 61.30 | Water demin. | Aqua dem. |
| D | 1.00 | Vitamin E acetate | Tocopheryl Acetate |
| | q.s. | Preservative | |

| | | | |
|---|---|---|---|
| A | 2.00 | Cremophor WO 7 | PEG-7 Hydrogenated Castor Oil |
| | 6.00 | Abil B 8839 | Cyclopentasiloxane, |
| | | | Cyclohexasiloxane |
| | 6.00 | T-Lite SF-S | Titanium Dioxide, Silica Hydrate, |
| | | | Alumina Hydrate, |
| | | | Methicone/Dimethicone Copolymer |
| | 4.00 | Isopropyl Palmitate | Isopropyl Palmitate |
| | 1.00 | Abil 350 | Dimethicone |
| B | 3.00 | Uvinul N 539 T | Octocrylene |
| | 1.00 | Mexoryl XL | Drometrizole trisiloxane |
| | 1.00 | Tinosorb S | Bis-Ethylhexyloxyphenol |
| | | | Methoxyphenyl Triazine |
| | 7.00 | Finsolv TN | C12-15 Alkyl Benzoate |
| | 4.00 | Abil WE 09 | Polyglyceryl-4 Isostearate, Cetyl |
| | | | PEG/PPG-10/1 Dimethicone, Hexyl |
| | | | Laurate |
| C | 0.50 | Sodium chloride | Sodium Chloride |
| | 0.20 | Edeta BD | Disodium EDTA |
| | 61.80 | Water demin. | Aqua dem. |
| D | 1.00 | Vitamin E acetate | Tocopheryl Acetate |
| | 0.5% | Fusion protein-KBD | |
| | q.s. | Preservative | |

| | | | |
|---|---|---|---|
| A | 4.50 | Uvinul MC 80 | Ethylhexyl Methoxycinnamate |
| | 3.00 | Uvinul N 539 T | Octocrylene |
| | 1.00 | Uvinul T 150 | Ethylhexyl Triazone |
| | 2.50 | Cosmacol EMI | Di-C12-13 Alkyl Malate |
| B | 0.50 | Vitamin E acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | Z-COTE MAX | Zinc Oxide (and) Diphenyl Capryl |
| | | | Methicone |
| D | 44.00 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1% | Fusion protein-KBD | |
| | 0.50 | Keltrol | Xanthan Gum |
| E | 1.00 | Phenonip | |

| | | | |
|---|---|---|---|
| A | 4.50 | Uvinul MC 80 | Ethylhexyl Methoxycinnamate |
| | 3.00 | Uvinul N 539 T | Octocrylene |
| | 1.00 | Tinosorb S | Bis-Ethoxylhexyloxyphenol |
| | | | Methoxyphenyl Triazine |
| | 2.50 | Cosmacol EMI | Di-C12-13 Alkyl Malate |
| B | 0.50 | Vitamin E acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | Z-COTE MAX | Zinc Oxide (and) Diphenyl Capryl |
| | | | Methicone |
| D | 44.90 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 0.1% | Fusion protein-KBD | |
| | 0.50 | Keltrol | Xanthan Gum |
| E | 1.00 | Phenonip | |

| | | | |
|---|---|---|---|
| A | 1.50 | Mexoryl XL | Drometrizole Trisiloxane |
| | 5.00 | Uvinul N 539 T | Octocrylene |
| | 1.00 | Tinosorb S | Bis-Ethylhexyloxyphenol |
| | | | Methoxyphenyl Triazine |
| | 2.50 | Cosmacol EMI | Di-C12-13 Alkyl Malate |
| B | 0.50 | Vitamin E acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | GanexV-220 | VP/Eicosene Copolymer |
| | 5.00 | Isohexadecane | Isohexadecane |
| | 2.50 | Cosmacol EMI | Di-C12-13 Alkyl Malate |
| | 5.00 | Z-COTE MAX | Zinc Oxide (and) Diphenyl |
| | | | Capryl Methicone |
| D | 44.50 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1.5% | Fusion protein-KBD | |
| | 0.50 | Keltrol | Xanthan Gum |
| E | 1.00 | Phenonip | |

| | | | |
|---|---|---|---|
| A | 1.50 | Mexoryl XL | Drometrizole Trisiloxane |
| | 5.00 | Uvinul N 539 T | Octocrylene |
| | 1.00 | Tinosorb S | Bis-Ethylhexyloxyphenol |
| | | | Methoxyphenyl Triazine |
| | 2.50 | Cosmacol EMI | Di-C12-13 Alkyl Malate |
| B | 0.50 | Vitamin E acetate | Tocopheryl Acetate |
| | 4.00 | Tego Care 450 | Polyglyceryl-3 Methyl Glucose |
| | | | Distearate |
| C | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 5.00 | T-Lite SF | Titanium Dioxide, Alumina Hydrate, |
| | | | Dimethicone/Methicone Copolymer |
| D | 45.00 | Water demin. | Aqua dem. |
| | 5.00 | Glycerin 87% | Glycerin |
| | 1.00 | Lanette E | Sodium Cetearyl Sulfate |
| | 15.00 | Luviquat Care | Polyquaternium-44 |
| | 1% | Fusion protein-KBD | |
| | 0.50 | Keltrol | Xanthan Gum |
| E | 1.00 | Phenonip | |

| | | | |
|---|---|---|---|
| A | 1.00 | Abil Care 85 | Bis-PEG/PPG-16/16 PEG/PPG-16/16 |
| | | | Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 5.00 | Uvinul MC 80 | Ethylhexyl Methoxycinnamate |
| | 10.00 | Witconol APM | PPG-3 Myristyl Ether |
| | 2.00 | Uvinul T 150 | Ethylhexyl Triazone |
| | 1.00 | Dow Corning 345 | Cyclopentasiloxane, |
| | | Fluid | Cyclohexasiloxane |
| | 5.00 | Uvinul N 539 T | Octocrylene |
| B | 5.00 | Z-COTE MAX | Zinc Oxide (and) Diphenyl Capryl |
| | | | Methicone |
| C | 5.00 | 1,2-Propylene | Propylene Glycol |
| | | glycol | |
| | 0.20 | Keltrol | Xanthan Gum |
| | 0.10 | Edeta BD | Disodium EDTA |
| | 2.0% | Fusion protein-KBD | |
| | 1.50 | Simulgel 600 | Acrylamide/Sodium |
| | | | Acryloyldimethyltaurate Copolymer, |
| | | | Isohexadecane, Polysorbate 80 |
| | 58.40 | Water demin. | Aqua dem. |
| D | q.s. | Perfume oil | |
| | 0.50 | Glydant | DMDM Hydantoin |

| | | | |
|---|---|---|---|
| A | 2.00 | Abil Care 85 | Bis-PEG/PPG-16/16 PEG/PPG-16/16 |
| | | | Dimethicone, Caprylic/Capric |
| | | | Triglyceride |
| | 4.00 | Finsolv TN | C12-15 Alkyl Benzoate |
| | 1.50 | Miglyol 812 | Caprylic/Capric Triglyceride |
| | 0.50 | Vitamin E acetate | Tocopheryl Acetate |
| | 7.50 | Uvinul MC 80 | Ethylhexyl Methoxycinnamate |
| | 4.00 | Cetiol B | Dibutyl Adipate |
| | 3.00 | Luvitol EHO | Cetearyl Ethylhexanoate |
| | 1.00 | Cremophor CO 40 | PEG-40 Hydrogenated Castor Oil |
| | 1.00 | Paraffin oil, | Mineral Oil |
| | | low viscosity | |
| | 3.00 | Plantacare 2000 | Decyl Glucoside |
| | 0.50 | Phenonip | |
| | q.s. | Perfume oil | |
| B | 4.00 | Z-COTE MAX | Zinc Oxide (and) Diphenyl Capryl |
| | | | Methicone |
| C | 2.00 | Simulgel NS | Hydroxyethyl Acrylate/Sodium |
| | | | Acryloyldimethyl Taurate Copolymer, |
| | | | Squalane, Polysorbate 60 |
| | 0.20 | Keltrol | Xanthan Gum |
| | 0.10 | Edeta BD | Disodium EDTA |
| | 1% | Fusion protein-KBD | |
| | 64.20 | Water demin. | Aqua dem. |

| | | | |
|---|---|---|---|
| A | 6.00 | Gilugel SIL 5 | Cyclomethicone (and) Aluminum/ |
| | | | Magnesium Hydroxide Stearate |
| | 5.00 | Uvinul MC 80 | Ethylhexyl Methoxycinnamate |
| | 2.00 | Uvinul A Plus | Diethylamino Hydroxybenzoyl |
| | | | Hexyl Benzoate |
| | 1.00 | Uvinul T 150 | Ethylhexyl Triazone |
| | 7.00 | Finsolv TN | C12-15 Alkyl Benzoate |
| | 4.00 | Abil WE 09 | Polyglyceryl-4 Isostearate, |
| | | | Cetyl PEG/PPG-10/1 |
| | | | Dimethicone, Hexyl Laurate |
| | 2.00 | Cosmacol EMI | Di-C12-13 Alkyl Malate |
| | 3.00 | Isopro palmitate | Isopropyl Palmitate |
| | 5.00 | Abil B 8839 | Cyclopentasiloxane, |
| | | | Cyclohexasiloxane |
| | 0.50 | Abil 350 | Dimethicone |
| B | 0.50 | Sodium chloride | Sodium Chloride |
| | 0.20 | Edeta BD | Disodium EDTA |
| | 0.2% | Fusion protein-KBD | |
| | 62.10 | Water demin. | Aqua dem. |
| C | 1.00 | Vitamin E acetate | Tocopheryl Acetate |
| | 0.50 | Phenonip | |

| | | | |
|---|---|---|---|
| A | 4.50 | Uvinul MC 80 | Ethylhexyl Methoxycinnamate |
| | 2.00 | Tinosorb S | Bis-Ethylhexyloxyphenol |
| | | | Methoxyphenyl Triazine |
| | 3.00 | Uvinul N 539 T | Octocrylene |
| | 2.50 | Cosmacol EMI | Di-C12-13 Alkyl Malate |
| | 0.50 | Vitamin E acetate | Tocopheryl Acetate |
| | 4.00 | Tego Care 450 | Polyglyceryl-3 Methyl Glucose |
| | | | Distearate |
| B | 3.50 | Cetiol SN | Cetearyl Isononanoate |
| | 1.00 | Ganex V-220 | VP/Eicosene Copolymer |
| | 5.00 | Isohexadecane | Isohexadecane |
| | 2.50 | Cosmacol EMI | Di-C12-13 Alkyl Malate |
| | 3.00 | T-Lite SF | Titanium Dioxide, Alumina Hydrate, |
| | | | Dimethicone/Methicone Copolymer |
| C | 5.00 | Glycerin 87% | Glycerin |
| | 1.00 | Lanette E | Sodium Cetearyl Sulfate |
| | 0.50 | Keltrol | Xanthan Gum |
| | 1% | Fusion protein-KBD | |
| | 59.70 | Water demin. | Aqua dem. |
| D | 1.00 | Phenonip | |
| | 0.30 | Bisabolol rac. | Bisabolol |

| | | | |
|---|---|---|---|
| A | 2.00 | Abil Care 85 | Bis-PEG/PPG-16/16 PEG/PPG-16/16 |
| | | | Dimethicone, Caprylic/Capric |
| | | | Triglyceride |
| | 4.00 | Finsolv TN | C12-15 Alkyl Benzoate |
| | 1.50 | Miglyol 812 | Caprylic/Capric Triglyceride |
| | 0.50 | Vitamin E acetate | Tocopheryl Acetate |
| | 7.50 | Uvinul MC 80 | Ethylhexyl Methoxycinnamate |
| | 4.00 | Cetiol B | Dibutyl Adipate |
| | 1.00 | Luvitol EHO | Cetearyl Ethylhexanoate |
| | 1.00 | Cremophor CO 40 | PEG-40 Hydrogenated Castor Oil |
| | 1.00 | Paraffin oil, | Mineral Oil |
| | | low viscosity | |
| | 3.00 | Plantacare 2000 | Decyl Glucoside |
| | 1.00 | Uvinul A Plus | Diethylamino Hydroxybenzoyl Hexyl |
| | | | Benzoate |
| | 0.50 | Phenonip | |
| | 2.50 | Uvinul T 150 | Ethylhexyl Triazone |
| | q.s. | Perfume oil | |
| B | 4.00 | Z-COTE MAX | Zinc Oxide (and) Diphenyl Capryl |
| | | | Methicone |
| C | 2.00 | Simulgel NS | Hydroxyethyl Acrylate/Sodium |
| | | | Acryloyldimethyl Taurate |
| | | | Copolymer, Squalane, |
| | | | Polysorbate 60 |
| | 0.10 | Keltrol | Xanthan Gum |
| | 0.10 | Edeta BD | Disodium EDTA |
| | 0.5% | Fusion protein-KBD | |
| | 59.90 | Water demin. | Aqua dem. |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol 331 | Cocoglycerides |
| | 8.00 | Finsolv TN | C12-15 Alkyl Benzoate |
| | 8.00 | Cetiol B | Dibutyl Adipate |
| B | 6.00 | T-Lite SF | Titanium Dioxide, Alumina Hydrate, |
| | | | Dimethicone/Methicone Copolymer |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 2% | Fusion protein-KBD | |
| | 0.30 | Keltrol | Xanthan Gum |
| | 45.10 | Water demin. | Water |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 12.00 | Myritol 331 | Cocoglycerides |
| | 8.00 | Finsolv TN | C12-15 Alkyl Benzoate |
| | 8.00 | Cetiol B | Dibutyl Adipate |
| B | 6.00 | Uvinul N 539 T | Octocrylene |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 0.30 | Carbopol Ultrez 10 P | Carbomer |
| | 2% | Fusion protein-KBD | |
| | 46.10 | Water demin. | Aqua dem. |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 4.00 | Eumulgin VL 75 | Lauryl Glucoside, Polyglyceryl-2 |
| | | | Dipolyhydroxystearate, Glycerin |
| | 2.00 | Lanette O | Cetearyl Alcohol |
| | 10.00 | Myritol 331 | Cocoglycerides |
| | 8.00 | Finsolv TN | C12-15 Alkyl Benzoate |
| | 8.00 | Cetiol B | Dibutyl Adipate |
| B | 2.00 | Uvinul A Plus | Diethylamino Hydroxybenzoyl |
| | | | Hexyl Benzoate |
| | 5.00 | Z-COTE MAX | Zinc Oxide (and) Diphenyl Capryl |
| | | | Methicone |
| C | 3.00 | Glycerin 87% | Glycerin |
| | 0.10 | Edeta BD | Disodium EDTA |
| | 1.50 | Veegum Ultra | Magnesium Aluminum Silicate |
| | 1.50 | Lanette E | Sodium Cetearyl Sulfate |
| | 5.0% | Fusion protein-KBD | |
| | 0.30 | Carbopol Ultrez 10 P | Carbomer |
| | | add Water demin. | Water |
| D | 1.00 | Phenonip | Phenoxyethanol, Methylparaben, |
| | | | Ethylparaben, Butylparaben, |
| | | | Propylparaben, Isobutylparaben |

| | | | |
|---|---|---|---|
| A | 3.50 | Cremophor A 6 | Ceteareth-6, Stearyl Alcohol |
| | 1.50 | Cremophor A 25 | Ceteareth-25 |
| | 7.50 | Uvinul MC 80 | Ethylhexyl Methoxycinnamate |
| | 2.00 | Uvinul A Plus | Diethylamino Hydroxybenzoyl Hexyl |
| | | | Benzoate |
| | 2.00 | Dow Corning 345 | Cyclopentasiloxane, |
| | | | Cyclohexasiloxane Fluid |
| | 0.50 | Beeswax 3044 PH | Beeswax |
| | 3.00 | Lanette O | Cetearyl Alcohol |
| | 10.00 | Miglyol 812 | Caprylic/Capric Triglyceride |
| B | 5.00 | T-Lite SF-S Titanium | Titanium Dioxide, Silica Hydrate, |
| | | | Alumina Hydrate, Methicone/ |
| | | | Dimethicone Copolymer |
| C | 3.00 | Glycerin 87% | Glycerin |
| | 0.20 | Edeta BD | Disodium EDTA |
| | 0.30 | Keltrol T | Xanthan Gum |
| | 1.00 | Plantacare 2000 | Decyl Glucoside |
| | 1% | Fusion protein-KBD | |
| | 57.30 | Water demin. | Aqua dem. |
| D | 1.00 | Vitamin E acetate | Tocopheryl Acetate |
| | 0.20 | Bisabolol rac. | Bisabolol |

| | | | |
|---|---|---|---|
| A | 10.00 | Uvinul A Plus B | Ethylhexyl Methoxycinnamate, |
| | | | Diethylamino Hydroxybenzoyl |
| | | | Hexyl Benzoate |
| | 10.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 3.00 | Z-COTE MAX | Zinc Oxide (and) Diphenyl |
| | | | Capryl Methicone |
| C | 35.08 | Water demin. | Aqua dem. |
| | 0.38 | Citric acid | Citric Acid |
| | 2.9 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 0.1% | Fusion protein-KBD | |
| | 1.50 | Veegum Ultra | Magnesium Aluminum Silicate |
| D | 0.50 | Phenonip | |
| | 1.00 | Vitamin E acetate | Tocopheryl Acetate |

| | | | |
|---|---|---|---|
| A | 0.70 | Cremophor A 25 | Ceteareth-25 |
| | 1.70 | Cremophor A 6 | Ceteareth-6, Stearyl Alcohol |
| | 2.00 | Uvinul A Plus | Diethylamino Hydroxybenzoyl Hexyl |
| | | | Benzoate |
| | 3.00 | Uvinul N 539 T | Octocrylene |
| | 3.00 | Z-COTE MAX | Zinc Oxide (and) Diphenyl Capryl |
| | | | methicone |
| | 2.00 | Abil B 8843 | PEG-14 Dimethicone |
| | 3.60 | Lanette O | Cetearyl Alcohol |
| | 4.00 | Uvinul MC 80 | Ethylhexyl Methoxycinnamate |
| | 2.00 | Cetiol B | Dibutyl Adipate |
| B | 4.00 | Glycerin 87% | Glycerin |
| | 0.20 | Edeta BD | Disodium EDTA |
| | 71.00 | Water demin. | Panthenol |
| C | 4.00 | Luvigel EM | Caprylic/Capric Triglyceride, Sodium |
| | | | Acrylates Copolymer |
| D | 1.00 | Vitamin E acetate | Tocopheryl Acetate |
| | 1% | Fusion protein-KBD | |
| | 0.20 | Bisabolol rac. | Bisabolol |
| | q.s. | Preservative | |

| | | | |
|---|---|---|---|
| A | 7.50 | Uvinul MC 80 | Ethylhexyl Methoxycinnamate |
| | 5.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.50 | Cremophor WO 7 | PEG-7 Hydrogenated Castor Oil |
| | 0.50 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl Capryl |
| | | | Methicone |
| | 0.03 | Sicomet Blue | C.I. 77 007, Ultramarines |
| | | P 77 007 | |
| C | 4.00 | 1,2-Propylene | Propylene Glycol |
| | | Glycol Care | |
| | 2.00 | D-Panthenol 50 P | Panthenol, Propylene Glycol |
| | 0.20 | Keltrol | Xanthan Gum |
| | 0.50 | Simulgel 600 | Acrylamide/Sodium |
| | | | Acryloyldimethyltaurate Copolymer, |
| | | | Isohexadecane, Polysorbate 80 |
| | 0.5% | Fusion protein-KBD | |
| | 65.27 | Water demin. | Aqua dem. |
| | q.s. | Preservative | |
| | 0.50 | Vitamin E acetate | Tocopheryl Acetate |

| | | | |
|---|---|---|---|
| A | 6.00 | Gilugel SIL 5 | Cyclomethicone (and) Aluminum/ |
| | | | Magnesium Hydroxide Stearate |
| | 5.00 | Uvinul N 539 T | Octocrylene |
| | 2.00 | Mexoryl XL | Drometrizole Trisiloxane |
| | 1.00 | Uvinul T 150 | Ethylhexyl Triazone |
| | 3.00 | T-Lite SF-S | Titanium Dioxide, Silica Hydrate, |
| | | | Alumina Hydrate, Methicone/ |
| | | | Dimethicone Copolymer |
| | 5.00 | Finsolv TN | C12-15 Alkyl Benzoate |
| | 4.00 | Abil WE 09 | Polyglyceryl-4 Isostearate, |
| | | | Cetyl PEG/PPG-10/1 |
| | | | Dimethicone, Hexyl Laurate |
| | 2.00 | Cosmacol EMI | Di-C12-13 Alkyl Malate |
| | 3.00 | Isopro palmitate | Isopropyl Palmitate |
| | 5.00 | Abil B 8839 | Cyclopentasiloxane, Cyclohexasiloxane |
| | 0.50 | Abil 350 | Dimethicone |
| B | 0.50 | Sodium chloride | Sodium Chloride |
| | 0.20 | Edeta BD | Disodium EDTA |
| | 1% | Fusion protein-KBD | |
| | 61.30 | Water demin. | Aqua dem. |
| C | 1.00 | Vitamin E acetate | Tocopheryl Acetate |
| | 0.50 | Phenonip | |

| | | | |
|---|---|---|---|
| A | 1.00 | Abil Care 85 | Bis-PEG/PPG-16/16 PEG/PPG-16/16 |
| | | | Dimethicone, Caprylic/Capric |
| | | | Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 5.00 | Uvinul N 539 T | Octocrylene |
| | 10.00 | Witconol APM | PPG-3 Myristyl Ether |
| | 2.00 | Uvinul T 150 | Ethylhexyl Triazone |
| | 1.00 | Dow Corning 345 | Cyclopentasiloxane, Cyclohexasiloxane |
| | | Fluid | |
| | 2.00 | Uvinul A Plus | Diethylamino Hydroxybenzoyl |
| | | | Hexyl Benzoate |
| B | 5.00 | Z-COTE MAX | Zinc Oxide (and) Diphenyl |
| | | | Capryl Silane |
| C | 5.00 | 1,2-Propylene | Propylene Glycol |
| | | glycol | |
| | 0.20 | Keltrol | Xanthan Gum |
| | 0.10 | Edeta BD | Disodium EDTA |
| | 1% | Fusion protein-KBD | |
| | 1.50 | Simulgel 600 | Acrylamide/Sodium |
| | | | Acryloyldimethyltaurate Copolymer, |
| | | | Isohexadecane, Polysorbate 80 |
| | 59.40 | Water demin. | Aqua dem. |
| D | q.s. | Perfume oil | |
| | 0.50 | Glydant | DMDM Hydantoin |

| | | | |
|---|---|---|---|
| A | 7.00 | Uvinul A Plus B | Ethylhexyl Methoxycinnamate, |
| | | | Diethylamino |
| | | | Hydroxybenzoyl Hexyl Benzoate |
| | 1.00 | Tinosorb S | Bis-Ethylhexyloxyphenol |
| | | | Methoxyphenyl |
| | | | Triazine |
| | 1.00 | Uvinul T 150 | Ethylhexyl Triazone |
| | 7.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 5.00 | Z-COTE MAX | Zinc Oxide (and) Diphenyl |
| | | | Capryl Methicone |
| C | 35.35 | Water demin. | Aqua dem. |
| | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 0.1% | Fusion protein-KBD | |
| | 1.50 | Veegum Ultra | Magnesium Aluminum Silicate |
| D | 0.50 | Phenonip | |
| | 1.00 | Vitamin E acetate | Tocopheryl Acetate |

| | | | |
|---|---|---|---|
| A | 7.00 | Uvinul A Plus B | Ethylhexyl Methoxycinnamate, |
| | | | Diethylamino |
| | | | Hydroxybenzoyl Hexyl Benzoate |
| | 1.00 | Tinosorb S | Bis-Ethylhexyloxyphenol |
| | | | Methoxyphenyl |
| | | | Triazine |
| | 1.00 | Uvinul T 150 | Ethylhexyl Triazone |
| | 7.00 | Uvinul N 539 T | Octocrylene |
| | 4.00 | Eumulgin VL 75 | Lauryl Glucoside, Polyglyceryl-2 |
| | | | Dipolyhydroxystearate, Glycerin |
| | 8.00 | Cetiol B | Dibutyl Adipate |
| | 8.00 | Finsolv TN | C12-15 Alkyl Benzoate |
| | 12.00 | Myritol 331 | Cocoglycerides |
| | 1.00 | Lanette E | Sodium Cetearyl Sulfate |
| | 2.00 | Lanette O | Cetearyl Alcohol |
| B | 5.00 | T-Lite SF | Titanium Dioxide, Alumina Hydrate, |
| | | | Dimethicone/Methicone Copolymer |
| C | 30.45 | Water demin. | Aqua dem. |
| | 3.00 | Glycerin 87% | Glycerin |
| | 0.05 | Edeta BD | Disodium EDTA |
| | 0.20 | Allantoin | Allantoin |
| | 0.30 | Keltrol | Xanthan Gum |
| | 5% | Fusion protein-KBD | |
| | 1.50 | Veegum Ultra | Magnesium Aluminum Silicate |
| D | 0.50 | Phenonip | |
| | 1.00 | Vitamin E acetate | Tocopheryl Acetate |

| | | |
|---|---|---|
| 5.00 | Z-COTE MAX | Zinc Oxide (and) Diphenyl Capryl |
| | | Methicone |
| 2.00 | Uvinul A Plus | Diethylamino Hydroxybenzoyl Hexyl |
| | | Benzoate |
| 2.00 | Mexoryl XL | Drometrizole Trisiloxane |
| 3.00 | Uvinul MC 80 | Ethylhexyl Methoxycinnamate |
| 0.50 | Abil 350 | Dimethicone |
| 2.75 | Carnico wax LT 20 | Carnauba (Copernica Cerifera) Wax, |
| | | Paraffin |
| 3.70 | Candelilla wax LT 281 LJ | Candelilla (Euphorbia Cerifera) Wax |
| 1.80 | Beeswax 3050 PH | Beeswax |
| 3.20 | TeCero wax 30445 | Microcrystalline Wax |
| 3.20 | TeCero wax 1030 K | Microcrystalline Wax |
| 1.34 | Cutina CP | Cetyl Palmitate |
| 6.40 | Vaseline | Petrolatum |
| 7.30 | Softisan 100 | Hydrogenated Coco-Glycerides |
| 10.00 | Luvitol EHO | Cetearyl Ethylhexanoate |
| 0.17 | Bisabolol nat. | Bisabolol |
| 1.84 | Vitamin E acetate | Tocopheryl Acetate |
| 1% | Fusion protein-KBD | |
| 0.42 | D,L-Alpha-Tocopherol | Tocopherol |
| 40.38 | castor oil | Castor (Ricinus Communis) Oil |

| | | | |
|---|---|---|---|
| A | 1.00 | Abil Care 85 | Bis-PEG/PPG-16/16 PEG/PPG-16/16 Dimethicone, |
| | | | Caprylic/Capric Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 Fluid | Cyclopentasiloxane, Cyclohexasiloxane |
| | 5.00 | Uvinul N 539 T | Octocrylene |
| B | 3.00 | T-Lite SF-S | Titanium Dioxide, Silica Hydrate, Alumina Hydrate, |
| | | | Methicone/Dimethicone Copolymer |
| C | 5.00 | 1,2-Propylene glycol | Propylene Glycol |
| | 1.00 | Mexoryl SX | Terephthalidene Dicamphor Sulfanic Acid |
| | 0.20 | Keltrol | Xanthan Gum |
| | 0.10 | Edeta BD | Disodium EDTA |
| | 1% | Fusion protein-KBD | |
| | 1.50 | Simulgel 600 | Acrylamide/Sodium Acryloyldimethyltaurate |
| | | | Copolymer, Isohexadecane, Polysorbate 80 |
| | 69.50 | Water demin. | Aqua dem. |
| D | q.s. | Perfume oil | |
| | 0.50 | Glydant | DMDM Hydantoin |

| | | | |
|---|---|---|---|
| A | 1.00 | Abil Care 85 | Bis-PEG/PPG-16/16 PEG/PPG-16/16 Dimethicone, |
| | | | Caprylic/Capric Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Parsol 1789 | Butyl Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 Fluid | Cyclopentasiloxane, Cyclohexasiloxane |
| | 5.00 | Uvinul N 539 T | Octocrylene |
| B | 3.00 | T-Lite SF-S | Titanium Dioxide, Silica Hydrate, Alumina Hydrate, |
| | | | Methicone/Dimethicone Copolymer |
| C | 5.00 | 1,2-Propylene glycol | Propylene Glycol |
| | 1.00 | Mexoryl SX | Terephthalidene Dicamphor Sulfanic Acid |
| | 0.20 | Keltrol | Xanthan Gum |
| | 0.10 | Edeta BD | Disodium EDTA |
| | 0.2% | Fusion protein-KBD | |
| | 1.50 | Simulgel 600 | Acrylamide/Sodium Acryloyldimethyltaurate |
| | | | Copolymer, Isohexadecane, Polysorbate 80 |
| | 68.30 | Water demin. | Aqua dem. |
| D | q.s. | Perfume oil | |
| | 0.50 | Glydant | DMDM Hydantoin |

| | | | |
|---|---|---|---|
| A | 0.70 | Cremophor A 25 | Ceteareth-25 |
| | 1.70 | Cremophor A 6 | Ceteareth-6, Stearyl Alcohol |
| | 2.00 | Parsol 1789 | Butyl Methoxydibenzoylmethane |
| | 5.00 | Uvinul N 539 T | Octocrylene |
| | 4.00 | Z-COTE MAX | Zinc Oxide (and) Diphenyl Capryl |
| | | | methicone |
| | 2.00 | Abil B 8843 | PEG-14 Dimethicone |
| | 3.60 | Lanette O | Cetearyl Alcohol |
| | 1.00 | Uvinul T150 | Ethylhexyl Triazone |
| | 2.00 | Cetiol B | Dibutyl Adipate |
| B | 4.00 | Glycerin 87% | Glycerin |
| | 0.20 | Edeta BD | Disodium EDTA |
| | 71.00 | Water demin. | Panthenol |
| C | 4.00 | Luvigel EM | Caprylic/Capric Triglyceride, Sodium |
| | | | Acrylates Copolymer |
| | 1% | Fusion protein-KBD | |
| D | 1.00 | Vitamin E acetate | Tocopheryl Acetate |
| | 0.20 | Bisabolol rac. | Bisabolol |
| | q.s. | Preservative | |

| | | | |
|---|---|---|---|
| A | 1.00 | Abil Care 85 | Bis-PEG/PPG-16/16 PEG/PPG-16/16 Dimethicone, |
| | | | Caprylic/Capric Triglyceride |
| | 3.00 | Cremophor CO 40 | PEG-40 Hydrogenated Castor Oil |
| | 0.30 | Cremophor WO 7 | PEG-7 Hydrogenated Castor Oil |
| | 2.00 | Parsol 1789 | Butyl Methoxydibenzoylmethane |
| | 2.00 | Mexoryl XL | Drometrizole Trisiloxane |
| | 10.00 | Witconol APM | PPG-3 Myristyl Ether |
| | 1.00 | Uvinul T 150 | Ethylhexyl Triazone |
| | 1.00 | Dow Corning 345 Fluid | Cyclopentasiloxane, Cyclohexasiloxane |
| | 5.00 | Uvinul N 539 T | Octocrylene |
| B | 3.00 | Z-COTE MAX | Zinc Oxide (and) Diphenyl Capryl Methicone |
| C | 5.00 | 1,2-Propylene glycol | Propylene Glycol |
| | 1.00 | Mexoryl SX | Terephthalidene Dicamphor Sulfanic Acid |
| | 0.20 | Keltrol | Xanthan Gum |
| | 0.10 | Edeta BD | Disodium EDTA |
| | 1.50 | Simulgel 600 | Acrylamide/Sodium Acryloyldimethyltaurate |
| | | | Copolymer, Isohexadecane, Polysorbate 80 |
| | 0.5% | Fusion protein-KBD | |
| | 68.00 | Water demin. | Aqua dem. |
| D | q.s. | Perfume oil | |
| | 0.50 | Glydant | DMDM Hydantoin |

| | | | |
|---|---|---|---|
| A | 2.00 | Uvinul A Plus | Diethylamino Hydroxybenzoyl Hexyl Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.5 | Cremophor WO 7 | PEG-7 Hydrogenated Castor Oil |
| | 0.5 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl Capryl methicone |
| C | 4.00 | 1,2-Propylene glycol Care | Propylene Glycol |
| | 0.20 | Keltrol | Xanthan Gum |
| | 0.50 | Simulgel 600 | Acrylamide/Sodium Acryloyldimethyltaurate |
| | | | Copolymer, Isohexadecane, Polysorbate 80 |
| | 65.80 | Water demin. | Aqua dem |
| D | 0.50 | Euxyl K 300 | |
| | 0.50 | Vitamin E acetate | Tocopheryl Acetate |
| | 1% | Fusion protein-KBD | |
| | | | Carprylic/Capric Triglyceride, Sodium |
| | | | Ascorbate, Tocopherol, Retinol |

| | | | |
|---|---|---|---|
| A | 2.00 | Uvinul A Plus | Diethylamino Hydroxybenzoyl Hexyl Benzoate |
| | 3.00 | Uvinul N 539 T | Octocrylene |
| | 3.00 | Emulgade PL 68/50 | Cetearyl Glucoside, Cetearyl Alcohol |
| | 2.00 | Dracorin 100 SE | Glyceryl Stearate, PEG-100 Stearate |
| | 1.00 | Fitoderm | Squalane |
| | 0.5 | Cremophor WO 7 | PEG-7 Hydrogenated Castor Oil |
| | 0.5 | Cremophor PS 20 | Polysorbate 20 |
| | 2.00 | Dry Flo Pure | Aluminum Starch Octenylsuccinate |
| B | 5.00 | Z-COTE MAX | Zinc Oxide (and) Diphenyl Capryl methicone |
| C | 4.00 | 1,2-Propylene glycol Care | Propylene Glycol |
| | 0.20 | Keltrol | Xanthan Gum |
| | 0.50 | Simulgel 600 | Acrylamide/Sodium Acryloyldimethyltaurate |
| | | | Copolymer, Isohexadecane, Polysorbate 80 |
| | 65.80 | Water demin. | Aqua dem |
| D | 0.50 | Euxyl K 300 | |
| | 0.50 | Vitamin E acetate | Tocopheryl Acetate |
| | 1% | Fusion protein-KBD | |
| | 1.00 | RetiSTAR | Caprylic/Capric Triglyceride, Sodium |
| | | | Ascorbate, Tocopherol, Retinol |

1. A chimeric keratin-binding effector protein comprising
(a) at least one keratin-binding polypeptide (i) and
(b) at least one further effector polypeptide (ii).

2. The keratin-binding effector protein according to claim 1, where the keratin-binding polypeptide (i) has binding affinity to human hair keratin, nail keratin or skin keratin.

3. The keratin-binding effector protein according to claim 1, where the keratin-binding polypeptide (i) used
a) comprises at least one of the sequences according to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215, or
b) corresponds to a polypeptide which is at least 40% identical to at least one of the sequences according to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215 and is able to bind keratin.

4. The keratin-binding effector protein according to claim 1, where the keratin-binding polypeptide (i) used is encoded by a nucleic acid molecule comprising at least one nucleic acid molecule chosen from the group consisting of:
a) a nucleic acid molecule which encodes a polypeptide comprising the sequence shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215;
b) a nucleic acid molecule which comprises at least one polynucleotide of the sequence shown in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 or 214;
c) a nucleic acid molecule which encodes a polypeptide according to the sequences SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215;
d) a nucleic acid molecule with a nucleic acid sequence corresponding to at least one of the sequences according to SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 145, 149, 152, 159, 161, 163, 165, 212 or 214 or a nucleic acid molecule derived therefrom by substitution, deletion or insertion which encodes a polypeptide which is at least 40% identical to at least one of the sequences according to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215 and is able to bind to keratin;
e) a nucleic acid molecule which encodes a polypeptide which is recognized by a monoclonal antibody directed toward a polypeptide which is encoded by the nucleic acid molecules according to (a) to (c);
f) a nucleic acid molecule coding for a keratin-binding protein which, under stringent conditions, hybridizes with a nucleic acid molecule according to (a) to (c);
g) a nucleic acid molecule coding for a keratin-binding protein which can be isolated from a DNA bank using a nucleic acid molecule according to (a) to (c) or part fragments thereof comprising at least 15 nucleotides as probe under stringent hybridization conditions; and
h) a nucleic acid molecule which can be produced by backtranslating one of the amino acid sequences shown in the sequences SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 146, 150, 153, 156, 157, 158, 160, 162, 164, 166, 213 or 215.

5. The keratin-binding effector protein according to claim 1, where the effector polypeptide (ii) is chosen from the group consisting of enzymes, antibodies, effector-binding proteins, fluorescent proteins, antimicrobial peptides, and self-assembling proteins.

6. The keratin-binding effector protein according to claim 5, where the effector polypeptide (ii) is an enzyme chosen from the group consisting of oxidases, peroxidases, proteases, tyrosinases, lactoperoxidase, lysozyme, amyloglycosidases, glucose oxidases, superoxide dismutases, photolyases and catalases.

7. The keratin-binding effector protein according to claim 6, where the effector polypeptide (ii) is a silk protein.

8. The keratin-binding effector protein according to claim 7, where the silk protein comprises at least one of the sequences according to SEQ ID NO: 151, 201, 202, 203, 204, 205, 206, 207, 208, 209 or 210, or corresponds to a polypeptide which is at least 40% identical to at least one of the sequences according to SEQ ID No.: 151, 201, 202, 203, 204, 205, 206, 207, 208, 209 or 210.

9. The keratin-binding effector protein according to claim 7, where the silk protein is encoded by a nucleic acid molecule comprising at least one nucleic acid molecule chosen from the group consisting of:
a) a nucleic acid molecule which encodes a polypeptide comprising the sequence shown in SEQ ID No.: 151, 201, 202, 203, 204, 205, 206, 207, 208, 209 or 210;
b) a nucleic acid molecule which comprises at least one polynucleotide of the sequence shown in SEQ ID No. 150;
c) a nucleic acid molecule which encodes a polypeptide according to the sequences SEQ ID No.: 151, 201, 202, 203, 204, 205, 206, 207, 208, 209 or 210;
d) a nucleic acid molecule with a nucleic acid sequence according to SEQ ID No.: 150 or a nucleic acid molecule derived therefrom by substitution, deletion or insertion which encodes a polypeptide which is at least 40% identical to the sequence according to SEQ ID No.: 151;
e) a nucleic acid molecule which encodes a polypeptide which is recognized by a monoclonal antibody directed toward a polypeptide which is encoded by the nucleic acid molecules according to (k) to (m);
f) a nucleic acid molecule coding for a keratin-binding protein which hybridizes under stringent conditions with a nucleic acid molecule according to (k) to (m);
g) a nucleic acid molecule coding for a keratin-binding protein which can be isolated from a DNA bank using a nucleic acid molecule according to (k) to (m) or part fragments thereof comprising at least 15 nucleotides as probe under stringent hybridization conditions; and
h) a nucleic acid molecule which can be produced by backtranslating one of the amino acid sequences shown in the sequences SEQ ID No.: 151, 201, 202, 203, 204, 205, 206, 207, 208, 209 or 210.

10. The keratin-binding effector protein according to claim 1, where the polypeptides (i) and (ii) are linked together by means of translation fusion.

11. The keratin-binding effector protein according to claim 1, where the polypeptides (i) and (ii) are linked together by means of a chemical coupling reaction.

12. The keratin-binding effector protein according to claim 11, where the effector polypeptide (ii) is covalently bonded to side chains of internal amino acids, the C-terminus or the N-terminus of the keratin-binding polypeptide (i).

13. The keratin-binding effector protein according to claim 1, where the effector polypeptide (ii) and the keratin binding polypeptide (i) are joined together by means of a spacer element.

14. The keratin-binding effector protein according to claim 13, where the spacer element is a crosslinker.

15. The keratin-binding effector protein according to claim 13, where the spacer element is an at least bifunctional linker which covalently joins together the keratin-binding polypeptide (i) and the effector polypeptide by binding to side chains of internal amino acids, the C-terminus or the N-terminus of said polypeptides.

16. The keratin-binding effector protein according to claim 13, where the spacer element is a polypeptide.

17. The use of the keratin-binding effector proteins described in claims 1-16 in dermocosmetics.

18. The use according to claim 17, where the dermocosmetic is a skin protection composition, skincare composition, skin cleansing composition, hair protection composition, haircare composition, hair cleansing composition, hair colorant or a decorative cosmetic.

19. A dermocosmetic comprising the keratin-binding effector protein of claim 1.

20. A protein comprising one of the amino acid sequences shown in SEQ ID NO: 168, 176, 182, 188, 194, or 200.

21. A nucleic acid molecule comprising the sequence shown in SEQ ID NO: 167, 175, 181, 187, 193 or 199.

22. A DNA expression cassette comprising a the nucleic acid molecule of claim 21.

23. A vector comprising the expression cassette claim 22.

24. A transgenic cell comprising the nucleic acid molecule of claim 21, an expression cassette comprising the nucleic acid molecule, or a vector comprising the expression cassette.

25. The dermocosmetic of claim 19, wherein the dermocosmetic is a skin protection composition, skincare composition, skin cleansing composition, hair protection composition, haircare composition, hair cleansing composition, hair colorant, or a decorative cosmetic.

26. A nucleic acid molecule encoding the protein of claim 20.

27. A transgenic cell comprising the nucleic acid molecule of claim 26, an expression cassette comprising the nucleic acid molecule, or a vector comprising the expression cassette.
